SEQUENCE LISTING (31 sequences)

SEQ ID NO: 1 - DNA, 1975 bp, Homo sapiens
  CDS (42)...(1556)
ccgggcaggtacgcggggagagctcaggacctctgagaagaatggagccctcctggcttcaggaactcatggctcaccccttcttgctgctgatcctcctctgcatgtctctgctgctgtttcaggtaatcaggttgtaccagaggaggagatggatgatcagagccctgcacctgtttcctgcaccccctgcccactggttctatggccacaaggagttttacccagtaaaggagtttgaggtgtatcataagctgatggaaaaatacccatgtgctgttcccttgtgggttggaccctttacgatgttcttcagtgtccatgacccagactatgccaagattctcctgaaaagacaagatcccaaaagtgctgttagccacaaaatccttgaatcctgggttggtcgaggacttgtgaccctggatggttctaaatggaaaaagcaccgccagattgtgaaacctggcttcaacatcagcattctgaaaatattcatcaccatgatgtctgagagtgttcggatgatgctgaacaaatgggaggaacacattgcccaaaactcacgtctggagctctttcaacatgtctccctgatgaccctggacagcatcatgaagtgtgccttcagccaccagggcagcatccagttggacagtaccctggactcatacctgaaagcagtgttcaaccttagcaaaatctccaaccagcgcatgaacaattttctacatcacaacgacctggttttcaaattcagctctcaaggccaaatcttttctaaatttaaccaagaacttcatcagttcacagagaaagtaatccaggaccggaaggagtctcttaaggataagctaaaacaagatactactcagaaaaggcgctgggattttctggacatacttttgagtgccaaaagcgaaaacaccaaagatttctctgaagcagatctccaggctgaagtgaaaacgttcatgtttgcaggacatgacaccacatccagtgctatctcctggatcctttactgcttggcaaagtaccctgagcatcagcagagatgccgagatgaaatcagggaactcctaggggatgggtcttctattacctgggaacacctgagccagatgccttacaccacgatgtgcatcaaggaatgcctccgcctctacgcaccggtagtaaacatatcccggttactcgacaaacccatcacctttccagatggacgctccttacctgcaggaataactgtgtttatcaatatttgggctcttcaccacaacccctatttctgggaagaccctcaggtctttaaccccttgagattctccagggaaaattctgaaaaaatacatccctatgccttcataccattctcagctggattaaggaactgcattgggcagcattttgccataattgagtgtaaagtggcagtggcattaactctgctccgcttcaagctggctccagaccactcaaggcctccccagcctgttcgtcaagttgtcctcaagtccaagaatggaatccatgtgtttgcaaaaaaagtttgctaattttaagtcctttcgtataagaattaatgagacaattttcctaccaaaggaagaacaaaaggataaatataatacaaaatatatgtatatggttgtttgacaaattatataacttaggatacttctgactggttttgacatccattaacagtaattttaatttctttgctgtatctggtgaaacccacaaaaacacctgaaaaaactcaagctgacttccactgcgaagggaaattattggtttgtgtaactagtggtagagtggctttcaagcatagtttgatcaaaactccactcagtatctgcattacttttatctctgcaaatatctgcatgatagctttattctcagttatctttccccataataaaaaatatctgccaaaaaaaaaaaaaaaaaaaaacgctcgaaaggg

SEQ ID NO: 2 - protein, 505 aa, Homo sapiens
MEPSWLQELMAHPFLLLILLCMSLLLFQVIRLYQRRRWMIRALHLFPAPPAHWFYGHKEFYPVKEFEVYHKLMEKYPCAVPLWVGPFTMFFSVHDPDYAKILLKRQDPKSAVSHKILESWVGRGLVTLDGSKWKKHRQIVKPGFNISILKIFITMMSESVRMMLNKWEEHIAQNSRLELFQHVSLMTLDSIMKCAFSHQGSIQLDSTLDSYLKAVFNLSKISNQRMNNFLHHNDLVFKFSSQGQIFSKFNQELHQFTEKVIQDRKESLKDKLKQDTTQKRRWDFLDILLSAKSENTKDFSEADLQAEVKTFMFAGHDTTSSAISWILYCLAKYPEHQQRCRDEIRELLGDGSSITWEHLSQMPYTTMCIKECLRLYAPVVNISRLLDKPITFPDGRSLPAGITVFINIWALHHNPYFWEDPQVFNPLRFSRENSEKIHPYAFIPFSAGLRNCIGQHFAIIECKVAVALTLLRFKLAPDHSRPPQPVRQVVLKSKNGIHVFAKKVC

SEQ ID NO: 3 - DNA, 1518 bp, Homo sapiens
atggagccctcctggcttcaggaactcatggctcaccccttcttgctgctgatcctcctctgcatgtctctgctgctgtttcaggtaatcaggttgtaccagaggaggagatggatgatcagagccctgcacctgtttcctgcaccccctgcccactggttctatggccacaaggagttttacccagtaaaggagtttgaggtgtatcataagctgatggaaaaatacccatgtgctgttcccttgtgggttggaccctttacgatgttcttcagtgtccatgacccagactatgccaagattctcctgaaaagacaagatcccaaaagtgctgttagccacaaaatccttgaatcctgggttggtcgaggacttgtgaccctggatggttctaaatggaaaaagcaccgccagattgtgaaacctggcttcaacatcagcattctgaaaatattcatcaccatgatgtctgagagtgttcggatgatgctgaacaaatgggaggaacacattgcccaaaactcacgtctggagctctttcaacatgtctccctgatgaccctggacagcatcatgaagtgtgccttcagccaccagggcagcatccagttggacagtaccctggactcatacctgaaagcagtgttcaaccttagcaaaatctccaaccagcgcatgaacaattttctacatcacaacgacctggttttcaaattcagctctcaaggccaaatcttttctaaatttaaccaagaacttcatcagttcacagagaaagtaatccaggaccggaaggagtctcttaaggataagctaaaacaagatactactcagaaaaggcgctgggattttctggacatacttttgagtgccaaaagcgaaaacaccaaagatttctctgaagcagatctccaggctgaagtgaaaacgttcatgtttgcaggacatgacaccacatccagtgctatctcctggatcctttactgcttggcaaagtaccctgagcatcagcagagatgccgagatgaaatcagggaactcctaggggatgggtcttctattacctgggaacacctgagccagatgccttacaccacgatgtgcatcaaggaatgcctccgcctctacgcaccggtagtaaacatatcccggttactcgacaaacccatcacctttccagatggacgctccttacctgcaggaataactgtgtttatcaatatttgggctcttcaccacaacccctatttctgggaagaccctcaggtctttaaccccttgagattctccagggaaaattctgaaaaaatacatccctatgccttcataccattctcagctggattaaggaactgcattgggcagcattttgccataattgagtgtaaagtggcagtggcattaactctgctccgcttcaagctggctccagaccactcaaggcctccccagcctgttcgtcaagttgtcctcaagtccaagaatggaatccatgtgtttgcaaaaaaagtttgctaa

SEQ ID NO: 4 - DNA, 1927 bp, Homo sapiens
  CDS (1)...(1512)
atggaggcgaccggcacctgggcgctgctgctggcgctggcgctgctcctgctgctgacgctggcgctgtccgggaccagggcccgaggccacctgccccccgggcccacgccgctaccactgctgggaaacctcctgcagctacggcccggggcgctgtattcagggctcatgcggctgagtaagaagtacggaccggtgttcaccatctacctgggaccgtggcggcctgtggtggtcctggttgggcaggaggctgtgcgggaggccctgggaggtcaggctgaggagttcagcggccggggaaccgtagcgatgctggaagggacttttgatggccatggggttttcttctccaacggggagcggtggaggcagctgaggaagtttaccatgcttgctctgcgggacctgggcatggggaagcgagaaggcgaggagctgatccaggcggaggcccggtgtctggtggagacattccaggggacagaaggacgcccattcgatccctccctgctgctggcccaggccacctccaacgtagtctgctccctcctctttggcctccgcttctcctatgaggataaggagttccaggccgtggtccgggcagctggtggtaccctgctgggagtcagctcccaggggggtcagacctacgagatgttctcctggttcctgcggcccctgccaggcccccacaagcagctcctccaccacgtcagcaccttggctgccttcacagtccggcaggtgcagcagcaccaggggaacctggatgcttcgggccccgcacgtgaccttgtcgatgccttcctgctgaagatggcacaggaggaacaaaacccaggcacagaattcaccaacaagaacatgctgatgacagtcatttatttgctgtttgctgggacgatgacggtcagcaccacggtcggctataccctcctgctcctgatgaaataccctcatgtccaaaagtgggtacgtgaggagctgaatcgggagctgggggctggccaggcaccaagcctaggggaccgtacccgcctcccttacaccgacgcggttctgcatgaggcgcagcggctgctggcgctggtgcccatgggaataccccgcaccctcatgcggaccacccgcttccgagggtacaccctgccccagggcacggaggtcttccccctccttggctccatcctgcatgaccccaacatcttcaagcacccagaagagttcaacccagaccgtttcctggatgcagatggacggttcaggaagcatgaggcgttcctgcccttctccttagggaagcgtgtctgccttggagagggcctggcaaaagcggagctcttcctcttcttcaccaccatcctacaagccttctccctggagagcccgtgcccgccggacaccctgagcctcaagcccaccgtcagtggccttttcaacattcccccagccttccagctgcaagtccgtcccactgaccttcactccaccacgcagaccagatgaaggaaggcaacttggaagtggtgggtgcccaggacggtgcctccagcctcaacagtgggcatggacagggttaatgtctccagagtgtacactgcaggcagccacatttacacgcctgcagttgttttccggagtctgtcccacggcccacacgctcacttgactcatgctgctaagatgcacaaccgcacacccatacacaactacaagggccacaaagcaactgctgggttagctttccacagacataaatatagtccatctgcaatcacaagcacatagccaggtaacccaccaactcccctggatctgcagcccacacgtgggagtctggctgtcaccttcacaagccacagaaacggccacacatgttcacagctcacacgccctctccattcatcgaacttctcag

SEQ ID NO: 5 - protein, 504 aa, Homo sapiens
MEATGTWALLLALALLLLLTLALSGTRARGHLPPGPTPLPLLGNLLQLRPGALYSGLMRLSKKYGPVFTIYLGPWRPVVVLVGQEAVREALGGQAEEFSGRGTVAMLEGTFDGHGVFFSNGERWRQLRKFTMLALRDLGMGKREGEELIQAEARCLVETFQGTEGRPFDPSLLLAQATSNVVCSLLFGLRFSYEDKEFQAVVRAAGGTLLGVSSQGGQTYEMFSWFLRPLPGPHKQLLHHVSTLAAFTVRQVQQHQGNLDASGPARDLVDAFLLKMAQEEQNPGTEFTNKNMLMTVIYLLFAGTMTVSTTVGYTLLLLMKYPHVQKWVREELNRELGAGQAPSLGDRTRLPYTDAVLHEAQRLLALVPMGIPRTLMRTTRFRGYTLPQGTEVFPLLGSILHDPNIFKHPEEFNPDRFLDADGRFRKHEAFLPFSLGKRVCLGEGLAKAELFLFFTTILQAFSLESPCPPDTLSLKPTVSGLFNIPPAFQLQVRPTDLHSTTQTR

SEQ ID NO: 6 - DNA, 1515 bp, Homo sapiens
atggaggcgaccggcacctgggcgctgctgctggcgctggcgctgctcctgctgctgacgctggcgctgtccgggaccagggcccgaggccacctgccccccgggcccacgccgctaccactgctgggaaacctcctgcagctacggcccggggcgctgtattcagggctcatgcggctgagtaagaagtacggaccggtgttcaccatctacctgggaccgtggcggcctgtggtggtcctggttgggcaggaggctgtgcgggaggccctgggaggtcaggctgaggagttcagcggccggggaaccgtagcgatgctggaagggacttttgatggccatggggttttcttctccaacggggagcggtggaggcagctgaggaagtttaccatgcttgctctgcgggacctgggcatggggaagcgagaaggcgaggagctgatccaggcggaggcccggtgtctggtggagacattccaggggacagaaggacgcccattcgatccctccctgctgctggcccaggccacctccaacgtagtctgctccctcctctttggcctccgcttctcctatgaggataaggagttccaggccgtggtccgggcagctggtggtaccctgctgggagtcagctcccaggggggtcagacctacgagatgttctcctggttcctgcggcccctgccaggcccccacaagcagctcctccaccacgtcagcaccttggctgccttcacagtccggcaggtgcagcagcaccaggggaacctggatgcttcgggccccgcacgtgaccttgtcgatgccttcctgctgaagatggcacaggaggaacaaaacccaggcacagaattcaccaacaagaacatgctgatgacagtcatttatttgctgtttgctgggacgatgacggtcagcaccacggtcggctataccctcctgctcctgatgaaataccctcatgtccaaaagtgggtacgtgaggagctgaatcgggagctgggggctggccaggcaccaagcctaggggaccgtacccgcctcccttacaccgacgcggttctgcatgaggcgcagcggctgctggcgctggtgcccatgggaataccccgcaccctcatgcggaccacccgcttccgagggtacaccctgccccagggcacggaggtcttccccctccttggctccatcctgcatgaccccaacatcttcaagcacccagaagagttcaacccagaccgtttcctggatgcagatggacggttcaggaagcatgaggcgttcctgcccttctccttagggaagcgtgtctgccttggagagggcctggcaaaagcggagctcttcctcttcttcaccaccatcctacaagccttctccctggagagcccgtgcccgccggacaccctgagcctcaagcccaccgtcagtggccttttcaacattcccccagccttccagctgcaagtccgtcccactgaccttcactccaccacgcagaccagatga

SEQ ID NO: 7 - DNA, 2099 bp, Homo sapiens
  CDS (78)...(1709)
ggcgccgcgggtcaggcagctgcgtgcgcgtctcctccaggcagcaaggggaacccgaggccgccggcgcccggaccatgtcgtctccggggccgtcgcagccgccggccgaggacccgccctggcccgcgcgcctcctgcgtgcgcctctggggctgctgcggctggaccccagcgggggcgcgctgctgctatgcggcctcgtagcgctgctgggctggagctggctgcggaggcgccgggcgcggggcatcccgcccgggcccacgccctggcctctggtgggcaacttcggtcacgtgctgctgcctcccttcctccggcggcggagctggctgagcagcaggaccagggccgcagggattgatccctcggtcataggcccgcaggtgctcctggctcacctagcccgcgtgtacggcagcatcttcagcttctttatcggccactacctggtggtggtcctcagcgacttccacagcgtgcgcgaggcgctggtgcagcaggccgaggtcttcagcgaccgcccgcgggtgccgctcatctccatcgtgaccaaggagaagggggttgtgtttgcacattatggtcccgtctggagacaacaaaggaagttctctcattcaactcttcgtcattttgggttgggaaaacttagcttggagcccaagattattgaggagttcaaatatgtgaaagcagaaatgcaaaagcacggagaagaccccttctgccctttctccatcatcagcaatgccgtctctaacatcatttgctccttgtgctttggccagcgctttgattacactaatagtgagttcaagaaaatgcttggttttatgtcacgaggcctagaaatctgtctgaacagtcaagtcctcctggtcaacatatgcccttggctttattaccttccctttggaccatttaaggaattaagacaaattgaaaaggatataaccagtttccttaaaaaaatcatcaaagaccatcaagagtctctggatagagagaaccctcaggacttcatagacatgtaccttctccacatggaagaggagaggaaaaataatagtaacagcagttttgatgaagagtacttattttatatcattggggatctctttattgctgggactgataccacaactaactctttgctctggtgcctgctgtatatgtcgctgaaccccgatgtacaagaaaaggttcatgaagaaattgaaagagtcattggcgccaaccgagctccttccctcacagacaaggcccagatgccctacacagaagccaccatcatggaagtgcagaggctaactgtggtggtgccgcttgccattcctcatatgacctcagagaacacagtgctccaagggtataccattcctaaaggcacattgatcttacccaacctgtggtcagtacatagagacccagccatttgggagaaaccggaggatttctaccctaatcgatttctggatgaccaaggacaactaattaaaaaagaaacctttattccttttgggatagggaagcgggtgtgtatgggagaacaactggcaaagatggaattattcctaatgtttgtgagcctaatgcagagtttcgcatttgctttacctgaggattctaagaagcccctcctgactggaagatttggtctaactttagccccacatccatttaatataactatttcaaggagatgaagagcatctccaagaagagatggtaaaaagatatataaatacatatccttctaagcagattcttcctactgcaaaggacagtgaatccagcaactcagtggatccaagctgggctcagaggtcggaaggagggtagagcacactgggaggtttcatcttggaggattcctcagcaggatacttcagccattttagtaatgcaggtctgtgatttgggggatagaaaacaaagtacctatgaaacgggatatctggattttacttgcagtggcttccaccgatgggccaatcttctcatttcttagtgcctcagacatcccatatgtaaaatgagagtaataaaacttggcttctctctaaaaaaaarmamtaaaaaaaaaaaaaaaa

SEQ ID NO: 8 - protein, 544 aa, Homo sapiens
MSSPGPSQPPAEDPPWPARLLRAPLGLLRLDPSGGALLLCGLVALLGWSWLRRRRARGIPPGPTPWPLVGNFGHVLLPPFLRRRSWLSSRTRAAGIDPSVIGPQVLLAHLARVYGSIFSFFIGHYLVVVLSDFHSVREALVQQAEVFSDRPRVPLISIVTKEKGVVFAHYGPVWRQQRKFSHSTLRHFGLGKLSLEPKIIEEFKYVKAEMQKHGEDPFCPFSIISNAVSNIICSLCFGQRFDYTNSEFKKMLGFMSRGLEICLNSQVLLVNICPWLYYLPFGPFKELRQIEKDITSFLKKIIKDHQESLDRENPQDFIDMYLLHMEEERKNNSNSSFDEEYLFYIIGDLFIAGTDTTTNSLLWCLLYMSLNPDVQEKVHEEIERVIGANRAPSLTDKAQMPYTEATIMEVQRLTVVVPLAIPHMTSENTVLQGYTIPKGTLILPNLWSVHRDPAIWEKPEDFYPNRFLDDQGQLIKKETFIPFGIGKRVCMGEQLAKMELFLMFVSLMQSFAFALPEDSKKPLLTGRFGLTLAPHPFNITISRR

SEQ ID NO: 9 - DNA, 1635 bp, Homo sapiens
atgtcgtctccggggccgtcgcagccgccggccgaggacccgccctggcccgcgcgcctcctgcgtgcgcctctggggctgctgcggctggaccccagcgggggcgcgctgctgctatgcggcctcgtagcgctgctgggctggagctggctgcggaggcgccgggcgcggggcatcccgcccgggcccacgccctggcctctggtgggcaacttcggtcacgtgctgctgcctcccttcctccggcggcggagctggctgagcagcaggaccagggccgcagggattgatccctcggtcataggcccgcaggtgctcctggctcacctagcccgcgtgtacggcagcatcttcagcttctttatcggccactacctggtggtggtcctcagcgacttccacagcgtgcgcgaggcgctggtgcagcaggccgaggtcttcagcgaccgcccgcgggtgccgctcatctccatcgtgaccaaggagaagggggttgtgtttgcacattatggtcccgtctggagacaacaaaggaagttctctcattcaactcttcgtcattttgggttgggaaaacttagcttggagcccaagattattgaggagttcaaatatgtgaaagcagaaatgcaaaagcacggagaagaccccttctgccctttctccatcatcagcaatgccgtctctaacatcatttgctccttgtgctttggccagcgctttgattacactaatagtgagttcaagaaaatgcttggttttatgtcacgaggcctagaaatctgtctgaacagtcaagtcctcctggtcaacatatgcccttggctttattaccttccctttggaccatttaaggaattaagacaaattgaaaaggatataaccagtttccttaaaaaaatcatcaaagaccatcaagagtctctggatagagagaaccctcaggacttcatagacatgtaccttctccacatggaagaggagaggaaaaataatagtaacagcagttttgatgaagagtacttattttatatcattggggatctctttattgctgggactgataccacaactaactctttgctctggtgcctgctgtatatgtcgctgaaccccgatgtacaagaaaaggttcatgaagaaattgaaagagtcattggcgccaaccgagctccttccctcacagacaaggcccagatgccctacacagaagccaccatcatggaagtgcagaggctaactgtggtggtgccgcttgccattcctcatatgacctcagagaacacagtgctccaagggtataccattcctaaaggcacattgatcttacccaacctgtggtcagtacatagagacccagccatttgggagaaaccggaggatttctaccctaatcgatttctggatgaccaaggacaactaattaaaaaagaaacctttattccttttgggatagggaagcgggtgtgtatgggagaacaactggcaaagatggaattattcctaatgtttgtgagcctaatgcagagtttcgcatttgctttacctgaggattctaagaagcccctcctgactggaagatttggtctaactttagccccacatccatttaatataactatttcaaggagatga

SEQ ID NO: 10 - protein, 496 aa, Artificial Sequence
  consensus sequence
PPGPPPLPLIGNLLQLGRAPGPIPHSLTKLRKAKRYGKPVFTLYLGPRPVVVLTGPEAVKEVLIDKGEEFAKGRGDFNPTFPWLSKGYREQGLLFSDNGPKWRKLRRFSLLTLRFHFGMGAYSKRSQKLEEPRIQEEARDLVERLRKEQAGSPIDITELLARLAPLNVICSLLFGVRFDYLRPEDPEFLKLIDKLLNEMFDRVSPWHQLLDIFPFLLRYLPGSLFRKAFKAAKDLKDYLDKLIEERRETLEPAGDPRRLDIGFLDSLLLEAKREGGNPKSELSDEELAATVLDLLFAGTETTSSTLSWALYLLAKHPEVQAKLREEIDEVIGRDRSPTYDVDARAQMPYLDAVIKETLRLYPVVPLLLPRVATKDTEIPDGYLIPKGTLVIVNLYSLHRDPKVFPNPEEFDPERFLDENGKFKKSYAFLPFGAGPRNCLGERLARMELFLFLATLLQRFPELELAVPPGDIPSLTPKPELGLPSKPPLYKVQLRPA

SEQ ID NO: 11 - protein, 13 aa, Artificial Sequence
  consensus sequence
PPGPPPLPLIGNL

SEQ ID NO: 12 - protein, 470 aa, Artificial Sequence
  consensus sequence
LTKLRKAKRYGKPVFTLYLGPRPVVVLTGPEAVKEVLIDKGEEFAKGRGDFNPTFPWLSKGYREQGLLFSDNGPKWRKLRRFSLLTLRFHFGMGAYSKRSQKLEEPRIQEEARDLVERLRKEQAGSPIDITELLARLAPLNVICSLLFGVRFDYLRPEDPEFLKLIDKLLNEMFDRVSPWHQLLDIFPFLLRYLPGSLFRKAFKAAKDLKDYLDKLIEERRETLEPAGDPRRLDIGFLDSLLLEAKREGGNPKSELSDEELAATVLDLLFAGTETTSSTLSWALYLLAKHPEVQAKLREEIDEVIGRDRSPTYDVDARAQMPYLDAVIKETLRLYPVVPLLLPRVATKDTEIPDGYLIPKGTLVIVNLYSLHRDPKVFPNPEEFDPERFLDENGKFKKSYAFLPFGAGPRNCLGERLARMELFLFLATLLQRFPELELAVPPGDIPSLTPKPELGLPSKPPLYKVQLRPA

SEQ ID NO: 13 - DNA, 1043 bp, Homo sapiens
  CDS (175)...(885)
cccacgcgtccgccagagtcgggccgcaggaggtgtcggtgccgagcggggttttttttttctgcgggttgccttttgtttttcctttggaaccgcggttgttcaaaagcttgacggaacttggaaggggactcccactctcctccctctttccgctgagtttgtgactccgagatggacaaagtgtgtgctatttttggaggctcccgaggcattggcagggctgtggcccagttaatggcccggaaaggctaccgcctggcgatcattgccagaaacctggaaggggccaaagccgccgccggtgacctcggcggagatcatttggcatttagctgtgatgttgctaaagaacatgatgttcaaaatacatttgaagagatggagaaacatttaggtcgagtaaatttcttggtaaatgcagctggtattaacagggatagtcttttagtaagaacaaaaactgaagatatggtatctcagcttcatactaacctcttgggttccatgctgacctgtaaagctgccatgagggctatgattcaacaacagggagggtctattgttaatgtaggaagcattgttggcttaaaaggcaactctggccagtccgtttacagtgccagtaaaggaggattagttggattttcacgtgctcttgctaaagaggtagcaagaaagaaaattagagtgaatgtagttgcaccaggatttgtacacacagatatgacgaaagacttgaaagaagaacatttaaagaaaaatattcctcttgggaggtttggagaaactattgaggtggcacatgcggttgtgtttcttttagaatcaccgtatattacagggcatgttctggtagtggatgggggattacaactcattttgtaatttgcagattattcagttataggggtgattagcatcaagggcacactttggctactgattagacaattatacctacatgggtaacatgtgctaatcaaacctgctgatgctacaaatgttaatttctgtctttataaaaatatgtctcaaaagaa

SEQ ID NO: 14 - protein, 237 aa, Homo sapiens
MDKVCAIFGGSRGIGRAVAQLMARKGYRLAIIARNLEGAKAAAGDLGGDHLAFSCDVAKEHDVQNTFEEMEKHLGRVNFLVNAAGINRDSLLVRTKTEDMVSQLHTNLLGSMLTCKAAMRAMIQQQGGSIVNVGSIVGLKGNSGQSVYSASKGGLVGFSRALAKEVARKKIRVNVVAPGFVHTDMTKDLKEEHLKKNIPLGRFGETIEVAHAVVFLLESPYITGHVLVVDGGLQLIL

SEQ ID NO: 15 - DNA, 714 bp, Homo sapiens
atggacaaagtgtgtgctatttttggaggctcccgaggcattggcagggctgtggcccagttaatggcccggaaaggctaccgcctggcgatcattgccagaaacctggaaggggccaaagccgccgccggtgacctcggcggagatcatttggcatttagctgtgatgttgctaaagaacatgatgttcaaaatacatttgaagagatggagaaacatttaggtcgagtaaatttcttggtaaatgcagctggtattaacagggatagtcttttagtaagaacaaaaactgaagatatggtatctcagcttcatactaacctcttgggttccatgctgacctgtaaagctgccatgagggctatgattcaacaacagggagggtctattgttaatgtaggaagcattgttggcttaaaaggcaactctggccagtccgtttacagtgccagtaaaggaggattagttggattttcacgtgctcttgctaaagaggtagcaagaaagaaaattagagtgaatgtagttgcaccaggatttgtacacacagatatgacgaaagacttgaaagaagaacatttaaagaaaaatattcctcttgggaggtttggagaaactattgaggtggcacatgcggttgtgtttcttttagaatcaccgtatattacagggcatgttctggtagtggatgggggattacaactcattttgtaa

SEQ ID NO: 16 - DNA, 2156 bp, Homo sapiens
  CDS (39)...(1499)
ctcagtcgtaaagaggaaaggcagaatttttccttgctatggctggaacaaacacacttttgatgctggaaaacttcatagatggaaaatttttaccttgtagctcatatatagattcttacgacccatcaacaggggaagtgtattgcagagtgccaaatagtggaaaagacgagatcgaagccgcggtcaaggccgccagagaagcctttcccagctggtcatcccgcagcccccaggagcgctcacgggtcctgaaccaggtggcggatttgctggagcagtccctggaggagtttgcccaggccgagtctaaagaccaagggaaaaccttagcactggcaagaaccatggacattccccggtctgtgcagaacttcaggttcttcgcttcctccagcctgcaccacacgtcagagtgcacgcagatggaccacctgggctgcatgcactacacggtgcgggccccggtgggagtcgctggtctgatcagcccctggaatttgccactctacttgctgacctggaagatagctccagcgatggctgcagggaacactgtgatagccaagcccagtgagctgacttcagtgactgcgtggatgttgtgcaaactcctggataaagcaggtgttccaccaggtgtggtcaatattgtgtttggaaccgggcccagggtgggtgaggccctggtgtcccacccagaggtgcccctgatctccttcaccgggagccagcccaccgctgagcggatcacccagctgagcgctccccactgcaaaaagctctccctggagctggggggcaagaatcctgccatcatctttgaggacgccaacctggatgagtgcattccggcaaccgtcaggtccagctttgccaaccagggtgaaatctgtctctgtaccagcaggatctttgtccagaagagcatctatagtgaatttttaaagagatttgtagaagctaccagaaagtggaaagtcggcattccctctgatccactggtgagcataggtgctctgataagtaaagcacatttggagaaagtcagaagttacgtcaagagagctcttgctgaaggtgcccaaatttggtgcggtgagggagtggataagttgagcctccctgccaggaaccaggcaggctactttatgcttcccacggtgataacagacattaaggatgaatcctgctgcatgacggaagagatatttggtccagtgacgtgtgtcgtcccctttgatagtgaagaggaggtgattgaaagagccaacaacgttaagtatgggctgggggctaccgtgtggtccagcaatgtggggcgcgtccaccgggtggctaagaagctgcagtctggcttggtctggaccaactgctggctcatcagggagctgaaccttcctttcggggggatgaagagttctggaataggtagagagggagccaaggactcttacgacttcttcactgagatcaaaaccatcaccgttaaacactgatctttgctaatggtggagccactatggccaatgcctggctgcaggcatcagttgttcaatgtggtagatgaaaatcatggcatgaattccagctatgccttgacttggcagaaggttatctctagcttatcctcagttcttagtaactttacccactagtgaagagatactgtctattttcaatgtggactcggaaaaaaagacttataagtaggaagatagaacaatgatgccagttgtcaggctcctcccaggttatgttttcatagtgtttctttcatcatcttcattgaactcttgggaatctccagataatcagattatttcatttggtaaattttaaaaaatatgcaatcaggcacagtgcctcatgcctataatcccagcactttgggaggccaaggtgggtggatcacttgagttcaggagttcgagatcagcctaggcaacatggtgaaatcctgtctttaccaaaagtttaaaaattagcttggtgtggtgccctctgcctatagccccagctacttgggaggctgaggtgggaggatcgcttgagcccaggcggttgaggctgcagtgagccatgatcattccactgcatttcagcctgggggatacagtgagaccttgtctttaaaaaaaaaaaaaaaaaaa

SEQ ID NO: 17 - protein, 487 aa, Homo sapiens
MAGTNTLLMLENFIDGKFLPCSSYIDSYDPSTGEVYCRVPNSGKDEIEAAVKAAREAFPSWSSRSPQERSRVLNQVADLLEQSLEEFAQAESKDQGKTLALARTMDIPRSVQNFRFFASSSLHHTSECTQMDHLGCMHYTVRAPVGVAGLISPWNLPLYLLTWKIAPAMAAGNTVIAKPSELTSVTAWMLCKLLDKAGVPPGVVNIVFGTGPRVGEALVSHPEVPLISFTGSQPTAERITQLSAPHCKKLSLELGGKNPAIIFEDANLDECIPATVRSSFANQGEICLCTSRIFVQKSIYSEFLKRFVEATRKWKVGIPSDPLVSIGALISKAHLEKVRSYVKRALAEGAQIWCGEGVDKLSLPARNQAGYFMLPTVITDIKDESCCMTEEIFGPVTCVVPFDSEEEVIERANNVKYGLGATVWSSNVGRVHRVAKKLQSGLVWTNCWLIRELNLPFGGMKSSGIGREGAKDSYDFFTEIKTITVKH

SEQ ID NO: 18 - DNA, 1464 bp, Homo sapiens
atggctggaacaaacacacttttgatgctggaaaacttcatagatggaaaatttttaccttgtagctcatatatagattcttacgacccatcaacaggggaagtgtattgcagagtgccaaatagtggaaaagacgagatcgaagccgcggtcaaggccgccagagaagcctttcccagctggtcatcccgcagcccccaggagcgctcacgggtcctgaaccaggtggcggatttgctggagcagtccctggaggagtttgcccaggccgagtctaaagaccaagggaaaaccttagcactggcaagaaccatggacattccccggtctgtgcagaacttcaggttcttcgcttcctccagcctgcaccacacgtcagagtgcacgcagatggaccacctgggctgcatgcactacacggtgcgggccccggtgggagtcgctggtctgatcagcccctggaatttgccactctacttgctgacctggaagatagctccagcgatggctgcagggaacactgtgatagccaagcccagtgagctgacttcagtgactgcgtggatgttgtgcaaactcctggataaagcaggtgttccaccaggtgtggtcaatattgtgtttggaaccgggcccagggtgggtgaggccctggtgtcccacccagaggtgcccctgatctccttcaccgggagccagcccaccgctgagcggatcacccagctgagcgctccccactgcaaaaagctctccctggagctggggggcaagaatcctgccatcatctttgaggacgccaacctggatgagtgcattccggcaaccgtcaggtccagctttgccaaccagggtgaaatctgtctctgtaccagcaggatctttgtccagaagagcatctatagtgaatttttaaagagatttgtagaagctaccagaaagtggaaagtcggcattccctctgatccactggtgagcataggtgctctgataagtaaagcacatttggagaaagtcagaagttacgtcaagagagctcttgctgaaggtgcccaaatttggtgcggtgagggagtggataagttgagcctccctgccaggaaccaggcaggctactttatgcttcccacggtgataacagacattaaggatgaatcctgctgcatgacggaagagatatttggtccagtgacgtgtgtcgtcccctttgatagtgaagaggaggtgattgaaagagccaacaacgttaagtatgggctgggggctaccgtgtggtccagcaatgtggggcgcgtccaccgggtggctaagaagctgcagtctggcttggtctggaccaactgctggctcatcagggagctgaaccttcctttcggggggatgaagagttctggaataggtagagagggagccaaggactcttacgacttcttcactgagatcaaaaccatcaccgttaaacactga

SEQ ID NO: 19 - protein, 203 aa, Artificial Sequence
  consensus sequence
KVALVTGASSGIGLAIAKRLAKEGAKVVVADRNEEKLEKGAVAKELKELGGNDKDRALAIQLDVTDEESVAAVEQAVERLGRLDVLVNNAGGIILLRPGPFAELSRTMEEDWDRVIDVNLTGVFLLTRAVLPLMAMKKRGGGRIVNISSVAGRKEGGLVGVPGGSAYSASKAAVIGLTRSLALELAPHGIRVNAVAPGGVDTD

SEQ ID NO: 20 - protein, 31 aa, Artificial Sequence
  consensus sequence
GRLGEPEEIANAVVFLASDAASYITGQTLVV

SEQ ID NO: 21 - protein, 493 aa, Artificial Sequence
  consensus sequence
EWVDSASGKTFEVVNPANKGEVIGRVPEATAEDVDAAVKAAKEAFKSGPWWAKVPASERARILRKLADLIEEREDELAALETLDLGKPLAEAKGDTEVGRAIDEIRYYAGWARKLMGERRVIPSLATDGDEELNYTRREPLGVVGVISPWNFPLLLALWKLAPALAAGNTVVLKPSEQTPLTALLLAELIEEAGANNLPKGVVNVVPGFGAEVGQALLSHPDIDKISFTGSTEVGKLIMEAAAAKNLKKVTLELGGKSPVIVFDDADLDKAVERIVFGAFGNAGQVCIAPSRLLVHESIYDEFVEKLKERVKKLKLIGDPLDSDTNIYGPLISEQQFDRVLWSYIEDGKEEGAKVLCGGERDESKEYLGGGYYVQPTIFTDVTPDMKIMKEEIFGPVLPIIKFKDLDEAIELANDTEYGLAAYVFTKDILARAFRVAKALEAGIVWVNDVCVHAAEPQLPFGGVKQSSGIGREHGGKYGLEEYTEIKTVTIRL

SEQ ID NO: 22 - DNA, 3320 bp, Homo sapiens
  CDS (459)...(2591)
ccggacacctgggctcccgcccaggatcctgcaggcccagggcggtcctggagcggaaagaatgccacgcggggcattcagaccctgtttgccggcgctgtatttcgctttcctgacctgccctactccagagcagagaatgcagtggaacccaggctcctgatatccatctgggtgagccagccagagggaccggctgtgtcagaggcaagcaaacaagtattagagtgcaagactgtgggcggagagaggaagcccgagccgccagcagggagcttcggagagagaaagcccaggaacatcccagagagagctgggcccatcctcagccctacccagccccgcagcccctagccctccgcccagaaacccagccctgtccggcgtgccgctcttctcctccaggccggctgctgctgcggccagcgttgccggggcatcccttcctccttcccatcatggcagtgtaccgcctgtgtgtgaccactggtccctacctgagggccggcacactggacaacatctctgtcacactggtgggcacgtgtggtgaaagccccaagcagcggctagatcgaatgggcagggacttcgcccctggatcggtacagaagtacaaggtgcgttgcacagcggagctgggtgagctcttgctgctgcgtgtacacaaggagcgctacgctttcttccgcaaggactcttggtactgtagccgcatctgtgtcaccgaaccggatggtagtgtatcccacttcccctgctatcagtggattgaaggctactgcaccgtggagctgaggccaggaacagcaagaactatttgtcaggactctcttcccctcctcctggatcacaggacacgggagctccgggcccgacaagaatgctaccgctggaagatctatgcccctggcttcccctgcatggtagacgtcaacagctttcaggagatggagtcagacaagaaatttgccttgacaaagacgacaacttgtgtagaccagggtgacagcagtgggaatcggtacctgcccggcttccccatgaaaattgacatcccatccctgatgtacatggagcccaatgttcgatactcagccaccaagacgatctcgctgctcttcaatgccatccctgcgtccttgggaatgaagcttcgagggctgttggatcgcaagggctcctggaagaagctggatgacatgcagaacatcttctggtgccataagaccttcacgacaaagtatgtcacagagcactggtgtgaagatcacttctttgggtaccagtacctgaatggtgtcaatcccgtcatgctccactgcatctctagcttgcccagcaagctgcctgtcaccaatgacatggtggcccccttgctgggacaggacacatgcctgcagacagagctagagagggggaacatcttcctagcggactactggatcctggcggaggcccccacccactgcctaaacggccgccagcagtacgtggccgccccactgtgcctgctgtggctcagcccccagggggcgctggtgcccttggccatccagctcagccagacccccgggcctgacagccccatcttcctgcccactgactccgaatgggactggctgctggccaagacgtgggtgcgcaactctgagttcctggtgcacgaaaacaacacgcactttctgtgcacgcatttgctgtgcgaggccttcgccatggccacgctgcgccagctgccgctctgccaccccatctacaagctcctactcccccacactcgatacacgctgcaggtgaacaccatcgcgagggccacgctgctcaaccccgagggcctcgtggaccaggtcacgtccatcgggaggcaaggcctcatctacctcatgagcacgggcctggcccacttcacctacaccaatttctgccttccggacagcctgcgggcccgcggcgtcctggctatccccaactaccactaccgagacgacggcctgaagatctgggcggccattgagagctttgtctcagaaatcgtgggctactattatcccagtgacgcatctgtgcagcaggattcggagctgcaggcctggactggcgagatttttgctcaggcgttcctgggccgggaaagctcaggtttcccaagccggctgtgcaccccaggagagatggtgaagttcctcactgcaatcatcttcaattgctctgcccagcacgctgctgtcaacagtgggcagcatgactttggggcctggatgcccaatgctccatcatccatgaggcagcccccaccccagaccaaggggaccaccaccctgaagacttacctagacaccctccctgaagtgaacatcagctgtaacaacctcctcctcttctggttggttagccaagaacccaaggaccagaggcccctgggcacctacccagatgagcacttcacagaggaggccccgaggcggagcatcgccgccttccagagccgcctggcccagatctcaagggacatccaggagcggaaccagggtctggcactgccctacacctacctggaccctcccctcattgagaacagtgtctccatctaaccacccccaaataccacccaagaagaaagaaaggtccaagcatgaggaggaccagttcctcaggtcctccagacccttccatcctccctgttctcagttcacctgaaccttctcttctgcacatggagacttttgcagccaagatggctctgacatcatacaaactgggccctgagctgtgagagaccagcacagcagcgtccaggttaaaagccgctgaccaaagtccaatgcacaatagcccctccgaaaggaaggaaccgcttcacttcttgccccacttggggcagcctcttgttccagcctcttggaatgcccagcttgggtttctgagcttttctccctcatcctcccccatccccaaactccttctcctaccatgcctttctacgttctctttcttccaagcctagagccaccagcccagcttccttctctggaaaagcctggaaactgggcacagaaggactgtgtgcctgggtctaacatgtggtcccctttgtccctagcacctttaaggggaggggaagaattggagggcagcttgcctggacccctaacggctgttctcaggaacaggttcccaggcctggggtgtttgtggagrtctgtctttctccaaagwtttcatccaactcccctttcwtcccmctccctttcwtcccatttttttctttctgtccttgagcccagtgagttcaataaaaaccaaaatatttggctatc

SEQ ID NO: 23 - protein, 711 aa, Homo sapiens
MAVYRLCVTTGPYLRAGTLDNISVTLVGTCGESPKQRLDRMGRDFAPGSVQKYKVRCTAELGELLLLRVHKERYAFFRKDSWYCSRICVTEPDGSVSHFPCYQWIEGYCTVELRPGTARTICQDSLPLLLDHRTRELRARQECYRWKIYAPGFPCMVDVNSFQEMESDKKFALTKTTTCVDQGDSSGNRYLPGFPMKIDIPSLMYMEPNVRYSATKTISLLFNAIPASLGMKLRGLLDRKGSWKKLDDMQNIFWCHKTFTTKYVTEHWCEDHFFGYQYLNGVNPVMLHCISSLPSKLPVTNDMVAPLLGQDTCLQTELERGNIFLADYWILAEAPTHCLNGRQQYVAAPLCLLWLSPQGALVPLAIQLSQTPGPDSPIFLPTDSEWDWLLAKTWVRNSEFLVHENNTHFLCTHLLCEAFAMATLRQLPLCHPIYKLLLPHTRYTLQVNTIARATLLNPEGLVDQVTSIGRQGLIYLMSTGLAHFTYTNFCLPDSLRARGVLAIPNYHYRDDGLKIWAAIESFVSEIVGYYYPSDASVQQDSELQAWTGEIFAQAFLGRESSGFPSRLCTPGEMVKFLTAIIFNCSAQHAAVNSGQHDFGAWMPNAPSSMRQPPPQTKGTTTLKTYLDTLPEVNISCNNLLLFWLVSQEPKDQRPLGTYPDEHFTEEAPRRSIAAFQSRLAQISRDIQERNQGLALPYTYLDPPLIENSVSI

SEQ ID NO: 24 - DNA, 2136 bp, Homo sapiens
atggcagtgtaccgcctgtgtgtgaccactggtccctacctgagggccggcacactggacaacatctctgtcacactggtgggcacgtgtggtgaaagccccaagcagcggctagatcgaatgggcagggacttcgcccctggatcggtacagaagtacaaggtgcgttgcacagcggagctgggtgagctcttgctgctgcgtgtacacaaggagcgctacgctttcttccgcaaggactcttggtactgtagccgcatctgtgtcaccgaaccggatggtagtgtatcccacttcccctgctatcagtggattgaaggctactgcaccgtggagctgaggccaggaacagcaagaactatttgtcaggactctcttcccctcctcctggatcacaggacacgggagctccgggcccgacaagaatgctaccgctggaagatctatgcccctggcttcccctgcatggtagacgtcaacagctttcaggagatggagtcagacaagaaatttgccttgacaaagacgacaacttgtgtagaccagggtgacagcagtgggaatcggtacctgcccggcttccccatgaaaattgacatcccatccctgatgtacatggagcccaatgttcgatactcagccaccaagacgatctcgctgctcttcaatgccatccctgcgtccttgggaatgaagcttcgagggctgttggatcgcaagggctcctggaagaagctggatgacatgcagaacatcttctggtgccataagaccttcacgacaaagtatgtcacagagcactggtgtgaagatcacttctttgggtaccagtacctgaatggtgtcaatcccgtcatgctccactgcatctctagcttgcccagcaagctgcctgtcaccaatgacatggtggcccccttgctgggacaggacacatgcctgcagacagagctagagagggggaacatcttcctagcggactactggatcctggcggaggcccccacccactgcctaaacggccgccagcagtacgtggccgccccactgtgcctgctgtggctcagcccccagggggcgctggtgcccttggccatccagctcagccagacccccgggcctgacagccccatcttcctgcccactgactccgaatgggactggctgctggccaagacgtgggtgcgcaactctgagttcctggtgcacgaaaacaacacgcactttctgtgcacgcatttgctgtgcgaggccttcgccatggccacgctgcgccagctgccgctctgccaccccatctacaagctcctactcccccacactcgatacacgctgcaggtgaacaccatcgcgagggccacgctgctcaaccccgagggcctcgtggaccaggtcacgtccatcgggaggcaaggcctcatctacctcatgagcacgggcctggcccacttcacctacaccaatttctgccttccggacagcctgcgggcccgcggcgtcctggctatccccaactaccactaccgagacgacggcctgaagatctgggcggccattgagagctttgtctcagaaatcgtgggctactattatcccagtgacgcatctgtgcagcaggattcggagctgcaggcctggactggcgagatttttgctcaggcgttcctgggccgggaaagctcaggtttcccaagccggctgtgcaccccaggagagatggtgaagttcctcactgcaatcatcttcaattgctctgcccagcacgctgctgtcaacagtgggcagcatgactttggggcctggatgcccaatgctccatcatccatgaggcagcccccaccccagaccaaggggaccaccaccctgaagacttacctagacaccctccctgaagtgaacatcagctgtaacaacctcctcctcttctggttggttagccaagaacccaaggaccagaggcccctgggcacctacccagatgagcacttcacagaggaggccccgaggcggagcatcgccgccttccagagccgcctggcccagatctcaagggacatccaggagcggaaccagggtctggcactgccctacacctacctggaccctcccctcattgagaacagtgtctccatctaa

SEQ ID NO: 25 - protein, 491 aa, Artificial Sequence
  consensus sequence
AWMTDEEFAREMLAGVNPVVIRRLQEFPPKSKLDPAVYGDQTSTITKEHLELNLGGLTVEEALQNGRLFILDHHDLFIPYLNKINSLTSTKLYATRTLLFLKDDGTLKPLAIELSLPHPDGDPFGAVSKVFLPADEGVESSIWLLAKAYVRVNDSGYHQLISHWLNTHAVVEPFVIATNRQLSVLHPIYKLLLPHYRDTMNINALARQSLINAGGIIEKTFLPGKYGAVEMSSAVYKKDWVFTDQALPADLVKRGLAVEDPSSPHGVRLLIEDYPYAVDGLEIWDAIKTWVQEYVSLYYKSDEAVKKDPELQAWWKEVREVGHGDKKDEPWWPKLQTREDLIEVCTIIIWIASALHAAVNFGQYPYGGYIPNRPTTSRRPMPEEGPVDTAEYEELAKNPEKALLKTITSQLQALLDLSVIEILSRHASDEVYLGQRDEPEWTSDKKALEAFKRFGKKLAEIEKKITERNKDESLKNRVGPVKLPYTLLKPS

SEQ ID NO: 26 - protein, 129 aa, Artificial Sequence
  consensus sequence
VSYQLIVATGDDSTFAGTTGKVGISLYGEKGESKKIKLLKGELKNLPTLGFGPGSTFSFEFDVDEDFGELGAVKIKNEHHSLNSNPTDDEWFLKSITVEDPGTQGEVHFPCNSWVYGKTPKEYLSLRIC

SEQ ID NO: 27 - protein, 157 aa, Artificial Sequence
  consensus sequence
AKYKVTVTLGKKNVLDFAGTTALGSLLDGLTDLLGRQSVSLSLIGAEGDDNTGRGKESKLAYLERPLTTLPSLFARGSTYEFEFDVDEDFGELGAVKIKNEHYGLFWSSPRHSEFFLKSITLKDLGPTGGKVHFPCNSWVYPKKKPGYKGKRIFFAN

SEQ ID NO: 28 - DNA, 1639 bp, Homo sapiens
  CDS (191)...(1099)
acggactgggcctggcctggggcgtccccgcgaagcctgggcctgtcaggcggttccgtccgggtctcggccaccgtcgagttccgtcgagttccgtcccggccctgctcacagcagcgccctcggagcgcccagcacctgcggccggccaggcagcgcgatcctgcggcgtctggccatcccgaatgctatggccgccgtcgccgtcttgcgggccttcggggcaagtgggcccatgtgtctccggcgcggcccctgggcccagctccccgcccgcttctgcagccgggacccggccggggcggggcggcgggagtcggagccgcggcccaccagcgcgcggcagctggacggcataaggaacatcgtcttgagcaatcccaagaagaggaacacgttgtcacttgcaatgctgaaatctctccaaagtgacattcttcatgacgctgacagcaacgatctgaaagtcattatcatctcggctgaggggcctgtgttttcttctgggcatgacttaaaggagctgacagaggagcaaggccgtgattaccatgccgaagtatttcagacctgttccaaggtcatgatgcacatccggaaccaccccgtccccgtcattgccatggtcaatggcctggccacggctgccggctgtcaactggttgccagctgcaacattgccgtggcgagcgacaagtcctcttttgccactcctggggtgaacgtcgggctcttctgttctacccctggggttgccttggcaagagcagtgcctagaaaggtggccttggagatgctctttactggtgagcccatttctgcccaggaggccctgctccacgggctgcttagcaaggtggtgccagaggcggagctgcaggaggagaccatgcggatcgctaggaagatcgcgtcactgagccgtccggtggtgtccctgggcaaagccaccttctacaagcagctgccccaggacctggggacggcttactacctcacctcccaggccatggtggacaacctggccctgcgggacgggcaggagggcatcacggccttcctccagaagagaaaacctgtctggtcacacgagccagtgtgagtggaggcagaggagtgaggcccacgggcagcgcccaggagcccaccttcccctctggcccagccaccactgcctctcagcttcaacaggtgacaggctgctttcgtgacttgatattggtgtcatagcatttggcctacattaaaagccacaatttcatggggaaaggacaaaatggagagtgactgaggtgctgacctcagtgcaaggctggtgaaccctgcagcgggccagctatggtgggaagcctggcatttggggtgctccttgcaacgtcttaagcaagcgacccccctgacatagcaaaaggtggcaacccatggaggcagaaagaaggacgccagcctgacccttatctgaaacgtcctaagcagagttaatcctggctgctcaggagaggcgacacatttcaaatctccacgagatattctccacacagaaaatcttcttgattctatagagacttaatcatgcctatggctttgaataatcttatgtgatttaaataaattaaatctttatagagaaaaaaaaaaa

SEQ ID NO: 29 - protein, 303 aa, Homo sapiens [sequence omitted]

SEQ ID NO: 30 - DNA, 912 bp, Homo sapiens
atggccgccgtcgccgtcttgcgggccttcggggcaagtgggcccatgtgtctccggcgcggcccctgggcccagctccccgcccgcttctgcagccgggacccggccggggcggggcggcgggagtcggagccgcggcccaccagcgcgcggcagctggacggcataaggaacatcgtcttgagcaatcccaagaagaggaacacgttgtcacttgcaatgctgaaatctctccaaagtgacattcttcatgacgctgacagcaacgatctgaaagtcattatcatctcggctgaggggcctgtgttttcttctgggcatgacttaaaggagctgacagaggagcaaggccgtgattaccatgccgaagtatttcagacctgttccaaggtcatgatgcacatccggaaccaccccgtccccgtcattgccatggtcaatggcctggccacggctgccggctgtcaactggttgccagctgcaacattgccgtggcgagcgacaagtcctcttttgccactcctggggtgaacgtcgggctcttctgttctacccctggggttgccttggcaagagcagtgcctagaaaggtggccttggagatgctctttactggtgagcccatttctgcccaggaggccctgctccacgggctgcttagcaaggtggtgccagaggcggagctgcaggaggagaccatgcggatcgctaggaagatcgcgtcactgagccgtccggtggtgtccctgggcaaagccaccttctacaagcagctgccccaggacctggggacggcttactacctcacctcccaggccatggtggacaacctggccctgcgggacgggcaggagggcatcacggccttcctccagaagagaaaacctgtctggtcacacgagccagtgtga

SEQ ID NO: 31 - protein, 176 aa, Homo sapiens
AVIKLDRPEEAVNALSAELLTELIEALEKLEQDPSVRAVVLTGAGPGAFSAGADIKEMAAGFKEPLAEQAQFSLEAQDLWSKLEDLPKPVIAAVNGYALGGGLELALACDYRIAADNAKYVFGLPEVKLGIIPGAGGTQRLPRIVGVSAALEMILTGRRIRAQEALKMGLVDKVVP

(19) United States
(12) Patent Application Publication
(10) Pub. No.: US 2003/0092658 A1
(43) Pub. Date: May 15, 2003

(54) Novel human enzyme family members and uses thereof

(76) Inventors: Rachel E. Meyers, Newton, MA (US); Maria Alexandra Glucksmann, Lexington, MA (US); Laura A. Rudolph-Owen, Jamaica Plain, MA (US)

Correspondence Address: Intellectual Property Group, MILLENNIUM PHARMACEUTICALS, INC., 75 Sidney Street, Cambridge, MA, 02139, US

(21) Appl. No.: 10/175,696

(22) Filed: Jun. 20, 2002

Related U.S. Application Data
(63) Continuation-in-part of application No. 10/067,668, filed on Feb. 4, 2002.
(60) Provisional application No. 60/266,140, filed on Feb. 2, 2001.

(51) Int. Cl. A61K 31/70; A01N 43/04; A01N 37/18; C12Q 1/68

(52) U.S. Cl. 514/44; 514/2; 424/130.1; 435/6

(57) ABSTRACT

The invention provides isolated nucleic acids molecules, designated 33312, 33303, 32579, 21509, 33770, 46638, and 50090 nucleic acid molecules, which encode novel G protein-coupled receptor family members, human thioredoxin family members, human leucine-rich repeat family members, and human ringfinger family member. The invention also provides antisense nucleic acid molecules, recombinant expression vectors containing 33312, 33303, 32579, 21509, 33770, 46638, or 50090 nucleic acid molecules, host cells into which the expression vectors have been introduced, and nonhuman transgenic animals in which a 33312, 33303, 32579, 21509, 33770, 46638, or 50090 gene has been introduced or disrupted. The invention still further provides isolated 33312, 33303, 32579, 21509, 33770, 46638, or 50090 proteins, fusion proteins, antigenic peptides and anti-33312, 33303, 32579, 21509, 33770, 46638, or 50090 antibodies. Diagnostic methods utilizing compositions of the invention are also provided.

### RELATED APPLICATIONS

This application is a continuation-in-part and claims priority to U.S. application Ser. No. 10/067,668, filed Feb. 4, 2002, which claims the benefit of U.S. Provisional Application Serial No. 60/266,140, filed Feb. 2, 2001; and U.S. application Ser. No. 09/823,901, filed Mar. 30, 2001, and International Application Serial No. PCT/US01/10720, filed Apr. 2, 2001, which claim the benefit of U.S. Provisional Application Serial No. 60/193,920, filed Mar. 31, 2000; and U.S. application Ser. No. 09/862,658, filed May 21, 2001, and International Application Serial No. PCT/US01/16380, filed May 21, 2001, which claim the benefit of U.S. Provisional Application Serial No. 60/205,675, filed May 19, 2000; and U.S. application Ser. No. 09/882,837, filed Jun. 15, 2001, and International Application Serial No. PCT/US01/19319, filed Jun. 15, 2001, which claim the benefit of U.S. Provisional Application Serial No. 60/211,727, filed Jun. 15, 2000, the contents of which are incorporated herein by reference.

### BACKGROUND OF THE 33312, 33303, AND 32579 INVENTION

Cytochrome P450s are members of a large superfamily of hemoproteins that are involved in the oxidative metabolism of a high number of natural compounds (such as steroids, fatty acids, metabolites, prostaglandins, leukotrienes, etc.), as well as drugs, carcinogens, antioxidants, and mutagens (Ioannides, C. (1996) Cytochromes P450: Metabolic and Toxicological Aspects. CRC Press Inc.; Johnson, E. F. & Waterman, M. R., Eds. (1996) Methods in Enzymology, vol. 272. Cytochrome P450 (Part B) Academic Press, San Diego). Usually, they act as terminal oxidases in multi-compound electron transfer chains, called P450-containing monooxygenase systems.

P450-containing systems can be categorized according to the number of protein components: (1) Mitochondrial and most bacterial P450 systems have three components: an FAD-containing flavoprotein (NADPH or NADH-dependent reductase), an iron-sulphur protein, and P450. (2) The eukaryotic microsomal P450 system contains two components: NADPH:P450 reductase (a flavoprotein containing both FAD and FMN) and P450. (3) A soluble monooxygenase P450BM-3 from Bacillus Megaterium exists as a single polypeptide chain with two functional parts, and represents a unique bacterial one-component system.

Cytochrome P450s catalyze oxidation reactions in the metabolism of endogenous and exogenous substrates. For example, they are involved in steroid biosynthesis pathways, as well as fatty acid metabolism (Capdevila et al. (1996) J. Biol. Chem. 271, 22663-22671). Furthermore, cytochrome P450s play important roles in the metabolic activation and detoxification of many low molecular weight molecules, such as carcinogens, metabolites, and other toxins (Lin et al. (1999) Toxicology & App. Pharm. 157, 117-124.) More importantly, Cytochrome P450s are involved in drug metabolism, mediating drug-drug interactions (Guengerich, F. P. (1997) Adv. Pharmacol. 43, 7-35).

The 3D structures of several P450s have been reported, e.g., P450cam (Poulos et al. (1987) J. Mol. Biol. 195, 687-700), and P450terp (Hasemann et al. (1994) J. Mol. Biol. 236 1169-1185). Although the sequence identity between any two P450s with known 3D structures reaches only 20% or less, the overall topology of the proteins is similar, with some differences in various helices orientations. The most dramatic variations between P450 structures are found in regions responsible for a substrate binding and access (Graham et al. (1999) Arch Biochem. Biophy. 369, 24-9). There is a highly conserved core, containing a cysteine residue in the C-terminal part involved in binding a heme iron having a ten residue motif: [FW]-[SGNH]-X-[GD]-X-[RKHPT]-X—C-[LIVMFAP]-[GAD].

### SUMMARY OF THE 33312, 33303, AND 32579 INVENTION

The present invention is based, in part, on the discovery of three novel cytochrome P450 family members, referred to herein as “33312,” “33303,” and “32579.” The nucleotide sequence of a cDNA encoding 33312 is shown in SEQ ID NO:1, and the amino acid sequence of a 33312 polypeptide is shown in SEQ ID NO:2. In addition, the nucleotide sequences of the coding region are depicted in SEQ ID NO:3. The nucleotide sequence of a cDNA encoding 33303 is shown in SEQ ID NO:4, and the amino acid sequence of a 33303 polypeptide is shown in SEQ ID NO:5. In addition, the nucleotide sequences of the coding region of 33303 are depicted in SEQ ID NO:6. The nucleotide sequence of a cDNA encoding 32579 is shown in SEQ ID NO:7, and the amino acid sequence of a 32579 polypeptide is shown in SEQ ID NO:8. In addition, the nucleotide sequences of the coding region are depicted in SEQ ID NO:9.

Accordingly, in one aspect, the invention features a nucleic acid molecule that encodes a 33312, 33303, or 32579 protein or polypeptide, e.g., a biologically active portion of a 33312, 33303, or 32579 protein. In a preferred embodiment the isolated nucleic acid molecule encodes a polypeptide having the amino acid sequence of SEQ ID NO:2, SEQ ID NO:5, or SEQ ID NO:8. In other embodiments, the invention provides isolated 33312, 33303, or 32579 nucleic acid molecules having the nucleotide sequence shown in SEQ ID NO:1, SEQ ID NO:3, SEQ ID NO:4, SEQ ID NO:6, SEQ ID NO:7, SEQ ID NO:9. In still other embodiments, the invention provides nucleic acid molecules that are substantially identical (e.g., naturally occurring allelic variants) to the nucleotide sequence shown in SEQ ID NO: 1, SEQ ID NO:3, SEQ ID NO:4, SEQ ID NO:6, SEQ ID NO:7, SEQ ID NO: 9. In other embodiments, the invention provides a nucleic acid molecule which hybridizes under stringent hybridization conditions to a nucleic acid molecule comprising the nucleotide sequence of SEQ ID NO: 1, SEQ ID NO:3, SEQ ID NO:4, SEQ ID NO:6, SEQ ID NO:7, SEQ ID NO:9, wherein the nucleic acid encodes a full length 33312, 33303, or 32579 protein or an active fragment thereof.

In a related aspect, the invention further provides nucleic acid constructs that include a 33312, 33303, or 32579 nucleic acid molecule described herein. In certain embodiments, the nucleic acid molecules of the invention are operatively linked to native or heterologous regulatory sequences. Also included, are vectors and host cells containing the 33312, 33303, or 32579 nucleic acid molecules of the invention e.g., vectors and host cells suitable for producing 33312, 33303, or 32579 nucleic acid molecules and polypeptides. The invention thus also provides vectors and host cells that express the 33312, 33303, or 32579 cytochrome P450 nucleic acid molecules and polypeptides of the invention. Transgenic animals expressing 33312, 33303, or 32579 cytochrome P450 nucleic acid molecules and polypeptides of the invention also are provided.

In another related aspect, the invention provides nucleic acid fragments suitable as primers or hybridization probes for the detection of 33312, 33303, or 32579-encoding nucleic acids.

In still another related aspect, isolated nucleic acid molecules that are antisense to a 33312, 33303, or 32579 encoding nucleic acid molecule are provided.

In another embodiment, the invention provides 33312, 33303, or 32579 polypeptides. Preferred polypeptides are 33312, 33303, or 32579 proteins having a 33312, 33303, or 32579 activity, e.g., a 33312, 33303, or 32579 activity as described herein. In another aspect, the invention features, 33312, 33303, or 32579 polypeptides, and biologically active or antigenic fragments thereof that are useful, e.g., as reagents or targets in assays applicable to treatment and diagnosis of 33312, 33303, or 32579 cytochrome P450 mediated or related disorders.

In other embodiments, the invention provides 33312, 33303, or 32579 polypeptides, e.g., a 33312, 33303, or 32579 polypeptide having the amino acid sequence shown in SEQ ID NO:2, SEQ ID NO:5, SEQ ID NO:8; an amino acid sequence that is substantially identical to the amino acid sequence shown in SEQ ID NO:2, SEQ ID NO:5, or SEQ ID NO:8; or an amino acid sequence encoded by a nucleic acid molecule having a nucleotide sequence which hybridizes under stringent hybridization conditions to a nucleic acid molecule comprising the nucleotide sequence of SEQ ID NO:1, SEQ ID NO:3, SEQ ID NO:4, SEQ ID NO:6, SEQ ID NO:7, SEQ ID NO:9, wherein the nucleic acid encodes a full length 33312, 33303, or 32579 protein or an active fragment thereof.

The 33312, 33303, or 32579 cytochrome P450 polypeptides are useful as reagents or targets in 33312, 33303, or 32579 cytochrome P450 activity assays and are applicable to treatment and diagnosis of 33312, 33303, or 32579 cytochrome P450-related disorders. The invention therefore also provides methods of treating a subject having or at risk of having a 33312, 33303, or 32579 cytochrome P450 disorder. In one embodiment, a method of the invention includes administering a 33312, 33303, or 32579 cytochrome P450 polypeptide, subsequence or variant sequence thereof, or a nucleic acid encoding the same, to a subject in an amount effective to treat or ameliorate one or more symptoms of the disorder. In one aspect, the disorder is associated with or results from undesirable or aberrant 33312, 33303, or 32579 cytochrome P450 expression or an activity. In another embodiment, the disorder is associated with or results from insufficient 33312, 33303, or 32579 cytochrome P450 expression or activity.

In a related aspect, the invention provides 33312, 33303, or 32579 polypeptides or fragments operatively linked to non-33312, 33303, or 32579 polypeptides to form fusion proteins.

In another aspect, the invention features antibodies and antigen-binding fragments thereof, that react with, or more preferably specifically bind 33312, 33303, or 32579 polypeptides or fragments thereof.

In another aspect, the invention provides methods of screening for compounds that modulate the expression or activity of the 33312, 33303, or 32579 polypeptides or nucleic acids. In yet another aspect, the invention provides antibodies or antigen-binding fragments thereof that selectively bind the 33312, 33303, or 32579 cytochrome P450 polypeptides and subsequences. Such antibodies and antigen binding fragments have use in the detection of a 33312, 33303, or 32579 cytochrome P450 polypeptide, and in prevention, diagnosis and treatment of 33312, 33303, or 32579 cytochrome P450 related disorders. Thus, an antibody that binds a 33312, 33303, or 32579 cytochrome P450 polypeptide and modulates expression or an activity of 33312, 33303, or 32579 cytochrome P450 polypeptide can be used for treating a disease treatable by modulating expression or the particular activity of 33312, 33303, or 32579 cytochrome P450 polypeptide.

In still another aspect, the invention provides a process for modulating 33312, 33303, or 32579 polypeptide or nucleic acid expression or activity, e.g. using the screened compounds. In certain embodiments, the methods involve treatment of conditions or disorders related to aberrant activity or expression of the 33312, 33303, or 32579 polypeptides or nucleic acids, such as e.g., conditions or disorders involving aberrant cytochrome P450 activity.

The invention also provides assays for determining the activity of or the presence or absence of 33312, 33303, or 32579 polypeptides or nucleic acid molecules in a biological sample, including for disease diagnosis. In addition, the invention provides assays for determining the presence of a mutation in the polypeptides or nucleic acid molecules, such mutations including those that increase or decrease expression or an activity of 33312, 33303, or 32579 cytochrome P450 polypeptide. Such assays are useful, for example, in disease diagnosis, in particular, where the disease causes or results in altered expression or activity of 33312, 33303, or 32579 cytochrome P450 polypeptide.

In further aspect the invention provides assays for determining the presence or absence of a genetic alteration in a 33312, 33303, or 32579 polypeptide or nucleic acid molecule, including for disease diagnosis.

In another aspect, the invention features a two dimensional array having a plurality of addresses, each address of the plurality being positionally distinguishable from each other address of the plurality, and each address of the plurality having a unique capture probe, e.g., a nucleic acid or peptide sequence. At least one address of the plurality has a capture probe that recognizes a 33312, 33303, or 32579 molecule. In one embodiment, the capture probe is a nucleic acid, e.g., a probe complementary to a 33312, 33303, or 32579. In another embodiment, the capture probe is a polypeptide, e.g., an antibody specific for 33312, 33303, or 32579 polypeptides. Also featured is a method of analyzing a sample by contacting the sample to the aforementioned array and detecting binding of the sample to the array.

Other features and advantages of the invention will be apparent from the following detailed description, and from the claims.

### DESCRIPTION OF THE DRAWINGS

FIG. 1 depicts a hydropathy plot of 33312 cytochrome P450. Relative hydrophobic residues are shown above the dashed horizontal line, and relative hydrophilic residues are below the dashed horizontal line. The cysteine residues (Cys) and N-glycosylation sites (Ngly) are indicated by short vertical lines just below the trace. The numbers corresponding to the amino acid sequence of human 33312 are indicated. Polypeptides of the invention include fragments which include: all or part of a hydrophobic sequence, i.e., a sequence above the dashed line, e.g., the sequence of about 82 to about 95, of about 145 to about 158, of about 321 to about 332, and of about 400 to about 411 of SEQ ID NO:2; all or part of a hydrophilic sequence, i.e., a sequence below the dashed line, e.g., the sequence of about 130 to about 142, and of about 325 to about 350 of SEQ ID NO:2; a sequence which includes a Cys or a glycosylation site.

FIGS. 2A-2B depict alignments of structural and functional domains of the amino acid sequence of human 33312 (the lower amino acid sequences) with consensus amino acid sequences derived from a hidden Markov model (HMM) from PFAM. The upper amino acid sequences is the consensus amino acid sequence for cytochrome P450 domains (SEQ ID NO: 10), while the lower sequence corresponds to amino acids of about 46 to about 501 of SEQ ID NO:2.

FIG. 3 depicts a hydropathy plot of 33303 cytochrome P450. Relative hydrophobic residues are shown above the dashed horizontal line, and relative hydrophilic residues are below the dashed horizontal line. The cysteine residues (Cys) are indicated by short vertical lines just below the trace. The numbers corresponding to the amino acid sequence of human 33303 are indicated. Polypeptides of the invention include fragments which include: all or part of a hydrophobic sequence, i.e., a sequence above the dashed line, e.g., the sequence of about 164 to about 190, of about 285 to about 320, and of about 445 to about 461 of SEQ ID NO:5; all or part of a hydrophilic sequence, i.e., a sequence below the dashed line, e.g., the sequence of about 120 to about 130, of about 272 to about 290, and of about 400 to about 425 of SEQ ID NO:5; a sequence which includes a Cys site.

FIGS. 4A-4B depict alignments of structural and functional domains of the amino acid sequence of human 33303 (the lower amino acid sequences) with consensus amino acid sequences derived from a hidden Markov model (HMM) from PFAM. The upper amino acid sequences is the consensus amino acid sequence for cytochrome P450 domains (SEQ ID NO: 10), while the lower sequence corresponds to amino acids of about 33 to about 493 of SEQ ID NO:5.

FIG. 5 depicts a hydropathy plot of 32579 cytochrome P450. Relative hydrophobic residues are shown above the dashed horizontal line, and relative hydrophilic residues are below the dashed horizontal line. The cysteine residues (Cys) and N-glycosylation sites (Ngly) are indicated by short vertical lines just below the trace. The numbers corresponding to the amino acid sequence of human 32579 are indicated. Polypeptides of the invention include fragments which include: all or part of a hydrophobic sequence, i.e., a sequence above the dashed line, e.g., the sequence of about 115 to about 132, of about 220 to about 237, of about 341 to about 355, and of about 410 to about 422 of SEQ ID NO:8; all or part of a hydrophilic sequence, i.e., a sequence below the dashed line, e.g., the sequence of about 241 to about 252, and of about 321 to about 341 of SEQ ID NO:8; a sequence which includes a Cys or a glycosylation site.

FIGS. 6A-6C depict alignments of structural and functional domains of the amino acid sequence of human 32579 (the lower amino acid sequences) with consensus amino acid sequences derived from a hidden Markov model (HMM) from PFAM. The upper amino acid sequences is the consensus amino acid sequence for cytochrome P450 domains (FIG. 6A, SEQ ID NO: 11; FIGS. 6B-6C, SEQ ID NO: 12), while the lower sequence corresponds to amino acids of about 60 to about 72, and of about 107 to about 543 of SEQ ID NO:8.

FIG. 7 depicts a cDNA sequence (SEQ ID NO: 13) and predicted amino acid sequence (SEQ ID NO: 14) of human 21509. The methionine-initiated open reading frame of human 21509 (without the 5′ and 3′ untranslated regions of SEQ ID NO: 13) is shown as coding sequence SEQ ID NO:15.

FIG. 8 depicts a hydropathy plot of human 21509. Relative hydrophobic residues are shown above the dashed horizontal line, and relative hydrophilic residues are below the dashed horizontal line. The numbers corresponding to the amino acid sequence of human 21509 or 33770 are indicated. Polypeptides of the invention include fragments which include: all or part of a hydrophobic sequence, i.e., a sequence above the dashed line, e.g., the sequence from about amino acid 125 to 135 or from about 205 to 220 of SEQ ID NO:14; all or part of a hydrophilic sequence, i.e., a sequence below the dashed line, e.g., the sequence of from about amino acid 55 to 70 or from about 180 to 195 of SEQ ID NO:14.

FIGS. 9A-9B depicts alignments of human 21509 (SEQ ID NO:14) with consensus amino acid sequences, derived from a hidden Markov model (PFAM Accession Number PF00106), from PFAM. A) The upper sequence is the consensus sequence of the short chain dehydrogenase family domain (SEQ ID NO:19), while the lower amino acid sequence corresponds to amino acids 3 to 184 of human 21509 (SEQ ID NO:14). B) The upper sequence is an alignment of the C-terminal portion of the short chain dehydrogenase family domain (SEQ ID NO:20), while the lower sequence corresponds to amino acids 201 to 229 of human 21509 (SEQ ID NO:14).

FIG. 10 depicts expression of 21509, detected using Taqman analysis, in a panel of human tissues, including blood vessels (arteries, veins, smooth muscle cells; columns 1, 5, and 6, respectively), heart (columns 2-4), neurons (columns 7-8), brain (columns 9-10), glial cells (columns 11-12), brest (columns 13-14), ovary (columns 15-16), pancreas (column 17), prostate (columns 18-19), colon (columns 20-21), kidney (column 22), liver (columns 24-26), lung (columns 27-28), spleen (column 29), tonsil (column 30), lymph node (column 31), thymus (column 32), epithelial cells (column 33), endothelial cells (column 34), skeletal muscle (column 35), dermal fibroblasts (column 36), skin (column 37), adipose (column 38), osteoblasts (columns 39-41), and osteoclasts (column 42), as well as some tumorous tissues, including glial (column 12), breast (column 14), ovary (column 16), prostate (column 19), and colon tumors (column 21). Expression of 21509 RNA was detected in all samples analyzed, with the most notable expression occurring in epithelial cell, brain, heart, liver, kidney, endothelial cell, skeletal muscle, and breast tissues. Increased expression of human 21509 RNA was detected in prostate (column 19) and colon (column 21) tumor samples, as compared to normal prostate (column 18) and colon (column 20) samples, respectively. Decreased expression of human 21509 RNA was detected in a glialblastoma (column 12) sample, as compared to normal glia (column 11).

FIG. 11 depicts expression of human 21509 RNA, detected using Taqman analysis, in a panel of human tissues, including blood vessels (arteries, veins, smooth muscle cells; columns 1-5), heart (columns 6-7), kidney (column 8), skeletal muscle (column 9), adipose (column 9), pancreas (column 10), osteoblasts (column 11), osteoclasts (12), skin (columns 13 and 42), neurons (columns 15 and 18-19), brain (columns 16-17), glial cells (columns 20-21), brest (columns 22-23), ovary (columns 24-25), prostate (columns 26-27), epithelial cells (column 28), colon (column 29-30 and 34), lung (columns 31-33), liver (columns 35-36), dermal fibroblasts (column 37), spleen (column 38), tonsil (column 39), lymph node (column 40), and bone marrow (column 44), as well as some tumorous tissues, including glial (column 21), breast (column 23), ovary (column 25), prostate (column 27), colon (column 30), and lung (column 32) tumors. Expression of 21509 RNA was detected in many of the samples analyzed, with the most notable expression occurring in brain, epithelial cell, kidney, endothelial cell, and glial cell tissues. Increased expression of human 21509 RNA was detected in colon (column 30) and lung (column 32) tumor samples, as compared to normal colon (column 29) and lung (column 31) samples, respectively. Decreased expression of human 21509 RNA was detected in a glialblastoma (column 21) and an ovary (column 25) tumor, as compared to normal glial (column 20) and ovary (column 24) tissues, respectively.

FIG. 12 depicts expression of 21509 RNA, detected using Taqman analysis, in a panel of normal human tissues and tumors derived from those tissues, including breast (columns 1-5, normal; columns 6-9, tumors), ovary (columns 10-11, normal; columns 12-16, tumors), lung (columns 17-19, normal; columns 20-26, tumors), and colon (columns 28-30, normal; columns 31-36, tumors). In all classes, at least one of the tumor samples contained elevated expression of human 21509 relative to the normal tissue samples, e.g., columns 7 and 9 (breast tumors), column 13 (ovary tumor), columns 20-21 and 24 (lung tumors), and columns 31-33 and 35-36 (colon tumors).

FIG. 13 depicts expression of human 21509 RNA, detected using Taqman analysis, in a panel of human tissues, including breast, lung, colon, and liver. “T” denotes a tumor sample; “N” denotes a normal sample, and “Met” denotes a metastatic tumor sample. In three lung tumor samples (columns 13, 16, and 18) and two colon tumor samples (columns 24 and 26) expression of human 21509 RNA exceeded the level of expression observed in any of the normal lung and colon tissue samples, respectively.

FIGS. 14A-14B depicts a cDNA sequence (SEQ ID NO:16) and predicted amino acid sequence (SEQ ID NO:17) of human 33770. The methionine-initiated open reading frame of human 33770 (without the 5′ and 3′ untranslated regions of SEQ ID NO: 16) is shown also as coding sequence SEQ ID NO:18.

FIG. 15 is a hydropathy plot of human 33770. Relative hydrophobic residues are indicated above the dashed horizontal line, and relative hydrophilic residues are indicated below the dashed horizontal line. Numbers correspond to the amino acid sequence of human 33770. Polypeptides of the invention include 33770 fragments which include: all or part of a hydrophobic sequence (a sequence above the dashed line, e.g., the sequence of 140-175); all or part of a hydrophillic sequence (a sequence below the dashed line, e.g., the sequence of 80-90 or 15-35).

FIG. 16 depicts an alignment of human 33770 (SEQ ID NO:17) with a consensus amino acid sequence, derived from a hidden Markov model (PFAM Accession Number PF00171), from PFAM. The upper sequence is the consensus sequence for an aldehyde dehydrogenase domain (SEQ ID NO:21), while the lower sequence corresponds to amino acids 17 to 487 of human 33770 (SEQ ID NO:17).

FIG. 17 depicts a hydropathy plot of human 46638. Relative hydrophobic residues are shown above the dashed horizontal line, and relative hydrophilic residues are below the dashed horizontal line. The cysteine residues (cys) are indicated by short vertical lines just below the hydropathy trace. The numbers corresponding to the amino acid sequence of human 46638 are indicated. Polypeptides of the invention include fragments which include: all or part of a hydrophobic sequence, i.e., a sequence above the dashed line, e.g., the sequence of from about amino acid residue 20 to 30, from 580 to 583, and from 643 to 645 of SEQ ID NO:23; all or part of a hydrophilic sequence, i.e., a sequence below the dashed line, e.g., the sequence from about amino acid residue 508 to 510 and from 603 to 621 of SEQ ID NO:23; or a sequence which includes a Cys, or an N-glycosylation site.

FIGS. 18A-18B depicts an alignment of the lipoxygenase domain of human 46638 with consensus amino acid sequences derived from a hidden Markov model (HMM) from PFAM. The upper sequence is the consensus amino acid sequence for a lipoxygenase domain (SEQ ID NO:25), while the lower amino acid sequence corresponds to amino acids 267 to 703 of SEQ ID NO:23.

FIGS. 19A-19B depict alignments of HMM consensus sequences for the PLAT (Polycystin-1, Lipoxygenase Alpha-Toxin) domain and LH2 (Lipoxygenase Homology) domain using PFAM and SMART programs, respectively, with the human 46638 amino acid sequence. In FIG. 19A, the upper sequence is the consensus amino acid sequence for a PLAT domain from PFAM (SEQ ID NO:26), while the lower amino acid sequence corresponds to amino acids 2 to 116 of SEQ ID NO:23. In FIG. 19B, the upper sequence is the HMM consensus amino acid sequence for an LH2 domain from SMART (SEQ ID NO:27), while the lower amino acid sequence corresponds to amino acids 2 to 116 of SEQ ID NO:23.

FIG. 20 depicts a hydropathy plot of human 50090. Relative hydrophobic residues are shown above the dashed horizontal line, and relative hydrophilic residues are below the dashed horizontal line. The cysteine residues (cys) are indicated by short vertical lines just below the hydropathy trace. The numbers corresponding to the amino acid sequence of human 50090 are indicated. Polypeptides of the invention include fragments that include: all or part of a hydrophobic sequence, i.e., a sequence above the dashed line, e.g., the sequence of from about amino acid residue 70 to 79, amino acid residue 91 to 105, and amino acid residue 235 to 251 of SEQ ID NO:29; all or part of a hydrophilic sequence, i.e., a sequence below the dashed line, e.g., the sequence from about amino acid residues 31 to 55, amino acid residues 106 to 123, and amino acid residues 215 to 235 of SEQ ID NO:29; or a sequence which includes a Cys residue.

FIG. 21 depicts alignment of the enoyl-CoA hydratase/isomerase domain of human 50090 with a consensus amino acid sequence derived from hidden Markov models using the PFAM (ECH) program. The upper sequence is the consensus amino acid sequence (SEQ ID NO:31), while the lower amino acid sequence corresponds to amino acids 57 to 255 of SEQ ID NO:29.

### DETAILED DESCRIPTION OF THE 33312, 33303, AND 32579 INVENTION HUMAN 33312

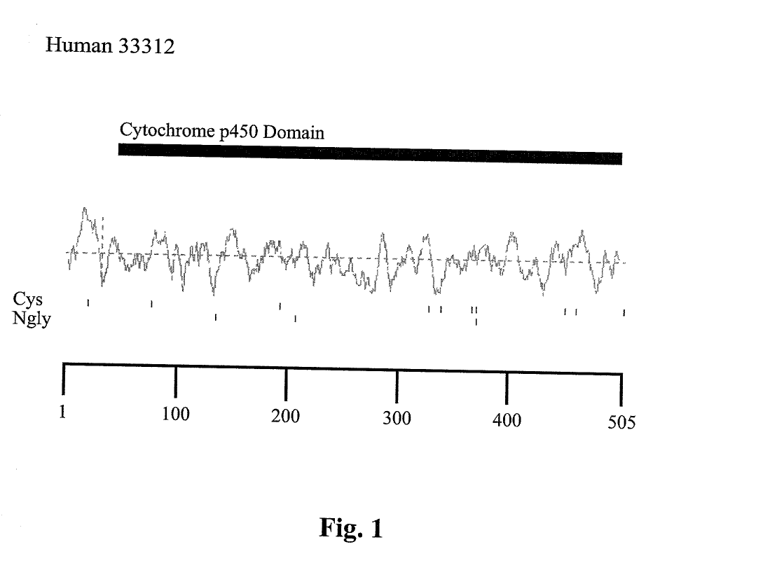

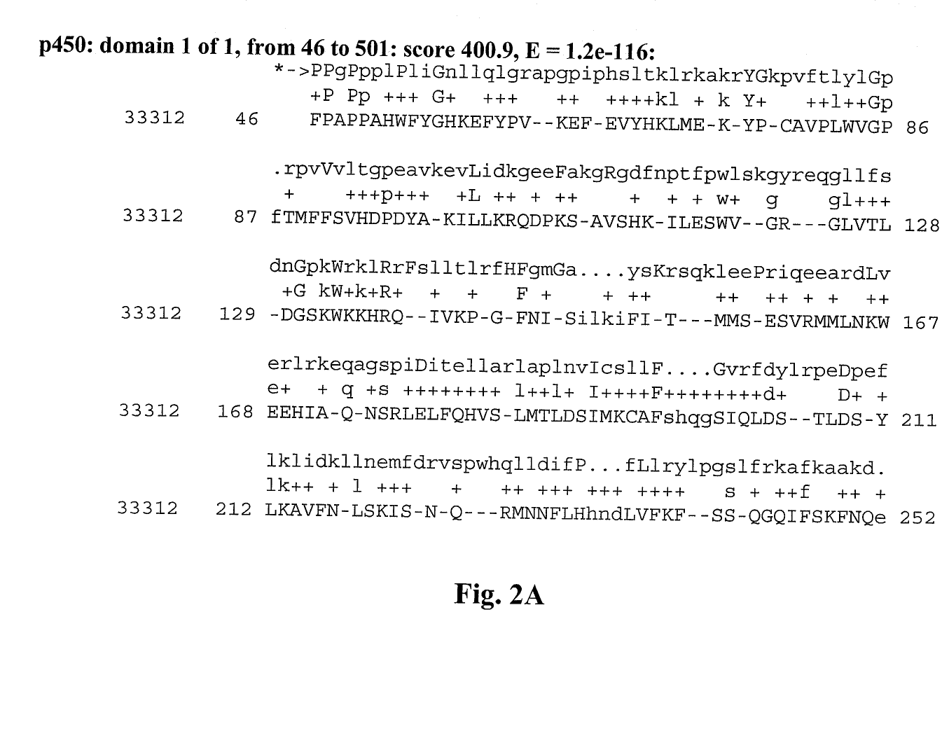

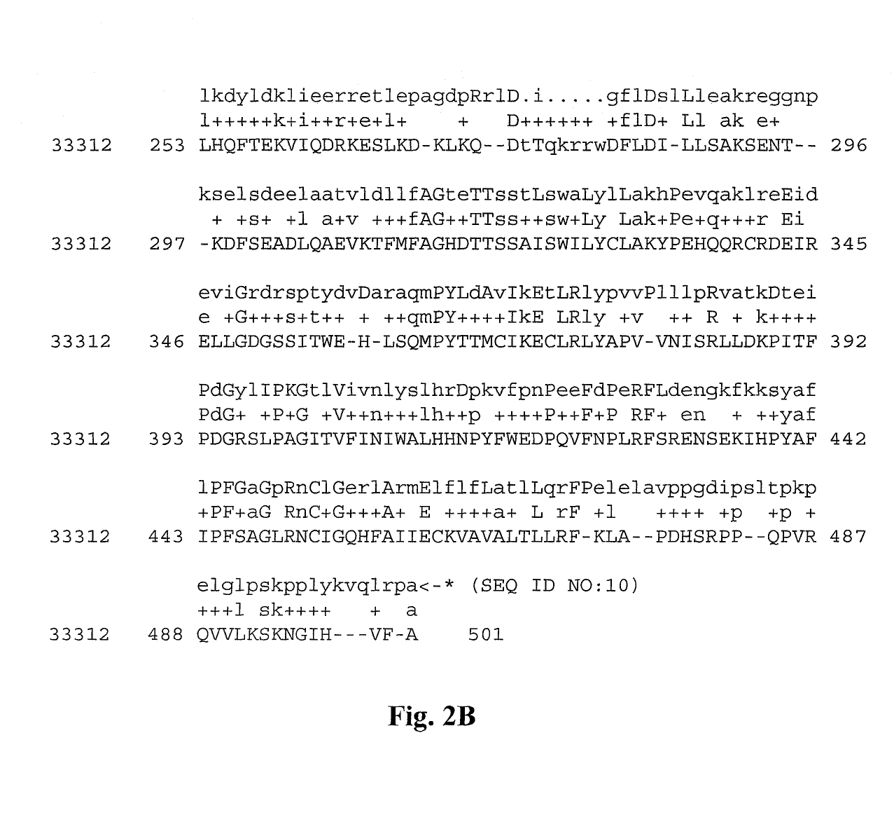

The human 33312 sequence (FIGS. 1 and 2; SEQ ID NO: 1), which is approximately 1975 nucleotides long including untranslated regions, contains a predicted methionine-initiated coding sequence of about 1518 nucleotides. The coding sequence encodes an 505 amino acid protein (SEQ ID NO:2). The human 33312 protein of SEQ ID NO:2 includes an amino-terminal hydrophobic amino acid sequence, consistent with a signal sequence, of about 33 amino acids (from amino acid 1 to about amino acid 33 of SEQ ID NO:2) (See FIG. 1), which upon cleavage results in the production of a mature protein form. This mature protein form is approximately 472 amino acid residues in length (from about amino acid 34 to amino acid 505 of SEQ ID NO:2).

The mature form of human 33312 contains the following regions or other structural features:

A cytochrome P450 domain located at about amino acid 46 to 501 of SEQ ID NO:2;

a cytochrome P450 cysteine heme-iron ligand signature (PS00086) from about amino acid 445 to 454 of SEQ ID NO:2;

three N-glycosylation sites (PS00001) located from about amino acid 145 to 148, from about amino acid 217 to 220, and from about amino acid 381 to 384, of SEQ ID NO:2;

one cAMP and cGMP-dependent protein kinase phorylation site (PS00004) from about amino acid 264 to 267 of SEQ ID NO:2;

seven protein kinase C phosphorylation sites (PS00005) from about amino acid 113 to 115, from about amino acid 159 to 161, from about amino acid 257 to 259, from about amino acid 267 to 269, from about amino acid 277 to 279, from about amino acid 290 to 292, and from about amino acid 434 to 436, of SEQ ID NO:2;

six casein kinase II phosphorylation sites (PS00006) from about amino acid 92 to 95, from about amino acid 175 to 178, from about amino acid 206 to 209, from about amino acid 267 to 270, from about amino acid 300 to 303, and from about amino acid 391 to 394, of SEQ ID NO:2; and

four N-myristoylation sites (PS00008) from about amino acid 243 to 248, from about amino acid 351 to 356, from about amino acid 448 to 453, and from about amino acid 454 to 459 of SEQ ID NO:2.

Human 33303

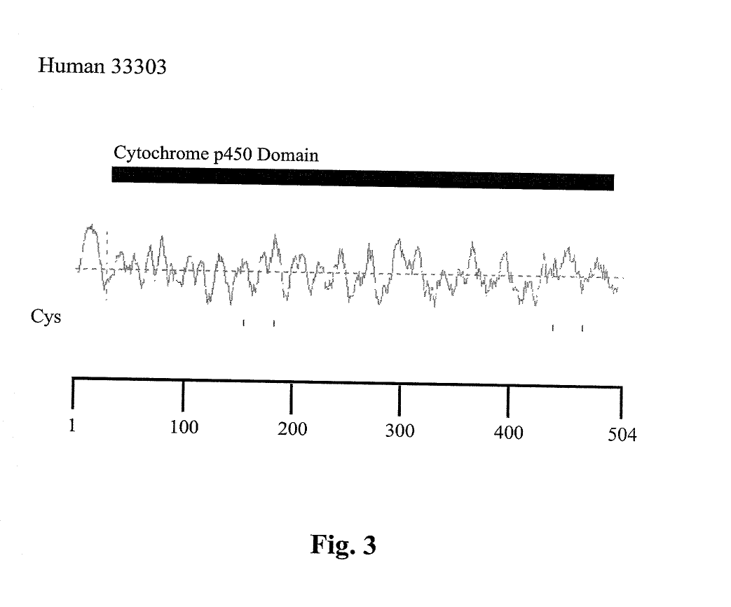

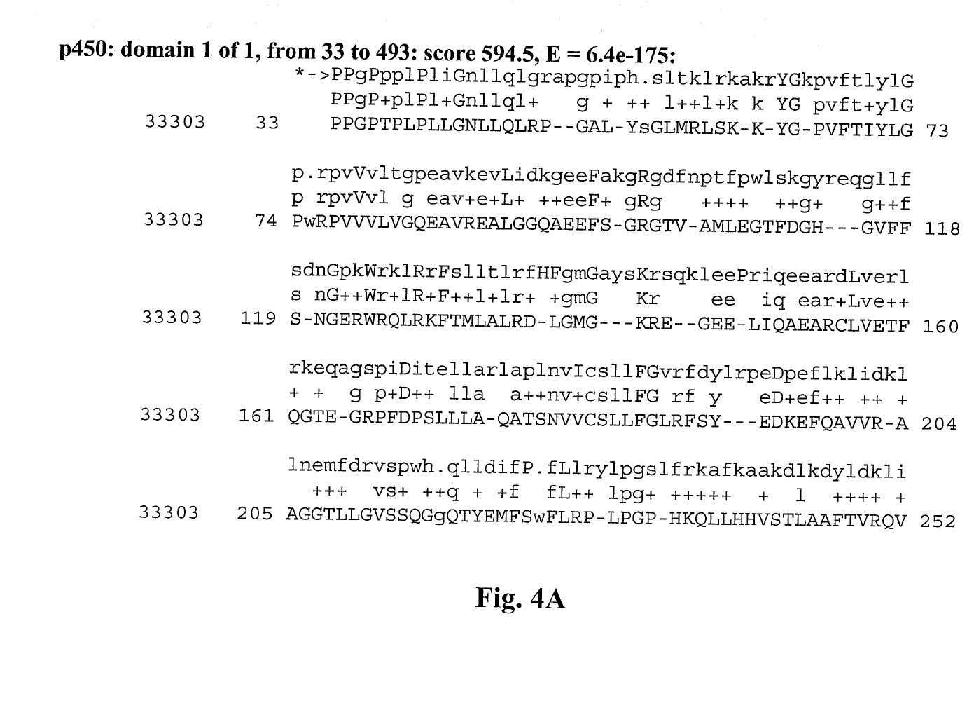

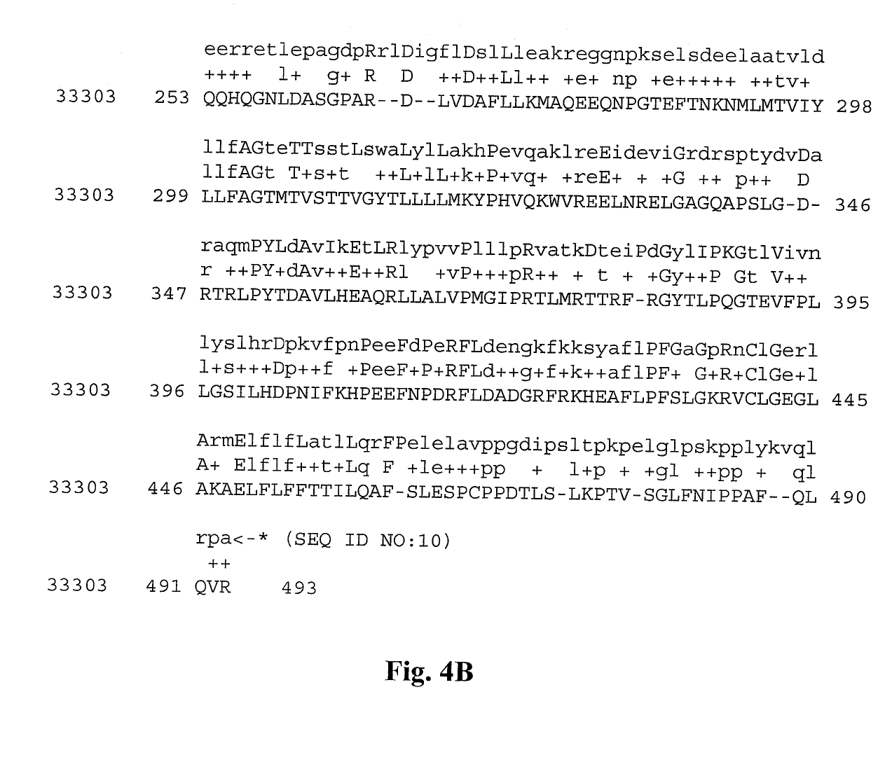

The human 33303 sequence (FIGS. 3 and 4; SEQ ID NO:4), which is approximately 1927 nucleotides long including untranslated regions, contains a predicted methionine-initiated coding sequence of about 1515 nucleotides. The coding sequence encodes an 504 amino acid protein (SEQ ID NO:5). The human 33303 protein of SEQ ID NO:5 includes an amino-terminal hydrophobic amino acid sequence, consistent with a signal sequence, of about 29 amino acids (from amino acid 1 to about amino acid 29 of SEQ ID NO:5) (See FIG. 3), which upon cleavage results in the production of a mature protein form. This mature protein form is approximately 474 amino acid residues in length (from about amino acid 30 to amino acid 504 of SEQ ID NO:5).

The mature form of human 33303 contains the following regions or other structural features:

A cytochrome P450 domain located at about amino acid 33 to 493 of SEQ ID NO:5;

a cytochrome P450 cysteine heme-iron ligand signature (PS00086) from about amino acid 433 to 442 of SEQ ID NO:5;

a leucine zipper pattern (PS00029) from about amino acid 32 to 53 of SEQ ID NO:5;

one glycosaminoglycan attachment site (PS00002) located from about amino acid 99 to 102 of SEQ ID NO:5;

one cAMP and cGMP-dependent protein kinase phorylation site (PS00004) from about amino acid 128 to 131 of SEQ ID NO:5;

six protein kinase C phosphorylation sites (PS00005) from about amino acid 61 to 63, from about amino acid 99 to 101, from about amino acid 248 to 250, from about amino acid 288 to 290, from about amino acid 378 to 380, and from about amino acid 473 to 475, of SEQ ID NO:5;

three casein kinase II phosphorylation sites (PS00006) from about amino acid 119 to 122, from about amino acid 192 to 195, and from about amino acid 343 to 346, of SEQ ID NO:5;

ten N-myristoylation sites (PS00008) from about amino acid 51 to 56, from about amino acid 109 to 114, from about amino acid 115 to 120, from about amino acid 188 to 193, from about amino acid 207 to 212, from about amino acid 257 to 261, from about amino acid 284 to 289, from about amino acid 339 to 344, from about amino acid 370 to 375, and from about amino acid 444 to 449, of SEQ ID NO:5; and

two amidation sites (PS00009) from about amino acid 140 to 143, and from about amino acid 435 to 438, of SEQ ID NO:5.

Human 32579

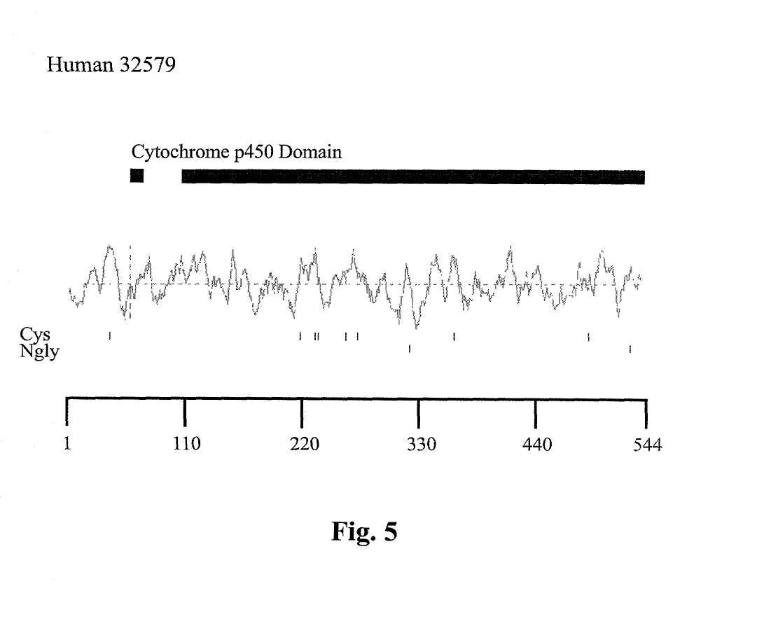

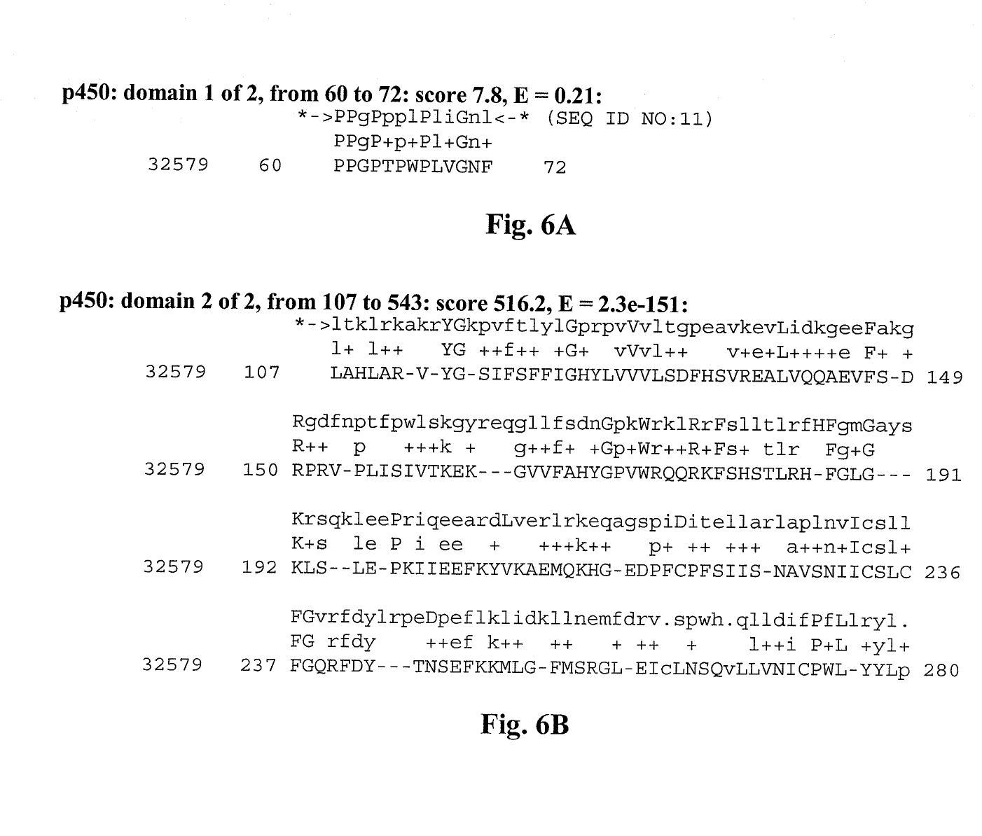

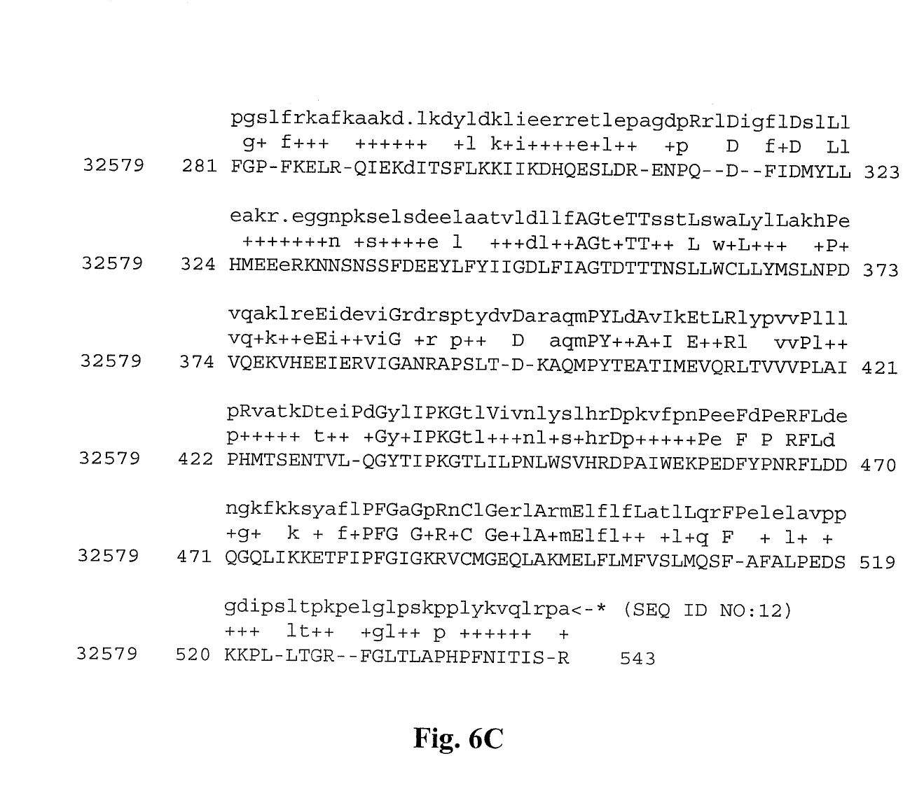

The human 32579 sequence (FIGS. 5 and 6; SEQ ID NO:7), which is approximately 2099 nucleotides long including untranslated regions, contains a predicted methionine-initiated coding sequence of about 1635 nucleotides. The coding sequence encodes an 544 amino acid protein (SEQ ID NO:8). The human 32579 protein of SEQ ID NO:8 includes an amino-terminal hydrophobic amino acid sequence, consistent with a signal sequence, of about 59 amino acids (from amino acid 1 to about amino acid 59 of SEQ ID NO:8) (See FIG. 5), which upon cleavage results in the production of a mature protein form. This mature protein form is approximately 484 amino acid residues in length (from about amino acid 60 to amino acid 544 of SEQ ID NO:8).

The mature form of human 32579 contains the following regions or other structural features:

one cytochrome P450 domain located at about amino acid 60 to about 543 of SEQ ID NO:8;

a cytochrome P450 cysteine heme-iron ligand signature (PS00086) from about amino acid 483 to 492 of SEQ ID NO:8;

a growth factor and cytokines receptors family signature (PS00241) from about amino acid 262 to 275 of SEQ ID NO:8;

two N-glycosylation sites (PS00001) from about amino acid 331 to 334, and from about amino acid 538 to 541, of SEQ ID NO:8;

three cAMP and cGMP-dependent protein kinase phorylation sites (PS00004) from about amino acid 82 to 85, from about amino acid 178 to 181, and from amino acid 476 to 479, of SEQ ID NO:8;

eight protein kinase C phosphorylation sites (PS00005) from about amino acid 88 to 90, from about amino acid 135 to 137, from about amino acid 148 to 150, from about amino acid 184 to 186, from about amino acid 395 to 397, from about amino acid 519 to 521, from about amino acid 525 to 527, and from about amino acid 542 to 544, of SEQ ID NO:8;

five casein kinase II phosphorylation sites (PS00006) from about amino acid 135 to 138, from about amino acid 244 to 247, from about amino acid 335 to 338, from about amino acid 393 to 396, and from about amino acid 406 to 409, of SEQ ID NO:8;

one tyrosine kinase phosphorylation site (PS00007) from about amino acid 198 to 205 of SEQ ID NO:8;

five N-myristoylation sites (PS00008) from about amino acid 95 to 100, from about amino acid 115 to 120, from about amino acid 164 to 169, from about amino acid 258 to 263, and from about amino acid 353 to 358 of SEQ ID NO:8; and

one amidation site (PS00009) from about amino acid 485 to 488 of SEQ ID NO:8.

For general information regarding PFAM identifiers, PS prefix and PF prefix domain identification numbers, refer to Sonnhammer et al. (1997) Protein 28:405-420 and http://www.psc.edu/general/software/packages/pfam/pfam.html.

The 33312, 33303, and 32579 molecules belong to the cytochrome P450 family of molecules having conserved structural and functional features. The term “family” when referring to the protein and nucleic acid molecules of the invention means two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin, or alternatively, can contain homologues of non-human origin, e.g., rat or mouse proteins. Members of a family can also have common functional characteristics.

Cytochrome P450 domain family members have at least one P450 domain, which is characterized by an approximately 400 to 530 amino acid sequence that typically has a signature motif which includes a conserved cysteine residue in the C-terminal region that is involved in binding a heme iron (Nebert et al. (1987) Annu. Rev. Biochem. 56, 945-993). P450 family proteins catalyze a variety of oxidative reactions in the metabolism of endogenous and exogenous hydrophobic substrates (Guengerich, F. P. (1991) J. Biol. Chem. 266, 10019-10022), and their physiological effects cover the spectrum from being required for normal growth and differentiation to the activation of carcinogenic compounds.

A 33312, 33303, or 32579 polypeptide can include at least one “cytochrome P450 domain” or regions homologous with a “cytochrome P450 domain.” As used herein, the term “cytochrome P450 domain” also refers to a protein domain having amino sequence of about 300 to about 600 amino acid resides in length, preferably of about 350 to 500, more preferably of about 400 to 490 amino acids and having a bit score for the alignment of the sequence to the P450 domain (HMM) of at least 300, preferably 350, more preferably 400 or greater. An alignment of the cytochrome P450 domain (amino acids 46 to 501, 33 to 493, 107 to 543 of SEQ ID NO:2, SEQ ID NO:5, or SEQ ID NO:8) of 33312, 33303, or 32579, respectively, with a consensus amino acid sequence derived from a hidden Markov model is depicted in FIGS. 2A-2B, 4A-4B, or 6A-6C.

Preferably, a cytochrome P450 domain contains the [FW]-[SGNH]-X-[GD]-X-[RKHPT]-X—C-[LIVMFAP]-[GAD] motif at its C-terminal part, wherein X can be any amino acid. For example, the P450 domain of a 33312 polypeptide has the sequence F-S-A-G-L-R-N-C-L-G which matches this motif at position about 445 to 454 of SEQ ID NO:2; the P450 domain of a 33303 polypeptide has the sequence F-S-L-G-K-R-V-C-L-G which matches this motif at position about 433 to 442 of SEQ ID NO:5; and the P450 domain of a 32579 polypeptide has the sequence F-G-1-G-K-R-V-C-M-G which matches this motif at position about 483 to 492 of SEQ ID NO:8.

In a preferred embodiment, a 33312, 33303, or 32579 cytochrome P450 polypeptide or protein has a “P450 domain” or a region which includes at least about 300 to 600, more preferably about 400 to 500 or 430 to 460 amino acid residues and has at least about 60%, 70%, 80%, 90%, 95%, 99%, or 100% homology with a “P450 domain,” e.g., the P450 domain of human 33312 (e.g., residues 46 to 501 of SEQ ID NO:2), the P450 domain of human 33303 (e.g., residues 33 to 493 of SEQ ID NO:5); or the P450 domain of human 32579 (e.g., residues 60 to 543 of SEQ ID NO:8).

A 32579 polypeptide can additionally include a second cytochrome P450 domain, an alignment of which (e.g., amino acids 60 to 72 of SEQ ID NO:8) with a consensus amino acid sequence derived from a hidden Markov model is depicted in FIG. 6.

To identify the presence of a “cytochrome P450” domain” in a 33312, 33303, or 32579 protein sequence, and make the determination that a polypeptide or protein of interest has a particular profile, the amino acid sequence of the protein can be searched against a database of HMMs (e.g., the Pfam database, release 2.1) using the default parameters (http://www.sanger.ac.uk/Software/Pfam/HMM_search). For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al.(1990) Meth. Enzymol. 183:146-159; Gribskov et al.(1987) Proc. Natl. Acad. Sci. USA 84:4355-4358; Krogh et al.(1994) J. Mol. Biol. 235:1501-1531; and Stultz et al.(1993) Protein Sci. 2:305-314, the contents of which are incorporated herein by reference.

A 33312, 33303, or 32579 protein can further include a signal sequence. As used herein, a “signal peptide” or “signal sequence” refers to a peptide of about 1-60, preferably about 1 to 59, more preferably, about 29, 33, or 59 amino acid residues in length which occurs at the N-terminus of secretory and integral membrane proteins and which contains a majority of hydrophobic amino acid residues. For example, the signal sequence has at least about 40-70%, preferably about 50-65%, and more preferably about 55-60% hydrophobic amino acid residues (e.g., alanine, valine, leucine, isoleucine, phenylalanine, tyrosine, tryptophan, or proline). Such a “signal sequence”, also referred to in the art as a “signal peptide”, serves to direct a protein containing such a sequence to a lipid bilayer. For example, in one embodiment, a 33312 protein contains a signal sequence of about amino acids 1 to 33 of SEQ ID NO:2. The “signal sequence” is cleaved during processing of the mature protein. The mature 33312 protein corresponds to amino acids 34 to 505 of SEQ ID NO:2. In another embodiment, a 33303 protein contains a signal sequence of about amino acids 1 to 29 of SEQ ID NO:5. The “signal sequence” is cleaved during processing of the mature protein. The mature 33303 protein corresponds to amino acids 30 to 504 of SEQ ID NO:5. In yet another embodiment, a 32579 protein contains a signal sequence of about amino acids 1 to 59 of SEQ ID NO:8. The “signal sequence” is cleaved during processing of the mature protein. The mature 32579 protein corresponds to amino acids 60 to 544 of SEQ ID NO:8.

A 33303 protein can further include a leucine zipper sequence. As used herein, a “leucine zipper peptide” or “leucine zipper sequence” refers to an amino acid sequence of about 10 to 40, preferably about 20 to 30, more preferably, 21 amino acid residues in length which contains various numbers of leucines at various positions. Leucine zipper patterns are typically present in many gene regulatory proteins, such as CCATT-box and enhancer binding protein (C/EBP), cAMP response element (CRE) binding proteins (CREB, CRE-BP1, ATFs), jun/AP1 family transcription factors, C-myc, L-myc and N-myc oncogenes and octamer-binding transcription factor 2 (Oct-2/OTF-2). These interactions are frequently required for the activity of the protein complex, e.g., transcriptional activation of a nucleic acid via binding to a gene regulatory sequence and subsequent formation of a transcription initiation complex. Leucine zippers therefore mediate protein-protein interactions in vivo and in particular, interactions between multi-subunit transcription factors (homodimers, heterodimers, etc.). In one embodiment, a 33303 protein contains a leucine zipper sequence of about amino acids 32 to 53 of SEQ ID NO:5.

A 32579 protein can further include a growth factor and cytokines receptors family signature sequence. As used herein, a “growth factor and cytokines receptors family signature peptide” or “growth factor and cytokines receptors family signature sequence” refers to a peptide of about 5 to 30, preferably about 10 to 20, more preferably, 13 amino acid residues in length and having a sequence at least 85%, 90%, 95%, 99% or more homologous to a cytokine receptor family signature sequence of about amino acids 262 to 275 of SEQ ID NO:8.

A 33312 polypeptide can optionally further include at least one, two and preferably three glycosylation site; at least one cAMP/cGMP phosphorylation site; at least one, two, three, four, five, six, and preferably seven protein kinase C phosphorylation sites; at least one, two, three, four, five, and preferably six casein kinase II phosphorylation sites; at least one, two, three, and preferably four N-myristylation sites.

A 33303 polypeptide can optionally further include at least one, glycosaminoglycan attachment site; at least one cAMP/cGMP phosphorylation site; at least one, two, three, four, five, and preferably six protein kinase C phosphorylation sites; at least one, two, and preferably three casein kinase II phosphorylation sites; at least one, two, three, four, five, six, seven, eight, nine, and preferably ten N-myristylation sites; and at least one, preferably two amidation sites.

A 32579 polypeptide can optionally further include at least one, and preferably two glycosylation sites; at least one, two, and preferably three cAMP/cGMP phosphorylation sites; at least one, two, three, four, five, six, seven, and preferably eight protein kinase C phosphorylation sites; at least one, two, three, four, and preferably five casein kinase II phosphorylation sites; at least one tyrosine phosphorylation site; at least one, two, three, four, and preferably five N-myristylation sites; and at least one amidation site.

As the 33312, 33303, or 32579 polypeptides of the invention may modulate 33312-, 33303-, 32579-mediated activities, they may be useful for developing novel diagnostic and therapeutic agents for treating disorders related to such activities, as described below.

Based on the above-described sequence similarities, the 33312, 33303, or 32579 molecules of the present invention are predicted to have similar biological activities as cytochrome P450 family members. Thus, in accordance with the invention, a 33312, 33303, or 32579 cytochrome P450 or subsequence or variant polypeptide may have one or more domains and, therefore, one or more activities or functions characteristic of a cytochrome P450 family member, including, but not limited to, a cytochrome P450 domain, a cysteine heme-iron ligand signature, leucine zipper pattern, and/or growth factor and cytokines receptors family signature. Thus, the 33312, 33303, or 32579 molecules can act as novel diagnostic targets and therapeutic agents for controlling cytochrome P450 associated disorders.

As used herein, the terms “33312, 33303, or 32579 activity,” or “33312, 33303, or 32579 function,” when used in reference to a 33312, 33303, or 32579 cytochrome P450 molecule means an activity or function exerted by a 33312, 33303, or 32579 cytochrome P450 molecule on another molecule (e.g., a target substrate or binding partner) or a cell, a tissue or an organism that responds to the particular 33312, 33303, or 32579 activity or function, as determined in vivo or in vitro. Activities or functions can be direct, e.g., through binding or modification of a target substrate or binding partner, providing a signal, etc., or indirect, e.g., through binding or modification of a substrate by 33312, 33303, or 32579 cytochrome P450 which, in turn, directly or indirectly (through one or more intermediates) confers a signal that results in effecting 33312, 33303, or 32579 cytochrome P450 molecule activity or function.

As used herein, the term “cytochrome P450 activity,” “biological activity of cytochrome P450”, or “functional activity of cytochrome P450” when used in reference to a protein, means a protein having the ability to oxidize a substrate in the presence of heme-iron complex. Thus, a 33312, 33303, or 32579 cytochrome P450 or subsequence or variant having cytochrome P450 activity is capable of oxidization of a substrate in the presence of heme-iron complex. Exemplary P450 activities mediated by the molecules of the invention include or more of the following activities: (1) modulating extracellular matrix environment; (2) acting as a structural component of extracellular matrix; (3) regulating cell signaling; (4) modulating metabolism of proteins, carbohydrates, and lipids; (5) catalyzing oxidation reactions in the metabolism of endogenous and exogenous substrates; (6) capable of modulating steroid metabolism; (7) capable of modulating fatty acids metabolism; (8) capable of activating and detoxifying low molecular carcinogens and other toxins; or (9) capable of regulating drug metabolism. Thus, the 33312, 33303, or 32579 molecules can act as novel diagnostic targets and therapeutic agents for controlling cytochrome P450 associated disorder.

The 33312, 33303, or 32579 cytochrome P450 molecules find use in modulating 33312, 33303, or 32579 cytochrome P450 function, activity, or expression, or related responses to cytochrome P450 function, activity or expression. As used herein, the term “modulate” or grammatical variations thereof means increasing or decreasing an activity, function, signal or response. That is, the 33312, 33303, or 32579 cytochrome P450 molecules of the invention affect the targeted activity in either a positive or negative fashion (e.g., increase or decrease activity, function, or signal).

As used herein, a “cytochrome P450 associated disorder” includes a disorder, disease or condition which is characterized by a misregulation of a cytochrome P450 mediated activity. Cytochrome P450 associated disorders can detrimentally affect cell proliferation, cell adhesion, cell motility and migration, inflammatory response, cell signaling, metabolism, steroid metabolism, fatty acids metabolism, harmful compounds detoxification, drug metabolism, and others. Thus, examples of cytochrome P450 associated disorders in which the 33312, 33303, or 32579 molecules of the invention may be directly or indirectly involved include cellular proliferative and/or differentiative disorders; disorders associated with undesirable or deficient cell adhesion, motility or migration; inflammatory disorders, cell signaling associated disorders, metabolism associated disorders, steroids associated disorders; and fatty acid associated disorders.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary.

The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease. 33312, 33303, or 32579 polypeptide may be involved controlling one or more of neurite outgrowth, central nervous system (CNS) development, psychiatric function, and neuronal repair. Examples of CNS disorders include neurodegenerative disorders, e.g., Alzheimer's disease, dementias related to Alzheimer's disease (such as Pick's disease), Parkinson's and other Lewy diffuse body diseases, multiple sclerosis, amyothrophic lateral sclerosis, progressive supranuclear palsy, epilepsy, and Jakob-Creutzfieldt disease; psychiatric disorders, e.g., depression, schizophrenic disorders, Korsakoffs psychosis, mania, anxiety disorders, or phobic disorders; learning or memory disorders, e.g., amnesia or age-related memory loss; and neurological disorders, e.g., migraine.

Additionally, 33312, 33303, or 32579 may play an important role in the regulation of metabolism, e.g., disorders related steroid metabolism, or fatty acids metabolism. Examples of metabolic disorders include, but are not limited to, obesity, anorexia nervosa, cachexia, lipid disorders, and diabetes.

The 33312, 33303, or 32579 nucleic acid and protein of the invention can be used to treat and/or diagnose a variety of immune or hematopoietic disorders. Examples of hematopoieitic disorders or diseases include, but are not limited to, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjogren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy such as, atopic allergy.

As the 33303 polypeptides contain a predicted leucine zipper, these polypeptides mediate protein-protein interactions in vivo and in particular, interactions between multi-subunit transcription factors (homodimers, heterodimers, etc.) Thus, in another embodiment, a polypeptide of the invention or subsequence or variant may have one or more activities of a leucine zipper motif, such as binding to another polypeptide that has a leucine zipper, for example, forming a dimer with a 33303 cytochrome P450 protein or subsequence or variant containing a leucine zipper. The presence of a leucine zipper indicates 33303 cytochrome P450 protein may participate in different pathways due to an ability to interact with different proteins via the leucine zipper. Therefore, the 33303 cytochrome P450 protein molecules of the invention may also be useful in modulating the various pathways in which this polypeptide participates.

In one embodiment, the invention provides methods and compositions for the treatment or control of 33312, 33303, or 32579 cytochrome P450 related disorders in cells/tissues that do not normally express 33312, 33303, or 32579 cytochrome P450.

The 33312, 33303, or 32579 cytochrome P450 molecules also find use in diagnosis of disorders involving an increase or decrease in 33312, 33303, or 32579 cytochrome P450 expression relative to normal expression, such as a proliferative disorder, a differentiative disorder (e.g., cancer), an immune disorder, a motility disorder, a vascular disorder, a bleeding or clotting disorder, or a developmental disorder. Thus, where expression or activity of 33312, 33303, or 32579 cytochrome P450 is greater or less than normal, this may indicate the presence of or a predisposition towards a 33312, 33303, or 32579 cytochrome P450 disorder. The presence of 33312, 33303, or 32579 cytochrome P450 RNA or protein, e.g., by hybridization of a 33312, 33303, or 32579 specific probe or with a 33312, 33303, or 32579 specific antibody, can be used to identify the amount of 33312, 33303, or 32579 present in a particular cell or tissue, or other biological sample. 33312, 33303, or 32579 activity (protease activity assays, adhesion assays, binding assays, motility/migration assays, vascularization assays, etc.) can be assessed using the various techniques described herein or otherwise known in the art. Thus, in another embodiment, the invention provides methods and compositions for detection of 33312, 33303, or 32579 cytochrome P450 in tissues that normally or do not normally express 33312, 33303, or 32579 cytochrome P450.

The compositions of the invention include, inter alia, 33312, 33303, or 32579 cytochrome P450 polypeptides, variants and subsequences thereof, referred to as “polypeptides or proteins of the invention” or “33312, 33303, or 32579 cytochrome P450 polypeptides or proteins;” nucleic acids that encode 33312, 33303, or 32579 cytochrome P450 variants and subsequences thereof, or that hybridize to such sequences, referred to as “nucleic acids of the invention” or “33312, 33303, or 32579 cytochrome P450 nucleic acids;” antibodies that bind cytochrome P450 polypeptides, variants and subsequences thereof; vectors including 33312, 33303, or 32579 cytochrome P450 nucleic acids, variants and subsequences thereof, referred to as “antibodies of the invention” or “33312, 33303, or 32579 cytochrome P450 antibodies;” and compounds that modulate expression or activity of the 33312, 33303, or 32579 cytochrome P450 polypeptides and polynucleotides, referred to as “compounds of the invention.” Collectively, these 33312, 33303, or 32579 cytochrome P450 related compositions are referred to as “33312, 33303, or 32579 cytochrome P450 molecules” or “molecules of the invention.

As used herein, the terms “nucleic acid,” “polynucleotides” or “oligonucleotides” include DNA molecules (e.g., cDNA or genomic DNA) and RNA molecules (e.g., an mRNA) and analogs of the DNA or RNA generated, e.g., by the use of nucleotide analogs. The nucleic acid molecule can be single- or double-stranded, linear or circular.

An “isolated nucleic acid” or “purified nucleic acid” is one that is separated from other nucleic acid present in the natural source of nucleic acid. Preferably, an “isolated” nucleic acid is free of sequences which naturally flank 33312, 33303, or 32579 cytochrome P450 nucleic acid (i.e., sequences located at the 5′ and 3′ ends of the nucleic acid) in the genomic DNA of the organism from which the nucleic acid is derived. However, there can be some flanking nucleotide sequences, for example up to about 5 Kb. For example, in various embodiments, the isolated nucleic acid can contain less than about 5 Kb, 4 Kb, 3 Kb, 2 Kb, 1 Kb, 0.5 Kb, 0.1 Kb of 5′ or 3′ nucleotide sequence that naturally flank the nucleic acid in genomic DNA. Moreover, an “isolated” nucleic acid molecule, such as a cDNA or RNA molecule, can be substantially free of other cellular material, or culture medium when produced by recombinant techniques, or chemical precursors or other chemicals when chemically synthesized. In one embodiment, the 33312, 33303, or 32579 cytochrome P450 nucleic acid comprises only the coding region. However, the nucleic acid molecule can be fused to other coding or regulatory sequences and still be considered isolated.

In some instances, the isolated material will form part of a composition (for example, a crude extract containing other substances), buffer system or reagent mix. For example, recombinant nucleic acid molecules contained in a vector are considered isolated. Further examples of isolated nucleic acid molecules include recombinant DNA molecules maintained in heterologous host cells or purified (partially or substantially) nucleic acid molecules in solution. Isolated RNA molecules include in vivo or in vitro RNA transcripts of the isolated DNA molecules of the invention. Isolated nucleic acid molecules according to the present invention further include such molecules produced synthetically. In other circumstances, the material may be purified to essential homogeneity, for example as determined by PAGE or column chromatography such as HPLC. Isolated nucleic acids typically comprise at least about 50, 80 or 90% (on a molar basis) of all macromolecular species present.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6×sodium chloride/sodium citrate (SSC) at about 45° C., followed by two washes in 0.2× SSC, 0.1% SDS at least at 50° C. (the temperature of the washes can be increased to 55° C. for low stringency conditions); 2) medium stringency hybridization conditions in 6× SSC at about 45° C., followed by one or more washes in 0.2× SSC, 0.1% SDS at 60° C.; 3) high stringency hybridization conditions in 6× SSC at about 45° C., followed by one or more washes in 0.2× SSC, 0.1% SDS at 65° C.; and preferably 4) very high stringency hybridization conditions are 0.5M sodium phosphate, 7% SDS at 65° C., followed by one or more washes at 0.2× SSC, 1% SDS at 65° C. Very high stringency conditions (4) are the preferred conditions and the ones that should be used unless otherwise specified.

As used herein, a “naturally-occurring” nucleic acid molecule refers to an RNA or DNA molecule having a nucleotide sequence that occurs in nature (e.g., encodes a natural protein).

As used herein, the terms “gene” and “recombinant gene” refer to nucleic acid molecules which include an open reading frame encoding a 33312, 33303, or 32579 cytochrome P450 protein, preferably a mammalian 33312, 33303, or 32579 cytochrome P450 protein, and can further include non-coding regulatory sequences, and introns.

As used herein, the terms “polypeptide,” peptide” or “protein” are used interchangeably to denote two or more amino acids covalently linked by an amide bond or equivalent (see, e.g., Spatola (1983) in Chemistry and Biochemistry of Amino Acids, Peptides and Proteins, Vol. 7, pp. 267-357, “Peptide and Backbone Modifications,” Marcel Decker, NY). The polypeptides of the invention are not limited with respect to their length. L- and D-isomers and sequences having combinations of L- and D-isomers also are included.

An “isolated” or “purified” polypeptide or protein is substantially free of contaminating material from which the polypeptide is obtained or derived. For example, when it is isolated from recombinant and non-recombinant cells, it is substantially free of cellular material or debris or culture medium, when it is chemically synthesized it is substantially free of chemical precursors or other chemicals. A polypeptide, however, can be joined to another polypeptide, covalently (a chimera or fusion) or non-covalently, with which it is not normally associated with in a cell and still be considered “isolated” or “purified.”

In one embodiment, the language “substantially free of cellular material” or “substantially free of chemical precursors or other chemicals” means preparations of 33312, 33303, or 32579 cytochrome P450 having less than about 30%, 20%, 10%, or more likely 5% (by dry weight) other (non-33312, 33303, or 32579 cytochrome P450) proteins (i.e., contaminating protein) or chemical precursors/other chemicals involved in its synthesis. When the polypeptide is recombinantly produced, it can also be substantially free of culture medium, i.e., culture medium represents less than about 20%, less than about 10%, or less than about 5% of the volume of the protein preparation. The invention includes isolated or purified preparations of at least 0.01, 0.1, 1.0 and 10 milligrams in dry weight. 33312, 33303, or 32579 cytochrome P450 polypeptides can be purified to homogeneity. It is understood, however, that preparations in which the polypeptide is not purified to homogeneity are useful and considered to contain an isolated form of the polypeptide. The critical feature is that the preparation allows for the desired function of the polypeptide, even in the presence of considerable amounts of other components. Thus, the invention encompasses various degrees of purity.

As used herein, the term “non-essential,” when used in reference to an amino acid residue means that the amino acid is not required for activity, i.e., substitution of the amino acid with another does not destroy activity of the 33312, 33303, or 32579 cytochrome P450. As used herein, the term “essential” means that the amino acid is required for activity, i.e., substitution of the amino acid with another may abolish one or more activities of the 33312, 33303, or 32579 cytochrome P450. For example, the catalytic heme binding site of 32579 is predicted to be unamenable to alteration without affecting heme binding function. In the example of a non-essential amino acid, both conservative and non-conservative substitutions are likely to be tolerated. In the example of an essential amino acid, a conservative substitution is likely to be tolerated, whereas a non-conservative substitution is unlikely to be tolerated.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 33312, 33303, or 32579 cytochrome P450 replaced with another amino acid residue from the same side chain family will likely have substantially the same activity.

Whether a particular amino acid of 33312, 33303, or 32579 cytochrome P450 is non-essential or essential can be determined using activity or functional assays described herein or known in the art. For example, mutations can be introduced randomly along all or part of a 33312, 33303, or 32579 cytochrome P450 coding sequence, such as by saturation mutagenesis (e.g., alanine-scanning mutagenesis, see, Cunningham et al. (1985) Science 244:1081-1085) or site-directed mutagenesis. The resulting variant is then tested for biological activity, such as peptide bond hydrolysis in vitro, or a related biological activity, such as proliferative, adhesion, motility/migration or vascularization activity to identify variants that retain activity or function. Thus, essential and non-essential amino acids can be identified empirically.

Guidance concerning which amino acid changes are likely to be tolerated also can be based upon the degree of sequence conservation in particular domains within the cytochrome P450 family. For example, a highly conserved sequence among many family members indicates that the amino acid are likely to be essential to a function. Less or non-conserved regions among family members are more likely to be composed of many non-essential amino acids. Guidance regarding amino acid substitutions also can be found in Bowie et al., Science 247:1306-1310 (1990). Sites that are critical for binding can also be determined by structural analysis such as crystallization, nuclear magnetic resonance or photoaffinity labeling (Smith et al. (1992) J. Mol. Biol. 224:899-904; de Vos et al. (1992) Science 255:306-312).

As used herein, a “biologically active portion” or “biologically active subsequence,” or “biologically functional portion” or “biologically functional subsequence” of a 33312,33303, or 32579 cytochrome P450 protein, includes a fragment of a 33312, 33303, or 32579 cytochrome P450 protein having one or more activities or functions of full length 33312, 33303, or 32579 cytochrome P450 set forth as SEQ ID NO:2, SEQ ID NO:5 and SEQ ID NO:8. For example, a biologically functional subsequence of a 33312, 33303, or 32579 cytochrome P450 may participate in an interaction with another molecule, such as a protein substrate. Biologically active portions of a 33312, 33303, or 32579 cytochrome P450 protein include peptides comprising amino acid sequences sufficiently homologous to or derived from the amino acid sequence of the 33312, 33303, or 32579 cytochrome P450 protein, e.g., the amino acid sequence shown in SEQ ID NO:2, SEQ ID NO:5 and SEQ ID NO:8, which include fewer amino acids than the full length 33312, 33303, or 32579 cytochrome P450 proteins, and exhibit at least one activity or function of a 33312, 33303, or 32579 cytochrome P450 protein, as set forth herein or otherwise known in the art for members of this family, e.g., monooxygenase, etc. A biologically active or functional portion of a 33312, 33303, or 32579 cytochrome P450 protein can be a polypeptide which is, for example, 10, 25, 50, 75, 100, 150, 200, 250, 300, 350, 400, 450, 500 or more amino acids in length. Biologically active portions of a 33312, 33303, or 32579 cytochrome P450 protein can be used as targets for developing agents which modulate a 33312, 33303, or 32579 cytochrome P450 mediated activity, e.g., protease, substrate binding, etc. Biologically active portions of a 33312, 33303, or 32579 cytochrome P450 protein also can be used as competitive inhibitors of an endogenous 33312, 33303, or 32579 cytochrome P450 which can therefore modulate a 33312, 33303, or 32579 cytochrome P450 mediated activity in vivo, e.g., monooxygenase, etc.

The term “substrate” is intended to refer not only to the peptide substrate that may be cleaved by cytochrome P450, but to refer to any component with which the 33312, 33303, or 32579 polypeptide interacts in order to produce an effect on that component or a subsequent biological effect that is a result of interacting with that component. This includes, but is not limited to, for example, interaction with extracellular matrix components, etc. However, it is understood that a substrate also includes peptides that are cleaved as a result of catalysis in a cytochrome P450 domain.

Particularly preferred 33312, 33303, 32579 polypeptides of the present invention have an amino acid sequence substantially identical to the amino acid sequence of SEQ ID NO:2. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 60%, or 65% identity, likely 75% identity, more likely 85%, 90%. 91%, 92%, 93%, 94%, 95%, 96%, 97%, 98% or 99% identity to SEQ ID NO:2 are termed substantially identical.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 60%, or 65% identity, likely 75% identity, more likely 85%, 90%. 91%, 92%, 93%, 94%, 95%, 96%, 97%, 98% or 99% identity to SEQ ID NO: 1 or 3 are termed substantially identical.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is typically at least 30%, or at least 40%, more typically at least 50%, even more typically at least 60%, or at least 70%, 80%, or 90% of the length of the reference sequence (e.g., when aligning a second sequence to the amino acid sequences herein having 1068 amino acid residues, at least 200, likely at least 300, more likely at least 400, even more likely at least 500, and most likely at least 600, 700, 800, or 900 amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In one embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particular set of parameters for identifying homologous sequences (and the one that should be used if the practitioner is uncertain about what parameters should be applied) is using a Blossum 62 scoring matrix with a gap open penalty of 12, a gap extend penalty of 4, and a frameshift gap penalty of 5.

The percent identity between two amino acid or nucleotide sequences can be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to 33312, 33303, or 32579 cytochrome P450 nucleic acid molecules of the invention. BLAST protein searches can be performed with the XBLAST program, score=50, wordlength=3 to obtain amino acid sequences homologous to 33312, 33303, or 32579 cytochrome P450 protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

“Misexpression or aberrant expression”, as used herein, refers to a non-wild type pattern of gene expression, at the RNA or protein level. It includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

“Subject”, as used herein, can refer to a mammal, e.g., a human, or to an experimental or animal or disease model. The subject can also be a non-human animal, e.g., a horse, cow, goat, or other domestic animal.

A “purified preparation of cells”, as used herein, refers to, in the case of plant or animal cells, an in vitro preparation of cells and not an entire intact plant or animal. In the case of cultured cells or microbial cells, it consists of a preparation of at least 10% and more preferably 50% of the subject cells.

Various aspects of the invention are described in further detail below.

Isolated Nucleic Acid Molecules of 33312, 33303, and 32579

The invention provides isolated or purified nucleic acid molecules that encode a 33312, 33303, or 32579 cytochrome P450 described herein, e.g., a full length 33312, 33303, or 32579 cytochrome P450 or fragment of SEQ ID NO:2, SEQ ID NO:5 or SEQ ID NO:8, e.g., a biologically active portion of 33312, 33303, or 32579 cytochrome P450. Also included are nucleic acid fragments suitable for use as a hybridization probe, which can be used, e.g., to identify a nucleic acid molecule encoding a polypeptide of the invention, such as 33312, 33303, or 32579 cytochrome P450 mRNA, and fragments suitable for use as primers, e.g., PCR primers for the amplification or mutation of nucleic acid molecules. The term “33312, 33303, or 32579 cytochrome P450 nucleic acid” or “33312, 33303, or 32579 cytochrome P450 polynucleotide” 1 includes variants and subsequences or fragments of 33312, 33303, or 32579 cytochrome P450 polynucleotides.

The specifically disclosed cDNA of 33312, 33303, or 32579 comprises the coding region and 5′ and 3′ untranslated sequences in SEQ ID NO:1, SEQ ID NO:4, and SEQ ID NO:7, respectively. The coding region of 33312, 33303, or 32579 begins with ATG and is shown as SEQ ID NO:3, SEQ ID NO:6, and SEQ ID NO:9, respectively. Thus, in one embodiment, an isolated nucleic acid molecule of the invention includes the nucleotide sequence shown in SEQ ID NO: 1, 4, or 7, or a portion of any of these nucleotide sequences. In another embodiment, the nucleic acid molecule includes sequences encoding the 33312, 33303, or 32579 cytochrome P450 protein (i.e., “the coding region”, SEQ ID NO:3, 6, or 9), as well as 5′ untranslated sequences. Alternatively, the nucleic acid molecule can include only the coding region of SEQ ID NO: 1 (e.g., SEQ ID NO:3, 6, or 9) and, e.g., no flanking sequences which normally accompany the subject sequence.

Thus, 33312, 33303, or 32579 cytochrome P450 polynucleotides include, but are not limited to, the sequence encoding the mature polypeptide alone, the sequence encoding the mature polypeptide and additional coding sequences, such as a leader or secretory sequence (e.g., a pre-pro or pro-protein sequence), the sequence encoding the mature polypeptide, with or without the additional coding sequences, plus additional non-coding sequences, for example introns and non-coding 5′ and 3′ sequences such as transcribed but non-translated sequences that play a role in transcription, RNA processing (including splicing and polyadenylation signals), ribosome binding and stability of mRNA. In addition, the polynucleotide may be fused to a marker sequence encoding, for example, a peptide that facilitates purification.

In yet another embodiment, an isolated nucleic acid molecule of the invention includes a nucleic acid molecule which is a complement of the nucleotide sequence shown in SEQ ID NO: 1 or 3, SEQ ID NO:4 or 6, or SEQ ID NO:7 or 9, or a portion of any of these nucleotide sequences. In still other embodiments, the nucleic acid molecule of the invention is sufficiently complementary to the nucleotide sequence shown in SEQ ID NO: 1 or 3, SEQ ID NO:4 or 6, or SEQ ID NO:7 or 9, such that it can hybridize to the nucleotide sequence shown in SEQ ID NO: 1 or 3, SEQ ID NO:4 or 6, or SEQ ID NO:7 or 9, thereby forming a stable duplex.

In a further embodiment, an isolated nucleic acid molecule of the present invention includes a nucleotide sequence which is at least about 60%, 70%, 80%, 90%, 95%, or more homologous to the entire length of the nucleotide sequence shown in SEQ ID NO: 1 or 3, SEQ ID NO:4 or 6, or SEQ ID NO:7 or 9, or a portion, preferably of the same length, of any of these nucleotide sequences.

33312, 33303, or 32579 Nucleic Acid Fragments

A nucleic acid of the invention can include a portion of the nucleic acid sequence of SEQ ID NO:1 or 3, SEQ ID NO:4 or 6, or SEQ ID NO:7 or 9. Such a nucleic acid molecule can include a fragment which can be used as a probe or primer or a fragment encoding a portion of a 33312, 33303, or 32579 cytochrome P450 protein, e.g., an immunogenic or biologically active portion of 33312, 33303, or 32579 cytochrome P450 protein. A fragment can comprise, e.g., amino acids 32-53 of SEQ ID NO:5, which encodes a leucine zipper pattern of 32579 cytochrome P450. The nucleotide sequence determined from the cloning of the 33312, 33303, or 32579 cytochrome P450 gene allows for the generation of probes and primers designed for use in identifying and/or cloning other 33312, 33303, or 32579 cytochrome P450 family members, or fragments thereof, as well as 33312, 33303, or 32579 cytochrome P450 homologues, or fragments thereof, from other species.

Thus, the present invention provides isolated nucleic acids that contain a single or double stranded subsequence or portion that hybridizes under stringent conditions to the nucleotide sequence of SEQ ID NO: 1 or 3, SEQ ID NO:4 or 6, or SEQ ID NO:7 or 9, or the complement of SEQ ID NO: 1 or 3, SEQ ID NO:4 or 6, or SEQ ID NO:7 or 9. In one embodiment, the nucleic acid consists of a portion of the nucleotide sequence of SEQ ID NO: 1, 4, or 7 and the complement of SEQ ID NO: 1, 4, or 7. Other subsequences include nucleotide sequences encoding the amino acid subsequences described herein up to along the entire length of the gene encoding the 33312, 33303, or 32579 cytochrome P450 polypeptide, including any 5′ or 3′ untranslated region. Accordingly, it could be derived from 5′ noncoding regions, the coding region, and 3′ noncoding regions. Nucleic acid subsequences, according to the invention, should not be construed as encompassing those fragments that may have been disclosed prior to the invention.

Thus, 33312, 33303, or 32579 cytochrome P450 nucleic acid subsequences further include sequences encoding the regions of 33312, 33303, or 32579 cytochrome P450 polypeptide described herein, subregions thereof, and sites having particular activity or function. 33312, 33303, or 32579 cytochrome P450 nucleic acid fragments also include combinations of the regions, segments, motifs, and other functional sites described above. It is understood that a 33312, 33303, or 32579 cytochrome P450 subsequence includes any nucleic acid sequence that does not include the entire gene. A person of ordinary skill in the art would be aware of the many permutations that are possible.

The nucleic acid subsequences of the invention are at least about 15, likely at least about 16, 17, 18, 19, 20, 23 or 25 contiguous nucleotides, and can be 30, 33, 35, 40, 50, 60, 70, 75, 80, 90, 100, 200, 500 or more nucleotides in length. Longer fragments, for example, 600, 700, 800 or more nucleotides in length, which encode antigenic proteins or polypeptides described herein are also useful. 33312, 33303, or 32579 cytochrome P450 probes and primers are provided. Typically a probe/primer is an isolated or purified oligonucleotide. The oligonucleotide typically includes a region of nucleotide sequence that hybridizes under stringent conditions to at least about 7, 12 or 15, preferably about 20 or 25, more preferably about 30, 35, 40, 45, 50, 55, 60, 65, 75 or more consecutive nucleotides of a sense or antisense sequence of SEQ ID NO: 1 or 3, SEQ ID NO:4 or 6, or SEQ ID NO:7 or 9, or of an allelic variant or mutant of SEQ ID NO: 1 or 3, SEQ ID NO:4 or 6, or SEQ ID NO:7 or 9. In a particular embodiment, the nucleic acid probe is at least 5 or 10, and less than 200, more likely less than 100, or less than 75, 50, 40, or 30 base pairs in length. It should be identical, or differ by 1, or less than in 5 or 10 bases, from a sequence disclosed herein. If alignment is needed for this comparison the sequences should be aligned for maximum homology. “Looped” out sequences from deletions or insertions, or mismatches, are considered differences.

As used herein, the term “primer” refers to a single-stranded oligonucleotide which acts as a point of initiation of template-directed DNA polymerization using well-known methods (e.g., PCR, LCR) including, but not limited to those described herein. “Probes” are oligonucleotides that hybridize to a complementary strand of nucleic acid. Such probes include polypeptide nucleic acids (PNAs), as described in Nielsen et al. (1991) Science 254:1497-1500. Typically, a probe comprises a nucleotide sequence region that hybridizes under highly stringent conditions to consecutive nucleotides of the nucleic acid sequence or a complement thereof. More typically, a probe further comprises a label, e.g., radioisotope, fluorescent or luminescent compound, enzyme, or enzyme co-factor.

In another embodiment a set of primers is provided, e.g., primers suitable for use in a PCR, which can be used to amplify a selected region of a 33312, 33303, or 32579 cytochrome P450 sequence, e.g., a domain, region, site or other sequence described herein. For example, a primer can be hybridized to any portion of an mRNA and a larger or full-length cDNA can be produced. The term “primer set” refers to a set of primers including a 5′ (upstream) primer that hybridizes with the 5′ end of the nucleic acid sequence to be amplified and a 3′ (downstream) primer that hybridizes with the complement of the sequence to be amplified. Template directed polymerization produces a double strand polymerization product of the intervening sequence including the primer set.

The appropriate length of the primer depends on the particular use, but typically ranges from about 10, 15, 25 to 50 base pairs in length and less than 100, or less than 200, base pairs in length. The primers should be identical, or differ by one or a few bases from a sequence disclosed herein or from a naturally occurring variant. For example, a nucleic acid fragment encoding a biologically active portion of 33312 includes a cytochrome P450 domain from about amino acid 46 to 501 of SEQ ID NO:2, and a cysteine heme-iron ligand signature from about amino acid 445 to 454 of SEQ ID NO:2. A nucleic acid fragment encoding a biologically active portion of 33303 includes includes a cytochrome P450 domain from about amino acid 33 to 493 of SEQ ID NO:5, a cysteine heme-iron ligand signature from about amino acid 433 to 442 of SEQ ID NO:5, and a leucine zipper pattern from about amino acid 32 to 53 of SEQ ID NO:5. A nucleic acid fragment encoding a biologically active portion of 32579 includes a cytochrome P450 domain from about amino acid 60 to 543 of SEQ ID NO:8, and a cysteine heme-iron ligand signature from about amino acid 483 to 492 of SEQ ID NO:8.

A nucleic acid fragment can encode an epitope bearing region of a polypeptide described herein.

A nucleic acid fragment encoding a “biologically active portion of a 33312, 33303, or 32579 cytochrome P450 polypeptide” can be prepared by isolating a portion of the nucleotide sequence of SEQ ID NO: 1 or 3, SEQ ID NO:4 or 6, or SEQ ID NO:7 or 9, which encodes a polypeptide having a 33312, 33303, or 32579 cytochrome P450 biological activity (e.g., several of the biological activities of 33312, 33303, or 32579 cytochrome P450 proteins are described herein), expressing the encoded portion of the 33312, 33303, or 32579 cytochrome P450 protein (e.g., by recombinant expression in vitro) and assessing the activity of the encoded portion of the 33312, 33303, or 32579 cytochrome P450 protein. For example, a nucleic acid fragment encoding a biologically active portion of 33312 includes a cytochrome P450 domain from about amino acid 46 to 501 of SEQ ID NO:2, and a cysteine heme-iron ligand signature from about amino acid 445 to 454 of SEQ ID NO:2. A nucleic acid fragment encoding a biologically active portion of 33303 includes includes a cytochrome P450 domain from about amino acid 33 to 493 of SEQ ID NO:5, a cysteine heme-iron ligand signature from about amino acid 433 to 442 of SEQ ID NO:5, and a leucine zipper pattern from about amino acid 32 to 53 of SEQ ID NO:5. A nucleic acid fragment encoding a biologically active portion of 32579 includes a cytochrome P450 domain from about amino acid 60 to 543 of SEQ ID NO:8, and a cysteine heme-iron ligand signature from about amino acid 483 to 492 of SEQ ID NO:8.

A nucleic acid subsequence encoding a biologically active portion of a 33312, 33303, or 32579 cytochrome P450 polypeptide, may comprise a nucleotide sequence which is greater than 9, 12 or 15, likely about 21 or 24, more likely about 30, 36, 45, 51, 60, 75, 90, 105, 120, 135, 150, 175, 190, 205, 220, 235, 250 or more nucleotides in length.

In preferred embodiments, nucleic acids include a nucleotide sequence which is about 300,400, 500,526, 532, 533,577, 600,629,700, 800,900, 1000, 1100, 1200, 1300, 1400, or 1500 nucleotides in length and hybridizes under stringent hybridization conditions to a nucleic acid molecule of SEQ ID NO: 1 or 3.

In a preferred embodiment, a nucleic acid fragment differs by at least 1, 2, 3, 10, 20, or more nucleotides from the sequence of AX067310 of WO 00/78960, or AX195182 of WO01/51638, or Genbank accession number AV700083, or Genbank accession number AI668594, or SEQ ID NO:23 of WO 01/90334, or SEQ ID NO:327 of WO01/77291. Differences can include differing in length or sequence identity. For example, a nucleic acid fragment can: include one or more nucleotides from SEQ ID NO:1 or SEQ ID NO:3 located outside the region of nucleotides 19to 1934, 122 to 618,421 to 1891, 1199 to 1919, 1305 to 1880, 1276 to 1904, or 1348 to 1891 of SEQ ID NO: 1; not include all of the nucleotides of AX067310 of WO 00/78960, or AX195182 of WO01/51638, or AV700083, or AI668594, or SEQ ID NO:23 of WO 01/90334, or SEQ ID NO:327 of WO01/77291, e.g., can be one or more nucleotides shorter (at one or both ends) than the sequence of AX067310 of WO 00/78960, or AX195182 of WO01/51638, or AV700083, or AI668594, or SEQ ID NO:23 of WO 01/90334, or SEQ ID NO:327 of WO01/77291; or can differ by one or more nucleotides in the region of overlap.

In preferred embodiments, nucleic acids include a nucleotide sequence which is about 300, 400, 500, 600, 700, 800, 900, 1000, 1100, 1200, 1300, 1400, or 1500 nucleotides in length and hybridizes under stringent hybridization conditions to a nucleic acid molecule of SEQ ID NO:4 or 6.

In a preferred embodiment, a nucleic acid fragment differs by at least 1, 2, 3, 10, 20, or more nucleotides from the sequence of Genbank accession number BE148597 or BG123000, or AC011510; or SEQ ID NO:16 of WO 01/79468, or SEQ ID NOs:27595, 22175,11282,11421, or 23872 of WO 01/57277; or a sequence disclosed in WO 01/55368, or WO 01/34644, or WO 01/62927, or WO 99/06439. Differences can include differing in length or sequence identity. For example, a nucleic acid fragment can: include one or more nucleotides from SEQ ID NO: 4 or SEQ ID NO:6 located outside the region of one or more of nucleotides 1 to 1927, 1 to 1433, 1 to 1211, 475 to 1165, 623 to 1081, 652 to 1927, 652 to 837, 655 to 834, or 1247 to 1820 of SEQ ID NO:4; not include all of the nucleotides of, e.g., can be one or more nucleotides shorter (at one or both ends) than the sequence of Genbank accession number BE148597 or BG123000, or AC011510; or SEQ ID NO:16 of WO 01/79468, or SEQ ID NOs:27595, 22175,11282, 11421, or 23872 of WO 01/57277; or a sequence disclosed in WO 01/55368, or WO 01/34644, or WO 01/62927, or WO 99/06439; or can differ by one or more nucleotides in the region of overlap.

In preferred embodiments, nucleic acids include a nucleotide sequence which is about 300, 400, 500, 600, 700, 800, 900, 1000, 1100, 1200, 1300, 1400, or 1500 nucleotides in length and hybridizes under stringent hybridization conditions to a nucleic acid molecule of SEQ ID NO:7 or 9. In a preferred embodiment, a nucleic acid fragment differs by at least 1, 2, 3, 10, 20, or more nucleotides from the sequence of Genbank accession number AW242436, or AF798940, or BE670378, or AF216236; or SEQ ID NO: 16 of WO 01/81588, or SEQ ID NO: 145 of WO 01/75068, or SEQ ID NOs:5 or 13 of WO 01/81585; or a sequence disclosed in WO 01/39335, or WO 01/77291, or WO 01/81585, or WO 99/37674. Differences can include differing in length or sequence identity. For example, a nucleic acid fragment can: include one or more nucleotides from SEQ ID NO: 7 or SEQ ID NO:9 located outside the region of one or more of nucleotides 1 to 481, 1 to 570, 19 to 355, 43 to 2085, 491 to 2023, 820 to 1377, 1251 to 2009, 1455 to 2009,1259 to 2023, 1437 to 2001, 1455 to 1841, 1546 to 1751, 1616 to 2006 of SEQ ID NO:7; not include all of the nucleotides of, e.g., can be one or more nucleotides shorter (at one or both ends) than the sequence of Genbank accession number AW242436, or AF798940, or BE670378, or AF216236; or SEQ ID NO: 16 of WO 01/81588, or SEQ ID NO: 145 of WO 01/75068, or SEQ ID NOs:5 or 13 of WO 01/81585; or a sequence disclosed in WO 01/39335, or WO 01/77291, or WO 01/81585, or WO 99/37674; or can differ by one or more nucleotides in the region of overlap.

33312, 33303, or 32579 Nucleic Acid Variants

The invention further provides variant 33312, 33303, or 32579 cytochrome P450 polynucleotides, and subsequences thereof, i.e., sequences that differ from the nucleotide sequence shown in SEQ ID NO:1 or 3, SEQ ID NO:4 or 6, or SEQ ID NO:7 or 9. Such differences can be due to degeneracy of the genetic code and thus encode the same protein as that encoded by the nucleotide sequence shown in SEQ ID NO:3, SEQ ID NO:6, or SEQ ID NO:9.

In another embodiment, an isolated nucleic acid molecule of the invention has a nucleotide sequence encoding a protein having an amino acid sequence which differs, by at least 1, but less than 5, 10, 20, 50, or 100 amino acid residues that shown in SEQ ID NO:2, SEQ ID NO:5, or SEQ ID NO:8. If alignment is needed for this comparison the sequences should be aligned for maximum homology. “Looped” out sequences from deletions or insertions, or mismatches, are considered differences.

Thus, the invention also provides 33312, 33303, or 32579 cytochrome P450 nucleic acid molecules encoding the variant polypeptides described herein. Such polynucleotides may be naturally occurring, such as allelic variants (same locus), homologs (different locus), and orthologs (different organism), or may be constructed by recombinant DNA methods or by chemical synthesis. Such non-naturally occurring variants may be made by mutagenesis techniques, including those applied to polynucleotides, cells, or organisms. Accordingly, as discussed above, the variants can contain nucleotide substitutions, deletions, and additions.

Typically, variants have a substantial identity with a nucleic acid molecules of SEQ ID NO:1, SEQ ID NO:4, or SEQ ID NO:7, and the complements thereof. Variation can occur in either or both the coding and non-coding regions. The variations can encode a protein having a conservative or non-conservative amino acid substitution of an essential or non-essential amino acid.

In one embodiment, the nucleic acid differs from that of SEQ ID NO: 1 or 3, SEQ ID NO:4 or 6, or SEQ ID NO:7 or 9, e.g., as follows: by at least one but less than 10, 20, 30, or 40 nucleotides; at least one but less than 1%, 5%, 10% or 20% of the in the subject nucleic acid. If necessary for this analysis the sequences should be aligned for maximum homology. “Looped” out sequences from deletions or insertions, or mismatches, are considered differences.

Orthologs, homologs, and allelic variants can be identified using methods known in the art. These variants comprise a nucleotide sequence encoding a 33312, 33303, or 32579 cytochrome P450 that is typically at least about 60-65%, 65-70%, 70-75%, more typically at least about 80-85%, and most typically at least about 90-95% or more homologous to the nucleotide sequence shown in SEQ ID NO:3, SEQ ID NO:6, or SEQ ID NO:9, or a subsequence of this sequence. Such nucleic acid molecules can readily be identified as being able to hybridize under stringent conditions, to the nucleotide sequence shown in SEQ ID NO: 1 or 3, SEQ ID NO:4 or 6, or SEQ ID NO:7 or 9 or a subsequence of the sequence. Nucleic acid molecules corresponding to orthologs, homologs, and allelic variants of 33312, 33303, or 32579 cytochrome P450 cDNAs of the invention can further be isolated by mapping to the same chromosome or locus as the 33312, 33303, or 32579 cytochrome P450 gene.

Preferred variants include those that are correlated with protease activity, adhesion, cell motility, substrate binding, etc.

It is understood that stringent hybridization does not indicate substantial homology where it is due to general homology, such as polyA+sequences, or sequences common to all or most proteins, cytochromes P450, leucine zipper pattern, or even all proteins in specific cytochrome P450 subfamilies, such as M12B, M13, or M20, etc. Moreover, it is understood that variants do not include any of the nucleic acid sequences that may have been disclosed prior to the invention.

Allelic variants of 33312, 33303, or 32579 cytochrome P450, e.g., human 33312, 33303, or 32579 cytochrome P450, include both functional and non-functional proteins. Functional allelic variants are naturally occurring amino acid sequence variants of the 33312, 33303, or 32579 cytochrome P450 protein within a population that maintain the ability to bind or hydrolyze substrate, for example. Functional allelic variants will typically contain a conservative substitution of one or more amino acids of SEQ ID NO:2, SEQ ID NO:5, or SEQ ID NO:8, or substitution, deletion or addition of non-critical residues in non-critical regions of the protein. Non-functional allelic variants are naturally-occurring amino acid sequence variants of the 33312, 33303, or 32579 cytochrome P450, e.g., human 33312, 33303, or 32579 cytochrome P450, protein within a population that do not have the ability to bind or hydrolyze substrate, for example. Non-functional allelic variants will typically contain one or more non-conservative substitutions, a deletion, or an addition, or premature truncation of the amino acid sequence of SEQ ID NO:2, SEQ ID NO:5, or SEQ ID NO:8, or a substitution, addition, or deletion in critical residues or critical regions of the protein.

Moreover, nucleic acid molecules encoding other 33312, 33303, or 32579 cytochrome P450 family members and, thus, which have a nucleotide sequence which differs from the 33312, 33303, or 32579 cytochrome P450 sequences of SEQ ID NO: 1 or 3, SEQ ID NO:4 or 6, or SEQ ID NO:7 or 9.

Antisense Nucleic Acid Molecules, Ribozymes and Modified 33312, 33303, or 32579 Cytochrome P450 Nucleic Acid Molecules

In another aspect, the invention features, an isolated nucleic acid molecule which is antisense to 33312, 33303, or 32579 cytochrome P450. An “antisense” nucleic acid can include a nucleotide sequence complementary to a “sense” nucleic acid encoding a protein, e.g., complementary to the coding strand of a double-stranded cDNA molecule or complementary to an mRNA sequence. The antisense nucleic acid can be complementary to an entire 33312, 33303, or 32579 cytochrome P450 coding strand, or to only a portion thereof (e.g., the coding region of 33312, 33303, or 32579 cytochrome P450 corresponding to SEQ ID NO:3, SEQ ID NO:6, or SEQ ID NO:9). In another embodiment, the antisense nucleic acid molecule is antisense to a “noncoding region” of the coding strand of a nucleotide sequence encoding 33312, 33303, or 32579 cytochrome P450 (e.g., the 5′ and 3′ untranslated regions).

An antisense nucleic acid can be designed such that it is complementary to the entire coding region of 33312, 33303, or 32579 cytochrome P450 mRNA, but more likely is an oligonucleotide which is antisense to only a portion of the coding or noncoding region of 33312, 33303, or 32579 cytochrome P450 mRNA. For example, the antisense oligonucleotide can be complementary to the region surrounding the translation start site of 33312, 33303, or 32579 cytochrome P450 mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

Antisense nucleic acids of the invention can be designed using the nucleotide sequences of SEQ ID NO: 1 or 3, SEQ ID NO:4 or 6, or SEQ ID NO:7 or 9, and constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

Examples of modified nucleotides which can be used to generate antisense nucleic acids include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

Additionally, 33312, 33303, or 32579 cytochrome P450 nucleic acid molecule can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4:5). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained.

PNAs of 33312, 33303, or 32579 cytochrome P450 nucleic acids can be used in therapeutic and diagnostic applications. For example, PNAs can be used as antisense or antigene agents for sequence-specific modulation of gene expression by, for example, inducing transcription or translation arrest or inhibiting replication. PNAs of 33312, 33303, or 32579 cytochrome P450 nucleic acids can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra). The neutral backbone of PNAs has been shown to allow for specific hybridization to DNA and RNA under conditions of low ionic strength. PNAs can be further modified, e.g., to enhance their stability, specificity or cellular uptake, by attaching lipophilic or other helper groups to PNA, by the formation of PNA-DNA chimeras, or by the use of liposomes or other techniques of drug delivery known in the art. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670, Finn et al. (1996) Nucleic Acids Res. 24(17):3357-63, Mag et al. (1989) Nucleic Acids Res. 17:5973, and Peterser et al. (1975) Bioorganic Med. Chem. Lett. 5:1119.

In yet another embodiment, the antisense nucleic acid molecule of the invention is an a-anomeric nucleic acid molecule. An a-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. A ribozyme having specificity for a 33312, 33303, or 32579 cytochrome P450-encoding nucleic acid can include one or more sequences complementary to the nucleotide sequence of a 33312, 33303, or 32579 cytochrome P450 cDNA disclosed herein (i.e., SEQ ID NO: 1 or 3, SEQ ID NO:4 or 6, or SEQ ID NO:7 or 9), and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a 33312, 33303, or 32579 cytochrome P450-encoding mRNA. See, e.g., Cech et al. U.S. Pat. No. 4,987,071; and Cech et al. U.S. Pat. No. 5,116,742. Alternatively, 33312, 33303, or 32579 cytochrome P450 mRNA can be used to select a catalytic RNA having a specific ribonuclease activity from a pool of RNA molecules. See, e.g., Bartel, D. and Szostak, J. W. (1993) Science 261:1411-1418.

33312, 33303, or 32579 cytochrome P450 gene expression can be inhibited by targeting nucleotide sequences complementary to the regulatory region of the 33312, 33303, or 32579 cytochrome P450 (e.g., the 33312, 33303, or 32579 cytochrome P450 promoter and/or enhancers) to form triple helical structures that prevent transcription of the 33312, 33303, or 32579 cytochrome P450 gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15. The potential sequences that can be targeted for triple helix formation can be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

The invention also includes molecular beacon oligonucleotide primer and probe molecules having at least one region which is complementary to a 33312, 33303, or 32579 cytochrome P450 nucleic acid of the invention, two complementary regions one having a fluorophore and one a quencher such that the molecular beacon is useful for quantitating the presence of the 33312, 33303, or 32579 cytochrome P450 nucleic acid of the invention in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

The antisense nucleic acid molecules of the invention are typically administered to a subject (e.g., by direct injection at a tissue site), or generated in situ such that they hybridize with or bind to cellular mRNA and/or genomic DNA encoding a 33312, 33303, or 32579 cytochrome P450 protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

Isolated 33312, 33303, or 32579 Polyeptides

In another aspect, the invention features, an isolated 33312, 33303, or 32579 cytochrome P450 protein, or fragment, e.g., a biologically active portion, for use as immunogens or antigens to raise or test (or more generally to bind) anti 33312, 33303, or 32579 cytochrome P450 antibodies. 33312, 33303, or 32579 cytochrome P450 protein can be isolated from cells or a tissue source using standard protein purification techniques. 33312, 33303, or 32579 cytochrome P450 protein or subsequences thereof can be produced by recombinant DNA techniques or synthesized chemically using known protein synthesis methods. In one embodiment, the protein is produced by recombinant DNA techniques. For example, a nucleic acid molecule encoding the 33312, 33303, or 32579 cytochrome P450 polypeptide is cloned into an expression vector, the expression vector introduced into a host cell and the protein expressed in the host cell. The protein can then be isolated from the cells by an appropriate purification scheme using standard protein purification techniques.

Polypeptides of the invention include those which arise as a result of the existence of multiple genes, alternative transcripts (e.g., due to different initiation sites), alternative RNA splicing events, and alternative translational and post-translational events. The polypeptide can be expressed in systems, e.g., cultured cells, which result in substantially the same postranslational modifications present when the polypeptide is expressed in a native cell, or in systems which result in the alteration or omission of post-translational modifications, e.g., gylcosylation or cleavage, present when expressed in a native cell.

In one embodiment, a 33312, 33303, or 32579 cytochrome P450 polypeptide has one or more of the following characteristics:

(i) it has the ability to oxidize a protein substrate;

(ii) it is capable of modulating steroid metabolism;

(iii) it is capable of modulating fatty acids metabolism;

(iv) it is capable of activating and detoxifying low molecular carcinogens and other toxins;

(v) it is capable of regulating drug metabolism;

(vi) it has an overall sequence similarity of at least 60% 70%, 80%, 90% or 95%, with the amino acid sequence of SEQ ID NO:2, SEQ ID NO:5 or SEQ ID NO:8;

(vii) it has a cytochrome P450 domain which is preferably about 70%, 80%, 90% or 95% homologous with one of the P450 domains described herein; or

(viii) it has a leucine zipper sequence which is preferably about 70%, 80%, 90% or 95% homologous with amino acid residues from about amino acid 32-53 of SEQ ID NO:5.

In one embodiment, the 33312, 33303, or 32579 cytochrome P450 protein or subsequence thereof, differs from the corresponding sequence in SEQ ID NO:2, 5 or 8. In another embodiment, the 33312, 33303, or 32579 cytochrome P450 protein or subsequence thereof differs by at least one but by less than 15, 10 or 5 amino acid residues. In yet another embodiment, the 33312, 33303, or 32579 cytochrome P450 protein or subsequence thereof differs from the corresponding sequence in SEQ ID NO:2, 5, or 8 by at least one residue but less than 20%, 15%, 10% or 5% of the total residues in it differ from the corresponding sequence in SEQ ID NO:2, 5 or 8 (If this comparison requires alignment the sequences should be aligned for maximum homology. “Looped” out sequences from deletions or insertions, or mismatches, are considered differences.). The differences may be differences or changes at a non-essential residue or alternatively, conservative substitution. Thus, in one embodiment, the differences are in the leucine zipper sequence of 33303 (amino acids from about 32 to 53 of SEQ ID NO:5).

Other embodiments include a protein that contains one or more changes in amino acid sequence, e.g., a change in an amino acid residue which is not essential for activity. Such 33312, 33303, or 32579 cytochrome P450 proteins differ in amino acid sequence from SEQ ID NO:2, 5, or 8, yet retain biological activity.

In one embodiment, the protein includes an amino acid sequence at least about 60%, 70%, 80%, 90%, 95%, or more homologous to SEQ ID NO:2, 5, or 8.

In one embodiment, a biologically active portion or subsequence of a 33312 cytochrome P450 protein includes a cytochrome P450 domain, or a leucine zipper sequence. In another embodiment, a biologically active portion or subsequence of a 33303 cytochrome P450 protein includes a leucine zipper sequence. Moreover, other biologically active portions, in which other regions of the protein are deleted, can be prepared by recombinant techniques and evaluated for one or more of the functions or activities of a 33312, 33303, or 32579 cytochrome P450 sequence protein.

In another embodiment, a 33312, 33303, or 32579 cytochrome P450 protein has an amino acid sequence shown in SEQ ID NO:2, 5 or 8. In other embodiments, a 33312, 33303, or 32579 cytochrome P450 protein is substantially homologous to SEQ ID NO:2, 5, or 8. In yet another embodiment, a 33312, 33303, or 32579 cytochrome P450 protein is substantially homologous to SEQ ID NO:2, 5, or 8, and retains the functional activity of the protein of SEQ ID NO:2, 5, or 8, as described in detail above.

As used herein, two proteins (or a region of the proteins) are substantially homologous when the amino acid sequences are at least about 60-65%, 65-70%, 70-75%, typically at least about 80-85%, and most typically at least about 90-95% or more homologous.

In a preferred embodiment, a fragment differs by at least 1, 2, 3, 10, 20, or more amino acid residues from SEQ ID NO:10 of WO01/90334, or SEQ ID NO:36481 of WO 01/75067 or a sequence present in WO 01/51638, or an amino acid sequence encoded by a sequence present in AX067310 of WO 00/78960, or AX195182 of WO01/51638, or Genbank accession number AV700083, or Genbank accession number AI668594, or SEQ ID NO:23 of WO 01/90334, or SEQ ID NO:327 of WO01/77291. Differences can include differing in length or sequence identity. For example, a fragment can: include one or more amino acid residues from SEQ ID NO:2 outside the region of amino acid residues 42 to 505, 186 to 506, or 211 to 400, of SEQ ID NO:2; not include all of the amino acid residues of a sequence present in SEQ ID NO: 10 of WO01/90334, or SEQ ID NO:36481 of WO 01/75067, or a sequence present in WO 01/51638, or an amino acid sequence encoded by a sequence present in AX067310 of WO 00/78960, or AX195182 of WO01/51638, or Genbank accession number AV700083, or Genbank accession number AI668594, or SEQ ID NO:23 of WO 01/90334, or SEQ ID NO:327 of WO01/77291, e.g., can be one or more amino acid residues shorter (at one or both ends) than a sequence present in SEQ ID NO:10 of WO01/90334, or SEQ ID NO:36481 of WO 01/75067 or a a sequence present in WO 01/51638, or an amino acid sequence encoded by a sequence present in AX067310 of WO 00/78960, or AX195182 of WO01/51638, or Genbank accession number AV700083, or Genbank accession number AI668594, or SEQ ID NO:23 of WO 01/90334, or SEQ ID NO:327 of WO01/77291; or can differ by one or more amino acid residues in the region of overlap.

In a preferred embodiment, a fragment differs by at least 1, 2, 3, 10, 20, or more amino acid residues from an amino acid disclosed in WO 01/40466, WO 01/62927, or WO 01/34644, or an amino acid sequence encoded by a sequence present in the sequence of Genbank accession number BE148597 or BG123000, or AC011510; or SEQ ID NO:16 of WO 01/79468, or SEQ ID NOs:27595, 22175, 11282, 11421, or 23872 of WO 01/57277; or a sequence disclosed in WO 01/55368, or WO 01/34644, or WO 01/62927, or WO 99/06439. Differences can include differing in length or sequence identity. For example, a fragment can: include one or more amino acid residues from SEQ ID NO:5 outside one or more regions of amino acid residues 1 to 504, 1 to 487, 217 to 491, 1 to 218, or 350 to 432 of SEQ ID NO:5; not include all of the amino acid residues of a sequence present in encoded by a sequence present in an amino acid disclosed in WO 01/40466, WO 01/62927, or WO 01/34644, or an amino acid sequence encoded by a sequence present in the sequence of Genbank accession number BE148597 or BG123000, or AC011510; or SEQ ID NO: 16 of WO 01/79468, or SEQ ID NOs:27595, 22175, 11282, 11421, or 23872 of WO 01/57277; or a sequence disclosed in WO 01/55368, or WO 01/34644, or WO 01/62927, or WO 99/06439, or, e.g., can be one or more amino acid residues shorter (at one or both ends) than a sequence present in an amino acid disclosed in WO 01/40466, WO 01/62927, or WO 01/34644, or an amino acid sequence encoded by a sequence present in the sequence of Genbank accession number BE148597 or BG123000, or AC011510; or SEQ ID NO:16 of WO 01/79468, or SEQ ID NOs:27595, 22175, 11282,11421, or 23872 of WO 01/57277; or a sequence disclosed in WO 01/55368, or WO 01/34644, or WO 01/62927, or WO 99/06439; or can differ by one or more amino acid residues in the region of overlap.

In a preferred embodiment, a fragment differs by at least 1, 2, 3, 10, 20, or more amino acid residues from an amino acid disclosed in WO 01/81585, or the sequence of SEQ ID NO:146 of WO 01/39335 or WO 01/75068, or an amino acid sequence encoded by a sequence present in the sequence of Genbank accession number AW242436, or AF798940, or BE670378, or AF216236; or SEQ ID NO:16 of WO 01/81588, or SEQ ID NO:145 of WO 01/75068, or SEQ ID NOs:5 or 13 of WO 01/81585; or a sequence disclosed in WO 01/39335, or WO 01/77291, or WO 01/81585, or WO 99/37674. Differences can include differing in length or sequence identity. For example, a fragment can: include one or more amino acid residues from SEQ ID NO:8 outside one or more regions of amino acid residues 1 to 544 or 164 to 544 of SEQ ID NO:8; not include all of the amino acid residues of a sequence present in encoded by a sequence present in an amino acid disclosed in WO 01/40466, WO 01/62927, or WO 01/34644, or an amino acid sequence encoded by a sequence present in the sequence of Genbank accession number AW242436, or AF798940, or BE670378, or AF216236; or SEQ ID NO: 16 of WO 01/81588, or SEQ ID NO:145 of WO 01/75068, or SEQ ID NOs:5 or 13 of WO 01/81585; or a sequence disclosed in WO 01/39335, or WO 01/77291, or WO 01/81585, or WO 99/37674, or, e.g., can be one or more amino acid residues shorter (at one or both ends) than a sequence present in an amino acid disclosed in WO 01/40466, WO 01/62927, or WO 01/34644, or an amino acid sequence encoded by a sequence present in the sequence of Genbank accession number AW242436, or AF798940, or BE670378, or AF216236; or SEQ ID NO:16 of WO 01/81588, or SEQ ID NO:145 of WO 01/75068, or SEQ ID NOs:5 or 13 of WO 01/81585; or a sequence disclosed in WO 01/39335, or WO 01/77291, or WO 01/81585, or WO 99/37674; or can differ by one or more amino acid residues in the region of overlap.

33312, 33303, or 32579 Chimeric or Fusion Proteins

In another aspect, the invention provides 33312, 33303, or 32579 chimeric or fusion proteins. As used herein, a 33312, 33303, or 32579 “chimeric protein” or “fusion protein” includes a 33312, 33303, or 32579 polypeptide linked to a non-33312, 33303, or 32579 polypeptide. A “non-33312, 33303, or 32579 polypeptide” refers to a polypeptide having an amino acid sequence corresponding to a protein which is not substantially homologous to the 33312, 33303, or 32579 protein, e.g., a protein which is different from the 33312, 33303, or 32579 protein and which is derived from the same or a different organism. The 33312, 33303, or 32579 polypeptide of the fusion protein can correspond to all or a portion e.g., a fragment described herein of a 33312, 33303, or 32579 amino acid sequence. In a preferred embodiment, a 33312, 33303, or 32579 fusion protein includes at least one (or two) biologically active portion of a 33312, 33303, or 32579 protein. The non-33312, 33303, or 32579 polypeptide can be fused to the N-terminus or C-terminus of the 33312, 33303, or 32579 polypeptide.

The fusion protein can include a moiety which has a high affinity for a ligand. For example, the fusion protein can be a GST-33312, 33303, or 32579 fusion protein in which the 33312, 33303, or 32579 sequences are fused to the C-terminus of the GST sequences. Such fusion proteins can facilitate the purification of recombinant 33312, 33303, or 32579. Alternatively, the fusion protein can be a 33312, 33303, or 32579 protein containing a heterologous signal sequence at its N-terminus. In certain host cells (e.g., mammalian host cells), expression and/or secretion of 33312, 33303, or 32579 can be increased through use of a heterologous signal sequence.

Fusion proteins can include all or a part of a serum protein, e.g., an IgG constant region, or human serum albumin.

The 33312, 33303, or 32579 fusion proteins of the invention can be incorporated into pharmaceutical compositions and administered to a subject in vivo. The 33312, 33303, or 32579 fusion proteins can be used to affect the bioavailability of a 33312, 33303, or 32579 substrate. 33312, 33303, or 32579 fusion proteins may be useful therapeutically for the treatment of disorders caused by, for example, (i) aberrant modification or mutation of a gene encoding a 33312, 33303, or 32579 protein; (ii) mis-regulation of the 33312, 33303, or 32579 gene; and (iii) aberrant post-translational modification of a 33312, 33303, or 32579 protein.

Moreover, the 33312, 33303, or 32579-fusion proteins of the invention can be used as immunogens to produce anti-33312, 33303, or 32579 antibodies in a subject, to purify 33312, 33303, or 32579 ligands and in screening assays to identify molecules which inhibit the interaction of 33312, 33303, or 32579 with a 33312, 33303, or 32579 substrate.

Expression vectors are commercially available that already encode a fusion moiety (e.g., a GST polypeptide). A 33312, 33303, or 32579-encoding nucleic acid can be cloned into such an expression vector such that the fusion moiety is linked in-frame to the 33312, 33303, or 32579 protein.

Variants of 33312, 33303, or 32579 Proteins

In another aspect, the invention also features a variant of a 33312, 33303, or 32579 polypeptide, e.g., which functions as an agonist (mimetics) or as an antagonist. Variants of the 33312, 33303, or 32579 proteins can be generated by mutagenesis, e.g., discrete point mutation, the insertion or deletion of sequences or the truncation of a 33312, 33303, or 32579 protein. An agonist of the 33312, 33303, or 32579 proteins can retain substantially the same, or a subset, of the biological activities of the naturally occurring form of a 33312, 33303, or 32579 protein. An antagonist of a 33312, 33303, or 32579 protein can inhibit one or more of the activities of the naturally occurring form of the 33312, 33303, or 32579 protein by, for example, competitively modulating a 33312, 33303, or 32579-mediated activity of a 33312, 33303, or 32579 protein. Thus, specific biological effects can be elicited by treatment with a variant of limited function. Preferably, treatment of a subject with a variant having a subset of the biological activities of the naturally occurring form of the protein has fewer side effects in a subject relative to treatment with the naturally occurring form of the 33312, 33303, or 32579 protein.

Variants of a 33312, 33303, or 32579 protein can be identified by screening combinatorial libraries of mutants, e.g., truncation mutants, of a 33312, 33303, or 32579 protein for agonist or antagonist activity.

Libraries of fragments e.g., N terminal, C terminal, or internal fragments, of a 33312, 33303, or 32579 protein coding sequence can be used to generate a variegated population of fragments for screening and subsequent selection of variants of a 33312, 33303, or 32579 protein.

Variants in which a cysteine residues is added or deleted or in which a residue which is glycosylated is added or deleted are particularly preferred.

Methods for screening gene products of combinatorial libraries made by point mutations or truncation, and for screening cDNA libraries for gene products having a selected property are known in the art. Recursive ensemble mutagenesis (REM), a new technique which enhances the frequency of functional mutants in the libraries, can be used in combination with the screening assays to identify 33312, 33303, or 32579 variants (Arkin and Yourvan (1992) Proc. Natl. Acad. Sci. USA 89:7811-7815; Delgrave et al. (1993) Protein Engineering 6(3):327-331).

Cell based assays can be exploited to analyze a variegated 33312, 33303, or 32579 library. For example, a library of expression vectors can be transfected into a cell line, e.g., a cell line, which ordinarily responds to 33312, 33303, or 32579 in a substrate-dependent manner. The transfected cells are then contacted with 33312, 33303, or 32579 and the effect of the expression of the mutant on signaling by the 33312, 33303, or 32579 substrate can be detected, e.g., by measuring cytochrome P450 activity. Plasmid DNA can then be recovered from the cells which score for inhibition, or alternatively, potentiation of signaling by the 33312, 33303, or 32579 substrate, and the individual clones further characterized.

In another aspect, the invention features a method of making a 33312, 33303, or 32579 polypeptide, e.g., a peptide having a non-wild type activity, e.g., an antagonist, agonist, or super agonist of a naturally occurring 33312, 33303, or 32579 polypeptide, e.g., a naturally occurring 33312, 33303, or 32579 polypeptide. The method includes: altering the sequence of a 33312, 33303, or 32579 polypeptide, e.g., altering the sequence, e.g., by substitution or deletion of one or more residues of a non-conserved region, a domain or residue disclosed herein, and testing the altered polypeptide for the desired activity.

In another aspect, the invention features a method of making a fragment or analog of a 33312, 33303, or 32579 polypeptide having a biological activity of a naturally occurring 33312, 33303, or 32579 polypeptide. The method includes: altering the sequence, e.g., by substitution or deletion of one or more residues, of a 33312, 33303, or 32579 polypeptide, e.g., altering the sequence of a non-conserved region, or a domain or residue described herein, and testing the altered polypeptide for the desired activity.

Anti-33312, 33303, or 32579 Antibodies

In another aspect, the invention provides an anti-33312, 33303 or 32579 antibody, or a fragment thereof (e.g., an antigen-binding fragment thereof). The term “antibody” as used herein refers to an immunoglobulin molecule or immunologically active portion thereof, i.e., an antigen-binding portion. As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4.

The anti-33312, 33303 or 32579 antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

As used herein, the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 KDa or 214 amino acids) are encoded by a variable region gene at the NH2-terminus (about 110 amino acids) and a kappa or lambda constant region gene at the COOH—terminus. Full-length immunoglobulin “heavy chains” (about 50 KDa or 446 amino acids), are similarly encoded by a variable region gene (about 116 amino acids) and one of the other aforementioned constant region genes, e.g., gamma (encoding about 330 amino acids).

The term “antigen-binding fragment” of an antibody (or simply “antibody portion,” or “fragment”), as used herein, refers to one or more fragments of a full-length antibody that retain the ability to specifically bind to the antigen, e.g., 33312, 33303 or 32579 polypeptide or fragment thereof. Examples of antigen-binding fragments of the anti-33312, 33303 or 32579 antibody include, but are not limited to: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “antigen-binding fragment” of an antibody. These antibody fragments are obtained using conventional techniques known to those with skill in the art, and the fragments are screened for utility in the same manner as are intact antibodies.

The anti-33312, 33303 or 32579 antibody can be a polyclonal or a monoclonal antibody. In other embodiments, the antibody can be recombinantly produced, e.g., produced by phage display or by combinatorial methods.

Phage display and combinatorial methods for generating anti-33312, 33303 or 32579 antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J. 12:725-734; Hawkins et al. (1992) J. Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the anti-33312, 33303 or 32579 antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An anti-33312, 33303 or 32579 antibody can be one in which the variable region, or a portion thereof, e.g., the CDR's, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDR's (of heavy and or light immuoglobulin chains) replaced with a donor CDR. The antibody may be replaced with at least a portion of a non-human CDR or only some of the CDR's may be replaced with non-human CDR's. It is only necessary to replace the number of CDR's required for binding of the humanized antibody to a 33312, 33303 or 32579 or a fragment thereof. Preferably, the donor will be a rodent antibody, e.g., a rat or mouse antibody, and the recipient will be a human framework or a human consensus framework. Typically, the immunoglobulin providing the CDR's is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art. Humanized antibodies can be generated by replacing sequences of the Fv variable region which are not directly involved in antigen binding with equivalent sequences from human Fv variable regions. General methods for generating humanized antibodies are provided by Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a 33312, 33303 or 32579 polypeptide or fragment thereof. The recombinant DNA encoding the humanized antibody, or fragment thereof, can then be cloned into an appropriate expression vector.

Humanized or CDR-grafted antibodies can be produced by CDR-grafting or CDR substitution, wherein one, two, or all CDR's of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Preferred humanized antibodies have amino acid substitutions in the framework region, such as to improve binding to the antigen. For example, a humanized antibody will have framework residues identical to the donor framework residue or to another amino acid other than the recipient framework residue. To generate such antibodies, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

In preferred embodiments an antibody can be made by immunizing with purified 33312, 33303 or 32579 antigen, or a fragment thereof, e.g., a fragment described herein.

A full-length 33312, 33303 or 32579 protein or, antigenic peptide fragment of 33312, 33303 or 32579 can be used as an immunogen or can be used to identify anti-33312, 33303 or 32579 antibodies made with other immunogens, e.g., cells, membrane preparations, and the like. The antigenic peptide of 33312, 33303 or 32579 should include at least 8 amino acid residues of the amino acid sequence shown in SEQ ID NO:2 and encompasses an epitope of 33312, 33303 or 32579. Preferably, the antigenic peptide includes at least 10 amino acid residues, more preferably at least 15 amino acid residues, even more preferably at least 20 amino acid residues, and most preferably at least 30 amino acid residues.

Fragments of 33312, 33303 or 32579 which include residues about 130 to 142, or about 325 to 350 of SEQ ID NO:2; about 120 to 130, 272 to 290, or about 400 to 425 of SEQ ID NO:5; or about 241 to 252, or about 321 to 341 of SEQ ID NO:8 can be used to make, e.g., used as immunogens or used to characterize the specificity of an antibody, antibodies against hydrophilic regions of the 33312, 33303 or 32579 protein. Similarly, fragments of 33312, 33303 or 32579 which include residues about 82 to 95, 145 to 158, or 321 to 332 of SEQ ID NO:2; or about 164 to 190, 285 to 320, 445 to 461 of SEQ ID NO:5; or about 115 to 132, about 220 to 237, about 341 to 355, or about 410 to 422 of SEQ ID NO:8 can be used to make an antibody against a hydrophobic region of the 33312, 33303 or 32579 protein; a fragment of 33312, 33303 or 32579 which include residues about 46 to 501 of SEQ ID NO:2 or a fragment thereof (e.g., about 46 to 100, 100 to 200, 200 to 300, 300 to 400, or 400 to 501 of SEQ ID NO:2); about 33 to 493 of SEQ ID NO:5 or a fragment thereof (e.g., about 33 to 100, 100 to 200, 200 to 300, 300 to 400, or 400 to 493 of SEQ ID NO:5); or about 60 to 543 of SEQ ID NO:8 or a fragment thereof (e.g., about 60 to 100, 100 to 200, 200 to 300, 300 to 400, 400 to 500, or 500 to 543 of SEQ ID NO:8) can be used to make an antibody against the cytochrome P450 region of the 33312, 33303 or 32579 protein.

Antibodies reactive with, or specific for, any of these regions, or other regions or domains described herein are provided.

Antibodies which bind only native 33312, 33303 or 32579 protein, only denatured or otherwise non-native 33312, 33303 or 32579 protein, or which bind both, are with in the invention. Antibodies with linear or conformational epitopes are within the invention. Conformational epitopes can sometimes be identified by identifying antibodies which bind to native but not denatured 33312, 33303 or 32579 protein.

Preferred epitopes encompassed by the antigenic peptide are regions of 33312, 33303 or 32579 are located on the surface of the protein, e.g., hydrophilic regions, as well as regions with high antigenicity. For example, an Emini surface probability analysis of the human 33312, 33303 or 32579 protein sequence can be used to indicate the regions that have a particularly high probability of being localized to the surface of the 33312, 33303 or 32579 protein and are thus likely to constitute surface residues useful for targeting antibody production.

The anti-33312, 33303 or 32579 antibody can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target 33312, 33303 or 32579 protein.

In a preferred embodiment the antibody has effector function and/or can fix complement. In other embodiments the antibody does not recruit effector cells; or fix complement.

In a preferred embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

In a preferred embodiment, an anti-33312, 33303 or 32579 antibody alters (e.g., increases or decreases) the activity of a 33312, 33303 or 32579 polypeptide. For example, the antibody can bind at or in proximity to the active site, e.g., to an epitope that includes a residue located from about 445 to 454 of SEQ ID NO:2, about 433 to 442 of SEQ ID NO:5, or about 483 to 492 of SEQ ID NO:8.

The antibody can be coupled to a toxin, e.g., a polypeptide toxin, e,g, ricin or diphtheria toxin or active fragment hereof, or a radioactive nucleus, or imaging agent, e.g. a radioactive, enzymatic, or other, e.g., imaging agent, e.g., a NMR contrast agent. Labels that produce detectable radioactive emissions or fluorescence are preferred.

An anti-33312, 33303 or 32579 antibody (e.g., monoclonal antibody) can be used to isolate 33312, 33303 or 32579 by standard techniques, such as affinity chromatography or immunoprecipitation. Moreover, an anti-33312, 33303 or 32579 antibody can be used to detect 33312, 33303 or 32579 protein (e.g., in a cellular lysate or cell supernatant) in order to evaluate the abundance and pattern of expression of the protein. Anti-33312, 33303 or 32579 antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labelling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

The invention also includes a nucleic acid that encodes an anti-33312, 33303 or 32579 antibody, e.g., an anti-33312, 33303 or 32579 antibody described herein. Also included are vectors which include the nucleic acid and cells transformed with the nucleic acid, particularly cells which are useful for producing an antibody, e.g., mammalian cells, e.g. CHO or lymphatic cells.

The invention also includes cell lines, e.g., hybridomas, which make an anti-33312, 33303 or 32579 antibody, e.g., an antibody described herein, and method of using said cells to make a 33312, 33303 or 32579 antibody.

33312, 33303, and 32579 Recombinant Expression Vectors, Host Cells and Genetically Engineered Cells

In another aspect, the invention includes, vectors, preferably expression vectors, containing a nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a 33312, 33303, or 32579 nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., 33312, 33303, or 32579 proteins, mutant forms of 33312, 33303, or 32579 proteins, fusion proteins, and the like).

The recombinant expression vectors of the invention can be designed for expression of 33312, 33303, or 32579 proteins in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in 33312, 33303, or 32579 activity assays, (e.g., direct assays or competitive assays described in detail below), or to generate antibodies specific for 33312, 33303, or 32579 proteins. In a preferred embodiment, a fusion protein expressed in a retroviral expression vector of the present invention can be used to infect bone marrow cells which are subsequently transplanted into irradiated recipients. The pathology of the subject recipient is then examined after sufficient time has passed (e.g., six (6) weeks).

To maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The 33312, 33303, or 32579 expression vector can be a yeast expression vector, a vector for expression in insect cells, e.g., a baculovirus expression vector or a vector suitable for expression in mammalian cells.

When used in mammalian cells, the expression vector's control functions are often provided by viral regulatory elements. For example, commonly used promoters are derived from polyoma, Adenovirus 2, cytomegalovirus and Simian Virus 40.

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the a-fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus. For a discussion of the regulation of gene expression using antisense genes see Weintraub, H. et al., Antisense RNA as a molecular tool for genetic analysis, Reviews—Trends in Genetics, Vol. 1(1) 1986.

Another aspect the invention provides a host cell which includes a nucleic acid molecule described herein, e.g., a 33312, 33303, or 32579 nucleic acid molecule within a recombinant expression vector or a 33312, 33303, or 32579 nucleic acid molecule containing sequences which allow it to homologously recombine into a specific site of the host cell's genome. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a 33312, 33303, or 32579 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation

A host cell of the invention can be used to produce (i.e., express) a 33312, 33303, or 32579 protein. Accordingly, the invention further provides methods for producing a 33312, 33303, or 32579 protein using the host cells of the invention. In one embodiment, the method includes culturing the host cell of the invention (into which a recombinant expression vector encoding a 33312, 33303, or 32579 protein has been introduced) in a suitable medium such that a 33312, 33303, or 32579 protein is produced. In another embodiment, the method further includes isolating a 33312, 33303, or 32579 protein from the medium or the host cell.

In another aspect, the invention features, a cell or purified preparation of cells which include a 33312, 33303, or 32579 transgene, or which otherwise misexpress 33312, 33303, or 32579. The cell preparation can consist of human or non human cells, e.g., rodent cells, e.g., mouse or rat cells, rabbit cells, or pig cells. In preferred embodiments, the cell or cells include a 33312, 33303, or 32579 transgene, e.g., a heterologous form of a 33312, 33303, or 32579, e.g., a gene derived from humans (in the case of a non-human cell). The 33312, 33303, or 32579 transgene can be misexpressed, e.g., overexpressed or underexpressed. In other preferred embodiments, the cell or cells include a gene which misexpress an endogenous 33312, 33303, or 32579, e.g., a gene the expression of which is disrupted, e.g., a knockout. Such cells can serve as a model for studying disorders which are related to mutated or mis-expressed 33312, 33303, or 32579 alleles or for use in drug screening.

In another aspect, the invention features, a human cell, e.g., a hematopoietic stem cell, transformed with nucleic acid which encodes a subject 33312, 33303, or 32579 polypeptide.

Also provided are cells, preferably human cells, e.g., human hematopoietic or fibroblast cells, in which an endogenous 33312, 33303, or 32579 is under the control of a regulatory sequence that does not normally control the expression of the endogenous 33312, 33303, or 32579 gene. The expression characteristics of an endogenous gene within a cell, e.g., a cell line or microorganism, can be modified by inserting a heterologous DNA regulatory element into the genome of the cell such that the inserted regulatory element is operably linked to the endogenous 33312, 33303, or 32579 gene. For example, an endogenous 33312, 33303, or 32579 gene which is “transcriptionally silent,” e.g., not normally expressed, or expressed only at very low levels, may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published in May 16, 1991.

33312, 33303, and 32579 Transgenic Animals

The invention provides non-human transgenic animals. Such animals are useful for studying the function and/or activity of a 33312, 33303, or 32579 protein and for identifying and/or evaluating modulators of 33312, 33303, or 32579 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA or a rearrangment, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous 33312, 33303, or 32579 gene has been altered by, e.g., by homologous recombination between the endogenous gene and an exogenous DNA molecule introduced into a cell of the animal, e.g., an embryonic cell of the animal, prior to development of the animal.

Intronic sequences and polyadenylation signals can also be included in the transgene to increase the efficiency of expression of the transgene. A tissue-specific regulatory sequence(s) can be operably linked to a transgene of the invention to direct expression of a 33312, 33303, or 32579 protein to particular cells. A transgenic founder animal can be identified based upon the presence of a 33312, 33303, or 32579 transgene in its genome and/or expression of 33312, 33303, or 32579 mRNA in tissues or cells of the animals. A transgenic founder animal can then be used to breed additional animals carrying the transgene. Moreover, transgenic animals carrying a transgene encoding a 33312, 33303, or 32579 protein can further be bred to other transgenic animals carrying other transgenes.

33312, 33303, or 32579 proteins or polypeptides can be expressed in transgenic animals or plants, e.g., a nucleic acid encoding the protein or polypeptide can be introduced into the genome of an animal. In preferred embodiments the nucleic acid is placed under the control of a tissue specific promoter, e.g., a milk or egg specific promoter, and recovered from the milk or eggs produced by the animal. Suitable animals are mice, pigs, cows, goats, and sheep.

The invention also includes a population of cells from a transgenic animal, as discussed, e.g., below.

Uses of 33312, 33303, and 32579

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic).

The isolated nucleic acid molecules of the invention can be used, for example, to express a 33312, 33303, or 32579 protein (e.g., via a recombinant expression vector in a host cell in gene therapy applications), to detect a 33312, 33303, or 32579 mRNA (e.g., in a biological sample) or a genetic alteration in a 33312, 33303, or 32579 gene, and to modulate 33312, 33303, or 32579 activity, as described further below. The 33312, 33303, or 32579 proteins can be used to treat disorders characterized by insufficient or excessive production of a 33312, 33303, or 32579 substrate or production of 33312, 33303, or 32579 inhibitors. In addition, the 33312, 33303, or 32579 proteins can be used to screen for naturally occurring 33312, 33303, or 32579 substrates, to screen for drugs or compounds which modulate 33312, 33303, or 32579 activity, as well as to treat disorders characterized by insufficient or excessive production of 33312, 33303, or 32579 protein or production of 33312, 33303, or 32579 protein forms which have decreased, aberrant or unwanted activity compared to 33312, 33303, or 32579 wild type protein (e.g., cytochrome P450 associated disorders). Moreover, the anti-33312, 33303, or 32579 antibodies of the invention can be used to detect and isolate 33312, 33303, or 32579 proteins, regulate the bioavailability of 33312, 33303, or 32579 proteins, and modulate 33312, 33303, or 32579 activity.

Uses are relevant for disorders involving an increase or decrease in 33312, 33303, or 32579 cytochrome P450 expression relative to normal, including proliferative disorders, differentiative or developmental disorders, cell adhesion, motility or migration disorders, vascularization/angiogenesis disorders, inflammatory disorders, gene expression disorders, neurite outgrowth disorders, or a hematopoietic disorders.

A method of evaluating a compound for the ability to interact with, e.g., bind, a subject 33312, 33303, or 32579 polypeptide is provided. The method includes: contacting the compound with the subject (33312, 33303, or 32579) polypeptide; and evaluating ability of the compound to interact with, e.g., to bind or form a complex with the subject (33312, 33303, or 32579) polypeptide. This method can be performed in vitro, e.g., in a cell free system, or in vivo, e.g., in a two-hybrid interaction trap assay. This method can be used to identify naturally occurring molecules which interact with subject (33312, 33303, or 32579) polypeptide. It can also be used to find natural or synthetic inhibitors of subject (33312, 33303, or 32579) polypeptide. Screening methods are discussed in more detail below.

33312, 33303. and 32579 Screening Assays

The invention provides methods (also referred to herein as “screening assays”) for identifying modulators, i.e., candidate or test compounds or agents (e.g., proteins, peptides, peptidomimetics, peptoids, small molecules or other drugs) which bind to 33312, 33303, or 32579 proteins, have a stimulatory or inhibitory effect on, for example, 33312, 33303, or 32579 expression or 33312, 33303, or 32579 activity, or have a stimulatory or inhibitory effect on, for example, the expression or activity of a 33312, 33303, or 32579 substrate. Compounds thus identified can be used to modulate the activity of target gene products (e.g., 33312, 33303, or 32579 genes) in a therapeutic protocol, to elaborate the biological function of the target gene product, or to identify compounds that disrupt normal target gene interactions.

In one embodiment, the invention provides assays for screening candidate or test compounds which are substrates of a 33312, 33303, or 32579 protein or polypeptide or a biologically active portion thereof. In another embodiment, the invention provides assays for screening candidate or test compounds which bind to or modulate the activity of a 33312, 33303, or 32579 protein or polypeptide or a biologically active portion thereof.

The test compounds of the present invention can be obtained using any of the numerous approaches in combinatorial library methods known in the art, including: biological libraries; peptoid libraries [libraries of molecules having the functionalities of peptides, but with a novel, non-peptide backbone which are resistant to enzymatic degradation but which nevertheless remain bioactive] (see, e.g., Zuckermann, R. N. et al. J. Med. Chem. 1994, 37: 2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or onphage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a 33312, 33303, or 32579 protein or biologically active portion thereof is contacted with a test compound, and the ability of the test compound to modulate 33312, 33303, or 32579 activity is determined. Determining the ability of the test compound to modulate 33312, 33303, or 32579 activity can be accomplished by monitoring, for example, cytochrome P450 activity. The cell, for example, can be of mammalian origin, e.g., human.

The ability of the test compound to modulate 33312, 33303, or 32579 binding to a compound, e.g., a 33312, 33303, or 32579 substrate, or to bind to 33312, 33303, or 32579 can also be evaluated. This can be accomplished, for example, by coupling the compound, e.g., the substrate, with a radioisotope or enzymatic label such that binding of the compound, e.g., the substrate, to 33312, 33303, or 32579 can be determined by detecting the labeled compound, e.g., substrate, in a complex. Alternatively, 33312, 33303, or 32579 could be coupled with a radioisotope or enzymatic label to monitor the ability of a test compound to modulate 33312, 33303, or 32579 binding to a 33312, 33303, or 32579 substrate in a complex. For example, compounds (e.g., 33312, 33303, or 32579 substrates) can be labeled with ¹²⁵I, ³⁵S, ¹⁴C, or 3H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a 33312, 33303, or 32579 substrate) to interact with 33312, 33303, or 32579 with or without the labeling of any of the interactants can be evaluated. For example, a microphysiometer can be used to detect the interaction of a compound with 33312, 33303, or 32579 without the labeling of either the compound or the 33312, 33303, or 32579. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 33312, 33303, or 32579.

In yet another embodiment, a cell-free assay is provided in which a 33312, 33303, or 32579 protein or biologically active portion thereof is contacted with a test compound and the ability of the test compound to bind to the 33312, 33303, or 32579 protein or biologically active portion thereof is evaluated. Preferred biologically active portions of the 33312, 33303, or 32579 proteins to be used in assays of the present invention include fragments which participate in interactions with non-33312, 33303, or 32579 molecules, e.g., fragments with high surface probability scores.

Soluble and/or membrane-bound forms of isolated proteins (e.g., 33312, 33303, or 32579 proteins or biologically active portions thereof) can be used in the cell-free assays of the invention. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Cell-free assays involve preparing a reaction mixture of the target gene protein and the test compound under conditions and for a time sufficient to allow the two components to interact and bind, thus forming a complex that can be removed and/or detected. The interaction between two molecules can also be detected, e.g., using fluorescence energy transfer (FET) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of the 33312, 33303, or 32579 protein to bind to a target molecule can be accomplished using real-time Biomolecular Interaction Analysis (BIA) (see, e.g., Sjolander, S. and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize either 33312, 33303, or 32579, an anti 33312, 33303, or 32579 antibody or its target molecule to facilitate separation of complexed from uncomplexed forms of one or both of the proteins, as well as to accommodate automation of the assay. Binding of a test compound to a 33312, 33303, or 32579 protein, or interaction of a 33312, 33303, or 32579 protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/33312, 33303, or 32579 fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or 33312, 33303, or 32579 protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of 33312, 33303, or 32579 binding or activity determined using standard techniques.

Other techniques for immobilizing either a 33312, 33303, or 32579 protein or a target molecule on matrices include using conjugation of biotin and streptavidin. Biotinylated 33312, 33303, or 32579 protein or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Iγ antibody).

In one embodiment, this assay is performed utilizing antibodies reactive with 33312, 33303, or 32579 protein or target molecules but which do not interfere with binding of the 33312, 33303, or 32579 protein to its target molecule. Such antibodies can be derivatized to the wells of the plate, and unbound target or 33312, 33303, or 32579 protein trapped in the wells by antibody conjugation. Methods for detecting such complexes, in addition to those described above for the GST-immobilized complexes, include immunodetection of complexes using antibodies reactive with the 33312, 33303, or 32579 protein or target molecule, as well as enzyme-linked assays which rely on detecting an enzymatic activity associated with the 33312, 33303, or 32579 protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., Trends Biochem Sci Aug. 18, 1993(8):284-7); chromatography (gel filtration chromatography, ion-exchange chromatography); electrophoresis (see, e.g., Ausubel, F. et al., eds. Current Protocols in Molecular Biology 1999, J. Wiley: New York.); and immunoprecipitation (see, for example, Ausubel, F. et al., eds. Current Protocols in Molecular Biology 1999, J. Wiley: New York). Such resins and chromatographic techniques are known to one skilled in the art (see, e.g., Heegaard, N. H., J Mol Recognit 1998 Winter;11(1-6):141-8; Hage, D. S., and Tweed, S. A. J Chromatogr B Biomed Sci Appl Oct. 10, 1997;699(1-2):499-525). Further, fluorescence energy transfer may also be conveniently utilized, as described herein, to detect binding without further purification of the complex from solution.

In a preferred embodiment, the assay includes contacting the 33312, 33303, or 32579 protein or biologically active portion thereof with a known compound which binds 33312, 33303, or 32579 to form an assay mixture, contacting the assay mixture with a test compound, and determining the ability of the test compound to interact with a 33312, 33303, or 32579 protein, wherein determining the ability of the test compound to interact with a 33312, 33303, or 32579 protein includes determining the ability of the test compound to preferentially bind to 33312, 33303, or 32579 or biologically active portion thereof, or to modulate the activity of a target molecule, as compared to the known compound.

The target gene products of the invention can, in vivo, interact with one or more cellular or extracellular macromolecules, such as proteins. For the purposes of this discussion, such cellular and extracellular macromolecules are referred to herein as “binding partners.”

Compounds that disrupt such interactions can be useful in regulating the activity of the target gene product. Such compounds can include, but are not limited to molecules such as antibodies, peptides, and small molecules. The preferred target genes/products for use in this embodiment are the 33312, 33303, or 32579 genes herein identified. In an alternative embodiment, the invention provides methods for determining the ability of the test compound to modulate the activity of a 33312, 33303, or 32579 protein through modulation of the activity of a downstream effector of a 33312, 33303, or 32579 target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the 33312, 33303, or 32579 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et a. (1993) Oncogene 8:1693-1696; and Brent WO94/10300), to identify other proteins, which bind to or interact with 33312, 33303, or 32579 (“33312, 33303, or 32579-binding proteins” or “33312, 33303, or 32579-bp”) and are involved in 33312, 33303, or 32579 activity. Such 33312, 33303, or 32579-bps can be activators or inhibitors of signals by the 33312, 33303, or 32579 proteins or 33312, 33303, or 32579 targets as, for example, downstream elements of a 33312, 33303, or 32579-mediated signaling pathway.

The two-hybrid system is based on the modular nature of most transcription factors, which consist of separable DNA-binding and activation domains. Briefly, the assay utilizes two different DNA constructs. In one construct, the gene that codes for a 33312, 33303, or 32579 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively the: 33312, 33303, or 32579 protein can be the fused to the activator domain.) If the “bait” and the “prey” proteins are able to interact, in vivo, forming a 33312, 33303, or 32579-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 33312, 33303, or 32579 protein.

In another embodiment, modulators of 33312, 33303, or 32579 expression are identified. For example, a cell or cell free mixture is contacted with a candidate compound and the expression of 33312, 33303, or 32579 mRNA or protein evaluated relative to the level of expression of 33312, 33303, or 32579 mRNA or protein in the absence of the candidate compound. When expression of 33312, 33303, or 32579 mRNA or protein is greater in the presence of the candidate compound than in its absence, the candidate compound is identified as a stimulator of 33312, 33303, or 32579 mRNA or protein expression. Alternatively, when expression of 33312, 33303, or 32579 mRNA or protein is less (statistically significantly less) in the presence of the candidate compound than in its absence, the candidate compound is identified as an inhibitor of 33312, 33303, or 32579 mRNA or protein expression. The level of 33312, 33303, or 32579 mRNA or protein expression can be determined by methods described herein for detecting 33312, 33303, or 32579 mRNA or protein.

In another aspect, the invention pertains to a combination of two or more of the assays described herein. For example, a modulating agent can be identified using a cell-based or a cell free assay, and the ability of the agent to modulate the activity of a 33312, 33303, or 32579 protein can be confirmed in vivo, e.g., in an animal such as an animal model for a neuronal disorder.

This invention further pertains to novel agents identified by the above-described screening assays. Accordingly, it is within the scope of this invention to further use an agent identified as described herein (e.g., a 33312, 33303, or 32579 modulating agent, an antisense 33312, 33303, or 32579 nucleic acid molecule, a 33312, 33303, or 32579-specific antibody, or a 33312, 33303, or 32579-binding partner) in an appropriate animal model to determine the efficacy, toxicity, side effects, or mechanism of action, of treatment with such an agent. Furthermore, novel agents identified by the above-described screening assays can be used for treatments as described herein.

33312, 33303, and 32579 Detection Assays

Portions or fragments of the nucleic acid sequences identified herein can be used as polynucleotide reagents. For example, these sequences can be used to: (i) map their respective genes on a chromosome e.g., to locate gene regions associated with genetic disease or to associate 33312, 33303, or 32579 with a disease; (ii) identify an individual from a minute biological sample (tissue typing); and (iii) aid in forensic identification of a biological sample. These applications are described in the subsections below.

33312, 33303, and 32579 Chromosome Mapping

The 33312, 33303, or 32579 nucleotide sequences or portions thereof can be used to map the location of the 33312, 33303, or 32579 genes on a chromosome. This process is called chromosome mapping. Chromosome mapping is useful in correlating the 33312, 33303, or 32579 sequences with genes associated with disease.

Briefly, 33312, 33303, or 32579 genes can be mapped to chromosomes by preparing PCR primers (preferably 15-25 bp in length) from the 33312, 33303, or 32579 nucleotide sequences. These primers can then be used for PCR screening of somatic cell hybrids containing individual human chromosomes. Only those hybrids containing the human gene corresponding to the 33312, 33303, or 32579 sequences will yield an amplified fragment.

A panel of somatic cell hybrids in which each cell line contains either a single human chromosome or a small number of human chromosomes, and a full set of mouse chromosomes, can allow easy mapping of individual genes to specific human chromosomes. (D'Eustachio P. et al. (1983) Science 220:919-924).

Other mapping strategies e.g., in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries can be used to map 33312, 33303, or 32579 to a chromosomal location.

Fluorescence in situ hybridization (FISH) of a DNA sequence to a metaphase chromosomal spread can further be used to provide a precise chromosomal location in one step. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the 33312, 33303, or 32579 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

33312, 33303, and 32579 Tissue Typing

33312, 33303, or 32579 sequences can be used to identify individuals from biological samples using, e.g., restriction fragment length polymorphism (RFLP). In this technique, an individual's genomic DNA is digested with one or more restriction enzymes, the fragments separated, e.g., in a Southern blot, and probed to yield bands for identification. The sequences of the present invention are useful as additional DNA markers for RFLP (described in U.S. Pat. No. 5,272,057).

Furthermore, the sequences of the present invention can also be used to determine the actual base-by-base DNA sequence of selected portions of an individual's genome. Thus, the 33312, 33303, or 32579 nucleotide sequences described herein can be used to prepare two PCR primers from the 5′ and 3′ ends of the sequences. These primers can then be used to amplify an individual's DNA and subsequently sequence it. Panels of corresponding DNA sequences from individuals, prepared in this manner, can provide unique individual identifications, as each individual will have a unique set of such DNA sequences due to allelic differences.

Allelic variation occurs to some degree in the coding regions of these sequences, and to a greater degree in the noncoding regions. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO: 1, SEQ ID NO:4, or SEQ ID NO:7 can provide positive individual identification with a panel of perhaps 10 to 1,000 primers which each yield a noncoding amplified sequence of 100 bases. If predicted coding sequences, such as those in SEQ ID NO:3, SEQ ID NO:6, or SEQ ID NO:9 are used, a more appropriate number of primers for positive individual identification would be 500-2,000.

If a panel of reagents from 33312, 33303, or 32579 nucleotide sequences described herein is used to generate a unique identification database for an individual, those same reagents can later be used to identify tissue from that individual. Using the unique identification database, positive identification of the individual, living or dead, can be made from extremely small tissue samples.

Use of Partial 33312, 33303, or 32579 Sequences in Forensic Biology

DNA-based identification techniques can also be used in forensic biology. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO: 1, SEQ ID NO:4, or SEQ ID NO:7 (e.g., fragments derived from the noncoding regions of SEQ ID NO:1, SEQ ID NO:4, or SEQ ID NO:7 having a length of at least 20 bases, preferably at least 30 bases) are particularly appropriate for this use.

The 33312, 33303, or 32579 nucleotide sequences described herein can further be used to provide polynucleotide reagents, e.g., labeled or labelable probes which can be used in, for example, an in situ hybridization technique, to identify a specific tissue, e.g., a tissue containing neurons. This can be very useful in cases where a forensic pathologist is presented with a tissue of unknown origin. Panels of such 33312, 33303, or 32579 probes can be used to identify tissue by species and/or by organ type.

In a similar fashion, these reagents, e.g., 33312, 33303, or 32579 primers or probes can be used to screen tissue culture for contamination (i.e. screen for the presence of a mixture of different types of cells in a culture).

Predictive Medicine of 33312, 33303, and 32579

The present invention also pertains to the field of predictive medicine in which diagnostic assays, prognostic assays, and monitoring clinical trials are used for prognostic (predictive) purposes to thereby treat an individual.

Generally, the invention provides, a method of determining if a subject is at risk for a disorder related to a lesion in or the misexpression of a gene which encodes 33312, 33303, or 32579.

Such disorders include, e.g., a disorder associated with the misexpression of 33312, 33303, or 32579; a disorder characterized by a misregulation of a cytochrome P450 mediated activity; a disorder of cell proliferation, cell adhesion, cell motility and migration, inflammatory response, or angiogenesis and vascularization, among others.

The method includes one or more of the following:

detecting, in a tissue of the subject, the presence or absence of a mutation which affects the expression of the 33312, 33303, or 32579 gene, or detecting the presence or absence of a mutation in a region which controls the expression of the gene, e.g., a mutation in the 5′ control region;

detecting, in a tissue of the subject, the presence or absence of a mutation which alters the structure of the 33312, 33303, or 32579 gene;

detecting, in a tissue of the subject, the misexpression of the 33312, 33303, or 32579 gene, at the mRNA level, e.g., detecting a non-wild type level of a mRNA;

detecting, in a tissue of the subject, the misexpression of the gene, at the protein level, e.g., detecting a non-wild type level of a 33312, 33303, or 32579 polypeptide.

In preferred embodiments the method includes: ascertaining the existence of at least one of: a deletion of one or more nucleotides from the 33312, 33303, or 32579 gene; an insertion of one or more nucleotides into the gene, a point mutation, e.g., a substitution of one or more nucleotides of the gene, a gross chromosomal rearrangement of the gene, e.g., a translocation, inversion, or deletion.

For example, detecting the genetic lesion can include: (i) providing a probe/primer including an oligonucleotide containing a region of nucleotide sequence which hybridizes to a sense or antisense sequence from SEQ ID NO: 1, 3, 4, 6, 7, 9, or naturally occurring mutants thereof or 5′ or 3′ flanking sequences naturally associated with the 33312, 33303, or 32579 gene; (ii) exposing the probe/primer to nucleic acid of the tissue; and detecting, by hybridization, e.g., in situ hybridization, of the probe/primer to the nucleic acid, the presence or absence of the genetic lesion.

In preferred embodiments detecting the misexpression includes ascertaining the existence of at least one of: an alteration in the level of a messenger RNA transcript of the 33312, 33303, or 32579 gene; the presence of a non-wild type splicing pattern of a messenger RNA transcript of the gene; or a non-wild type level of 33312, 33303, or 32579.

Methods of the invention can be used prenatally or to determine if a subject's offspring will be at risk for a disorder.

In preferred embodiments the method includes determining the structure of a 33312, 33303, or 32579 gene, an abnormal structure being indicative of risk for the disorder.

In preferred embodiments the method includes contacting a sample form the subject with an antibody to the 33312, 33303, or 32579 protein or a nucleic acid, which hybridizes specifically with the gene. There and other embodiments are discussed below.

Diagnostic and Prognostic Assays of 33312, 33303, and 32579

Diagnostic and prognostic assays of the invention include method for assessing the expression level of 33312, 33303, or 32579 molecules and for identifying variations and mutations in the sequence of 33312, 33303, or 32579 molecules.

### Expression Monitoring and Profiling:

The presence, level, or absence of 33312, 33303, or 32579 protein or nucleic acid in a biological sample can be evaluated by obtaining a biological sample from a test subject and contacting the biological sample with a compound or an agent capable of detecting 33312, 33303, or 32579 protein or nucleic acid (e.g., mRNA, genomic DNA) that encodes 33312, 33303, or 32579 protein such that the presence of 33312, 33303, or 32579 protein or nucleic acid is detected in the biological sample. The term “biological sample” includes tissues, cells and biological fluids isolated from a subject, as well as tissues, cells and fluids present within a subject. A preferred biological sample is serum. The level of expression of the 33312, 33303, or 32579 gene can be measured in a number of ways, including, but not limited to: measuring the mRNA encoded by the 33312, 33303, or 32579 genes; measuring the amount of protein encoded by the 33312, 33303, or 32579 genes; or measuring the activity of the protein encoded by the 33312, 33303, or 32579 genes.

The level of mRNA corresponding to the 33312, 33303, or 32579 gene in a cell can be determined both by in situ and by in vitro formats.

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length 33312, 33303, or 32579 nucleic acid, such as the nucleic acid of SEQ ID NO: 1, or a portion thereof, such as an oligonucleotide of at least 7, 15, 30, 50, 100, 250 or 500 nucleotides in length and sufficient to specifically hybridize under stringent conditions to 33312, 33303, or 32579 mRNA or genomic DNA. The probe can be disposed on an address of an array, e.g., an array described below. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array described below. A skilled artisan can adapt known mRNA detection methods for use in detecting the level of mRNA encoded by the 33312, 33303, or 32579 genes.

The level of mRNA in a sample that is encoded by one of 33312, 33303, or 32579 can be evaluated with nucleic acid amplification, e.g., by rtPCR (Mullis (1987) U.S. Pat. No. 4,683,202), ligase chain reaction (Barany (1991) Proc. Natl. Acad. Sci. USA 88:189-193), self sustained sequence replication (Guatelli et al., (1990) Proc. Natl. Acad. Sci. USA 87:1874-1878), transcriptional amplification system (Kwoh et al., (1989), Proc. Natl. Acad. Sci. USA 86:1173-1177), Q-Beta Replicase (Lizardi et al., (1988) Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the 33312, 33303, or 32579 gene being analyzed.

In another embodiment, the methods further contacting a control sample with a compound or agent capable of detecting 33312, 33303, or 32579 mRNA, or genomic DNA, and comparing the presence of 33312, 33303, or 32579 mRNA or genomic DNA in the control sample with the presence of 33312, 33303, or 32579 mRNA or genomic DNA in the test sample. In still another embodiment, serial analysis of gene expression, as described in U.S. Pat. No. 5,695,937, is used to detect 33312, 33303, or 32579 transcript levels.

A variety of methods can be used to determine the level of protein encoded by 33312, 33303, or 32579. In general, these methods include contacting an agent that selectively binds to the protein, such as an antibody with a sample, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect 33312, 33303, or 32579 protein in a biological sample in vitro as well as in vivo. In vitro techniques for detection of 33312, 33303, or 32579 protein include enzyme linked immunosorbent assays (ELISAs), immunoprecipitations, immunofluorescence, enzyme immunoassay (EIA), radioimmunoassay (RIA), and Western blot analysis. In vivo techniques for detection of 33312, 33303, or 32579 protein include introducing into a subject a labeled anti-33312, 33303, or 32579 antibody. For example, the antibody can be labeled with a radioactive marker whose presence and location in a subject can be detected by standard imaging techniques. In another embodiment, the sample is labeled, e.g., biotinylated and then contacted to the antibody, e.g., an anti-33312, 33303, or 32579 antibody positioned on an antibody array (as described below). The sample can be detected, e.g., with avidin coupled to a fluorescent label.

In another embodiment, the methods further include contacting the control sample with a compound or agent capable of detecting 33312, 33303, or 32579 protein, and comparing the presence of 33312, 33303, or 32579 protein in the control sample with the presence of 33312, 33303, or 32579 protein in the test sample.

The invention also includes kits for detecting the presence of 33312, 33303, or 32579 in a biological sample. For example, the kit can include a compound or agent capable of detecting 33312, 33303, or 32579 protein or mRNA in a biological sample; and a standard. The compound or agent can be packaged in a suitable container. The kit can further comprise instructions for using the kit to detect 33312, 33303, or 32579 protein or nucleic acid.

For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can include: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also includes a buffering agent, a preservative, or a protein stabilizing agent. The kit can also includes components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, along with instructions for interpreting the results of the assays performed using the kit.

The diagnostic methods described herein can identify subjects having, or at risk of developing, a disease or disorder associated with misexpressed or aberrant or unwanted 33312, 33303, or 32579 expression or activity. As used herein, the term “unwanted” includes an unwanted phenomenon involved in a biological response such as pain or deregulated cell proliferation.

In one embodiment, a disease or disorder associated with aberrant or unwanted 33312, 33303, or 32579 expression or activity is identified. A test sample is obtained from a subject and 33312, 33303, or 32579 protein or nucleic acid (e.g., mRNA or genomic DNA) is evaluated, wherein the level, e.g., the presence or absence, of 33312, 33303, or 32579 protein or nucleic acid is diagnostic for a subject having or at risk of developing a disease or disorder associated with aberrant or unwanted 33312, 33303, or 32579 expression or activity. As used herein, a “test sample” refers to a biological sample obtained from a subject of interest, including a biological fluid (e.g., serum), cell sample, or tissue.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted 33312, 33303, or 32579 expression or activity. For example, such methods can be used to determine whether a subject can be effectively treated with an agent for a cell experiencing a misexpressed or aberrant or unwanted 33312, 33303, or 32579 expression or activity.

In another aspect, the invention features a computer medium having a plurality of digitally encoded data records. Each data record includes a value representing the level of expression of 33312, 33303, or 32579 in a sample, and a descriptor of the sample. The descriptor of the sample can be an identifier of the sample, a subject from which the sample was derived (e.g., a patient), a diagnosis, or a treatment (e.g., a preferred treatment). In a preferred embodiment, the data record further includes values representing the level of expression of genes other than 33312, 33303, or 32579 (e.g., other genes associated with a 33312, 33303, or 32579-disorder, or other genes on an array). The data record can be structured as a table, e.g., a table that is part of a database such as a relational database (e.g., a SQL database of the Oracle or Sybase database environments).

Also featured is a method of evaluating a sample. The method includes providing a sample, e.g., from the subject, and determining a gene expression profile of the sample, wherein the profile includes a value representing the level of 33312, 33303, or 32579 expression. The method can further include comparing the value or the profile (i.e., multiple values) to a reference value or reference profile. The gene expression profile of the sample can be obtained by any of the methods described herein (e.g., by providing a nucleic acid from the sample and contacting the nucleic acid to an array). The method can be used to diagnose a disorder in a subject wherein the disorder is associated with a misexpressed or aberrant or unwanted 33312, 33303, or 32579 expression or activity. The method can be used to monitor a treatment for misexpressed or aberrant or unwanted 33312, 33303, or 32579 expression or activity in a subject. For example, the gene expression profile can be determined for a sample from a subject undergoing treatment. The profile can be compared to a reference profile or to a profile obtained from the subject prior to treatment or prior to onset of the disorder (see, e.g., Golub et al. (1999) Science 286:531).

In yet another aspect, the invention features a method of evaluating a test compound (see also, “Screening Assays”, above). The method includes providing a cell and a test compound; contacting the test compound to the cell; obtaining a subject expression profile for the contacted cell; and comparing the subject expression profile to one or more reference profiles. The profiles include a value representing the level of 33312, 33303, or 32579 expression. In a preferred embodiment, the subject expression profile is compared to a target profile, e.g., a profile for a normal cell or for desired condition of a cell. The test compound is evaluated favorably if the subject expression profile is more similar to the target profile than an expression profile obtained from an uncontacted cell.

In another aspect, the invention features, a method of evaluating a subject. The method includes: a) obtaining a sample from a subject, e.g., from a caregiver, e.g., a caregiver who obtains the sample from the subject; b) determining a subject expression profile for the sample. Optionally, the method further includes either or both of steps: c) comparing the subject expression profile to one or more reference expression profiles; and d) selecting the reference profile most similar to the subject reference profile. The subject expression profile and the reference profiles include a value representing the level of 33312, 33303, or 32579 expression. A variety of routine statistical measures can be used to compare two reference profiles. One possible metric is the length of the distance vector that is the difference between the two profiles. Each of the subject and reference profile is represented as a multi-dimensional vector, wherein each dimension is a value in the profile.

The method can further include transmitting a result to a caregiver. The result can be the subject expression profile, a result of a comparison of the subject expression profile with another profile, a most similar reference profile, or a descriptor of any of the aforementioned. The result can be transmitted across a computer network, e.g., the result can be in the form of a computer transmission, e.g., a computer data signal embedded in a carrier wave.

Also featured is a computer medium having executable code for effecting the following steps: receive a subject expression profile; access a database of reference expression profiles; and either i) select a matching reference profile most similar to the subject expression profile or ii) determine at least one comparison score for the similarity of the subject expression profile to at least one reference profile. The subject expression profile, and the reference expression profiles each include a value representing the level of 33312, 33303, or 32579 expression.

33312, 33303, and 32579 Arrays and Uses Thereof

In another aspect, the invention features an array that includes a substrate having a plurality of addresses. At least one address of the plurality includes a capture probe that binds specifically to a 33312, 33303, or 32579 molecule (e.g., a 33312, 33303, or 32579 nucleic acid or a 33312, 33303, or 32579 polypeptide). The array can have a density of at least than 10, 50, 100, 200, 500, 1,000, 2,000, or 10,000 or more addresses/cm², and ranges between. In a preferred embodiment, the plurality of addresses includes at least 10, 100, 500, 1,000, 5,000, 10,000, 50,000 addresses. In a preferred embodiment, the plurality of addresses includes equal to or less than 10, 100, 500, 1,000, 5,000, 10,000, or 50,000 addresses. The substrate can be a two-dimensional substrate such as a glass slide, a wafer (e.g., silica or plastic), a mass spectroscopy plate, or a three-dimensional substrate such as a gel pad. Addresses in addition to address of the plurality can be disposed on the array.

In a preferred embodiment, at least one address of the plurality includes a nucleic acid capture probe that hybridizes specifically to a 33312, 33303, or 32579 nucleic acid, e.g., the sense or anti-sense strand. In one preferred embodiment, a subset of addresses of the plurality of addresses has a nucleic acid capture probe for 33312, 33303, or 32579. Each address of the subset can include a capture probe that hybridizes to a different region of a 33312, 33303, or 32579 nucleic acid. In another preferred embodiment, addresses of the subset include a capture probe for a 33312, 33303, or 32579 nucleic acid. Each address of the subset is unique, overlapping, and complementary to a different variant of 33312, 33303, or 32579 (e.g., an allelic variant, or all possible hypothetical variants). The array can be used to sequence 33312, 33303, or 32579 by hybridization (see, e.g., U.S. Pat. No. 5,695,940).

An array can be generated by various methods, e.g., by photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145).

In another preferred embodiment, at least one address of the plurality includes a polypeptide capture probe that binds specifically to a 33312, 33303, or 32579 polypeptide or fragment thereof. The polypeptide can be a naturally-occurring interaction partner of 33312, 33303, or 32579 polypeptide. Preferably, the polypeptide is an antibody, e.g., an antibody described herein (see “Anti-33312, 33303, or 32579 Antibodies,” above), such as a monoclonal antibody or a single-chain antibody.

In another aspect, the invention features a method of analyzing the expression of 33312, 33303, or 32579. The method includes providing an array as described above; contacting the array with a sample and detecting binding of a 33312, 33303, or 32579-molecule (e.g., nucleic acid or polypeptide) to the array. In a preferred embodiment, the array is a nucleic acid array. Optionally the method further includes amplifying nucleic acid from the sample prior or during contact with the array.

In another embodiment, the array can be used to assay gene expression in a tissue to ascertain tissue specificity of genes in the array, particularly the expression of 33312, 33303, or 32579. If a sufficient number of diverse samples is analyzed, clustering (e.g., hierarchical clustering, k-means clustering, Bayesian clustering and the like) can be used to identify other genes which are co-regulated with 33312, 33303, or 32579. For example, the array can be used for the quantitation of the expression of multiple genes. Thus, not only tissue specificity, but also the level of expression of a battery of genes in the tissue is ascertained. Quantitative data can be used to group (e.g., cluster) genes on the basis of their tissue expressionperse and level of expression in that tissue.

For example, array analysis of gene expression can be used to assess the effect of cell-cell interactions on 33312, 33303, or 32579 expression. A first tissue can be perturbed and nucleic acid from a second tissue that interacts with the first tissue can be analyzed. In this context, the effect of one cell type on another cell type in response to a biological stimulus can be determined, e.g., to monitor the effect of cell-cell interaction at the level of gene expression.

In another embodiment, cells are contacted with a therapeutic agent. The expression profile of the cells is determined using the array, and the expression profile is compared to the profile of like cells not contacted with the agent. For example, the assay can be used to determine or analyze the molecular basis of an undesirable effect of the therapeutic agent. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor expression of one or more genes in the array with respect to time. For example, samples obtained from different time points can be probed with the array. Such analysis can identify and/or characterize the development of a 33312, 33303, or 32579-associated disease or disorder; and processes, such as a cellular transformation associated with a 33312, 33303, or 32579-associated disease or disorder. The method can also evaluate the treatment and/or progression of a 33312, 33303, or 32579-associated disease or disorder

The array is also useful for ascertaining differential expression patterns of one or more genes in normal and abnormal cells. This provides a battery of genes (e.g., including 33312, 33303, or 32579) that could serve as a molecular target for diagnosis or therapeutic intervention.

In another aspect, the invention features an array having a plurality of addresses. Each address of the plurality includes a unique polypeptide. At least one address of the plurality has disposed thereon a 33312, 33303, or 32579 polypeptide or fragment thereof. Methods of producing polypeptide arrays are described in the art, e.g., in De Wildt et al. (2000). Nature Biotech. 18, 989-994; Lueking et al. (1999). Anal. Biochem. 270, 103-111; Ge, H. (2000). Nucleic Acids Res. 28, e3, I-VII; MacBeath, G., and Schreiber, S. L. (2000). Science 289, 1760-1763; and WO 99/51773A1. In a preferred embodiment, each addresses of the plurality has disposed thereon a polypeptide at least 60, 70, 80,85, 90, 95 or 99% identical to a 33312, 33303, or 32579 polypeptide or fragment thereof. For example, multiple variants of a 33312, 33303, or 32579 polypeptide (e.g., encoded by allelic variants, site-directed mutants, random mutants, or combinatorial mutants) can be disposed at individual addresses of the plurality. Addresses in addition to the address of the plurality can be disposed on the array.

The polypeptide array can be used to detect a 33312, 33303, or 32579 binding compound, e.g., an antibody in a sample from a subject with specificity for a 33312, 33303, or 32579 polypeptide or the presence of a 33312, 33303, or 32579-binding protein or ligand.

The array is also useful for ascertaining the effect of the expression of a gene on the expression of other genes in the same cell or in different cells (e.g. ascertaining the effect of 33312, 33303, or 32579 expression on the expression of other genes). This provides, for example, for a selection of alternate molecular targets for therapeutic intervention if the ultimate or downstream target cannot be regulated.

In another aspect, the invention features a method of analyzing a plurality of probes. The method is useful, e.g., for analyzing gene expression. The method includes: providing a two dimensional array having a plurality of addresses, each address of the plurality being positionally distinguishable from each other address of the plurality having a unique capture probe, e.g., wherein the capture probes are from a cell or subject which express 33312, 33303, or 32579 or from a cell or subject in which a 33312, 33303, or 32579 mediated response has been elicited, e.g., by contact of the cell with 33312, 33303, or 32579 nucleic acid or protein, or administration to the cell or subject 33312, 33303, or 32579 nucleic acid or protein; providing a two dimensional array having a plurality of addresses, each address of the plurality being positionally distinguishable from each other address of the plurality, and each address of the plurality having a unique capture probe, e.g., wherein the capture probes are from a cell or subject which does not express 33312, 33303, or 32579 (or does not express as highly as in the case of the 33312, 33303, or 32579 positive plurality of capture probes) or from a cell or subject which in which a 33312, 33303, or 32579 mediated response has not been elicited (or has been elicited to a lesser extent than in the first sample); contacting the array with one or more inquiry probes (which is preferably other than a 33312, 33303, or 32579 nucleic acid, polypeptide, or antibody), and thereby evaluating the plurality of capture probes. Binding, e.g., in the case of a nucleic acid, hybridization with a capture probe at an address of the plurality, is detected, e.g., by signal generated from a label attached to the nucleic acid, polypeptide, or antibody.

In another aspect, the invention features a method of analyzing a plurality of probes or a sample. The method is useful, e.g., for analyzing gene expression. The method includes: providing a two dimensional array having a plurality of addresses, each address of the plurality being positionally distinguishable from each other address of the plurality having a unique capture probe, contacting the array with a first sample from a cell or subject which express or mis-express 33312, 33303, or 32579 or from a cell or subject in which a 33312, 33303, or 32579-mediated response has been elicited, e.g., by contact of the cell with 33312, 33303, or 32579 nucleic acid or protein, or administration to the cell or subject 33312, 33303, or 32579 nucleic acid or protein; providing a two dimensional array having a plurality of addresses, each address of the plurality being positionally distinguishable from each other address of the plurality, and each address of the plurality having a unique capture probe, and contacting the array with a second sample from a cell or subject which does not express 33312, 33303, or 32579 (or does not express as highly as in the case of the 33312, 33303, or 32579 positive plurality of capture probes) or from a cell or subject which in which a 33312, 33303, or 32579 mediated response has not been elicited (or has been elicited to a lesser extent than in the first sample); and comparing the binding of the first sample with the binding of the second sample. Binding, e.g., in the case of a nucleic acid, hybridization with a capture probe at an address of the plurality, is detected, e.g., by signal generated from a label attached to the nucleic acid, polypeptide, or antibody. The same array can be used for both samples or different arrays can be used. If different arrays are used the plurality of addresses with capture probes should be present on both arrays.

In another aspect, the invention features a method of analyzing 33312, 33303, or 32579, e.g., analyzing structure, function, or relatedness to other nucleic acid or amino acid sequences. The method includes: providing a 33312, 33303, or 32579 nucleic acid or amino acid sequence; comparing the 33312, 33303, or 32579 sequence with one or more preferably a plurality of sequences from a collection of sequences, e.g., a nucleic acid or protein sequence database; to thereby analyze 33312, 33303, or 32579.

Detection of 33312, 33303, and 32579 Variations or Mutations

The methods of the invention can also be used to detect genetic alterations in a 33312, 33303, or 32579 gene, thereby determining if a subject with the altered gene is at risk for a disorder characterized by misregulation in 33312, 33303, or 32579 protein activity or nucleic acid expression. Examples of cytochrome P450 associated disorders in which the 33312, 33303, or 32579 molecules of the invention may be directly or indirectly involved include cellular proliferative and/or differentiative disorders; disorders associated with undesirable or deficient cell adhesion, motility or migration; inflammatory disorders, cell signaling associated disorders, metabolism associated disorders, steroids associated disorders; and fatty acid associated disorders. In preferred embodiments, the methods include detecting, in a sample from the subject, the presence or absence of a genetic alteration characterized by at least one of an alteration affecting the integrity of a gene encoding a 33312, 33303, or 32579-protein, or the mis-expression of the 33312, 33303, or 32579 gene. For example, such genetic alterations can be detected by ascertaining the existence of at least one of 1) a deletion of one or more nucleotides from a 33312, 33303, or 32579 gene; 2) an addition of one or more nucleotides to a 33312, 33303, or 32579 gene; 3) a substitution of one or more nucleotides of a 33312, 33303, or 32579 gene, 4) a chromosomal rearrangement of a 33312, 33303, or 32579 gene; 5) an alteration in the level of a messenger RNA transcript of a 33312, 33303, or 32579 gene, 6) aberrant modification of a 33312, 33303, or 32579 gene, such as of the methylation pattern of the genomic DNA, 7) the presence of a non-wild type splicing pattern of a messenger RNA transcript of a 33312, 33303, or 32579 gene, 8) a non-wild type level of a 33312, 33303, or 32579-protein, 9) allelic loss of a 33312, 33303, or 32579 gene, and 10) inappropriate post-translational modification of a 33312, 33303, or 32579-protein.

An alteration can be detected without a probe/primer in a polymerase chain reaction, such as anchor PCR or RACE PCR, or, alternatively, in a ligation chain reaction (LCR), the latter of which can be particularly useful for detecting point mutations in the 33312, 33303, or 32579-gene. This method can include the steps of collecting a sample of cells from a subject, isolating nucleic acid (e.g., genomic, mRNA or both) from the sample, contacting the nucleic acid sample with one or more primers which specifically hybridize to a 33312, 33303, or 32579 gene under conditions such that hybridization and amplification of the 33312, 33303, or 32579-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein. Alternatively, other amplification methods described herein or known in the art can be used.

In another embodiment, mutations in a 33312, 33303, or 32579 gene from a sample cell can be identified by detecting alterations in restriction enzyme cleavage patterns. For example, sample and control DNA is isolated, amplified (optionally), digested with one or more restriction endonucleases, and fragment length sizes are determined, e.g., by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 33312, 33303, or 32579 can be identified by hybridizing a sample and control nucleic acids, e.g., DNA or RNA, two-dimensional arrays, e.g., chip based arrays. Such arrays include a plurality of addresses, each of which is positionally distinguishable from the other. A different probe is located at each address of the plurality. A probe can be complementary to a region of a 33312, 33303, or 32579 nucleic acid or a putative variant (e.g., allelic variant) thereof. A probe can have one or more mismatches to a region of a 33312, 33303, or 32579 nucleic acid (e.g., a destabilizing mismatch). The arrays can have a high density of addresses, e.g., can contain hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in 33312, 33303, or 32579 can be identified in two-dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 33312, 33303, or 32579 gene and detect mutations by comparing the sequence of the sample 33312, 33303, or 32579 with the corresponding wild-type (control) sequence. Automated sequencing procedures can be utilized when performing the diagnostic assays ((1995) Biotechniques 19:448), including sequencing by mass spectrometry.

Other methods for detecting mutations in the 33312, 33303, or 32579 gene include methods in which protection from cleavage agents is used to detect mismatched bases in RNA/RNA or RNA/DNA heteroduplexes (Myers et al. (1985) Science 230:1242; Cotton et al. (1988) Proc. Natl Acad Sci USA 85:4397; Saleeba et al. (1992) Methods Enzymol. 217:286-295).

In still another embodiment, the mismatch cleavage reaction employs one or more proteins that recognize mismatched base pairs in double-stranded DNA (so called “DNA mismatch repair” enzymes) in defined systems for detecting and mapping point mutations in 33312, 33303, or 32579 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662; U.S. Pat. No. 5,459,039).

In other embodiments, alterations in electrophoretic mobility will be used to identify mutations in 33312, 33303, or 32579 genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control 33312, 33303, or 32579 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension (Saiki et al. (1986) Nature 324:163); Saiki et al. (1989) Proc. Natl. Acad. Sci USA 86:6230). A further method of detecting point mutations is the chemical ligation of oligonucleotides as described in Xu et al. ((2001) Nature Biotechnol. 19:148). Adjacent oligonucleotides, one of which selectively anneals to the query site, are ligated together if the nucleotide at the query site of the sample nucleic acid is complementary to the query oligonucleotide; ligation can be monitored, e.g., by fluorescent dyes coupled to the oligonucleotides.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

In another aspect, the invention features a set of oligonucleotides. The set includes a plurality of oligonucleotides, each of which is at least partially complementary (e.g., at least 50%, 60%, 70%, 80%, 90%, 92%, 95%, 97%, 98%, or 99% complementary) to a 33312, 33303, or 32579 nucleic acid.

In a preferred embodiment the set includes a first and a second oligonucleotide. The first and second oligonucleotide can hybridize to the same or to different locations of SEQ ID NO: 1, 3, 4, 6, 7 or 9, or the complement of SEQ ID NO: 1, 3, 4, 6, 7 or 9. Different locations can be different but overlapping or or nonoverlapping on the same strand. The first and second oligonucleotide can hybridize to sites on the same or on different strands.

The set can be useful, e.g., for identifying SNP's, or identifying specific alleles of 33312, 33303, or 32579. In a preferred embodiment, each oligonucleotide of the set has a different nucleotide at an interrogation position. In one embodiment, the set includes two oligonucleotides, each complementary to a different allele at a locus, e.g., a biallelic or polymorphic locus.

In another embodiment, the set includes four oligonucleotides, each having a different nucleotide (e.g., adenine, guanine, cytosine, or thymidine) at the interrogation position. The interrogation position can be a SNP or the site of a mutation. In another preferred embodiment, the oligonucleotides of the plurality are identical in sequence to one another (except for differences in length). The oligonucleotides can be provided with differential labels, such that an oligonucleotide that hybridizes to one allele provides a signal that is distinguishable from an oligonucleotide that hybridizes to a second allele. In still another embodiment, at least one of the oligonucleotides of the set has a nucleotide change at a position in addition to a query position, e.g., a destabilizing mutation to decrease the Tm of the oligonucleotide. In another embodiment, at least one oligonucleotide of the set has a non-natural nucleotide, e.g., inosine. In a preferred embodiment, the oligonucleotides are attached to a solid support, e.g., to different addresses of an array or to different beads or nanoparticles.

In a preferred embodiment the set of oligo nucleotides can be used to specifically amplify, e.g., by PCR, or detect, a 33312, 33303, or 32579 nucleic acid.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a 33312, 33303, or 32579 gene.

Use of 33312, 33303, or 32579 Molecules as Surrogate Markers

The 33312, 33303, or 32579 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the 33312, 33303, or 32579 molecules of the invention may be detected, and may be correlated with one or more biological states in vivo. For example, the 33312, 33303, or 32579 molecules of the invention may serve as surrogate markers for one or more disorders or disease states or for conditions leading up to disease states. As used herein, a “surrogate marker” is an objective biochemical marker that correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The 33312, 33303, or 32579 molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., a 33312, 33303, or 32579 marker) transcription or expression, the amplified marker may be in a quantity which is more readily detectable than the drug itself. Also, the marker may be more easily detected due to the nature of the marker itself; for example, using the methods described herein, anti-33312, 33303, or 32579 antibodies may be employed in an immune-based detection system for a 33312, 33303, or 32579 protein marker, or 33312, 33303, or 32579-specific radiolabeled probes may be used to detect a 33312, 33303, or 32579 mRNA marker. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am, J Health-Syst. Pharm. 56 Suppl. 3: S16-S20.

The 33312, 33303, or 32579 molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35:1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or protein (e.g., 33312, 33303, or 32579 protein or RNA) for specific tumor markers in a subject, a drug or course of treatment may be selected that is optimized for the treatment of the specific tumor likely to be present in the subject. Similarly, the presence or absence of a specific sequence mutation in 33312, 33303, or 32579 DNA may correlate 33312, 33303, or 32579 drug response. The use of pharmacogenomic markers therefore permits the application of the most appropriate treatment for each subject without having to administer the therapy.

Pharmaceutical Compositions of 33312, 33303, and 32579

The nucleic acid and polypeptides, fragments thereof, as well as anti-33312, 33303, or 32579 antibodies (also referred to herein as “active compounds”) of the invention can be incorporated into pharmaceutical compositions. Such compositions typically include the nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indeces are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

For antibodies, the preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The present invention encompasses agents which modulate expression or activity. An agent may, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

An antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin, maytansinoids, e.g., maytansinol (see U.S. Pat. No. 5,208,020), CC-1065 (see U.S. Pat. Nos. 5,475,092, 5,585,499, 5,846,545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids). Radioactive ions include, but are not limited to iodine, yttrium, lutetium and praseodymium.

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, .alpha.-interferon, beta.-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Methods of Treatment for 33312, 33303, and 32579

The 33312, 33303, or 32579 cytochrome P450 molecules can be used to treat disorders in which modulating activity or expression of 33312, 33303, or 32579 cytochrome P450 polypeptide or nucleic acid can ameliorate one or more symptoms of the disorder. The present invention thus provides for both prophylactic and therapeutic methods of treating a subject at risk of (or susceptible to) a disorder or having a disorder associated with aberrant or unwanted 33312, 33303, or 32579 expression or activity. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides.

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the 33312, 33303, or 32579 molecules of the present invention or 33312, 33303, or 32579 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

In one aspect, the invention provides a method for preventing in a subject, a disease or condition associated with an aberrant or unwanted 33312, 33303, or 32579 expression or activity, by administering to the subject a 33312, 33303, or 32579 or an agent which modulates 33312, 33303, or 32579 expression or at least one 33312, 33303, or 32579 activity. Subjects at risk for a disease which is caused or contributed to by aberrant or unwanted 33312, 33303, or 32579 expression or activity can be identified by, for example, any or a combination of diagnostic or prognostic assays as described herein. Administration of a prophylactic agent can occur prior to the manifestation of symptoms characteristic of the 33312, 33303, or 32579 aberrance, such that a disease or disorder is prevented or, alternatively, delayed in its progression. Depending on the type of 33312, 33303, or 32579 aberrance, for example, a 33312, 33303, or 32579 agonist or 33312, 33303, or 32579 antagonist agent can be used for treating the subject. The appropriate agent can be determined based on screening assays described herein.

It is possible that some 33312, 33303, or 32579 disorders can be caused, at least in part, by an abnormal level of gene product, or by the presence of a gene product exhibiting abnormal activity. As such, the reduction in the level and/or activity of such gene products would bring about the amelioration of disorder symptoms.

The 33312, 33303, or 32579 molecules can act as novel diagnostic targets and therapeutic agents for controlling one or more of cellular proliferative and/or differentiative disorders, hematopoietic or immune disorders, or metabolic disorders as described above, as well as disorders associated with bone metabolism, erythroid cell-associated disorders, cardiovascular disorders, liver disorders, viral diseases, or pain disorders.

As used herein, the term “erythroid associated disorders” or “erythroid cell-associated disorders” include disorders involving aberrant (increased or deficient) erythroblast proliferation, e.g., an erythroleukemia, and aberrant (increased or deficient) erythroblast differentiation, e.g., an anemia. Erythrocyte-associated disorders include anemias such as, for example, hemolytic anemias due to hereditary cell membrane abnormalities, such as hereditary spherocytosis, hereditary elliptocytosis, and hereditary pyropoikilocytosis; hemolytic anemias due to acquired cell membrane defects, such as paroxysmal nocturnal hemoglobinuria and spur cell anemia; hemolytic anemias caused by antibody reactions, for example to the RBC antigens, or antigens of the ABO system, Lewis system, Ii system, Rh system, Kidd system, Duffy system, and Kell system; methemoglobinemia; a failure of erythropoiesis, for example, as a result of aplastic anemia, pure red cell aplasia, myelodysplastic syndromes, sideroblastic anemias, and congenital dyserythropoietic anemia; secondary anemia in nonhematolic disorders, for example, as a result of chemotherapy, alcoholism, or liver disease; anemia of chronic disease, such as chronic renal failure; and endocrine deficiency diseases.

Aberrant expression and/or activity of 33312, 33303, or 32579 molecules may mediate disorders associated with bone metabolism. “Bone metabolism” refers to direct or indirect effects in the formation or degeneration of bone structures, e.g., bone formation, bone resorption, etc., which may ultimately affect the concentrations in serum of calcium and phosphate. This term also includes activities mediated by 33312, 33303, or 32579 molecules effects in bone cells, e.g. osteoclasts and osteoblasts, that may in turn result in bone formation and degeneration. For example, 33312, 33303, or 32579 molecules may support different activities of bone resorbing osteoclasts such as the stimulation of differentiation of monocytes and mononuclear phagocytes into osteoclasts. Accordingly, 33312, 33303, or 32579 molecules that modulate the production of bone cells can influence bone formation and degeneration, and thus may be used to treat bone disorders. Examples of such disorders include, but are not limited to, osteoporosis, osteodystrophy, osteomalacia, rickets, osteitis fibrosa cystica, renal osteodystrophy, osteosclerosis, anti-convulsant treatment, osteopenia, fibrogenesis-imperfecta ossium, secondary hyperparathyrodism, hypoparathyroidism, hyperparathyroidism, cirrhosis, obstructive jaundice, drug induced metabolism, medullary carcinoma, chronic renal disease, rickets, sarcoidosis, glucocorticoid antagonism, malabsorption syndrome, steatorrhea, tropical sprue, idiopathic hypercalcemia and milk fever.

Examples of disorders involving the heart or “cardiovascular disorder” include, but are not limited to, a disease, disorder, or state involving the cardiovascular system, e.g., the heart, the blood vessels, and/or the blood. A cardiovascular disorder can be caused by an imbalance in arterial pressure, a malfunction of the heart, or an occlusion of a blood vessel, e.g., by a thrombus. Examples of such disorders include hypertension, atherosclerosis, coronary artery spasm, congestive heart failure, coronary artery disease, valvular disease, arrhythmias, and cardiomyopathies.

Disorders which may be treated or diagnosed by methods described herein include, but are not limited to, disorders associated with an accumulation in the liver of fibrous tissue, such as that resulting from an imbalance between production and degradation of the extracellular matrix accompanied by the collapse and condensation of preexisting fibers. The methods described herein can be used to diagnose or treat hepatocellular necrosis or injury induced by a wide variety of agents including processes which disturb homeostasis, such as an inflammatory process, tissue damage resulting from toxic injury or altered hepatic blood flow, and infections (e.g., bacterial, viral and parasitic). For example, the methods can be used for the early detection of hepatic injury, such as portal hypertension or hepatic fibrosis. In addition, the methods can be employed to detect liver fibrosis attributed to inborn errors of metabolsim, for example, fibrosis resulting from a storage disorder such as Gaucher's disease (lipid abnormalities) or a glycogen storage disease, A1-antitrypsin deficiency; a disorder mediating the accumulation (e.g., storage) of an exogenous substance, for example, hemochromatosis (iron-overload syndrome) and copper storage diseases (Wilson's disease), disorders resulting in the accumulation of a toxic metabolite (e.g., tyrosinemia, fructosemia and galactosemia) and peroxisomal disorders (e.g., Zellweger syndrome). Additionally, the methods described herein may be useful for the early detection and treatment of liver injury associated with the administration of various chemicals or drugs, such as for example, methotrexate, isonizaid, oxyphenisatin, methyldopa, chlorpromazine, tolbutamide or alcohol, or which represents a hepatic manifestation of a vascular disorder such as obstruction of either the intrahepatic or extrahepatic bile flow or an alteration in hepatic circulation resulting, for example, from chronic heart failure, veno-occlusive disease, portal vein thrombosis or Budd-Chiari syndrome.

Additionally, 33312, 33303, or 32579 molecules may play an important role in the etiology of certain viral diseases, including, but not limited to, Hepatitis B, Hepatitis C and Herpes Simplex Virus (HSV). Modulators of 33312, 33303, or 32579 activity could be used to control viral diseases. The modulators can be used in the treatment and/or diagnosis of viral infected tissue or virus-associated tissue fibrosis, especially liver and liver fibrosis. Also, 33312, 33303, or 32579 modulators can be used in the treatment and/or diagnosis of virus-associated carcinoma, especially hepatocellular cancer.

Additionally, 33312, 33303, or 32579 may play an important role in the regulation of pain disorders. Examples of pain disorders include, but are not limited to, pain response elicited during various forms of tissue injury, e.g., inflammation, infection, and ischemia, usually referred to as hyperalgesia (described in, for example, Fields, H. L. (1987) Pain, New York:McGraw-Hill); pain associated with muscoloskeletal disorders, e.g., joint pain; tooth pain; headaches; pain associated with surgery; pain related to irritable bowel syndrome; or chest pain.

As discussed, successful treatment of 33312, 33303, or 32579 disorders can be brought about by techniques that serve to inhibit the expression or activity of target gene products. For example, compounds, e.g., an agent identified using an assays described above, that proves to exhibit negative modulatory activity, can be used in accordance with the invention to prevent and/or ameliorate symptoms of 33312, 33303, or 32579 disorders. Such molecules can include, but are not limited to peptides, phosphopeptides, small organic or inorganic molecules, or antibodies (including, for example, polyclonal, monoclonal, humanized, anti-idiotypic, chimeric or single chain antibodies, and Fab, F(ab′)₂ and Fab expression library fragments, scFV molecules, and epitope-binding fragments thereof).

Further, antisense and ribozyme molecules that inhibit expression of the target gene can also be used in accordance with the invention to reduce the level of target gene expression, thus effectively reducing the level of target gene activity. Still further, triple helix molecules can be utilized in reducing the level of target gene activity. Antisense, ribozyme and triple helix molecules are discussed above.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances in that the target gene encodes an extracellular protein, it can be preferable to co-administer normal target gene protein into the cell or tissue in order to maintain the requisite level of cellular or tissue target gene activity.

Another method by which nucleic acid molecules may be utilized in treating or preventing a disease characterized by 33312, 33303, or 32579 expression is through the use of aptamer molecules specific for 33312, 33303, or 32579 protein. Aptamers are nucleic acid molecules having a tertiary structure which permits them to specifically bind to protein ligands (see, e.g., Osborne, et al. Curr. Opin. Chem Biol. 1997, 1(1): 5-9; and Patel, D. J. Curr Opin Chem Biol 1997 June;1(1):32-46). Since nucleic acid molecules may in many cases be more conveniently introduced into target cells than therapeutic protein molecules may be, aptamers offer a method by which 33312, 33303, or 32579 protein activity may be specifically decreased without the introduction of drugs or other molecules which may have pluripotent effects.

Antibodies can be generated that are both specific for target gene product and that reduce target gene product activity. Such antibodies may, therefore, by administered in instances whereby negative modulatory techniques are appropriate for the treatment of 33312, 33303, or 32579 disorders. For a description of antibodies, see the Antibody section above.

In circumstances wherein injection of an animal or a human subject with a 33312, 33303, or 32579 protein or epitope for stimulating antibody production is harmful to the subject, it is possible to generate an immune response against 33312, 33303, or 32579 through the use of anti-idiotypic antibodies (see, for example, Herlyn, D. Ann Med 1999;31(1):66-78; and Bhattacharya-Chatterjee, M., and Foon, K. A. Cancer Treat Res 1998;94:51-68). If an anti-idiotypic antibody is introduced into a mammal or human subject, it should stimulate the production of anti-anti-idiotypic antibodies, which should be specific to the 33312, 33303, or 32579 protein. Vaccines directed to a disease characterized by 33312, 33303, or 32579 expression may also be generated in this fashion.

In instances where the target antigen is intracellular and whole antibodies are used, internalizing antibodies may be preferred. Lipofectin or liposomes can be used to deliver the antibody or a fragment of the Fab region that binds to the target antigen into cells. Where fragments of the antibody are used, the smallest inhibitory fragment that binds to the target antigen is preferred. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al. (1993), Proc. Natl. Acad. Sci. USA 90:7889-7893).

The identified compounds that inhibit target gene expression, synthesis and/or activity can be administered to a patient at therapeutically effective doses to prevent, treat or ameliorate 33312, 33303, or 32579 disorders. A therapeutically effective dose refers to that amount of the compound sufficient to result in amelioration of symptoms of the disorders.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays may utilize antibody mimics and/or “biosensors” that have been created through molecular imprinting techniques. The compound which is able to modulate 33312, 33303, or 32579 activity is used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell, R. J. et al (1996) Current Opinion in Biotechnology 7:89-94 and in Shea, K. J. (1994) Trends in Polymer Science 2:166-173. Such “imprinted” affinity matrixes are amenable to ligand-binding assays, whereby the immobilized monoclonal antibody component is replaced by an appropriately imprinted matrix. An example of the use of such matrixes in this way can be seen in Vlatakis, G. et al (1993) Nature 361:645-647. Through the use of isotope-labeling, the “free” concentration of compound which modulates the expression or activity of 33312, 33303, or 32579 can be readily monitored and used in calculations of IC50.

Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC50. An rudimentary example of such a “biosensor” is discussed in Kriz, D. et al (1995) Analytical Chemistry 67:2142-2144.

Another aspect of the invention pertains to methods of modulating 33312, 33303, or 32579 expression or activity for therapeutic purposes. Accordingly, in an exemplary embodiment, the modulatory method of the invention involves contacting a cell with a 33312, 33303, or 32579 or agent that modulates one or more of the activities of 33312, 33303, or 32579 protein activity associated with the cell. An agent that modulates 33312, 33303, or 32579 protein activity can be an agent as described herein, such as a nucleic acid or a protein, a naturally-occurring target molecule of a 33312, 33303, or 32579 protein (e.g., a 33312, 33303, or 32579 substrate or receptor), a 33312, 33303, or 32579 antibody, a 33312, 33303, or 32579 agonist or antagonist, a peptidomimetic of a 33312, 33303, or 32579 agonist or antagonist, or other small molecule.

In one embodiment, the agent stimulates one or 33312, 33303, or 32579 activities. Examples of such stimulatory agents include active 33312, 33303, or 32579 protein and a nucleic acid molecule encoding 33312, 33303, or 32579. In another embodiment, the agent inhibits one or more 33312, 33303, or 32579 activities. Examples of such inhibitory agents include antisense 33312, 33303, or 32579 nucleic acid molecules, anti-33312, 33303, or 32579 antibodies, and 33312, 33303, or 32579 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 33312, 33303, or 32579 protein or nucleic acid molecule. In one embodiment, the method involves administering an agent (e.g., an agent identified by a screening assay described herein), or combination of agents that modulates (e.g., upregulates or downregulates) 33312, 33303, or 32579 expression or activity. In another embodiment, the method involves administering a 33312, 33303, or 32579 protein or nucleic acid molecule as therapy to compensate for reduced, aberrant, or unwanted 33312, 33303, or 32579 expression or activity.

Stimulation of 33312, 33303, or 32579 activity is desirable in situations in which 33312, 33303, or 32579 is abnormally downregulated and/or in which increased 33312, 33303, or 32579 activity is likely to have a beneficial effect. For example, stimulation of 33312, 33303, or 32579 activity is desirable in situations in which a 33312, 33303, or 32579 is downregulated and/or in which increased 33312, 33303, or 32579 activity is likely to have a beneficial effect. Likewise, inhibition of 33312, 33303, or 32579 activity is desirable in situations in which 33312, 33303, or 32579 is abnormally upregulated and/or in which decreased 33312, 33303, or 32579 activity is likely to have a beneficial effect.

### 33312, 33303, and 32579 Pharmacogenomics

The 33312, 33303, or 32579 molecules of the present invention, as well as agents, or modulators which have a stimulatory or inhibitory effect on 33312, 33303, or 32579 activity (e.g., 33312, 33303, or 32579 gene expression) as identified by a screening assay described herein can be administered to individuals to treat (prophylactically or therapeutically) 33312, 33303, or 32579 associated disorders (e.g., cytochrome P450 associated disorders) associated with aberrant or unwanted 33312, 33303, or 32579 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a 33312, 33303, or 32579 molecule or 33312, 33303, or 32579 modulator as well as tailoring the dosage and/or therapeutic regimen of treatment with a 33312, 33303, or 32579 molecule or 33312, 33303, or 32579 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11):983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drug's target is known (e.g., a 33312, 33303, or 32579 protein of the present invention), all common variants of that gene can be fairly easily identified in the population and it can be determined if having one version of the gene versus another is associated with a particular drug response.

Alternatively, a method termed the “gene expression profiling”, can be utilized to identify genes that predict drug response. For example, the gene expression of an animal dosed with a drug (e.g., a 33312, 33303, or 32579 molecule or 33312, 33303, or 32579 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a 33312, 33303, or 32579 molecule or 33312, 33303, or 32579 modulator, such as a modulator identified by one of the exemplary screening assays described herein.

The present invention further provides methods for identifying new agents, or combinations, that are based on identifying agents that modulate the activity of one or more of the gene products encoded by one or more of the 33312, 33303, or 32579 genes of the present invention, wherein these products may be associated with resistance of the cells to a therapeutic agent. Specifically, the activity of the proteins encoded by the 33312, 33303, or 32579 genes of the present invention can be used as a basis for identifying agents for overcoming agent resistance. By blocking the activity of one or more of the resistance proteins, target cells, e.g., neuronal cells, will become sensitive to treatment with an agent that the unmodified target cells were resistant to.

Monitoring the influence of agents (e.g., drugs) on the expression or activity of a 33312, 33303, or 32579 protein can be applied in clinical trials. For example, the effectiveness of an agent determined by a screening assay as described herein to increase 33312, 33303, or 32579 gene expression, protein levels, or upregulate 33312, 33303, or 32579 activity, can be monitored in clinical trials of subjects exhibiting decreased 33312, 33303, or 32579 gene expression, protein levels, or downregulated 33312, 33303, or 32579 activity. Alternatively, the effectiveness of an agent determined by a screening assay to decrease 33312, 33303, or 32579 gene expression, protein levels, or downregulate 33312, 33303, or 32579 activity, can be monitored in clinical trials of subjects exhibiting increased 33312, 33303, or 32579 gene expression, protein levels, or upregulated 33312, 33303, or 32579 activity. In such clinical trials, the expression or activity of a 33312, 33303, or 32579 gene, and preferably, other genes that have been implicated in, for example, a 33312, 33303, or 32579 associated disorder can be used as a “read out” or markers of the phenotype of a particular cell.

33312, 33303, or 32579 Informatics

The sequence of a 33312, 33303, or 32579 molecule is provided in a variety of media to facilitate use thereof. A sequence can be provided as a manufacture, other than an isolated nucleic acid or amino acid molecule, which contains a 33312, 33303, or 32579. Such a manufacture can provide a nucleotide or amino acid sequence, e.g., an open reading frame, in a form which allows examination of the manufacture using means not directly applicable to examining the nucleotide or amino acid sequences, or a subset thereof, as they exists in nature or in purified form. The sequence information can include, but is not limited to, 33312, 33303, or 32579 full-length nucleotide and/or amino acid sequences, partial nucleotide and/or amino acid sequences, polymorphic sequences including single nucleotide polymorphisms (SNPs), epitope sequence, and the like. In a preferred embodiment, the manufacture is a machine-readable medium, e.g., a magnetic, optical, chemical or mechanical information storage device.

As used herein, “machine-readable media” refers to any medium that can be read and accessed directly by a machine, e.g., a digital computer or analogue computer. Non-limiting examples of a computer include a desktop PC, laptop, mainframe, server (e.g., a web server, network server, or server farm), handheld digital assistant, pager, mobile telephone, and the like. The computer can be stand-alone or connected to a communications network, e.g., a local area network (such as a VPN or intranet), a wide area network (e.g., an Extranet or the Internet), or a telephone network (e.g., a wireless, DSL, or ISDN network). Machine-readable media include, but are not limited to: magnetic storage media, such as floppy discs, hard disc storage medium, and magnetic tape; optical storage media such as CD-ROM; electrical storage media such as RAM, ROM, EPROM, EEPROM, flash memory, and the like; and hybrids of these categories such as magnetic/optical storage media.

A variety of data storage structures are available to a skilled artisan for creating a machine-readable medium having recorded thereon a nucleotide or amino acid sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. The skilled artisan can readily adapt any number of data processor structuring formats (e.g., text file or database) in order to obtain computer readable medium having recorded thereon the nucleotide sequence information of the present invention.

In a preferred embodiment, the sequence information is stored in a relational database (such as Sybase or Oracle). The database can have a first table for storing sequence (nucleic acid and/or amino acid sequence) information. The sequence information can be stored in one field (e.g., a first column) of a table row and an identifier for the sequence can be store in another field (e.g., a second column) of the table row. The database can have a second table, e.g., storing annotations. The second table can have a field for the sequence identifier, a field for a descriptor or annotation text (e.g., the descriptor can refer to a functionality of the sequence, a field for the initial position in the sequence to which the annotation refers, and a field for the ultimate position in the sequence to which the annotation refers. Non-limiting examples for annotation to nucleic acid sequences include polymorphisms (e.g., SNP's) translational regulatory sites and splice junctions. Non-limiting examples for annotations to amino acid sequence include polypeptide domains, e.g., a domain described herein; active sites and other functional amino acids; and modification sites.

By providing the nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or target structural motif with the sequence information stored within the data storage means. A search is used to identify fragments or regions of the sequences of the invention which match a particular target sequence or target motif. The search can be a BLAST search or other routine sequence comparison, e.g., a search described herein.

Thus, in one aspect, the invention features a method of analyzing 33312, 33303, or 32579, e.g., analyzing structure, function, or relatedness to one or more other nucleic acid or amino acid sequences. The method includes: providing a 33312, 33303, or 32579 nucleic acid or amino acid sequence; comparing the 33312, 33303, or 32579 sequence with a second sequence, e.g., one or more preferably a plurality of sequences from a collection of sequences, e.g., a nucleic acid or protein sequence database to thereby analyze 33312, 33303, or 32579. The method can be performed in a machine, e.g., a computer, or manually by a skilled artisan.

The method can include evaluating the sequence identity between a 33312, 33303, or 32579 sequence and a database sequence. The method can be performed by accessing the database at a second site, e.g., over the Internet.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. Typical sequence lengths of a target sequence are from about 10 to 100 amino acids or from about 30 to 300 nucleotide residues. However, it is well recognized that commercially important fragments, such as sequence fragments involved in gene expression and protein processing, may be of shorter length.

Computer software is publicly available which allows a skilled artisan to access sequence information provided in a computer readable medium for analysis and comparison to other sequences. A variety of known algorithms are disclosed publicly and a variety of commercially available software for conducting search means are and can be used in the computer-based systems of the present invention. Examples of such software include, but are not limited to, MacPattern (EMBL), BLASTN and BLASTX (NCBI).

Thus, the invention features a method of making a computer readable record of a sequence of a 33312, 33303, or 32579 sequence which includes recording the sequence on a computer readable matrix. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′ end of the translated region.

In another aspect, the invention features, a method of analyzing a sequence. The method includes: providing a 33312, 33303, or 32579 sequence, or record, in machine-readable form; comparing a second sequence to the 33312, 33303, or 32579 sequence; thereby analyzing a sequence. Comparison can include comparing to sequences for sequence identity or determining if one sequence is included within the other, e.g., determining if the 33312, 33303, or 32579 sequence includes a sequence being compared. In a preferred embodiment the 33312, 33303, or 32579 or second sequence is stored on a first computer, e.g., at a first site and the comparison is performed, read, or recorded on a second computer, e.g., at a second site. E.g., the 33312, 33303, or 32579 or second sequence can be stored in a public or proprietary database in one computer, and the results of the comparison performed, read, or recorded on a second computer. In a preferred embodiment the record includes one or more of the following: identification of an ORF; identification of a domain, region, or site; identification of the start of transcription; identification of the transcription terminator; the full length amino acid sequence of the protein, or a mature form thereof, the 5′ end of the translated region.

In another aspect, the invention provides a machine-readable medium for holding instructions for performing a method for determining whether a subject has a 33312, 33303, or 32579-associated disease or disorder or a pre-disposition to a 33312, 33303, or 32579-associated disease or disorder, wherein the method comprises the steps of determining 33312, 33303, or 32579 sequence information associated with the subject and based on the 33312, 33303, or 32579 sequence information, determining whether the subject has a 33312, 33303, or 32579-associated disease or disorder or a pre-disposition to a 33312, 33303, or 32579-associated disease or disorder and/or recommending a particular treatment for the disease, disorder or pre-disease condition.

The invention further provides in an electronic system and/or in a network, a method for determining whether a subject has a 33312, 33303, or 32579-associated disease or disorder or a pre-disposition to a disease associated with a 33312, 33303, or 32579 wherein the method comprises the steps of determining 33312, 33303, or 32579 sequence information associated with the subject, and based on the 33312, 33303, or 32579 sequence information, determining whether the subject has a 33312, 33303, or 32579-associated disease or disorder or a pre-disposition to a 33312, 33303, or 32579-associated disease or disorder, and/or recommending a particular treatment for the disease, disorder or pre-disease condition. In a preferred embodiment, the method further includes the step of receiving information, e.g., phenotypic or genotypic information, associated with the subject and/or acquiring from a network phenotypic information associated with the subject. The information can be stored in a database, e.g., a relational database. In another embodiment, the method further includes accessing the database, e.g., for records relating to other subjects, comparing the 33312, 33303, or 32579 sequence of the subject to the 33312, 33303, or 32579 sequences in the database to thereby determine whether the subject as a 33312, 33303, or 32579-associated disease or disorder, or a pre-disposition for such.

The present invention also provides in a network, a method for determining whether a subject has a 33312, 33303, or 32579 associated disease or disorder or a pre-disposition to a 33312, 33303, or 32579-associated disease or disorder associated with 33312, 33303, or 32579, said method comprising the steps of receiving 33312, 33303, or 32579 sequence information from the subject and/or information related thereto, receiving phenotypic information associated with the subject, acquiring information from the network corresponding to 33312, 33303, or 32579 and/or corresponding to a 33312, 33303, or 32579-associated disease or disorder and based on one or more of the phenotypic information, the 33312, 33303, or 32579 information (e.g., sequence information and/or information related thereto), and the acquired information, determining whether the subject has a 33312, 33303, or 32579-associated disease or disorder or a pre-disposition to a 33312, 33303, or 32579-associated disease or disorder. The method may further comprise the step of recommending a particular treatment for the disease, disorder or pre-disease condition.

The present invention also provides a method for determining whether a subject has a 33312, 33303, or 32579-associated disease or disorder or apre-disposition to a 33312, 33303, or 32579-associated disease or disorder, said method comprising the steps of receiving information related to 33312, 33303, or 32579 (e.g., sequence information and/or information related thereto), receiving phenotypic information associated with the subject, acquiring information from the network related to 33312, 33303, or 32579 and/or related to a 33312, 33303, or 32579-associated disease or disorder, and based on one or more of the phenotypic information, the 33312, 33303, or 32579 information, and the acquired information, determining whether the subject has a 33312, 33303, or 32579-associated disease or disorder or a pre-disposition to a 33312, 33303, or 32579-associated disease or disorder. The method may further comprise the step of recommending a particular treatment for the disease, disorder or pre-disease condition.

This invention is further illustrated by the following examples that should not be construed as limiting. The contents of all references, patents and published patent applications cited throughout this application are incorporated herein by reference.

### Background of the 21509 and 33770 Invention

Short-chain dehydrogenases/reductases (SDRs) constitute a large and diverse collection of enzymes grouped into a superfamily of over 700 different enzymes including isomerases, lyases and oxidoreductases (Opperman et al (1999) Enzymology and Molecular Biology of Carbonyl Metabolism 7, ed. Weiner et al., Plenum Publishers, NY p. 365-371). Members of the SDR superfamily appear to have similar activities though they function via different mechanisms. The enzymes of this family cover a wide range of substrate specificities including sugars, steroids, alcohols, prostaglandins, metabolites (e.g., lipids), and aromatic compounds (Opperman et al. (1999) Enzymology and Molecular Biology of Carbonyl Metabolism 7, ed. Weiner et al., Plenum Publishers, NY p. 373-377).

SDRs function as dimers or tetramers. The subunits are composed of approximately 250 amino acid residues, an N-terminal co-enzyme binding pattern of GxxxGxG, and an active-site pattern of YxxK (Opperman et al. (1999) Enzymology and Molecular Biology of Carbonyl Metabolism 7, ed. Weiner et al., Plenum Publishers, NY p. 373-377). Although identity between different SDR members is at the 15-30% level, three-dimensional structures thus far analyzed reveal a highly similar conformation with a one-domain subunit composed of seven to eight β-strands.

One particular class of SDRs includes 3-ketoacyl-ACP synthases (KASs), enzymes that are involved in the biosynthesis of fatty acid molecules. These proteins catalyze the stepwise condensation of an acyl group, bound either to an acyl carrier protein (ACP) or a Coenzyme A (CoA) molecule, with molonyl-ACP. Several different types of KASs (e.g., KAS I, II, and III) have been identified based on their substrate specificity. KAS I enzymes catalyze the majority of condensations, using as substrates acyl-ACP molecules containing fatty acid precursor chains of up to 14 carbons. KAS II enzymes further lengthen the hydrocarbon chains produced by KAS I enzymes, resulting in the production of long-chain fatty acid precursors for stearic acid (18 carbons) and arachidonic acid (20 carbons). In contrast, KAS III enzymes have a role at the beginning of fatty acid synthesis, catalyzing the condensation of acetyl CoA and malonyl-ACP to form 3-ketobutyryl-ACP, which is subsequently converted into butyryl-ACP, a substrate for KAS I enzymes. Overexpression of KAS III in cells has been shown to lead to changes in the distribution of fatty acid chain lengths within the cells(Dehesh et al. (2001), Plant Physiology 125, 1103-14), and the activity of KAS III enzymes can be negatively regulated by medium chain acyl-ACP end products (e.g., lauroyl-ACP, a 12 carbon fatty acid precursor).

In humans, an X-linked recessive disorder, adrenomyeloneuropathy, is associated with the accumulation of very-long-chain fatty acids and cerebellar demyelination, resulting in progressive neurodegeneration. This suggests that the type of fatty acids present in a cell can have a major impact on cellular behavior. One possible explanation for this is the interaction between fatty acids and the endocrine system. Hormones affect the fatty acid composition of tissue lipids and, in turn, fatty acids influence the concentrations of hormones and neuropeptides produced by cells, as well as the concentrations of their receptors.

Another class of SDRs is the 17-β-hydroxysteroid dehydrogenases, (17-β-HSDs), which composes a group of at least eight distinct enzymes that interconvert androgens or estrogens between their active and relatively inactive forms. These enzymes have unique tissue distribution patterns and serve as either dehydrogenases or reductases, but typically not as both (Su et al. (1999) Endocrinology 140(11):5275-5284). Some act predominantly upon estrogen substrates, others act predominantly upon androgen substrates, and others act upon multiple substrates. For example, SDR 17-β-HSD2 serves as a 17-β-HSD for estrogen and multiple androgen substrates and as a 20-α-HSD for 20α-dihydroprogesterone (Wu et al. (1993) J. Biol. Chem. 268:12964-12969). Members of the 17-β-HSD family regulate active hormone levels in extraglandular tissues (Tremblay, M. R. (1999) Biorganic & Medicinal Chemistry 7:1013-1023). These peripheral tissues contribute to a large proportion of steroid hormone formation from the adrenal precursor dehydroepiandroesterone (DHEA) and its conjugated sulfate (DHEAS).

Reductive 17-β-HSDs are essential for the biosynthesis of E2 and testosterone in the gonads and, in addition, they modulate the activity of these steroids in a subset of extragonal tissues found in several species, especially primates (Nokelainen et al. (1998) Mol. Endocrinology 12(7):1048-1059). Males express 17-β-HSD3 which, in the testis, functions as a reductase to convert androstenedione to testosterone (Su et al. (1999) Endocrinology 140(11):5275-5284). Both males and females express 17 β-HSD2, which functions as a dehydrogenase in liver, placenta, prostrate and other tissues, but not in testis, to convert estradiol and testosterone into estrone and androstenedione, respectively, with equivalent efficiency (Su et al. (1999) Endocrinology 140(11):5275-5284).

Estrogenic 17 β-hydroxysteroid dehydrogenase (17 β-HSD1) controls the last step in the formation of all estrogens, and has been shown to use NADPH and NADH as cofactors (Jin et al. (1999) Biochem. and Biophys. Comm. 259:489-493). It belongs to the SDR family and has a characteristic Tyr-X-X-X-Lys sequence motif at the active site (Ghosh et al. (1995) Structure 3:503-513). Females express 17-β-HSD1 which, in the human ovary, placenta, and breast, acts as a reductase to convert estrone into estradiol. Estradiol is a potent stimulator of certain endocrine-dependent forms of breast cancer (Jin et al. (1999) Biochem. and Biophys. Comm. 259:489-493). Therefore, 17-β-HSD1 is a target for the design of inhibitors of estradiol formation for breast cancer therapy.

Members of the alcohol dehydrogenase and short-chain dehydrogenase/reductase families also catalyze the reversible, rate limiting conversion of retinol to retinal, while the oxidation of retinal to retinoic acid is catalyzed by members of the aldehyde dehydrogenase or P450 enzyme families (Deuster et al. (1996) Biochemistry 35:12221-12227). Other SDR/retinol dehydrogenases function in the visual cycle by converting either 11-cis-retinol to 11-cis-retinal or all trans-retinal to all trans-retinol (Simon et al. (1995) J. Biol. Chem. 270:1107-1112). Retinoic acid plays a key role in the regulation of embryonic development, spermatogenesis, and epithelial differentiation (Chambon et al. (1996) FASEB J. 10:940-954 and Mangelsdorf et al. (1995) Cell 83:841-850).

Alcohol dehydrogenases play fundamental roles in degradative, synthetic, and detoxification pathways and have been implicated in a variety of developmental processes and pathophysiological disease states. For example, allelic variations of ADH2 and ADH3 appear to influence the susceptibility of Asians to alcoholism and alcoholic liver cirrhosis (Thomasson et al. (1991) Am. J. Hum Genet. 48:677-681, Chao et al. (1994) Hepatology 19:360-366, and Higuchi et al. (1995) Am. J. Psychiatry 152:1219-1221). Furthermore, first-pass metabolism, the difference between the quantity of ethanol that reaches the systemic circulation by the intravenous route and the quantity that reaches the systemic circulation by an oral route, may occur in the liver via the activity of members of the mammalian ADH family (Yin et al. (1999) Enzymology and Molecular Biology of Carbonyl Metabolism 7, Plenum Publishers, New York).

Aldehyde dehydrogenases are enzymes that oxidize a wide variety of aliphatic and aromatic aldehydes. In mammals at least four different forms of the enzyme are known: class-1 (or Ald C) a tetrameric cytosolic enzyme, class-2 (or Ald M) a tetrameric mitochondrial enzyme, class-3 (or Ald D) a dimeric cytosolic enzyme, and class IV a microsomal enzyme. Aldehyde dehydrogenases have also been sequenced from fungal and bacterial species. Enzymes of the aldehyde dehydrogenase family share a conserved glutamic acid and a conserved cysteine residue. These residues have been implicated in the catalytic activity of mammalian aldehyde dehydrogenases. For example, mutation of the conserved cysteine to alanine destroyed dehydrogenase activity of rat 10-formyltetrahydrofolate dehydrogenase (FDH) while hydrolase activity and binding of NADP+ were unchanged.

Aldehyde dehydrogenases modify a wide variety of substrates in diverse pathways. For example, the liver cytosolic enzyme, 10-formyltetrahydrofolate dehydrogenase, a tetramer consisting of identical 99 kDa subunits, catalyzes two reactions: the NADP+-dependent oxidation of 10-formyltetrahydrofolate to tetrahydrofolate and CO₂ and the NADP+-independent hydrolase reaction of 10-formyltetrahydrofolate to tetrahydrofolate and formate. The physiological role of the enzyme is probably to recycle 10-formyltetrahydrofolate not required for purine synthesis back to tetrahydrofolate where it is available for other one-carbon reactions. Loss of 10-formyltetrahydrofolate dehydrogenase in transgenic knockout mice decreased the total folate pool while markedly depleting the level of tetrahydrofolate.

Short chain dehydrogenase/reductases, alcohol dehydrogenases and aldehyde dehydrogenases, inter alia, are important in metabolism of small molecules, production/removal of biologically important molecules that modulate development and growth, elimination of toxins, and associated physiological processes and pathological conditions. Accordingly, there is a need to identify short chain dehydrogenase/reductases, alcohol dehydrogenases and aldehyde dehydrogenases in order to better understand processes and pathological conditions in these proteins participate in or are associated with. The present invention addresses this need and provides related benefits including potential therapeutics for treating short chain dehydrogenase/reductase, alcohol dehydrogenase and aldehyde dehydrogenase associated pathological conditions.

### Summary of the 21509 and 33770 Invention

The present invention is based, in part, on the discovery of two novel dehydrogenase/ reductase genes, referred to herein as “21509” and “33770”. The nucleotide sequence of a DNA encoding 21509 and 33770 are shown in SEQ ID NOs:13 and 16, respectively. The amino acid sequence of a 21509 and 33770 polypeptide are shown in SEQ ID NOs:14 and 17, respectively. In addition, the nucleotide sequences of the 21509 and 33770 coding regions are depicted in SEQ ID NOs:15 and 18, respectively.

Accordingly, in one aspect, the invention features a nucleic acid molecule which encodes a 21509 or 33770 protein or polypeptide, e.g., a biologically active subsequence of the 21509 or 33770 protein. In one embodiment, isolated nucleic acid molecule encodes a polypeptide having the amino acid sequence of SEQ ID NO:14 or 17. In other embodiments, the invention provides isolated 21509 or 33770 nucleic acid molecules having the nucleotide sequence shown in SEQ ID NO: 13, SEQ ID NO: 15, SEQ ID NO: 16, SEQ ID NO: 18, or the sequence of the DNA insert of the plasmid deposited with ATCC Accession Number ______ or ______. In still other embodiments, the invention provides nucleic acid molecules that are substantially identical (e.g., naturally occurring allelic variants) to the nucleotide sequence shown in SEQ ID NO:13, SEQ ID NO:15, SEQ ID NO:16, SEQ ID NO:18, or the sequence of the DNA insert of the plasmid deposited with ATCC Accession Number ______ or ______. In other embodiments, the invention provides a nucleic acid molecule which hybridizes under stringent hybridization conditions to a nucleic acid molecule comprising the nucleotide sequence of SEQ ID NO: 13 or 15, or SEQ ID NO: 16 or 18, or the sequence of the DNA insert of the plasmid deposited with ATCC Accession Number ______ or ______, wherein the nucleic acid encodes a full length 21509 or 33770 protein or an active fragment thereof.

In a related aspect, the invention further provides nucleic acid constructs that include a 21509 or 33770 nucleic acid molecule described herein. In certain embodiments, the nucleic acid molecules of the invention are operatively linked to native or heterologous regulatory sequences. Also included, are vectors and host cells containing the 21509 or 33770 nucleic acid molecules of the invention e.g., vectors and host cells suitable for producing 21509 or 33770 nucleic acid molecules and polypeptides.

In another related aspect, the invention provides nucleic acid fragments suitable as primers or hybridization probes for the detection of 21509 or 33770-encoding nucleic acids.

In still another related aspect, isolated nucleic acid molecules that are antisense to a 21509 or 33770 encoding nucleic acid molecule are provided.

In another aspect, the invention features, 21509 or 33770 polypeptides, and biologically active or antigenic fragments thereof that are useful, e.g., as reagents or targets in assays applicable to treatment and diagnosis of 21509- or 33770-mediated or -related disorders. In another embodiment, the invention provides 21509 or 33770 polypeptides having a 21509 or 33770 activity. Preferred polypeptides are 21509 or 33770 proteins including at least one dehydrogenase/reductase domain, and, preferably, having a 21509 or 33770 activity, e.g., a 21509 or 33770 activity as described herein.

In other embodiments, the invention provides 21509 or 33770 polypeptides, e.g., a 21509 or 33770 polypeptide having the amino acid sequence shown in SEQ ID NO:14 or 17, or the amino acid sequence encoded by the cDNA insert of the plasmid deposited with ATCC Accession Number ______or ______; an amino acid sequence that is substantially identical to the amino acid sequence shown in SEQ ID NO:14 or 17, or the amino acid sequence encoded by the cDNA insert of the plasmid deposited with ATCC Accession Number ______ or ______; or an amino acid sequence encoded by a nucleic acid molecule having a nucleotide sequence which hybridizes under a stringency condition described herein to a nucleic acid molecule comprising the nucleotide sequence of SEQ ID NO:13 or 15, SEQ ID NO:16 or 18, or the sequence of the DNA insert of the plasmid deposited with ATCC Accession Number ______ or ______, wherein the nucleic acid encodes a full length 21509 or 33770 protein or an active fragment thereof.

In a related aspect, the invention provides 21509 or 33770 polypeptides or fragments operatively linked to non-21509 or non-33770 polypeptides to form fusion proteins.

In another aspect, the invention features antibodies, and antigen-binding fragments thereof, that react with or, more preferably, specifically bind 21509 or 33770 polypeptides.

In another aspect, the invention provides methods of screening for compounds that modulate the expression or activity of the 21509 or 33770 polypeptides or nucleic acids.

In still another aspect, the invention provides a process for modulating 21509 or 33770 polypeptide or nucleic acid expression or activity, e.g. using the screened compounds. In certain embodiments, the methods involve treatment of conditions related to aberrant activity or expression of the 21509 or 33770 polypeptides or nucleic acids, such as conditions involving aberrant or deficient cellular proliferation or differentiation, abnormal fatty acid metabolism, abnormal hormonal regulation, or pathophysiological diseases related to an impaired breakdown of toxins.

In yet another aspect, the invention provides methods for inhibiting the proliferation or migration, or inducing the killing, of a 21509- or 33770-expressing cell, e.g., a hyperproliferative and/or metastatic cell. The methods include contacting the cell with a compound (e.g., a compound identified using the methods described herein) that modulates the activity or expression of the 21509 or 33770 polypeptide or nucleic acid.

In a preferred embodiment, the 21509-expressing cell is found in the prostate, brain (nerve or glial cell), heart, liver, kidney, blood vessels (e.g., artery, vein, vascular smooth muscle, endothelia), skeletal muscle, breast, bone, ovary, colon, or lung.

In another preferred embodiment, the 21509- or 33770-expressing cells are hyperproliferative and/or metastatic, e.g., cells of a solid tumor, a soft tissue tumor, or a metastatic lesion. Preferably, the tumor is a sarcoma, a carcinoma, or an adenocarcinoma. Preferably, the hyperproliferative and/or metastatic cells are found in a cancerous or pre-cancerous tissue, e.g., a cancerous or pre-cancerous tissue where a 21509 or 33770 polypeptide or nucleic acid is expressed, e.g., the prostate, brain, heart, liver, bone, kidney, blood vessels (e.g., artery, vein, vascular smooth muscle, endothelia), skeletal muscle, breast, ovary, colon, or lung. More preferably, the hyperproliferative and/or metastatic cells are of ovarian, colon, lung, or breast tissue origin.

In a preferred embodiment, the contacting step is effective in vitro or ex vivo. In other embodiments, the contacting step is effected in vivo, e.g., in a subject (e.g., a mammal, e.g., a human), as part of a therapeutic or prophylactic protocol.

In one embodiment, the compound can be an inhibitor of a 21509 or 33770 polypeptide. Preferably, the inhibitor is chosen from a peptide, a phosphopeptide, a small organic molecule, and an antibody (e.g., an antibody conjugated to a therapeutic moiety selected from a cytotoxin, a cytotoxic agent, and a radioactive metal ion). In one preferred embodiment, the inhibitor is an analog or a derivative of a fatty acid, e.g., palmitic acid. In another preferred embodiment, the inhibitor is an analog or a derivative of 9-cis-retinal.

In another embodiment, the compound can be an activator of a 21509 or 33770 polypeptide. Preferably, the activator is chosen from a peptide, a phosphopeptide, a small organic molecule, and an antibody. The activator can also be an allosteric effector that stimulates dehydrogenase or reductase activity.

In another embodiment, the compound is an inhibitor of a 21509 or a 33770 nucleic acid, e.g., an antisense, ribozyme, or triple helix molecule.

In another embodiment, the compound is administered in combination with a cytotoxic agent. Examples of cytotoxic agents include an anti-microtubule agent, a topoisomerase I inhibitor, a topoisomerase II inhibitor, an anti-metabolite, a mitotic inhibitor, an alkylating agent, an intercalating agent, and agent capable of interfering with a signal transduction pathway, an agent that promotes apoptosis or necrosis, and radiation.

In another embodiment, the compound is administered in an amount sufficient to alter fatty acid biosynthesis within a cell. For example, the compound may alter the conversion of acetyl CoA and malonyl-acyl carrier protein (ACP) into 3-ketobutyryl-ACP.

In another embodiment, the compound is administered in an amount sufficient to alter the biosynthesis of a hormone within a cell. For example, the compound may alter the conversion of 9-cis-retinal to 9-cis-retinoic acid.

In another aspect, the invention features a method of modulating fatty acid or hormone biosynthesis in a 21509- or 33770-expressing cell (e.g., a prostate, brain (nerve or glial cell), heart, liver, kidney, blood vessels (e.g., artery, vein, vascular smooth muscle, endothelia), skeletal muscle, bone, breast, ovary, colon, lung, or cancer cell). The method includes, contacting the cell with a compound that modulates the activity or expression of a 21509 or 33770 polypeptide as described herein, in an amount which is sufficient to alter the biosynthesis of fatty acids or morphogens in the cell.

In a preferred embodiment, the compound is administered in an amount sufficient to alter (e.g., enhance or inhibit) the conversion of acetyl CoA and malonyl-acyl carrier protein (ACP) into 3-ketobutyryl-ACP, or 9-cis-retinal into 9-cis-retinoic acid, thereby mediating signaling between or within cells.

In a preferred embodiment, the contacting step is effective in vitro or ex vivo. In other embodiments, the contacting step is effected in vivo, e.g., in a subject (e.g., a mammal, e.g., a human), as part of a therapeutic or prophylactic protocol.

In a preferred embodiment, the 21509- or 33770-expressing cell is found in the prostate, brain (nerve or glial cell), heart, liver, kidney, blood vessels (e.g., artery, vein, vascular smooth muscle, endothelia), skeletal muscle, breast, ovary, colon, or lung.

In a preferred embodiment, the 21509- or 33770-expressing cell is found in a solid tumor, a soft tissue tumor, or a metastatic lesion. Preferably, the 21509 or 33770 expressing cells are hyperproliferative and/or metastatic. Preferably, the tumor is a sarcoma, a carcinoma, or an adenocarcinoma. Preferably, the hyperproliferative and/or metastatic cells are found in a cancerous or pre-cancerous tissue, e.g., a cancerous or pre-cancerous tissue where a 21509 or 33770 polypeptide or nucleic acid is expressed, e.g., prostate, brain (nerve or glial cell), heart, liver, kidney, blood vessels (e.g., artery, vein, vascular smooth muscle, endothelia), skeletal muscle, breast, ovary, colon, or lung tissue. More preferably, the hyperproliferative and/or metastatic cells are found in an ovarian, colon, lung, or breast cancer.

In another aspect, the invention features a method for treating or preventing a disorder characterized by aberrant cellular proliferation, migration, or differentiation of a 21509- or a 33770-expressing cell, in a subject. Preferably, the method includes administering to the subject (e.g., a mammal, e.g., a human) an effective amount of a compound (e.g., a compound identified using the methods described herein) that modulates the activity, or expression of the 21509 or 33770 polypeptide or nucleic acid.

In a preferred embodiment, the 21509- or 33770-expressing cell is found in the prostate, brain (nerve or glial cell), heart, liver, bone, kidney, blood vessels (e.g., artery, vein, vascular smooth muscle, endothelia), skeletal muscle, breast, ovary, colon, or lung.

In a preferred embodiment, the disorder is a neurological, cardiovascular, hepatic, renal, endothelial, bone, breast, immune, or skeletal muscular disorder.

In a preferred embodiment, the disorder is a cancerous or pre-cancerous condition. Most preferably, the disorder is a cancer, e.g., a solid tumor, a soft tissue tumor, or a metastatic lesion. Preferably, the cancer is a sarcoma, a carcinoma, or an adenocarcinoma. Preferably, the cancer is found in a tissue where a 21509 or 33770 polypeptide or nucleic acid is expressed, e.g., prostate, brain (nerve or glial cell), heart, liver, kidney, blood vessels (e.g., artery, vein, vascular smooth muscle, endothelia), skeletal muscle, breast, ovary, colon, or lung. Most preferably, the cancer is of ovary, colon, lung, or breast tissue origin.

In one embodiment, the compound can be an inhibitor of a 21509 or 33770 polypeptide. Preferably, the inhibitor is chosen from a peptide, a phosphopeptide, a small organic molecule, a small inorganic molecule, and an antibody (e.g., an antibody conjugated to a therapeutic moiety selected from a cytotoxin, a cytotoxic agent, and a radioactive metal ion). In a preferred embodiment, the inhibitor is a fatty acid analog or derivative, e.g., an analog or derivative of palmitic acid. In another embodiment, the inhibitor is a 9-cis-retinal analog or derivative.

In another embodiment, the compound can be an activator of a 21509 or 33770 polypeptide. Preferably, the activator is chosen from a peptide, a phosphopeptide, a small organic molecule, and an antibody. The activator can also be an allosteric effector that stimulates dehydrogenase or reductase activity.

In another embodiment, the compound is an inhibitor of a 21509 or a 33770 nucleic acid, e.g., an antisense, ribozyme, or triple helix molecule.

In another embodiment, the compound is administered in an amount sufficient to alter fatty acid biosynthesis within a cell. For example, the compound may alter the conversion of acetyl CoA and malonyl-ACP into 3-ketobutyryl-ACP.

In another embodiment, the compound is administered in an amount sufficient to alter the biosynthesis of a hormone within a cell. For example, the compound may alter the conversion of 9-cis-retinal to 9-cis-retinoic acid.

In another embodiment, the compound is administered in combination with a cytotoxic agent. Examples of cytotoxic agents include an anti-microtuble agent, a topoisomerase I inhibitor, a topoisomerase II inhibitor, an anti-metabolite, a mitotic inhibitor, an alkylating agent, an intercalating agent, and agent capable of interfering with a signal transduction pathway, an agent that promotes apoptosis or necrosis, and radiation.

The invention also provides assays for determining the activity of, or the presence or absence of, 21509 or 33770 polypeptides or nucleic acid molecules in a biological sample, including for disease diagnosis. Preferably, the biological sample includes a diseased cell or tissue. In one embodiment, the diseased cell or tissue is obtained from a subject having a neurological, cardiovascular, hepatic, renal, or skeletal muscular disorder. In other embodiments, the biological sample includes cancerous or pre-cancerous cell or tissue. For example, the cancerous tissue can be a solid tumor, a soft tissue tumor, or a metastatic lesion. Preferably, the cancerous tissue is a sarcoma, a carcinoma, or an adenocarcinoma. Preferably, the cancerous tissue is from the prostate, brain (nerve or glial cell), heart, liver, kidney, blood vessels (e.g., artery, vein, vascular smooth muscle, endothelia), skeletal muscle, breast, ovary, colon, or lung. Most preferably, the cancerous tissue is from the ovary, colon, lung, or breast. The activity of 21509 or 33770 polypeptides or nucleic acid molecules can be determined using a method described herein.

In a further aspect the invention provides assays for determining the presence or absence of a genetic alteration in a 21509 or 33770 polypeptide or nucleic acid molecule in a sample, for, e.g., disease diagnosis. Preferably, the biological sample includes a diseased cell or tissue. In one embodiment, the diseased cell or tissue is obtained from a subject having a neurological, cardiovascular, immune, bone, hepatic, renal or skeletal muscular disorder. In other embodiments, the biological sample includes cancerous or pre-cancerous cell or tissue. For example, the cancerous tissue can be a solid tumor, a soft tissue tumor, or a metastatic lesion. Preferably, the cancerous tissue is a sarcoma, a carcinoma, or an adenocarcinoma. Preferably, the cancerous tissue is from prostate, brain (nerve or glial cell), heart, liver, kidney, blood vessels (e.g., artery, vein, vascular smooth muscle, endothelia), skeletal muscle, breast, ovary, colon, or lung tissue. Most preferably, the cancerous tissue is from the ovary, colon, lung, or breast.

In a still further aspect, the invention provides methods for evaluating the efficacy of a treatment of a disorder, e.g., a neurological, cardiovascular, immune, bone, hepatic, renal or skeletal muscular disorder, or a hyperproliferative and/or metastatic disorder, e.g., cancer (e.g., ovarian, colon, lung, or breast cancer). The method includes: treating the subject, e.g., a patient or an animal, with a protocol under evaluation (e.g., treating a subject with one or more of: chemotherapy, radiation, and/or a compound identified using the methods described herein); and evaluating the activity of a 21509 or 33770 polypeptide, or the expression of a 21509 or 33770 polypeptide or nucleic acid, before and after treatment. A change, e.g., a decrease or increase, in the activity of a 21509 or 33770 polypeptide, or the expression of a 21509 or 33770 polypeptide or nucleic acid, relative to the level of activity or expression before treatment, is indicative of the efficacy of the treatment.

In a preferred embodiment, the disorder is a neurological, immune, bone, cardiovascular, hepatic, renal or skeletal muscular disorder.

In another preferred embodiment, the disorder is a cancer of the prostate, nervous system, heart, liver, kidney, blood vessels, skeletal muscle, breast, ovary, colon, or lung. Most preferably, the disorder is a cancer of the ovary, colon, lung, or breast. The activity of a 21509 or 33770 polypeptide, or the expression of a 21509 or 33770 polypeptide or nucleic acid, can be assayed, e.g., by a method described herein.

In a preferred embodiment, the evaluating step includes obtaining a sample (e.g., a tissue sample, e.g., a biopsy, or a fluid sample) from the subject, before and after treatment and comparing the level of activity and/or expression of a 21509 or 33770 polypeptide or nucleic acid before and after treatment.

In another aspect, the invention provides methods for evaluating the efficacy of a therapeutic or prophylactic agent (e.g., an anti-neoplastic and/or anti-metastatic agent). The method includes: contacting a sample with an agent (e.g., a compound identified using the methods described herein); and evaluating the activity and/or expression of a 21509 or 33770 polypeptide or nucleic acid in the sample, before and after the contacting step. A change, e.g., a decrease or increase in the level of 21509 or 33770 polypeptide or nucleic acid in the sample obtained after the contacting step, relative to the level of activity and/or expression in the sample before the contacting step, is indicative of the efficacy of the agent. The activity or expression level of 21509 or 33770 polypeptide or nucleic acid can be detected by any method described herein.

In a preferred embodiment, the sample includes cells obtained from a cancerous tissue where a 21509 or 33770 polypeptide or nucleic acid is expressed, e.g., a cancer of the ovary, colon, lung, or breast.

In a preferred embodiment, the sample is a tissue sample (e.g., a biopsy), a bodily fluid, or cultured cells (e.g., a tumor cell line).

In another aspect, the invention features a two dimensional array having a plurality of addresses, each address of the plurality being positionally distinguishable from each other address of the plurality, and each address of the plurality having a unique capture probe, e.g., a nucleic acid or peptide sequence. At least one address of the plurality has a capture probe that recognizes a 21509 or 33770 molecule. In one embodiment, the capture probe is a nucleic acid, e.g., a probe complementary to a 21509 or 33770 nucleic acid sequence. In another embodiment, the capture probe is a polypeptide, e.g., an antibody specific for 21509 or 33770 polypeptides. Also featured is a method of analyzing a sample by contacting the sample to the aforementioned array and detecting binding of the sample to the array.

Other features and advantages of the invention will be apparent from the following detailed description, and from the claims.

### DETAILED DESCRIPTION OF 21509 AND 33770

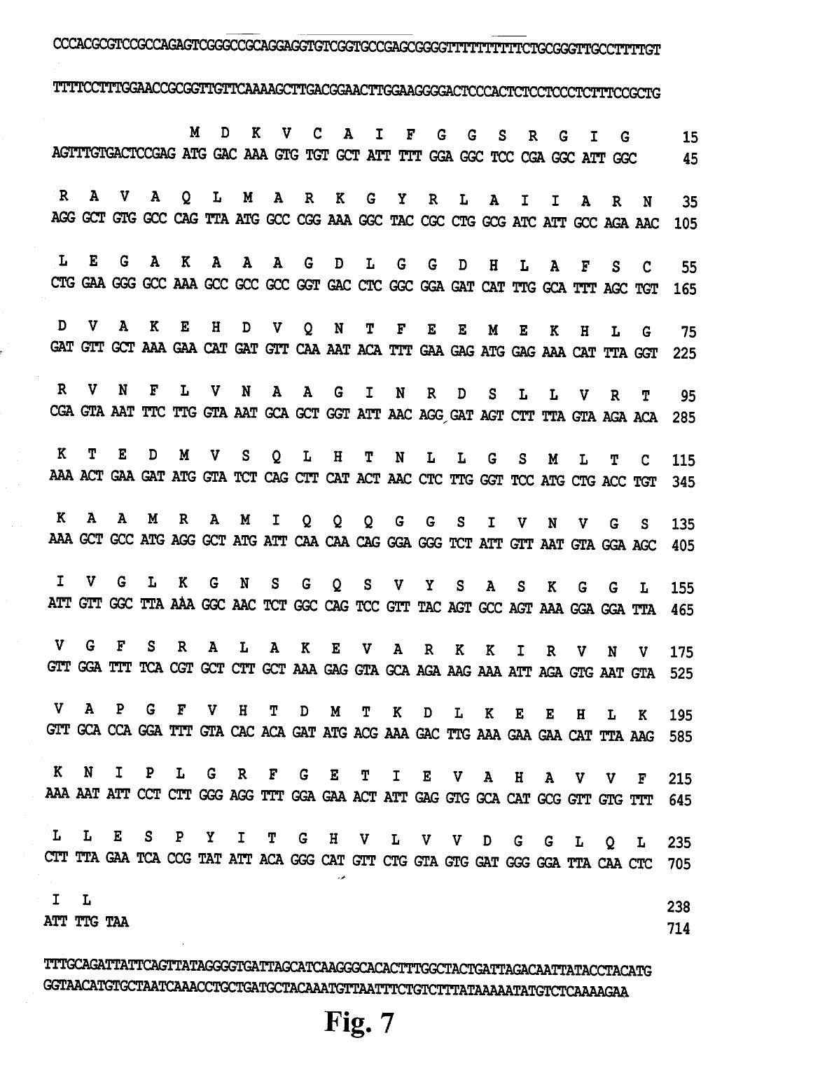

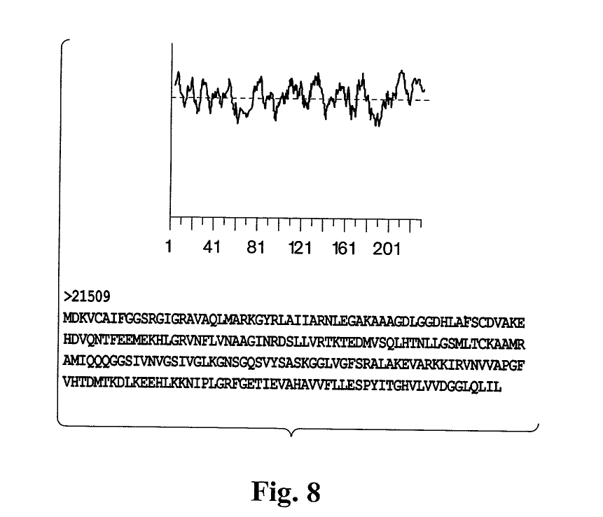

The human 21509 sequence (FIG. 7; SEQ ID NO:13), which is approximately 1043 nucleotides long including untranslated regions, contains a predicted methionine-initiated coding sequence of about 714 nucleotides, including the termination codon (nucleotides indicated as coding of SEQ ID NO:13 in FIG. 7; SEQ ID NO:15). The coding sequence encodes a 237 amino acid protein (SEQ ID NO:14).

Human 21509 contains the following regions or other structural features:

a short-chain alcohol dehydrogenase domain (PFAM Accession Number PF00106) located at about amino acid residues 3 to 229 of SEQ ID NO:14, which includes a short chain alcohol dehydrogenase family signature sequence, “YSASKGGLVGF”, located at about amino acid residues 148 to 158 of SEQ ID NO:14;

one predicted Protein Kinase C phosphorylation site (PS00005) located at about amino acid residues 114 to 116 of SEQ ID NO:14;

two predicted Casein Kinase II phosphorylation sites (PS00006) located at about amino acid residues 66 to 69 and 95 to 98 of SEQ ID NO: 14;

and six predicted N-myristylation sites (PS00008) located at about amino acids 9 to 14, 38 to 43, 110 to 115, 128 to 133, 134 to 139, and 153 to 158 of SEQ ID NO:14.

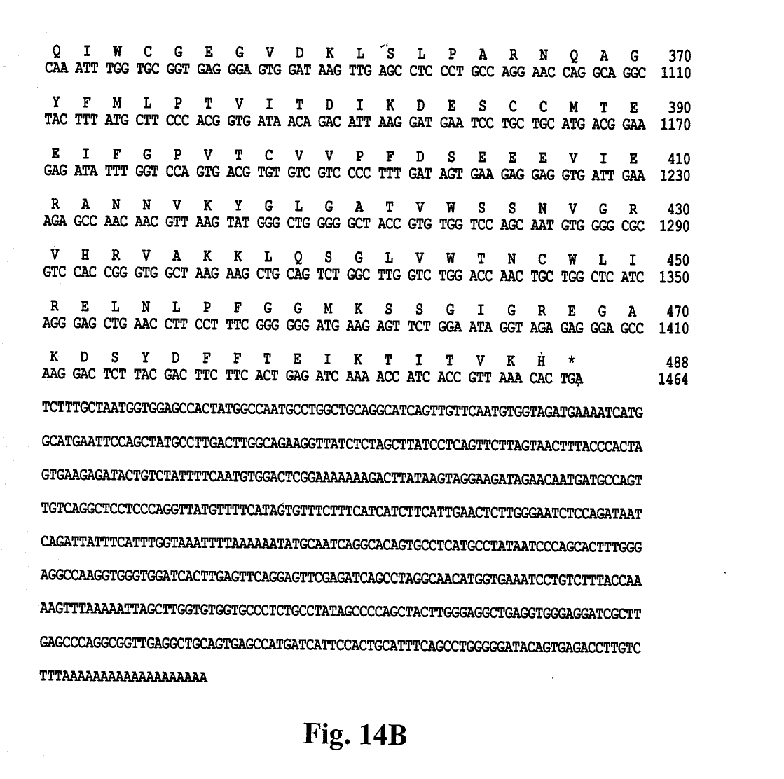

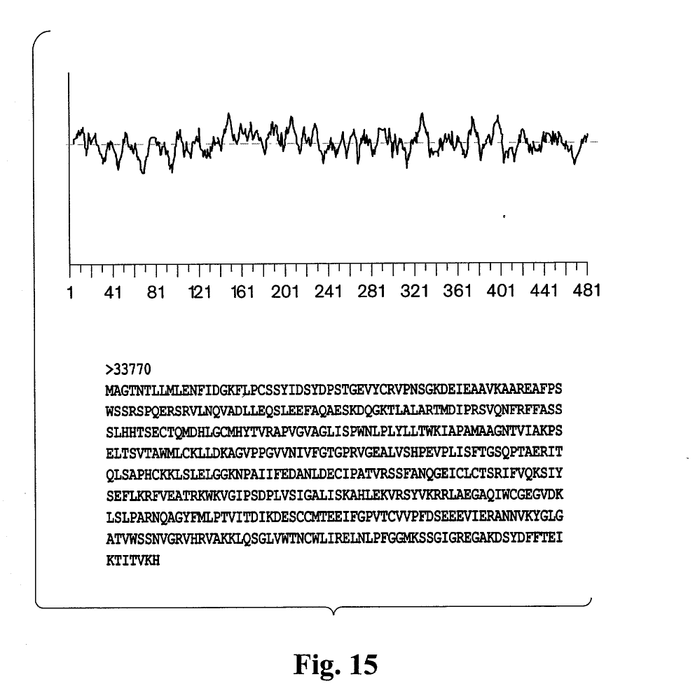

The human 33770 sequence (FIG. 14; SEQ ID NO: 16), which is approximately 2156 nucleotides long, including untranslated regions, contains a predicted methionine-initiated coding sequence of about 1464 nucleotides, including the termination codon (nucleotides indicated as coding of SEQ ID NO: 16 in FIG. 14; SEQ ID NO: 18). The coding sequence encodes a 487 amino acid protein (SEQ ID NO:17).

Human 33770 contains the following regions or other structural features:

an aldehyde dehydrogenase domain (PFAM Accession Number PF00171) located at about amino acid residues 17 to 487 of SEQ ID NO: 17, which includes a predicted aldehyde dehydrogenase cysteine active site (PS00070), “FANQGEICLCTS”, located at about amino acid residues 280 to 291 or SEQ ID NO: 17, and a predicted aldehyde dehydrogenase glutamic acid active site (PS00687), “LELGGKNP”, located at about amino acid residues 252 to 259 of SEQ ID NO:17;

eight predicted Protein Kinase C phosphorylation sites (PS00005) located at about amino acid residues 42 to 44, 62 to 64, 140 to 142, 162 to 164, 275 to 277, 290 to 292, 311 to 313, and 484 to 486 of SEQ ID NO:17;

eight predicted Casein Kinase II phosphorylation sites (PS00006) located at about amino acid residues 23 to 26, 31 to 34, 42 to 45, 65 to 68, 83 to 86, 129 to 132, 220 to 223, and 404 to 407 of SEQ ID NO: 17;

one predicted cAMP/cGMP-dependent protein kinase phosphorylation sites (PS00004) located at about amino acid residues 248 to 251 of SEQ ID NO:17;

seven predicted N-myristylation sites (PS00008) located at about amino acid residues 198 to 203, 231 to 236, 327 to 332, 418 to 423, 441 to 446, 458 to 463, and 469 to 474 of SEQ ID NO: 17;

and one predicted glycosaminoglycan attachment site (PS00002) located at about amino acid residues 463 to 466;

For general information regarding PFAM identifiers, PS prefix and PF prefix domain identification numbers, refer to Sonnhammer et al. (1997) Protein 28:405-420 and http://www.psc.edu/general/software/packages/pfam/pfam.html.

Plasmids containing the nucleotide sequences encoding human 21509 and 33770 (clone “Fbh21509FL” or “Fbh33770FL”) were deposited with American Type Culture Collection (ATCC), 10801 University Boulevard, Manassas, Va. 20110-2209, on ______ and assigned Accession Number ______ and ______, respectively. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

TABLE 1
Summary of Sequence Information for 21509 and 33770

| | | | | | ATCC |
|---|---|---|---|---|---|
| | | | | | Accession |
| Gene | cDNA | ORF | Polypeptide | FIG. | Number |
| 21509 | SEQ ID | SEQ ID | SEQ ID | | |
| | NO:13 | NO:15 | NO:14 | | |
| 33770 | SEQ ID | SEQ ID | SEQ ID | | |
| | NO:16 | NO:18 | NO:17 | | |

The 21509 and 33770 proteins contain a significant number of structural characteristics in common with members of the dehydrogenase/oxidoreductase family. The term “family” when referring to protein and nucleic acid molecules of the invention means two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin, or alternatively, can contain homologues of non-human origin, e.g., rat or mouse proteins. Members of a family can also have common functional characteristics.

As used herein, the term “dehydrogenase activity” means an activity that catalyzes directly or indirectly the removal of a hydride from a substrate. Typically, after removal of a hydride from a substrate, electrons of the hydride are transferred to NAD+, NADP+, or other coenzyme (e.g., 3-acetylpyridine adenine dinucleotide phosphate) or hydride acceptor. For example, if the substrate has hydroxyl, dehydrogenation converts the hydroxyl to a keto group and produces NADH or NADPH and a proton. Hydride removal from substrate however does not require the presence of an acceptor. Free hydride can be detected optically by H+ binding to a dye molecule, for example.

As used herein, the term “reductase activity” means a catalytic activity for the addition of one or more hydrides to a substrate having, for example, a keto group. Thus, reductase activity means the reverse of dehydrogenase activity. Typically, the hydride is provided by NADH, NADPH, or other coenzyme or hydride donor. For example, in the biological conversion of 4-androstenedione to testosterone, a hydrogen ion is transferred from NADPH to the substrate thereby forming NADP⁺ product. Coenzymes of 21509 and 33770 polypeptide also include, but are not limited to NAD⁺ and NAD⁺ analogues (Plapp et al. (1986) Biochemistry 25:5396-5402 and Yamazaki et al. (1984) J. Biochem. 95:109-115), NADH, NADP⁺, and NADPH (LaRhee et al. (1984) Biochemistry 23:486-491 and Pollow et al. (1976) J Steroid Biochem. 7:45-50).

Thus, a 21509 and 33770 polypeptide can include a domain having dehydrogenase or a reductase activity. Furthermore, as with 10-formyltetrahydrofolate dehydrogenase (FDH) discussed above, a 21509 and 33770 polypeptide can have domain(s) that confer both dehydrogenase and reductase activity. The particular activity of such a polypeptide, i.e., whether it functions as a dehydrogenase or a reductase will depend upon the conditions, coenzyme availability, etc. Because of the reversibility of the reaction, the dehydrogenase and reductase domains of a 21509 or 33770 polypeptide may be the same. Alternatively, the proteins may be bi-functional in that two separate domains confer dehydrogenase and reductase activity. The domains that confer these activities may therefore be located in the same or different regions of the polypeptide. Similarly, subsequences or fragments of 21509 and 33770 can be capable of one of either of the activities, or can be capable of both dehydrogenase and reductase activity.

Amino acid residues of 21509 that can have dehydrogenase or reductase activity include, for example, amino acid residues 3-184 of SEQ ID NO:14, or a subsequence thereof, which include a short chain adh family signature, “YSASKGGLVGF” (located at about amino acid residues 148 to 158 of SEQ ID NO:14). Additional structural domains that may confer or contribute to dehydrogenase or reductase activity(ies) include, for example, amino acid residues located at about 201-229; 182-237; 141-184; 54-176; 171-184; and 3-37 of 21509 (SEQ ID NO: 14), as well as combinations thereof or subsequences thereof.

Amino acid residues of 33770 that can have dehydrogenase or reductase activity include, for example, amino acid residues 17-487 (SEQ ID NO:17), or a subsequence thereof, which include an aldehyde dehydrogenase cysteine active site, “FANQGEICLCTS” (located at about amino acid residues 280 to 291 or SEQ ID NO:17), or an aldehyde dehydrogenase glutamic acid active site, “LELGGKNP” (located at about amino acid residues 252 to 259 of SEQ ID NO:17). Additional structural domains that may confer or contribute to dehydrogenase or reductase activity(ies) include, for example, amino acid residues located at about 29-487; 11-48; 28-58; 280-281 and 252-259 of 33770 (SEQ ID NO: 17), as well as combinations thereof or subsequences thereof.

As used herein, the term “short chain dehydrogenase domain” includes an amino acid sequence of about 100 to 240 amino acid residues in length and having a bit score for the alignment of the sequence to the short chain dehydrogenase domain domain profile (Pfam HMM PF00106) of at least 50. A 21509 polypeptide including this exemplary sequence (e.g., amino acid residues 3 to 184 of 21509 set forth as SEQ ID NO:14) has a bit score for alignment with short chain dehydrogenase domain (HMM PFAM Accession PF00106) of at least 50, preferably at least 100, more preferably at least 200. The short chain alcohol dehydrogenase family signature domain (HMM) has been assigned the PFAM Accession PS00061 (http;//genome.wustl.edu/Pfam/.html). A 21509 polypeptide including an short chain dehydrogenase domain can include at least about 117-200 amino acids, more typically about 148-190 amino acid residues, about 148-185, or about 183 amino acids. The domain can further include a “short chain alcohol dehydrogenase family signature domain” of 21509, e.g., the amino acid sequence YSASKGGLVGF (located at about amino acid residues 148 to 158 of SEQ ID NO:14).

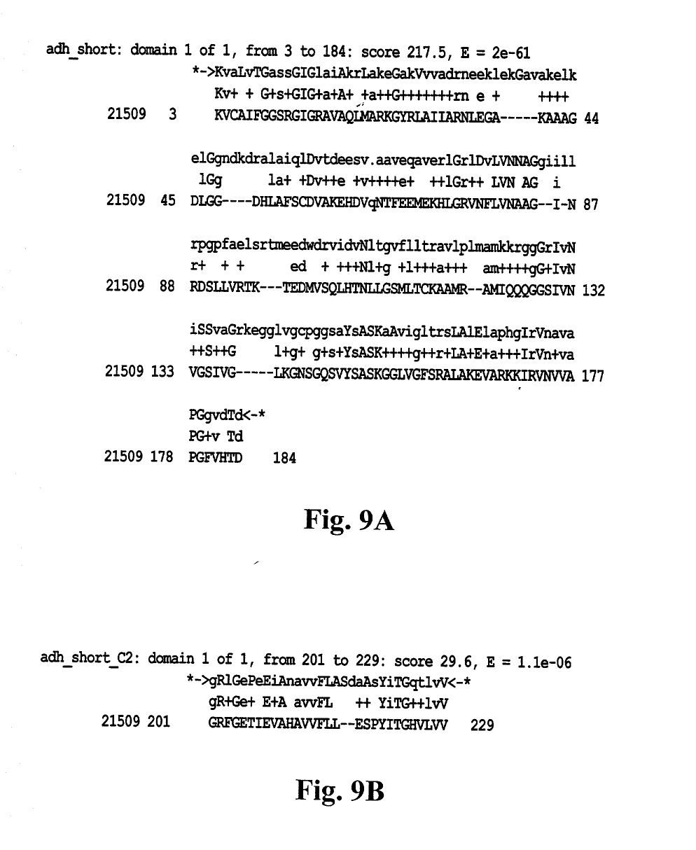

A predicted “short chain alcohol dehydrogenase C2 domain” or “adh short C2 domain” of 21509 polypeptide is located at amino acid residues 201-229 of SEQ ID NO:14. A 21509 polypeptide including this domain has a bit score for the alignment of the sequence to the adh short C2 domain (HMM from the SMART database (Simple Modular Architecture Research Tool, http://smart. embl-heidelberg.de/) of HMMs as described in Schultz et al. (1998), Proc. Natl. Acad. Sci. USA 95:5857 and Schultz et al. (200) Nucl. Acids Res 28:231) of at least 10, preferably 15, or more preferably 20. Alignments of a short chain alcohol dehydrogenase domain and a short C2 alcohol dehydrogenase domain of human 21509 (amino acids 3-184 and 201-229 of SEQ ID NO: 14, respectively) with consensus amino acid sequences derived from hidden Markov models are depicted in FIGS. 9A and 9B.

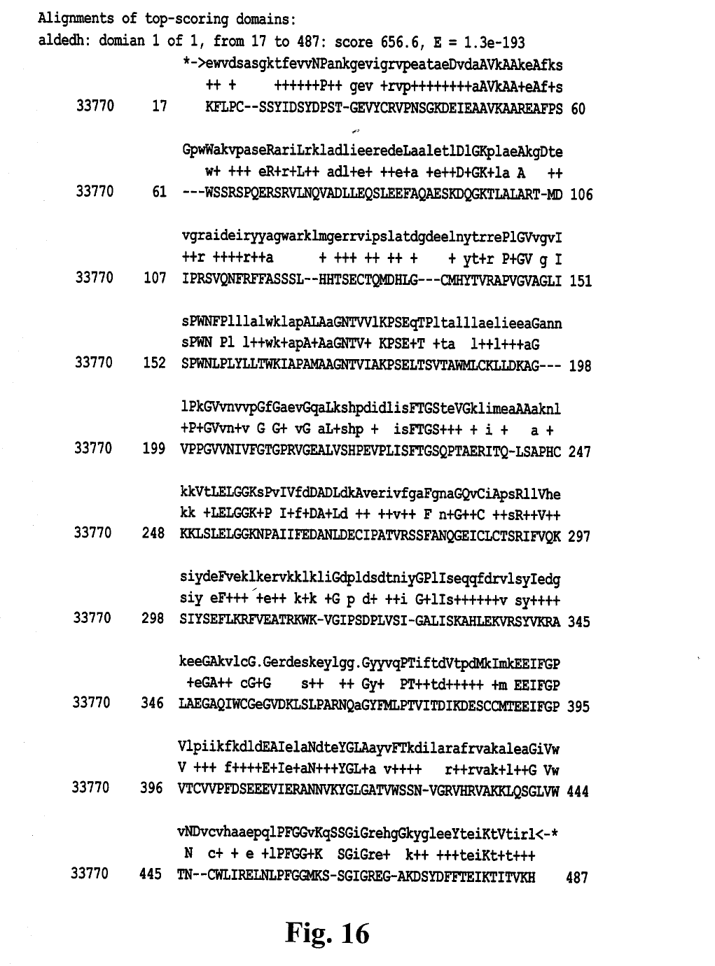

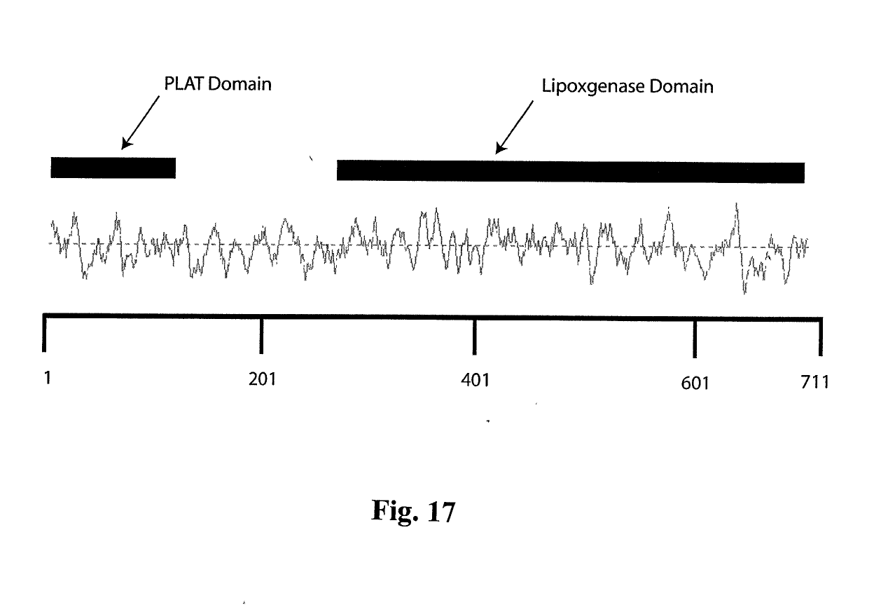

As used herein, the term “aldehyde dehydrogenase domain” (also “aldedh”) includes an amino acid sequence of about 270 to 500 amino acid residues in length and having a bit score for the alignment of the sequence to the aldehyde dehydrogenase domain profile (Pfam HMM PF00171) of at least 200. In one embodiment, a 33770 polypeptide including an aldedh domain (e.g., amino acid residues 17-487 of 33770 set forth as SEQ ID NO: 17) has a bit score for alignment with the aldehyde dehydrogenase family domain (HMM) of at least 200, preferably at least 400, more preferably at least 600. Preferably, an aldehyde dehydrogenase domain includes at least about 270 to 500 amino acids, more preferably about 350 to 490 amino acid residues, or about 400 to 490 amino acids and has a cysteine or glutamic acid active site (e.g., amino acid residues 280-291 and 252-259 of 33770 set forth as SEQ ID NO:17). The aldehyde dehydrogenase cysteine and glutamic acid active site domains have been assigned Accession numbers PS00070 and PS00687, respectively (http;//genome.wustl.edu/Pfam/.html). An alignment of an aldehyde dehydrogenase domain (amino acids 17-487 of SEQ ID NO:17, respectively) of human 33770 with a consensus amino acid sequence derived from a hidden Markov model is depicted in FIG. 16.

The alignments of exemplary 21509 and 33770 polypeptides (see, e.g., FIGS. 9 and 16) also predict that there are likely to be preferred substrates (targets) dehydrogenated or reduced. By “substrate” is intended to mean any molecule that can be oxidized or reduced by 21509 or 33770 polypeptides, as well as combinations thereof or subsequences thereof. For a 21509 polypeptide, likely substrates include those having an alcohol group; for a 33770 polypeptide, likely substrates include those having an aldehyde group. Alcohols include but are not limited to, primary or secondary alcohols or hemiacetals, and cyclic secondary alcohols, or ketones. Particular examples of substrates are steroids and other molecules having a cholesterol backbone or in which cholesterol is a biological precursor.

Due to the reversibility of the dehydrogenase/reductase reaction, and that many enzymes of the dehydrogenase/reductase family can carry out both reactions depending upon the conditions, substrates also include the products resulting from either oxidation or reduction of any of the molecules so modified. Thus, a substrate oxidized by a 21509 or 33770 polypeptide can also be reduced by a 21509 or 33770 polypeptide, and vice versa.

In one embodiment, a 21509 polypeptide or protein has a “dehydrogenase domain” or a “reductase domain,” or a region which includes at least about 117 to 250, more likely about 148 to 235, or 208 to 235 amino acid residues and has at least about 60%, 70% 80% 90% 95%, 99%, or 100% homology with an “alcohol dehydrogenase domain,” e.g., the signature domain of human 21509 (e.g., residues 148-158 of SEQ ID NO:14), or a sequence including the signature domain (e.g., residues 3-184 of SEQ ID NO: 14).

In another embodiment, a 33770 polypeptide or protein has a “dehydrogenase domain” or a “reductase domain,” or a region which includes at least about 270 to 500, more likely about 350 to 490, or 400 to 490 amino acid residues and has at least about 60%, 70% 80% 90% 95%, 99%, or 100% homology with an “aldehyde dehydrogenase domain,” e.g., the aldehyde dehydrogenase domain of human 33770 (e.g., residues 17-487 of SEQ ID NO: 17), or a sequence including cysteine or glutamic acid active sites (e.g., residues 153-158 and 148-158, respectively of SEQ ID NO: 17).

To identify the presence of a “short chain dehydrogenase” or “aldehyde dehydrogenase” domain in a 21509 or 33770 protein sequence, and make the determination that a polypeptide or protein of interest has a particular profile, the amino acid sequence of the protein can be searched against a database of HMMs (e.g., the Pfam database, release 2.1) using the default parameters (http://www.sanger.ac.uk/Software/Pfam/HMM_search). For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al.(1990) Meth. Enzymol. 183:146-159; Gribskov et al.(1987) Proc. Natl. Acad. Sci. USA 84:4355-4358; Krogh et al.(1994) J. Mol. Biol. 235:1501-1531; and Stultz et al.(1993) Protein Sci. 2:305-314, the contents of which are incorporated herein by reference.

A 21509 or 33770 molecule can include domains that confer or contribute to dehydrogenase or reductase activity as set forth herein. In addition, 21509 or 33770 molecules can further include sites that are phosphorylated, myristylated, contain glycosaminglycan attachment sites, etc. Such sites may contribute to dehydrogenase or reductase activity. 21509 or 33770 polypeptides and subsequences thereof including such sites, and nucleic acids that encode them, also are useful as immunogens for raising antibodies, or as competitive inhibitors, for example. Thus, a 21509 or 33770 polypeptide or subsequence that has a substrate recognition/binding site which lacks dehydrogenase or reductase activity can interfere with dehydrogenation or reduction of the substrate by binding the substrate thereby inhibiting naturally occurring 21509 or 33770 polypeptide binding/modification of the substrate. Similarly, a 21509 or 33770 subsequence that has a phosphorylation, myristylation, or glycosaminglycan attachment site can interfere with phosphorylation, myristylation, or glycosaminglycan attachment to endogenously expressed 21509 or 33770 polypeptides. Such 21509 or 33770 polypeptide or subsequences need only be large enough to function as a recognition/binding site for the enzyme, such as a kinase. A 21509 or 33770 subsequence that is inactive but forms an oligomer (e.g., dimer, tetramer) with an active full length form of a 21509 or 33770 polypeptide can inhibit one or more activities of the 21509 or 33770 oligomer.

A 21509 or 33770 family member can include one or more domains or sites described herein (e.g., signature domain, dehydrogenase or reductase domains, phosphorylation or myristylation sites, etc.), or other domains known in the art to be present in dehydrogenase/reductase gene family members. Of course, a 21509 or 33770 family member can also include a substrate (target) recognition/binding site and a coenzyme binding site to facilitate binding/interaction and subsequent dehydrogenation and/or reduction of the target.

Identification of such domains can be determined through sequence comparisons to domains of proteins having known function. Alternatively, functional assays can be used to ascertain function (e.g., dehydrogenase or reductase activity), using in vitro assays known in the art (see also, “Screening Assays,” below). As the 21509 or 33770 polypeptides of the invention may modulate 21509 or 33770-mediated activities, they may be useful for developing novel diagnostic and therapeutic agents for 21509 or 33770-mediated or related disorders, e.g., a disorder described below.

As used herein, a “21509 or 33770 activity,” “biological activity of 21509 or 33770” or “functional activity of 21509 or 33770,” refers to an activity exerted by a 21509 or 33770 protein, polypeptide or nucleic acid molecule on e.g., a 21509- or 33770-responsive cell or a 21509 or 33770 substrate, e.g., an alcohol or aldehyde substrate, as determined in vivo or in vitro. In one embodiment, a 21509 or 33770 activity is a direct activity, such as an association with a 21509- or 33770-target molecule and subsequent dehydrogenation or reduction. A “target molecule” or “binding partner” is a molecule with which a 21509 or 33770 protein binds or interacts in nature. In an exemplary embodiment, 21509 or 33770 acts enzymatically on a substrate, e.g., an alcohol- or aldehyde-containing molecule.

A 21509 or 33770 activity can also be an indirect activity, e.g., a cellular signaling activity mediated by interaction of the 21509 or 33770 protein with a 21509 or 33770 substrate, or modification of the substrate. Based on the above-described sequence similarities, 21509 or 33770 proteins of the present invention are predicted to have similar biological activities as dehydrogenase/oxidoreductase family members. For example, the 21509 or 33770 proteins of the present invention can be involved in one or more of the following processes: (1) fatty acid biosynthesis or metabolism (breakdown); (2) cellular changes associated with fatty acid biosynthesis or metabolism; (3) biosynthesis or metabolism of retinoic acids, e.g. 9-cis-retinoic acid; (4) developmental changes associated with retinoic acid biosynthesis or metabolism; (5) steroid biosynthesis or metabolism; (6) developmental changes associated with steroid biosynthesis or metabolism (e.g., sex trait development); (7) metabolism or removal of natural or xenobiotic substances (e.g., ethanol, toxins, etc.); (8) cellular proliferation or differentiation; or (9) cellular degeneration (e.g., neurodegeneration).

Thus, the 21509 or 33770 molecules can be useful as diagnostic agents, therapeutic targets, or therapeutic agents for detecting or controlling medical disorders, e.g., medical disorders relating to the synthesis or metabolism of fatty acids, retinoic acids, or steroids and associated proliferative/differentiative programs that lead to developmental changes, tumor induction, promotion or inhibition, or cellular degeneration, by directly or indirectly modulating the amounts of fatty acids (e.g., palmitic or stearic acid), hormones (e.g., retinoids, estrogen, androgen), or toxins present in or around a cell.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth. Examples of such cells include cells having an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Examples of cancers or neoplastic conditions, in addition to the ones described above, include, but are not limited to, a fibrosarcoma, myosarcoma, liposarcoma, chondrosarcoma, osteogenic sarcoma, chordoma, angiosarcoma, endotheliosarcoma, lymphangiosarcoma, lylnphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, or Kaposi sarcoma.

Examples of cellular proliferative and/or differentiative disorders of the breast include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Examples of cellular proliferative and/or differentiative disorders of the lung include, but are not limited to, bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma. Examples of cellular proliferative and/or differentiative disorders of the colon include, but are not limited to, non-neoplastic polyps, adenomas, familial syndromes, colorectal carcinogenesis, colorectal carcinoma, and carcinoid tumors.

Examples of cellular proliferative and/or differentiative disorders of the liver include, but are not limited to, nodular hyperplasias, adenomas, and malignant tumors, including primary carcinoma of the liver and metastatic tumors.

Examples of cellular proliferative and/or differentiative disorders of the ovary include, but are not limited to, ovarian tumors such as, tumors of coelomic epithelium, serous tumors, mucinous tumors, endometeriod tumors, clear cell adenocarcinoma, cystadenofibroma, brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecoma-fibromas, androblastomas, hill cell tumors, and gonadoblastoma; and metastatic tumors such as Krukenberg tumors.

Disorders involving the prostate include, but are not limited to, inflammations, benign enlargement, for example, nodular hyperplasia (benign prostatic hypertrophy or hyperplasia), and tumors such as carcinoma.

Disorders associated with abnormal fatty acid biosynthesis or metabolism include, but are not limited to, adrenomyeloneuropathy, ethylmalonic aciduria, diabetes, and cardiovascular disease. Examples of disorders involving the heart or “cardiovascular disorder” include, but are not limited to, a disease, disorder, or state involving the cardiovascular system, e.g., the heart, the blood vessels, and/or the blood. A cardiovascular disorder can be caused by an imbalance in arterial pressure, a malfunction of the heart, or an occlusion of a blood vessel, e.g., by a thrombus. Examples of such disorders include hypertension, atherosclerosis, coronary artery spasm, congestive heart failure, coronary artery disease, valvular disease, arrhythmias, and cardiomyopathies. In addition, fatty acids can influence the effective concentrations of both hormones and neuropeptides, and their receptors.

Additional examples of cardiovascular disorders, include but are not limited to, heart failure, including but not limited to, cardiac hypertrophy, left-sided heart failure, and right-sided heart failure; ischemic heart disease, including but not limited to angina pectoris, myocardial infarction, chronic ischemic heart disease, and sudden cardiac death; hypertensive heart disease, including but not limited to, systemic (left-sided) hypertensive heart disease and pulmonary (right-sided) hypertensive heart disease; valvular heart disease, including but not limited to, valvular degeneration caused by calcification, such as calcific aortic stenosis, calcification of a congenitally bicuspid aortic valve, and mitral annular calcification, and myxomatous degeneration of the mitral valve (mitral valve prolapse), rheumatic fever and rheumatic heart disease, infective endocarditis, and noninfected vegetations, such as nonbacterial thrombotic endocarditis and endocarditis of systemic lupus erythematosus (Libman-Sacks disease), carcinoid heart disease, and complications of artificial valves; myocardial disease, including but not limited to dilated cardiomyopathy, hypertrophic cardiomyopathy, restrictive cardiomyopathy, and myocarditis; pericardial disease, including but not limited to, pericardial effusion and hemopericardium and pericarditis, including acute pericarditis and healed pericarditis, and rheumatoid heart disease; neoplastic heart disease, including but not limited to, primary cardiac tumors, such as myxoma, lipoma, papillary fibroelastoma, rhabdomyoma, and sarcoma, and cardiac effects of noncardiac neoplasms; congenital heart disease, including but not limited to, left-to-right shunts—late cyanosis, such as atrial septal defect, ventricular septal defect, patent ductus arteriosus, and atrioventricular septal defect, right-to-left shunts—early cyanosis, such as tetralogy of fallot, transposition of great arteries, truncus arteriosus, tricuspid atresia, and total anomalous pulmonary venous connection, obstructive congenital anomalies, such as coarctation of aorta, pulmonary stenosis and atresia, and aortic stenosis and atresia, and disorders involving cardiac transplantation.

Disorders involving blood vessels include, but are not limited to, responses of vascular cell walls to injury, such as endothelial dysfunction and endothelial activation and intimal thickening; vascular diseases including, but not limited to, congenital anomalies, such as arteriovenous fistula, atherosclerosis, and hypertensive vascular disease, such as hypertension; inflammatory disease—the vasculitides, such as giant cell (temporal) arteritis, Takayasu arteritis, polyarteritis nodosa (classic), Kawasaki syndrome (mucocutaneous lymph node syndrome), microscopic polyanglitis (microscopic polyarteritis, hypersensitivity or leukocytoclastic anglitis), Wegener granulomatosis, thromboanglitis obliterans (Buerger disease), vasculitis associated with other disorders, and infectious arteritis; Raynaud disease; aneurysms and dissection, such as abdominal aortic aneurysms, syphilitic (luetic) aneurysms, and aortic dissection (dissecting hematoma); disorders of veins and lymphatics, such as varicose veins, thrombophlebitis and phlebothrombosis, obstruction of superior vena cava (superior vena cava syndrome), obstruction of inferior vena cava (inferior vena cava syndrome), and lymphangitis and lymphedema; tumors, including benign tumors and tumor-like conditions, such as hemangioma, lymphangioma, glomus tumor (glomangioma), vascular ectasias, and bacillary angiomatosis, and intermediate-grade (borderline low-grade malignant) tumors, such as Kaposi sarcoma and hemangloendothelioma, and malignant tumors, such as angiosarcoma and hemangiopericytoma; and pathology of therapeutic interventions in vascular disease, such as balloon angioplasty and related techniques and vascular replacement, such as coronary artery bypass graft surgery.

Additional disorders include those involving cells responsive to hormones (e.g., receptor containing cells) due to modulation of retinoid (e.g., 9-cis-retinoic acid) or steroid levels (e.g., androgens, estrogens, progesterones, mineral corticoids, glucocorticoids) by 21509 or 33770 polypeptides. Such disorders therefore include disorders in estrogen and androgen metabolism, for example, and their physiological consequences including male pseudohemaphroditism, proximal hypospadias, and polycystic kidney disease.

Disorders also include those treatable by 21509 or 33770 gene or protein replacement therapy, such as retinoid or steroid hormone deficiency, toxin elimination deficiency or accumulation of undesirable amounts of metabolites or intermediates, alcohol sensitivity, folate/tetrahydrofolate deficiency, due to inactivity/deficiency of an endogenous dehydrogenase or reductase protein.

The 21509 or 33770 protein, fragments thereof, and derivatives and other variants of the sequence in SEQ ID NO: 14 or SEQ ID NO: 17 thereof are collectively referred to as “polypeptides or proteins of the invention” or “21509 or 33770 polypeptides or proteins”. Nucleic acid molecules encoding such polypeptides or proteins are collectively referred to as “nucleic acids of the invention” or “21509 or 33770 nucleic acids.” 21509 or 33770 molecules refer to 21509 or 33770 nucleic acids, polypeptides, and antibodies.

As used herein, the term “nucleic acid molecule” includes DNA molecules (e.g., a cDNA or genomic DNA), RNA molecules (e.g., an mRNA) and analogs of the DNA or RNA. A DNA or RNA analog can be synthesized from nucleotide analogs. The nucleic acid molecule can be single-stranded or double-stranded, but preferably is double-stranded DNA.

The term “isolated nucleic acid molecule” or “purified nucleic acid molecule” includes nucleic acid molecules that are separated from other nucleic acid molecules present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2× SSC, 0.1% SDS at least at 50° C. (the temperature of the washes can be increased to 55° C. for low stringency conditions); 2) medium stringency hybridization conditions in 6× SSC at about 45° C., followed by one or more washes in 0.2× SSC, 0.1% SDS at 60° C.; 3) high stringency hybridization conditions in 6× SSC at about 45° C., followed by one or more washes in 0.2× SSC, 0.1% SDS at 65° C.; and preferably 4) very high stringency hybridization conditions are 0.5M sodium phosphate, 7% SDS at 65° C., followed by one or more washes at 0.2× SSC, 1% SDS at 65° C. Very high stringency conditions (4) are the preferred conditions and the ones that should be used unless otherwise specified.

Preferably, an isolated nucleic acid molecule of the invention that hybridizes under a stringency condition described herein to the sequence of SEQ ID NO:13, SEQ ID NO:15, SEQ ID NO:16, or SEQ ID NO:18 corresponds to a naturally-occurring nucleic acid molecule.

As used herein, a “naturally-occurring” nucleic acid molecule refers to an RNA or DNA molecule having a nucleotide sequence that occurs in nature. For example a naturally occurring nucleic acid molecule can encode a natural protein.

As used herein, the terms “gene” and “recombinant gene” refer to nucleic acid molecules which include at least an open reading frame encoding a 21509 or 33770 protein. The gene can optionally further include non-coding sequences, e.g., regulatory sequences and introns. Preferably, a gene encodes a mammalian 21509 or 33770 protein or derivative thereof.

An “isolated” or “purified” polypeptide or protein is substantially free of cellular material or other contaminating proteins from the cell or tissue source from which the protein is derived, or substantially free from chemical precursors or other chemicals when chemically synthesized. “Substantially free” means that a preparation of 21509 or 33770 protein is at least 10% pure. In a preferred embodiment, the preparation of 21509 or 33770 protein has less than about 30%, 20%, 10% and more preferably 5% (by dry weight), of non-21509 or 33770 protein (also referred to herein as a “contaminating protein”), or of chemical precursors or non-21509 or 33770 chemicals. When the 21509 or 33770 protein or biologically active portion thereof is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight.

A “non-essential” amino acid residue is a residue that can be altered from the wild-type sequence of 21509 or 33770 without abolishing or substantially altering a 21509 or 33770 activity. Preferably the alteration does not substantially alter the 21509 or 33770 activity, e.g., the activity is at least 20%, 40%, 60%, 70% or 80% of wild-type. An “essential” amino acid residue is a residue that, when altered from the wild-type sequence of 21509 or 33770, results in abolishing a 21509 or 33770 activity such that less than 20% of the wild-type activity is present. For example, conserved amino acid residues in 21509 or 33770 are predicted to be particularly unamenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 21509 or 33770 protein is preferably replaced with another amino acid residue from the same side chain family. Alternatively, in another embodiment, mutations can be introduced randomly along all or part of a 21509 or 33770 coding sequence, such as by saturation mutagenesis, and the resultant mutants can be screened for 21509 or 33770 biological activity to identify mutants that retain activity. Following mutagenesis of SEQ ID NO: 13, SEQ ID NO: 15, SEQ ID NO: 16, or SEQ ID NO: 18, the encoded protein can be expressed recombinantly and the activity of the protein can be determined.

As used herein, a “biologically active portion” of a 21509 or 33770 protein includes a fragment of a 21509 or 33770 protein which participates in an interaction, e.g., an intramolecular or an inter-molecular interaction. An inter-molecular interaction can be a specific binding interaction or an enzymatic interaction (e.g., the interaction can be transient and a covalent bond is formed or broken). An inter-molecular interaction can be between a 21509 or 33770 molecule and a non-21509 or 33770 molecule or between a first 21509 or 33770 molecule and a second 21509 or 33770 molecule (e.g., a dimerization interaction). Biologically active portions of a 21509 or 33770 protein include peptides comprising amino acid sequences sufficiently homologous to or derived from the amino acid sequence of the 21509 or 33770 protein, e.g., the amino acid sequence shown in SEQ ID NO:14 or 17, respectively, which include less amino acids than the full length 21509 or 33770 proteins, and exhibit at least one activity of a 21509 or 33770 protein. Typically, biologically active portions comprise a domain or motif with at least one activity of the 21509 or 33770 protein, e.g, dehydrognase or reductase activity. A biologically active portion of a 21509 or 33770 protein can be a polypeptide which is, for example, 10, 25, 50, 100, 200 or more amino acids in length. Biologically active portions of a 21509 or 33770 protein can be used as targets for developing agents which modulate a 21509 or 33770 mediated activity, e.g., dehydrognase or reductase activity.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossunm 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to 21509 or 33770 nucleic acid molecules of the invention. BLAST protein searches can be performed with the XBLAST program, score=50, wordlength=3 to obtain amino acid sequences homologous to 21509 or 33770 protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

Particular 21509 or 33770 polypeptides of the present invention have an amino acid sequence substantially identical to the amino acid sequence of SEQ ID NO: 14 or SEQ ID NO: 17. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 60%, or 65% identity, likely 75% identity, more likely 85%, 90%. 91%, 92%, 93%, 94%, 95%, 96%, 97%, 98% or 99% identity to SEQ ID NO: 14 or SEQ ID NO: 17 are termed substantially identical.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 60%, or 65% identity, likely 75% identity, more likely 85%, 90%. 91%, 92%, 93%, 94%, 95%, 96%, 97%, 98% or 99% identity to SEQ ID NO: 13, SEQ ID NO: 15, SEQ ID NO: 16, or SEQ ID NO: 18 are termed substantially identical.

“Misexpression or aberrant expression”, as used herein, refers to a non-wildtype pattern of gene expression at the RNA or protein level. It includes: expression at non-wild type levels, i.e., over- or under-expression; a pattern of expression that differs from wild type in terms of the time or stage at which the gene is expressed, e.g., increased or decreased expression (as compared with wild type) at a predetermined developmental period or stage; a pattern of expression that differs from wild type in terms of altered, e.g., increased or decreased, expression (as compared with wild type) in a predetermined cell type or tissue type; a pattern of expression that differs from wild type in terms of the splicing size, translated amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

“Subject,” as used herein, refers to human and non-human animals. The term “non-human animals” of the invention includes all vertebrates, e.g., mammals, such as non-human primates (particularly higher primates), sheep, dog, rodent (e.g., mouse or rat), guinea pig, goat, pig, cat, rabbits, cow, and non-mammals, such as chickens, amphibians, reptiles, etc. In a preferred embodiment, the subject is a human. In another embodiment, the subject is an experimental animal or animal suitable as a disease model.

A “purified preparation of cells”, as used herein, refers to an in vitro preparation of cells. In the case cells from multicellular organisms (e.g., plants and animals), a purified preparation of cells is a subset of cells obtained from the organism, not the entire intact organism. In the case of unicellular microorganisms (e.g., cultured cells and microbial cells), it consists of a preparation of at least 10% and more preferably 50% of the subject cells.

Various aspects of the invention are described in further detail below.

Isolated Nucleic Acid Molecules of 21509 and 33770

In one aspect, the invention provides, an isolated or purified, nucleic acid molecule that encodes a 21509 or 33770 polypeptide described herein, e.g., a full-length 21509 or 33770 protein or a fragment thereof, e.g., a biologically active portion of 21509 or 33770 protein. Also included is a nucleic acid fragment suitable for use as a hybridization probe, which can be used, e.g., to identify a nucleic acid molecule encoding a polypeptide of the invention, 21509 or 33770 mRNA, and fragments suitable for use as primers, e.g., PCR primers for the amplification or mutation of nucleic acid molecules.

In one embodiment, an isolated nucleic acid molecule of the invention includes the nucleotide sequence shown in SEQ ID NO: 13 or SEQ ID NO: 16, or a portion of any of these nucleotide sequences. In one embodiment, the nucleic acid molecule includes sequences encoding the human 21509 or 33770 protein (i.e., “the coding region” of SEQ ID NO:13 or SEQ ID NO: 16, as shown in SEQ ID NO: 15 or SEQ ID NO: 18, respectively), as well as 5′ untranslated sequences. Alternatively, the nucleic acid molecule can include only the coding region of SEQ ID NO:13 or SEQ ID NO:16 (e.g., SEQ ID NO:15 or SEQ ID NO:18) and, e.g., no flanking sequences which normally accompany the subject sequence. In another embodiment, the nucleic acid molecule encodes a sequence corresponding to a fragment of the protein from about amino acid 3 to 229 of SEQ ID NO: 14, or from about amino acid 17 to 487 of SEQ ID NO:17.

In another embodiment, an isolated nucleic acid molecule of the invention includes a nucleic acid molecule which is a complement of the nucleotide sequence shown in SEQ ID NO: 13, SEQ ID NO: 15, SEQ ID NO: 16, SEQ ID NO: 18, or a portion of any of these nucleotide sequences. In other embodiments, the nucleic acid molecule of the invention is sufficiently complementary to the nucleotide sequence shown in SEQ ID NO:13, SEQ ID NO:15, SEQ ID NO:16, or SEQ ID NO:18, such that it can hybridize (e.g., under a stringency condition described herein) to the nucleotide sequence shown in SEQ ID NO:13, SEQ ID NO:15, SEQ ID NO:16, or SEQ ID NO:18, thereby forming a stable duplex.

In one embodiment, an isolated nucleic acid molecule of the present invention includes a nucleotide sequence which is at least about: 70%, 80%, 85%, 90%, 91%, 92%, 93%, 94%, 95%, 96%, 97%, 98%, 99%, or more homologous to the entire length of the nucleotide sequence shown in SEQ ID NO:13, SEQ ID NO:15, SEQ ID NO:16, SEQ ID NO:18, or a portion, preferably of the same length, of any of these nucleotide sequences.

21509 or 33770 Nucleic Acid Fragments

A nucleic acid molecule of the invention can include only a portion of the nucleic acid sequence of SEQ ID NO:13, SEQ ID NO:15, SEQ ID NO:16, SEQ ID NO:18. For example, such a nucleic acid molecule can include a fragment which can be used as a probe or primer or a fragment encoding a portion of a 21509 or 33770 protein, e.g., an immunogenic or biologically active portion of a 21509 or 33770 protein. A fragment can comprise those nucleotides of SEQ ID NO:13 or SEQ ID NO:16 which encode a dehydrogenase domain of human 21509 or 33770. The nucleotide sequence determined from the cloning of the 21509 or 33770 gene allows for the generation of probes and primers designed for use in identifying and/or cloning other 21509 or 33770 family members, or fragments thereof, as well as 21509 or 33770 homologues, or fragments thereof, from other species.

In another embodiment, a nucleic acid includes a nucleotide sequence that includes part, or all, of the coding region and extends into either (or both) the 5′ or 3′ noncoding region. Other embodiments include a fragment which includes a nucleotide sequence encoding an amino acid fragment described herein. Nucleic acid fragments can encode a specific domain or site described herein or fragments thereof, particularly fragments thereof which are at least 117, preferably 148, or more preferably 208 amino acids from SEQ ID NO: 14, or at least 270, preferably 300, or more preferably 350 amino acids from SEQ ID NO:17. Fragments also include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a domain, region, or functional site described herein. A nucleic acid fragment can also include one or more domain, region, or functional site described herein. Thus, for example, a 21509 or 33770 nucleic acid fragment can include a sequence corresponding to a dehydrogenase or reductase domain. 21509 or 33770 probes and primers are provided. Typically a probe/primer is an isolated or purified oligonucleotide. The oligonucleotide typically includes a region of nucleotide sequence that hybridizes under a stringency condition described herein to at least about 7, 12 or 15, preferably about 20 or 25, more preferably about 30, 35, 40, 45, 50, 55, 60, 65, or 75 consecutive nucleotides of a sense or antisense sequence of SEQ ID NO: 13, SEQ ID NO: 15, SEQ ID NO: 16, or SEQ ID NO: 18, or of a naturally occurring allelic variant or mutant of SEQ ID NO:13, SEQ ID NO:15, SEQ ID NO:16, or SEQ ID NO:18.

In a preferred embodiment the nucleic acid is a probe which is at least 5 or 10, and less than 200, more preferably less than 100, or less than 50, base pairs in length. It should be identical, or differ by 1, or less than in 5 or 10 bases, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid which encodes: about amino acids 3-184, 201-229, 33-37, 36-238, 209-229, 114-116, 66-69, 95-98, 9-14, 38-43, 110-115, 128-133, 134-139, 153-158 or 148-158 of SEQ ID NO:14, or combinations containing contiguous sequences thereof; or about amino acids 17-487, 483-487, 145-163, 314-330, 463-466, 280-291, or 252-259 of SEQ ID NO:17, or combinations containing contiguous sequences thereof.

In another embodiment a set of primers is provided, e.g., primers suitable for use in a PCR, which can be used to amplify a selected region of a 21509 or 33770 sequence, e.g., a domain, region, site or other sequence described herein. The primers should be at least 5, 10, 25, or 50 base pairs in length and less than 100, or less than 200, base pairs in length. The primers should be identical, or differ by one base from a sequence disclosed herein or from a naturally occurring variant. For example, primers suitable for amplifying all or a portion of any of the following regions are provided: about amino acids 3-184, 201-229, 33-37, 36-238, 209-229, 114-116, 66-69, 95-98, 9-14, 38-43, 110-115, 128-133, 134-139, 153-158 or 148-158 of SEQ ID NO: 14, or combinations containing contiguous sequences thereof; or about amino acids 17-487, 483-487, 145-163, 314-330, 463-466, 280-291, or 252-259 of SEQ ID NO:17, or combinations containing contiguous sequences thereof.

A nucleic acid fragment can encode an epitope bearing region of a polypeptide described herein.

A nucleic acid fragment encoding a “biologically active portion of a 21509 or 33770 polypeptide” can be prepared by isolating a portion of the nucleotide sequence of SEQ ID NO:13, SEQ ID NO:15, SEQ ID NO:16, SEQ ID NO:18, or the nucleotide sequence of the DNA insert of the plasmid deposited with ATCC as Accession Number ______ or ______, which encodes a polypeptide having a 21509 or 33770 biological activity (e.g., the biological activities of the 21509 or 33770 proteins are described herein), expressing the encoded portion of the 21509 or 33770 protein (e.g., by recombinant expression in vitro) and assessing the activity of the encoded portion of the 21509 or 33770 protein. For example, a nucleic acid fragment encoding a biologically active portion of 21509 or 33770 includes a dehydrogenase or reductase domain, e.g., amino acid residues 3 to 184 of SEQ ID NO:14 or amino acid residues 17 to 487 of SEQ ID NO: 17.

A nucleic acid fragment encoding a biologically active portion of a 21509 polypeptide, may include a nucleotide sequence which is greater than 460, 500, 600, 700, 800, 900, 1000, or more nucleotides in length. In a preferred embodiment, the nucleic acid fragment includes at least one contiguous nucleotide from about nucleotides: 1 to 74, 74 to 157, 570-800, 400-710, of SEQ ID NO:13. Preferably, the nucleic acid fragment is 100% identical to about nucleotides 1 to 74, 74 of 157, 74 to 265 of SEQ ID NO: 13.

A nucleic acid fragment encoding a biologically active portion of a 33770 polypeptide, may include a nucleotide sequence which is greater than 300, 400, 500, 600, 700, 810, 900, 1000, 1100, 1200, 1300, 1400, 1500, 1600, 1700, 1800, 1900, 2000, 2100, or more nucleotides in length. In a preferred embodiment, the nucleic acid fragment includes at least one contiguous nucleotide from about nucleotides: 1 to 300, 300 to 440, 1 to 450, 500 to 1000, or 1400 to 2000 of SEQ ID NO: 16. In another preferred embodiment the nucleic acid fragment encodes a polypeptide fragment which includes 10 or more amino acid from the region of about 1 to 100, or 50 to 150 of SEQ ID NO:17.

In preferred embodiments, a nucleic acid includes a nucleotide sequence that is: about 460, 500, 600, 700, 800, 900, 1000, or more nucleotides in length and hybridizes under a stringency condition described herein to a nucleic acid molecule of SEQ ID NO:13, or SEQ ID NO:15; or about 300, 400, 500, 600, 700, 810, 900, 1000, 1100, 1200, 1300 1400,1500, 1600, 1700, 1800, 1900, 2000, 2100, or more nucleotides in length and hybridizes under a stringency condition described herein to a nucleic acid molecule of SEQ ID NO:16, or SEQ ID NO:18.

21509 or 33770 Nucleic Acid Variants

The invention further encompasses nucleic acid molecules that differ from the nucleotide sequence shown in SEQ ID NO:13, SEQ ID NO:15, SEQ ID NO:16, or SEQ ID NO: 18. Such differences can be due to degeneracy of the genetic code (and result in a nucleic acid which encodes the same 21509 or 33770 proteins as those encoded by the nucleotide sequence disclosed herein. In another embodiment, an isolated nucleic acid molecule of the invention has a nucleotide sequence encoding a protein having an amino acid sequence which differs, by at least 1, but less than 5, 10, 20, 50, or 100 amino acid residues that shown in SEQ ID NO: 14 or SEQ ID NO: 17. If alignment is needed for this comparison the sequences should be aligned for maximum homology. “Looped” out sequences from deletions or insertions, or mismatches, are considered differences.

Nucleic acids of the inventor can be chosen for having codons, which are preferred, or non-preferred, for a particular expression system. E.g., the nucleic acid can be one in which at least one codon, at preferably at least 10%, or 20% of the codons has been altered such that the sequence is optimized for expression in E. coli, yeast, human, insect, or CHO cells.

Nucleic acid variants can be naturally occurring, such as allelic variants (same locus), homologs (different locus), and orthologs (different organism) or can be non naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

In a preferred embodiment, the nucleic acid differs from that of SEQ ID NO: 13, SEQ ID NO: 15, SEQ ID NO: 16, or SEQ ID NO: 18, e.g., as follows: by at least one but less than 10, 20, 30, or 40 nucleotides; at least one but less than 1%, 5%, 10% or 20% of the nucleotides in the subject nucleic acid. If necessary for this analysis the sequences should be aligned for maximum homology. “Looped” out sequences from deletions or insertions, or mismatches, are considered differences.

Orthologs, homologs, and allelic variants can be identified using methods known in the art. These variants comprise a nucleotide sequence encoding a polypeptide that is about 90-95%, 96%, 97%, 98%, 99%, or more identical to the nucleotide sequence shown in SEQ ID NO: 14, SEQ ID NO: 17, or a fragment of these sequences. Such nucleic acid molecules can readily be identified as being able to hybridize under a stringency condition described herein, to the nucleotide sequence shown in SEQ ID NO: 14, SEQ ID NO: 17, or a fragment of the sequences. Nucleic acid molecules corresponding to orthologs, homologs, and allelic variants of the 21509 or 33770 cDNAs of the invention can further be isolated by mapping to the same chromosome or locus as the 21509 or 33770 gene.

Preferred variants include those that are correlated with dehydrogenase or reductase activity.

Allelic variants of 21509 or 33770, e.g., human 21509 or 33770, include both functional and non-functional proteins. Functional allelic variants are naturally occurring amino acid sequence variants of the 21509 or 33770 protein within a population that maintain the ability to bind substrates, e.g., acetyl CoA and malonyl-ACP, or 9-cis-retinal. Functional allelic variants will typically contain only conservative substitution of one or more amino acids of SEQ ID NO: 14 or SEQ ID NO: 17, or substitution, deletion or insertion of non-critical residues in non-critical regions of the protein. Non-functional allelic variants are naturally-occurring amino acid sequence variants of the 21509 or 33770, e.g., human 21509 or 33770, protein within a population that do not have the ability to catalyze dehydrogenase or reductase reactions. Non-functional allelic variants will typically contain a non-conservative substitution, a deletion, or insertion, or premature truncation of the amino acid sequence of SEQ ID NO: 14 or SEQ ID NO: 17, or a substitution, insertion, or deletion in critical residues or critical regions of the protein.

Moreover, nucleic acid molecules encoding other 21509 or 33770 family members and, thus, which have a nucleotide sequence which differs from the 21509 or 33770 sequences of SEQ ID NO:13, SEQ ID NO:15, SEQ ID NO:16, or SEQ ID NO:18 are intended to be within the scope of the invention.

Antisense Nucleic Acid Molecules, Ribozymes and Modified 21509 or 33770 Nucleic Acid Molecules

In another aspect, the invention features, an isolated nucleic acid molecule which is antisense to 21509 or 33770. An “antisense” nucleic acid can include a nucleotide sequence which is complementary to a “sense” nucleic acid encoding a protein, e.g., complementary to the coding strand of a double-stranded cDNA molecule or complementary to an mRNA sequence. The antisense nucleic acid can be complementary to an entire 21509 or 33770 coding strand, or to only a portion thereof (e.g., the coding region of human 21509 or 33770 corresponding to SEQ ID NO: 15 or SEQ ID NO: 18, respectively). In another embodiment, the antisense nucleic acid molecule is antisense to a “noncoding region” of the coding strand of a nucleotide sequence encoding 21509 or 33770 (e.g., the 5′ and 3′ untranslated regions).

An antisense nucleic acid can be designed such that it is complementary to the entire coding region of 21509 or 33770 mRNA, but more preferably is an oligonucleotide which is antisense to only a portion of the coding or noncoding region of 21509 or 33770 mRNA. For example, the antisense oligonucleotide can be complementary to the region surrounding the translation start site of 21509 or 33770 mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject (e.g., by direct injection at a tissue site), or generated in situ such that they hybridize with or bind to cellular mRNA and/or genomic DNA encoding a 21509 or 33770 protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. A ribozyme having specificity for a 21509 or 33770-encoding nucleic acid can include one or more sequences complementary to the nucleotide sequence of a 21509 or 33770 cDNA disclosed herein (i.e., SEQ ID NO:13, SEQ ID NO:15, SEQ ID NO:16, or SEQ ID NO:18), and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a 21509 or 33770-encoding mRNA. See, e.g., Cech et al. U.S. Pat. No. 4,987,071; and Cech et al. U.S. Pat. No. 5,116,742. Alternatively, 21509 or 33770 mRNA can be used to select a catalytic RNA having a specific ribonuclease activity from a pool of RNA molecules. See, e.g., Bartel, D. and Szostak, J. W. (1993) Science 261:1411-1418.

21509 or 33770 gene expression can be inhibited by targeting nucleotide sequences complementary to the regulatory region of the 21509 or 33770 (e.g., the 21509 or 33770 promoter and/or enhancers) to form triple helical structures that prevent transcription of the 21509 or 33770 gene in target cells. See generally, Helene, C. (1991) Anticancer Drug Des. 6:569-84; Helene, C. i (1992) Ann. N.Y. Acad. Sci. 660:27-36; and Maher, L. J. (1992) Bioassays 14:807-15. The potential sequences that can be targeted for triple helix formation can be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

The invention also provides detectably labeled oligonucleotide primer and probe molecules. Typically, such labels are chemiluminescent, fluorescent, radioactive, or colorimetric.

A 21509 or 33770 nucleic acid molecule can be modified at the base moiety, sugar moiety or phosphate backbone to improve, e.g., the stability, hybridization, or solubility of the molecule. For non-limiting examples of synthetic oligonucleotides with modifications see Toulme (2001) Nature Biotech. 19:17 and Faria et al. (2001) Nature Biotech. 19:40-44. Such phosphoramidite oligonucleotides can be effective antisense agents.

For example, the deoxyribose phosphate backbone of the nucleic acid molecules can be modified to generate peptide nucleic acids (see Hyrup B. et al. (1996) Bioorganic & Medicinal Chemistry 4: 5-23). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup B. et al. (1996) supra and Perry-O'Keefe et al. Proc. Natl. Acad. Sci. 93: 14670-675.

PNAs of 21509 or 33770 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of 21509 or 33770 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al. (1996) supra)); or as probes or primers for DNA sequencing or hybridization (Hyrup B. et al. (1996) supra; Perry-O'Keefe supra).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (see, e.g., Krol et al. (1988) Bio-Techniques 6:958-976) or intercalating agents. (see, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

The invention also includes molecular beacon oligonucleotide primer and probe molecules having at least one region which is complementary to a 21509 or 33770 nucleic acid of the invention, two complementary regions one having a fluorophore and one a quencher such that the molecular beacon is useful for quantitating the presence of the 21509 or 33770 nucleic acid of the invention in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

Isolated 21509 or 33770 Polypeptides

In another aspect, the invention features, an isolated 21509 or 33770 protein, or fragment, e.g., a biologically active portion, for use as immunogens or antigens to raise or test (or more generally to bind) anti-21509 or 33770 antibodies. 21509 or 33770 protein can be isolated from cells or tissue sources using standard protein purification techniques. 21509 or 33770 protein or fragments thereof can be produced by recombinant DNA techniques or synthesized chemically.

Polypeptides of the invention include those which arise as a result of the existence of multiple genes, alternative transcription events, alternative RNA splicing events, and alternative translational and post-translational events. The polypeptide can be expressed in systems, e.g., cultured cells, which result in substantially the same post-translational modifications present when expressed the polypeptide is expressed in a native cell, or in systems which result in the alteration or omission of post-translational modifications, e.g., glycosylation or cleavage, present when expressed in a native cell.

In one embodiment, a 21509 or 33770 polypeptide has one or more of the following characteristics:

(i) it has a dehydrogenase or reductase activity;

(ii) it regulates fatty acid biosynthesis or metabolism;

(iii) it regulates retinoid biosynthesis or metabolism;

(iv) it regulates steroid biosynthesis or metabolism;

(v) it regulates the metabolism or removal of natural or xenobiotic substances (e.g., ethanol, toxins, etc.);

(vi) it modulates cellular proliferation and/or differentiation;

(vii) it modulates cellular degeneration (e.g., neurodegeneration);

(viii) it has a molecular weight of a 21509 polypeptide, e.g., a polypeptide of SEQ ID NO: 14 (e.g., 31 kDa); or a 33770 polypeptide, e.g., a polypeptide of SEQ ID NO: 17 (e.g., 54 kDa);

(ix) it has an overall sequence similarity of at least 60%, 70%, 80%, 90%, 95%, 96%, 97%, 98%, or 99%, with a polypeptide of SEQ ID NO: 14 or SEQ ID NO: 17;

(x) it can be found in the prostate, brain (nerve or glial cell), heart, liver, kidney, blood vessels, fetal liver, bone, (e.g., artery, vein, vascular smooth muscle, endothelia), skeletal muscle, breast, ovary, colon, or lung; or

(xi) it has a dehydrogenase or reductase domain which preferably includes about 70%, 80%, 90% or 95% of the amino acid residues 3-184 of SEQ ID NO: 14, or amino acid residues 17-487 of SEQ ID NO:17

In one embodiment the 21509 or 33770 protein, or fragment thereof, differs from the corresponding sequence in SEQ ID NO:14 or SEQ ID NO:17. In one aspect it differs by at least one but by less than 15, 10 or 5 amino acid residues. In another aspect it differs from the corresponding sequence in SEQ ID NO:14 or SEQ ID NO:17 by at least one residue but less than 20%, 15%, 10% or 5% of the residues in it differ from the corresponding sequence in SEQ ID NO:14 or SEQ ID NO:17. (If this comparison requires alignment the sequences should be aligned for maximum homology. “Looped” out sequences from deletions or insertions, or mismatches, are considered differences.) The differences are likely differences or changes at a non-essential residue or a conservative substitution. In some embodiments, the differences are in non-essential regions. In other embodiments, one or more differences are in amino acid residues 3-184 of SEQ ID NO:14, or amino acid residues 17-487 of SEQ ID NO:17

Other embodiments include a protein that contain one or more changes in amino acid sequence, e.g., a change in an amino acid residue which is not essential for activity. Such 21509 or 33770 proteins differ in amino acid sequence from SEQ ID NO:14 and SEQ ID NO: 17, yet retain biological activity.

In one embodiment, the protein includes an amino acid sequence at least about 60%, 70%, 80%, 90%, 95%, 96%, 97%, 98%, 99%, or more homologous to SEQ ID NO:14 or SEQ ID NO: 17. In one embodiment, the protein includes a peptide sequence that is homologous to about amino acids 1 to 100 or 50 to 150 of SEQ ID NO:17.

A 21509 or 33770 protein or fragment is provided which varies from the sequence of SEQ ID NO:14 or SEQ ID NO:17 in amino acid residues 3-184 of SEQ ID NO:14, or amino acid residues 17-487 of SEQ ID NO:17, by at least one but by less than 15, 10 or 5 amino acid residues in the protein or fragment. (If this comparison requires alignment the sequences should be aligned for maximum homology. “Looped” out sequences from deletions or insertions, or mismatches, are considered differences.) In some embodiments the difference is at a non essential residue or is a conservative substitution, while in others the difference is at an essential residue or is a non conservative substitution.

In one embodiment, a biologically active portion of a 21509 or 33770 protein includes a dehydrogenase or reductase domain. Moreover, other biologically active portions, in which other regions of the protein are deleted, can be prepared by recombinant techniques and evaluated for one or more of the functional activities of a native 21509 or 33770 protein.

In another embodiment, the 21509 or 33770 protein has an amino acid sequence shown in SEQ ID NO:14 or SEQ ID NO:17. In other embodiments, the 21509 or 33770 protein is substantially identical to SEQ ID NO:14 or SEQ ID NO:17. In yet another embodiment, the 21509 or 33770 protein is substantially identical to SEQ ID NO:14 or SEQ ID NO:17 and retains the functional activity of the protein of SEQ ID NO: 14 or SEQ ID NO: 17, as described herein.

21509 or 33770 Chimeric or Fusion Proteins

In another aspect, the invention provides 21509 or 33770 chimeric or fusion proteins. As used herein, a 21509 or 33770 “chimeric protein” or “fusion protein” includes a 21509 or 33770 polypeptide linked to a non-21509 or 33770 polypeptide. A “non-21509 or 33770 polypeptide” refers to a polypeptide having an amino acid sequence corresponding to a protein which is not substantially homologous to the 21509 or 33770 protein, e.g., a protein which is different from the 21509 or 33770 protein and which is derived from the same or a different organism. The 21509 or 33770 polypeptide of the fusion protein can correspond to all or a portion e.g., a fragment described herein of a 21509 or 33770 amino acid sequence. In a preferred embodiment, a 21509 or 33770 fusion protein includes at least one (or two) biologically active portion of a 21509 or 33770 protein. The non-21509 or 33770 polypeptide can be fused to the N-terminus or C-terminus of the 21509 or 33770 polypeptide.

The fusion protein can include a moiety which has a high affinity for a ligand. For example, the fusion protein can be a GST-21509 or 33770 fusion protein in which the 21509 or 33770 sequences are fused to the C-terminus of the GST sequences. Such fusion proteins can facilitate the purification of recombinant 21509 or 33770. Alternatively, the fusion protein can be a 21509 or 33770 protein containing a heterologous signal sequence at its N-terminus. In certain host cells (e.g., mammalian host cells), expression and/or secretion of 21509 or 33770 can be increased through use of a heterologous signal sequence.

Fusion proteins can include all or a part of a serum protein, e.g., an IgG constant region, or human serum albumin.

The 21509 or 33770 fusion proteins of the invention can be incorporated into pharmaceutical compositions and administered to a subject in vivo. The 21509 or 33770 fusion proteins can be used to affect the bioavailability of a 21509 or 33770 substrate. 21509 or 33770 fusion proteins may be useful therapeutically for the treatment of disorders caused by, for example, (i) aberrant modification or mutation of a gene encoding a 21509 or 33770 protein; (ii) mis-regulation of the 21509 or 33770 gene; and (iii) aberrant post-translational modification of a 21509 or 33770 protein.

Moreover, the 21509 or 33770-fusion proteins of the invention can be used as immunogens to produce anti-21509 or 33770 antibodies in a subject, to purify 21509 or 33770 ligands and in screening assays to identify molecules which inhibit the interaction of 21509 or 33770 with a 21509 or 33770 substrate.

Expression vectors are commercially available that already encode a fusion moiety (e.g., a GST polypeptide). A 21509 or 33770-encoding nucleic acid can be cloned into such an expression vector such that the fusion moiety is linked in-frame to the 21509 or 33770 protein.

Variants of 21509 or 33770 Proteins

In another aspect, the invention also features a variant of a 21509 or 33770 polypeptide, e.g., which functions as an agonist (mimetics) or as an antagonist. Variants of the 21509 or 33770 proteins can be generated by mutagenesis, e.g., discrete point mutation, the insertion or deletion of sequences or the truncation of a 21509 or 33770 protein. An agonist of the 21509 or 33770 proteins can retain substantially the same, or a subset, of the biological activities of the naturally occurring form of a 21509 or 33770 protein. An antagonist of a 21509 or 33770 protein can inhibit one or more of the activities of the naturally occurring form of the 21509 or 33770 protein by, for example, competitively modulating a 21509 or 33770-mediated activity of a 21509 or 33770 protein. Thus, specific biological effects can be elicited by treatment with a variant of limited function. Preferably, treatment of a subject with a variant having a subset of the biological activities of the naturally occurring form of the protein has fewer side effects in a subject relative to treatment with the naturally occurring form of the 21509 or 33770 protein.

Variants of a 21509 or 33770 protein can be identified by screening combinatorial libraries of mutants, e.g., truncation mutants, of a 21509 or 33770 protein for agonist or antagonist activity.

Libraries of fragments e.g., N terminal, C terminal, or internal fragments, of a 21509 or 33770 protein coding sequence can be used to generate a variegated population of fragments for screening and subsequent selection of variants of a 21509 or 33770 protein. Variants in which a cysteine residues is added or deleted or in which a residue which is glycosylated is added or deleted are particularly preferred.

Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of 21509 or 33770 proteins. Recursive ensemble mutagenesis (REM), a new technique which enhances the frequency of functional mutants in the libraries, can be used in combination with the screening assays to identify 21509 or 33770 variants (Arkin and Yourvan (1992) Proc. Natl. Acad. Sci. USA 89:7811-7815; Delgrave et al. (1993) Protein Engineering 6:327-331).

Cell based assays can be exploited to analyze a variegated 21509 or 33770 library. For example, a library of expression vectors can be transfected into a cell line, e.g., a cell line, which ordinarily responds to 21509 or 33770 in a substrate-dependent manner. The transfected cells are then contacted with 21509 or 33770 and the effect of the expression of the mutant on signaling by the 21509 or 33770 substrate can be detected, e.g., by measuring dehydrogenation or reduction of the substrate. Plasmid DNA can then be recovered from the cells which score for inhibition, or alternatively, potentiation of signaling by the 21509 or 33770 substrate, and the individual clones further characterized.

In another aspect, the invention features a method of making a 21509 or 33770 polypeptide, e.g., a peptide having a non-wild type activity, e.g., an antagonist, agonist, or super agonist of a naturally occurring 21509 or 33770 polypeptide, e.g., a naturally occurring 21509 or 33770 polypeptide. The method includes: altering the sequence of a 21509 or 33770 polypeptide, e.g., altering the sequence, e.g., by substitution or deletion of one or more residues of a non-conserved region, a domain or residue disclosed herein, and testing the altered polypeptide for the desired activity.

In another aspect, the invention features a method of making a fragment or analog of a 21509 or 33770 polypeptide a biological activity of a naturally occurring 21509 or 33770 polypeptide. The method includes: altering the sequence, e.g., by substitution or deletion of one or more residues, of a 21509 or 33770 polypeptide, e.g., altering the sequence of a non-conserved region, or a domain or residue described herein, and testing the altered polypeptide for the desired activity.

Anti-21509 or 33770 Antibodies

In another aspect, the invention provides an anti-21509 or 33770 antibody, or a fragment thereof (e.g., an antigen-binding fragment thereof). The term “antibody” as used herein refers to an immunoglobulin molecule or immunologically active portion thereof, i.e., an antigen-binding portion. As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4.

The anti-21509 or 33770 antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

As used herein, the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 KDa or 214 amino acids) are encoded by a variable region gene at the NH2-terminus (about 110 amino acids) and a kappa or lambda constant region gene at the COOH—terminus. Full-length immunoglobulin “heavy chains” (about 50 KDa or 446 amino acids), are similarly encoded by a variable region gene (about 116 amino acids) and one of the other aforementioned constant region genes, e.g., gamma (encoding about 330 amino acids).

The term “antigen-binding fragment” of an antibody (or simply “antibody portion,” or “fragment”), as used herein, refers to one or more fragments of a full-length antibody that retain the ability to specifically bind to the antigen, e.g., 21509 or 33770 polypeptide or fragment thereof. Examples of antigen-binding fragments of the anti-21509 or 33770 antibody include, but are not limited to: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “antigen-binding fragment” of an antibody. These antibody fragments are obtained using conventional techniques known to those with skill in the art, and the fragments are screened for utility in the same manner as are intact antibodies.

The anti-21509 or 33770 antibody can be a polyclonal or a monoclonal antibody. In other embodiments, the antibody can be recombinantly produced, e.g., produced by phage display or by combinatorial methods.

Phage display and combinatorial methods for generating anti-21509 or 33770 antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the anti-21509 or 33770 antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Method of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An anti-21509 or 33770 antibody can be one in which the variable region, or a portion thereof, e.g., the CDR's, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDR's (of heavy and or light immuoglobulin chains) replaced with a donor CDR. An antibody may be replaced with at least a portion of a non-human CDR or only some of the CDR's may be replaced with non-human CDR's. It is only necessary to replace the number of CDR's required for binding of the humanized antibody to a 21509 or 33770 or a fragment thereof. Preferably, the donor will be a rodent antibody, e.g., a rat or mouse antibody, and the recipient will be a human framework or a human consensus framework. Typically, the immunoglobulin providing the CDR's is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art. Humanized antibodies can be generated by replacing sequences of the Fv variable region which are not directly involved in antigen binding with equivalent sequences from human Fv variable regions. General methods for generating humanized antibodies are provided by Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a 21509 or 33770 polypeptide or fragment thereof. The recombinant DNA encoding the humanized antibody, or fragment thereof, can then be cloned into an appropriate expression vector.

Humanized or CDR-grafted antibodies can be produced by CDR-grafting or CDR substitution, wherein one, two, or all CDR's of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Preferred humanized antibodies have amino acid substitutions in the framework region, such as to improve binding to the antigen. For example, a humanized antibody will have framework residues identical to the donor framework residue or to another amino acid other than the recipient framework residue. To generate such antibodies, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

In preferred embodiments an antibody can be made by immunizing with purified 21509 or 33770 antigen, or a fragment thereof, e.g., a fragment described herein, membrane associated antigen, tissue, e.g., crude tissue preparations, whole cells, preferably living cells, lysed cells, or cell fractions, e.g., membrane fractions.

A full-length 21509 or 33770 protein or, antigenic peptide fragment of 21509 or 33770 can be used as an immunogen or can be used to identify anti-21509 or 33770 antibodies made with other immunogens, e.g., cells, membrane preparations, and the like. The antigenic peptide of 21509 or 33770 should include at least 8 amino acid residues of the amino acid sequence shown in SEQ ID NO:14 or 17 and encompasses an epitope of 21509 or 33770. Preferably, the antigenic peptide includes at least 10 amino acid residues, more preferably at least 15 amino acid residues, even more preferably at least 20 amino acid residues, and most preferably at least 30 amino acid residues.

Fragments of 21509 or 33770 which include, e.g., residues 3-184,201-229, 33-37, 36-238, 209-229, 114-116, 66-69, 95-98, 9-14, 38-43, 110-115, 128-133, 134-139, 153-158 or 148-158 of SEQ ID NO:14, or combinations containing contiguous sequences thereof; or residues 17-487, 483-487, 145-163, 314-330, 463-466, 248-251, 42-44, 62-62, 140-142, 162-164, 275-277, 290-292, 211-313, 484-486, 23-26, 31-34, 42-45, 65-68, 83-86, 129-132, 220-223, 404-407, 198-203, 231-236, 327-332, 418-423, 441-446, 458-463, 469-474, 280-291, or 252-259 of SEQ ID NO: 17, or combinations containing contiguous sequences thereof can be used, e.g., as immunogens or used to characterize the specificity of an antibody. A fragment of residues 180-200 of SEQ ID NO: 14 can be used to produce antibodies against hydrophilic regions of the 21509 protein; a fragment of residues 15-35 or 80-90 of SEQ ID NO:17 can be used as immunogens to produce antibodies against hydrophilic regions of the 33770 protein. Similarly, a fragment of 21509 which includes residues 130-140 or 210-220 of SEQ ID NO: 14 can be used to make an antibody against a hydrophobic region of the 21509 protein; a fragment of 33770 which includes residues 140-175 of SEQ ID NO:17 can be used to make an antibody against a hydrophobic region of the 33770 protein. Antibodies reactive with, or specific for, any of these regions, or other regions or domains described herein are therefore provided.

Antibodies reactive with, or specific for, any of these regions, or other regions or domains described herein are provided.

Antibodies which bind only native 21509 or 33770 protein, only denatured or otherwise non-native 21509 or 33770 protein, or which bind both, are with in the invention. Antibodies with linear or conformational epitopes are within the invention. Conformational epitopes can sometimes be identified by identifying antibodies which bind to native but not denatured 21509 or 33770 protein.

Preferred epitopes encompassed by the antigenic peptide are regions of 21509 or 33770 are located on the surface of the protein, e.g., hydrophilic regions, as well as regions with high antigenicity. For example, an Emini surface probability analysis of the human 21509 or 33770 protein sequence can be used to indicate the regions that have a particularly high probability of being localized to the surface of the 21509 or 33770 protein and are thus likely to constitute surface residues useful for targeting antibody production.

The anti-21509 or 33770 antibody can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target 21509 or 33770 protein.

In a preferred embodiment the antibody has: effector function; and can fix complement. In other embodiments the antibody does not; recruit effector cells; or fix complement.

In a preferred embodiment, the antibody has reduced or no ability to bind an Fc receptor. For example., it is a isotype or subtype, fragment or other mutant, which does not support binding to an Fc receptor, e.g., it has a mutagenized or deleted Fc receptor binding region.

In a preferred embodiment, an anti-21509 or 33770 antibody alters (e.g., increases or decreases) the dehydrogenase or reductase activity of a 21509 or 33770 polypeptide. For example, the antibody can bind at or in proximity to the active site, e.g., to an epitope that includes a residue located from about 148 to 158 of SEQ ID NO: 14 or about 252 to 259 or 280 to 291 of SEQ ID NO: 17.

The antibody can be coupled to a toxin, e.g., a polypeptide toxin, e,g, ricin or diphtheria toxin or active fragment hereof, or a radioactive nucleus, or imaging agent, e.g. a radioactive, enzymatic, or other, e.g., imaging agent, e.g., a NMR contrast agent. Labels which produce detectable radioactive emissions or fluorescence are preferred.

An anti-21509 or 33770 antibody (e.g., monoclonal antibody) can be used to isolate 21509 or 33770 by standard techniques, such as affinity chromatography or immunoprecipitation. Moreover, an anti-21509 or 33770 antibody can be used to detect 21509 or 33770 protein (e.g., in a cellular lysate or cell supernatant) in order to evaluate the abundance and pattern of expression of the protein. Anti-21509 or 33770 antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labelling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

The invention also includes a nucleic acid which encodes an anti-21509 or 33770 antibody, e.g., an anti-21509 or 33770 antibody described herein. Also included are vectors which include the nucleic acid and cells transformed with the nucleic acid, particularly cells which are useful for producing an antibody, e.g., mammalian cells, e.g. CHO or lymphatic cells.

The invention also includes cell lines, e.g., hybridomas, which make an anti-21509 or 33770 antibody, e.g., and antibody described herein, and method of using said cells to make a 21509 or 33770 antibody.

21509 and 33770 Recombinant Expression Vectors, Host Cells and Genetically Engineered Cells

In another aspect, the invention includes, vectors, preferably expression vectors, containing a nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a 21509 or 33770 nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., 21509 or 33770 proteins, mutant forms of 21509 or 33770 proteins, fusion proteins, and the like).

The recombinant expression vectors of the invention can be designed for expression of 21509 or 33770 proteins in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in 21509 or 33770 activity assays, (e.g., direct assays or competitive assays described in detail below), or to generate antibodies specific for 21509 or 33770 proteins. In a preferred embodiment, a fusion protein expressed in a retroviral expression vector of the present invention can be used to infect bone marrow cells which are subsequently transplanted into irradiated recipients. The pathology of the subject recipient is then examined after sufficient time has passed (e.g., six weeks).

To maximize recombinant protein expression in E. coli is to express the protein in a host bacteria with an impaired capacity to proteolytically cleave the recombinant protein (Gottesman, S., (1990) Gene Expression Technology: Methods in Enzymology 185, Academic Press, San Diego, Calif. 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The 21509 or 33770 expression vector can be a yeast expression vector, a vector for expression in insect cells, e.g., a baculovirus expression vector or a vector suitable for expression in mammalian cells.

When used in mammalian cells, the expression vector's control functions can be provided by viral regulatory elements. For example, commonly used promoters are derived from polyoma, Adenovirus 2, cytomegalovirus and Simian Virus 40.

In another embodiment, the promoter is an inducible promoter, e.g., a promoter regulated by a steroid hormone, by a polypeptide hormone (e.g., by means of a signal transduction pathway), or by a heterologous polypeptide (e.g., the tetracycline-inducible systems, “Tet-On” and “Tet-Off”; see, e.g., Clontech Inc., CA, Gossen and Bujard (1992) Proc. Natl. Acad. Sci. USA 89:5547, and Paillard (1989) Human Gene Therapy 9:983).

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus.

Another aspect the invention provides a host cell which includes a nucleic acid molecule described herein, e.g., a 21509 or 33770 nucleic acid molecule within a recombinant expression vector or a 21509 or 33770 nucleic acid molecule containing sequences which allow it to homologously recombine into a specific site of the host cell's genome. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a 21509 or 33770 protein can be expressed in bacterial cells (such as E. coli), insect cells, yeast or mammalian cells (such as Chinese hamster ovary cells (CHO) or COS cells). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell of the invention can be used to produce (i.e., express) a 21509 or 33770 protein. Accordingly, the invention further provides methods for producing a 21509 or 33770 protein using the host cells of the invention. In one embodiment, the method includes culturing the host cell of the invention (into which a recombinant expression vector encoding a 21509 or 33770 protein has been introduced) in a suitable medium such that a 21509 or 33770 protein is produced. In another embodiment, the method further includes isolating a 21509 or 33770 protein from the medium or the host cell.

In another aspect, the invention features, a cell or purified preparation of cells which include a 21509 or 33770 transgene, or which otherwise misexpress 21509 or 33770. The cell preparation can consist of human or non-human cells, e.g., rodent cells, e.g., mouse or rat cells, rabbit cells, or pig cells. In preferred embodiments, the cell or cells include a 21509 or 33770 transgene, e.g., a heterologous form of a 21509 or 33770, e.g., a gene derived from humans (in the case of a non-human cell). The 21509 or 33770 transgene can be misexpressed, e.g., overexpressed or underexpressed. In other preferred embodiments, the cell or cells include a gene that mis-expresses an endogenous 21509 or 33770, e.g., a gene the expression of which is disrupted, e.g., a knockout. Such cells can serve as a model for studying disorders that are related to mutated or mis-expressed 21509 or 33770 alleles or for use in drug screening.

In another aspect, the invention features, a human cell, e.g., a neural or hepatic stem cell, transformed with nucleic acid which encodes a subject 21509 or 33770 polypeptide.

Also provided are cells, preferably human cells, e.g., human neuronal, liver, or fibroblastic cells, in which an endogenous 21509 or 33770 is under the control of a regulatory sequence that does not normally control the expression of the endogenous 21509 or 33770 gene. The expression characteristics of an endogenous gene within a cell, e.g., a cell line or microorganism, can be modified by inserting a heterologous DNA regulatory element into the genome of the cell such that the inserted regulatory element is operably linked to the endogenous 21509 or 33770 gene. For example, an endogenous 21509 or 33770 gene which is “transcriptionally silent,” e.g., not normally expressed, or expressed only at very low levels, may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published in May 16, 1991.

In a preferred embodiment, recombinant cells described herein can be used for replacement therapy in a subject. For example, a nucleic acid encoding a 21509 or 33770 polypeptide operably linked to an inducible promoter (e.g., a steroid hormone receptor-regulated promoter) is introduced into a human or nonhuman, e.g., mammalian, e.g., porcine recombinant cell. The cell is cultivated and encapsulated in a biocompatible material, such as poly-lysine alginate, and subsequently implanted into the subject. See, e.g., Lanza (1996) Nat. Biotechnol. 14:1107; Joki et al (2001) Nat. Biotechnol. 19:35; and U.S. Pat. No. 5,876,742. Production of 21509 or 33770 polypeptide can be regulated in the subject by administering an agent (e.g., a steroid hormone) to the subject. In another preferred embodiment, the implanted recombinant cells express and secrete an antibody specific for a 21509 or 33770 polypeptide. The antibody can be any antibody or any antibody derivative described herein.

21509 and 33770 Transgenic Animals

The invention provides non-human transgenic animals. Such animals are useful for studying the function and/or activity of a 21509 or 33770 protein and for identifying and/or evaluating modulators of 21509 or 33770 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous 21509 or 33770 gene has been altered by, e.g., by homologous recombination between the endogenous gene and an exogenous DNA molecule introduced into a cell of the animal, e.g., an embryonic cell of the animal, prior to development of the animal.

Intronic sequences and polyadenylation signals can also be included in the transgene to increase the efficiency of expression of the transgene. A tissue-specific regulatory sequence(s) can be operably linked to a transgene of the invention to direct expression of a 21509 or 33770 protein to particular cells. A transgenic founder animal can be identified based upon the presence of a 21509 or 33770 transgene in its genome and/or expression of 21509 or 33770 mRNA in tissues or cells of the animals. A transgenic founder animal can then be used to breed additional animals carrying the transgene. Moreover, transgenic animals carrying a transgene encoding a 21509 or 33770 protein can further be bred to other transgenic animals carrying other transgenes. 21509 or 33770 proteins or polypeptides can be expressed in transgenic animals or plants, e.g., a nucleic acid encoding the protein or polypeptide can be introduced into the genome of an animal. In preferred embodiments the nucleic acid is placed under the control of a tissue specific promoter, e.g., a milk or egg specific promoter, and recovered from the milk or eggs produced by the animal. Suitable animals are mice, pigs, cows, goats, and sheep.

The invention also includes a population of cells from a transgenic animal, as discussed, e.g., below.

Uses of 21509 and 33770

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic).

The isolated nucleic acid molecules of the invention can be used, for example, to express a 21509 or 33770 protein (e.g., via a recombinant expression vector in a host cell in gene therapy applications), to detect a 21509 or 33770 mRNA (e.g., in a biological sample) or a genetic alteration in a 21509 or 33770 gene, and to modulate 21509 or 33770 activity, as described further below. The 21509 or 33770 proteins can be used to treat disorders characterized by insufficient or excessive production of a 21509 or 33770 substrate or production of 21509 or 33770 inhibitors. In addition, the 21509 or 33770 proteins can be used to screen for naturally occurring 21509 or 33770 substrates, to screen for drugs or compounds which modulate 21509 or 33770 activity, as well as to treat disorders characterized by insufficient or excessive production of 21509 or 33770 protein or production of 21509 or 33770 protein forms which have decreased, aberrant or unwanted activity compared to 21509 or 33770 wild type protein (e.g., disorders related to aberrant fatty acid synthesis or metabolism, e.g., diabetes or cardiovascular disease, or disorders related to hormonal imbalances involving, e.g., retinoids, estrogen, or androgen). Moreover, the anti-21509 or 33770 antibodies of the invention can be used to detect and isolate 21509 or 33770 proteins, regulate the bioavailability of 21509 or 33770 proteins, and modulate 21509 or 33770 activity.

A method of evaluating a compound for the ability to interact with, e.g., bind, a subject 21509 or 33770 polypeptide is provided. The method includes: contacting the compound with the subject 21509 or 33770 polypeptide; and evaluating ability of the compound to interact with, e.g., to bind or form a complex with the subject 21509 or 33770 polypeptide. This method can be performed in vitro, e.g., in a cell free system, or in vivo, e.g., in a two-hybrid interaction trap assay. This method can be used to identify naturally occurring molecules that interact with subject 21509 or 33770 polypeptide. It can also be used to find natural or synthetic inhibitors of subject 21509 or 33770 polypeptide. Screening methods are discussed in more detail below.

21509 and 33770 Screening Assays

The invention provides methods (also referred to herein as “screening assays”) for identifying modulators, i.e., candidate or test compounds or agents (e.g., proteins, peptides, peptidomimetics, peptoids, small molecules or other drugs) which bind to 21509 or 33770 proteins, have a stimulatory or inhibitory effect on, for example, 21509 or 33770 expression or 21509 or 33770 activity, or have a stimulatory or inhibitory effect on, for example, the expression or activity of a 21509 or 33770 substrate. Compounds thus identified can be used to modulate the activity of target gene products (e.g., 21509 or 33770 genes) in a therapeutic protocol, to elaborate the biological function of the target gene product, or to identify compounds that disrupt normal target gene interactions.

In one embodiment, the invention provides assays for screening candidate or test compounds which are substrates of a 21509 or 33770 protein or polypeptide or a biologically active portion thereof. In another embodiment, the invention provides assays for screening candidate or test compounds that bind to or modulate an activity of a 21509 or 33770 protein or polypeptide or a biologically active portion thereof.

The assays for dehydrogenase activity are well known in the art and can be found, for example, in Oppermann et al. (1999) FEBS 451:238-242, Thomasson et al. (1993) Behavior Genetics 23:131-136, and Zubey (1988) Macmillan Publishing Company, New York. These assays include, for example, determination of the Michaelis constants (Kₘ) or the dissociation constant for the dehydrogenase/substrate complex. Analysis of enzyme activity may be performed spectrophotometrically by recording the change in absorbance of NAD⁺, for example.

In one embodiment, an activity of a 21509 protein can be assayed in vitro according to the method of Post-Beittenmiller et al., (1991) J. Biol. Chem 266, 1858-65, the contents of which are hereby incorporated by reference. In another embodiment, an activity of a 33770 protein can be assayed according to the method of Lin and Napoli (2000), J. Biol. Chem. 275, 40106-12, the contents of which are hereby incorporated by reference.

The test compounds of the present invention can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In one embodiment, an assay is a cell-based assay in which a cell which expresses a 21509 or 33770 protein or biologically active portion thereof is contacted with a test compound, and the ability of the test compound to modulate 21509 or 33770 activity is determined. Determining the ability of the test compound to modulate 21509 or 33770 activity can be accomplished by monitoring, for example, hydrogenase or reductase activity. The cell, for example, can be of mammalian origin, e.g., human.

The ability of the test compound to modulate 21509 or 33770 binding to a compound, e.g., a 21509 or 33770 substrate, or to bind to 21509 or 33770 can also be evaluated. This can be accomplished, for example, by coupling the compound, e.g., the substrate, with a radioisotope or enzymatic label such that binding of the compound, e.g., the substrate, to 21509 or 33770 can be determined by detecting the labeled compound, e.g., substrate, in a complex. Alternatively, 21509 or 33770 could be coupled with a radioisotope or enzymatic label to monitor the ability of a test compound to modulate 21509 or 33770 binding to a 21509 or 33770 substrate in a complex. For example, compounds (e.g., 21509 or 33770 substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a 21509 or 33770 substrate) to interact with 21509 or 33770 with or without the labeling of any of the interactants can be evaluated. For example, a microphysiometer can be used to detect the interaction of a compound with 21509 or 33770 without the labeling of either the compound or the 21509 or 33770. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 21509 or 33770.

In yet another embodiment, a cell-free assay is provided in which a 21509 or 33770 protein or biologically active portion thereof is contacted with a test compound and the ability of the test compound to bind to the 21509 or 33770 protein or biologically active portion thereof is evaluated. Preferred biologically active portions of the 21509 or 33770 proteins to be used in assays of the present invention include fragments which participate in interactions with non-21509 or 33770 molecules, e.g., fragments with high surface probability scores.

Soluble and/or membrane-bound forms of isolated proteins (e.g., 21509 or 33770 proteins or biologically active portions thereof) can be used in the cell-free assays of the invention. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate. Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of the 21509 or 33770 protein to bind to a target molecule can be accomplished using real-time Biomolecular Interaction Analysis (BIA) (see, e.g., Sjolander, S. and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BLAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize either 21509 or 33770, an anti-21509 or 33770 antibody or its target molecule to facilitate separation of complexed from uncomplexed forms of one or both of the proteins, as well as to accommodate automation of the assay. Binding of a test compound to a 21509 or 33770 protein, or interaction of a 21509 or 33770 protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/21509 or 33770 fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or 21509 or 33770 protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of 21509 or 33770 binding or activity determined using standard techniques.

Other techniques for immobilizing either a 21509 or 33770 protein or a target molecule on matrices include using conjugation of biotin and streptavidin. Biotinylated 21509 or 33770 protein or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with 21509 or 33770 protein or target molecules but which do not interfere with binding of the 21509 or 33770 protein to its target molecule. Such antibodies can be derivatized to the wells of the plate, and unbound target or 21509 or 33770 protein trapped in the wells by antibody conjugation. Methods for detecting such complexes, in addition to those described above for the GST-immobilized complexes, include immunodetection of complexes using antibodies reactive with the 21509 or 33770 protein or target molecule, as well as enzyme-linked assays which rely on detecting an enzymatic activity associated with the 21509 or 33770 protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., (1993) Trends Biochem Sci 18:284-7); chromatography (gel filtration chromatography, ion-exchange chromatography); electrophoresis (see, e.g., Ausubel, F. et al., eds. Current Protocols in Molecular Biology 1999, J. Wiley: New York.); and immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N. H., (1998) J Mol Recognit 11: 141-8; Hage, D. S., and Tweed, S. A. (1997) J Chromatogr B Biomed Sci Appl. 699:499-525). Further, fluorescence energy transfer may also be conveniently utilized, as described herein, to detect binding without further purification of the complex from solution.

In a preferred embodiment, the assay includes contacting the 21509 or 33770 protein or biologically active portion thereof with a known compound which binds 21509 or 33770 to form an assay mixture, contacting the assay mixture with a test compound, and determining the ability of the test compound to interact with a 21509 or 33770 protein, wherein determining the ability of the test compound to interact with a 21509 or 33770 protein includes determining the ability of the test compound to preferentially bind to 21509 or 33770 or biologically active portion thereof, or to modulate the activity of a target molecule, as compared to the known compound.

The target gene products of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, peptides, and small molecules. The preferred target genes/products for use in this embodiment are the 21509 or 33770 genes herein identified. In an alternative embodiment, the invention provides methods for determining the ability of the test compound to modulate the activity of a 21509 or 33770 protein through modulation of the activity of a downstream effector of a 21509 or 33770 target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the 21509 or 33770 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with 21509 or 33770 (“21509 or 33770-binding proteins” or “21509 or 33770-bp”) and are involved in 21509 or 33770 activity. Such 21509 or 33770-bps can be activators or inhibitors of signals by the 21509 or 33770 proteins or 21509 or 33770 targets as, for example, downstream elements of a 21509 or 33770-mediated signaling pathway.

The two-hybrid system is based on the modular nature of most transcription factors, which consist of separable DNA-binding and activation domains. Briefly, the assay utilizes two different DNA constructs. In one construct, the gene that codes for a 21509 or 33770 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively the: 21509 or 33770 protein can be the fused to the activator domain.) If the “bait” and the “prey” proteins are able to interact, in vivo, forming a 21509 or 33770-dependent complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 21509 or 33770 protein.

In another embodiment, modulators of 21509 or 33770 expression are identified. For example, a cell or cell free mixture is contacted with a candidate compound and the expression of 21509 or 33770 mRNA or protein evaluated relative to the level of expression of 21509 or 33770 mRNA or protein in the absence of the candidate compound. When expression of 21509 or 33770 mRNA or protein is greater in the presence of the candidate compound than in its absence, the candidate compound is identified as a stimulator of 21509 or 33770 mRNA or protein expression. Alternatively, when expression of 21509 or 33770 mRNA or protein is less ¢ 10 (statistically significantly less) in the presence of the candidate compound than in its absence, the candidate compound is identified as an inhibitor of 21509 or 33770 mRNA or protein expression. The level of 21509 or 33770 mRNA or protein expression can be determined by methods described herein for detecting 21509 or 33770 mRNA or protein.

In another aspect, the invention pertains to a combination of two or more of the assays described herein. For example, a modulating agent can be identified using a cell-based or a cell free assay, and the ability of the agent to modulate the activity of a 21509 or 33770 protein can be confirmed in vivo, e.g., in an animal such as an animal model for diseases associated with abnormal lipid biosynthesis or metabolism, or hormonal imbalances.

This invention further pertains to novel agents identified by the above-described screening assays. Accordingly, it is within the scope of this invention to further use an agent identified as described herein (e.g., a 21509 or 33770 modulating agent, an antisense 21509 or 33770 nucleic acid molecule, a 21509 or 33770-specific antibody, or a 21509 or 33770-binding partner) in an appropriate animal model to determine the efficacy, toxicity, side effects, or mechanism of action, of treatment with such an agent. Furthermore, novel agents identified by the above-described screening assays can be used for treatments as described herein.

21509 and 33770 Detection Assays

Portions or fragments of the nucleic acid sequences identified herein can be used as polynucleotide reagents. For example, these sequences can be used to: (i) map their respective genes on a chromosome e.g., to locate gene regions associated with genetic disease or to associate 21509 or 33770 with a disease; (ii) identify an individual from a minute biological sample (tissue typing); and (iii) aid in forensic identification of a biological sample. These applications are described in the subsections below.

21509 and 33770 Chromosome Mapping

The 21509 or 33770 nucleotide sequences or portions thereof can be used to map the location of the 21509 or 33770 genes on a chromosome. This process is called chromosome mapping. Chromosome mapping is useful in correlating the 21509 or 33770 sequences with genes associated with disease.

Briefly, 21509 or 33770 genes can be mapped to chromosomes by preparing PCR primers (preferably 15-25 bp in length) from the 21509 or 33770 nucleotide sequences. These primers can then be used for PCR screening of somatic cell hybrids containing individual human chromosomes. Only those hybrids containing the human gene corresponding to the 21509 or 33770 sequences will yield an amplified fragment.

A panel of somatic cell hybrids in which each cell line contains either a single human chromosome or a small number of human chromosomes, and a full set of mouse chromosomes, can allow easy mapping of individual genes to specific human chromosomes. (D'Eustachio P. et al. (1983) Science 220:919-924).

Other mapping strategies e.g., in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries can be used to map 21509 or 33770 to a chromosomal location.

Fluorescence in situ hybridization (FISH) of a DNA sequence to a metaphase chromosomal spread can further be used to provide a precise chromosomal location in one step. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques ((1988) Pergamon Press, New York).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the 21509 or 33770 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

21509 and 33770 Tissue Typing

21509 or 33770 sequences can be used to identify individuals from biological samples using, e.g., restriction fragment length polymorphism (RFLP). In this technique, an individual's genomic DNA is digested with one or more restriction enzymes, the fragments separated, e.g., in a Southern blot, and probed to yield bands for identification. The sequences of the present invention are useful as additional DNA markers for RFLP (described in U.S. Pat. No. 5,272,057).

Furthermore, the sequences of the present invention can also be used to determine the actual base-by-base DNA sequence of selected portions of an individual's genome. Thus, the 21509 or 33770 nucleotide sequences described herein can be used to prepare two PCR primers from the 5′ and 3′ ends of the sequences. These primers can then be used to amplify an individual's DNA and subsequently sequence it. Panels of corresponding DNA sequences from individuals, prepared in this manner, can provide unique individual identifications, as each individual will have a unique set of such DNA sequences due to allelic differences.

Allelic variation occurs to some degree in the coding regions of these sequences, and to a greater degree in the noncoding regions. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO: 13 or SEQ ID NO: 16 can provide positive individual identification with a panel of perhaps 10 to 1,000 primers which each yield a noncoding amplified sequence of 100 bases. If predicted coding sequences, such as those in SEQ ID NO:15 are used, a more appropriate number of primers for positive individual identification would be 500-2,000.

If a panel of reagents from 21509 or 33770 nucleotide sequences described herein is used to generate a unique identification database for an individual, those same reagents can later be used to identify tissue from that individual. Using the unique identification database, positive identification of the individual, living or dead, can be made from extremely small tissue samples.

Use of Partial 21509 or 33770 Sequences in Forensic Biology

DNA-based identification techniques can also be used in forensic biology. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:13 or SEQ ID NO:16 (e.g., fragments derived from the noncoding regions of SEQ ID NO: 13 or SEQ ID NO: 16 having a length of at least 20 bases, preferably at least 30 bases) are particularly appropriate for this use.

The 21509 or 33770 nucleotide sequences described herein can further be used to provide polynucleotide reagents, e.g., labeled or labelable probes which can be used in, for example, an in situ hybridization technique, to identify a specific tissue. This can be very useful in cases where a forensic pathologist is presented with a tissue of unknown origin. Panels of such 21509 or 33770 probes can be used to identify tissue by species and/or by organ type.

In a similar fashion, these reagents, e.g., 21509 or 33770 primers or probes can be used to screen tissue culture for contamination (i.e. screen for the presence of a mixture of different types of cells in a culture).

Predictive Medicine of 21509 and 33770

The present invention also pertains to the field of predictive medicine in which diagnostic assays, prognostic assays, and monitoring clinical trials are used for prognostic (predictive) purposes to thereby treat an individual.

Generally, the invention provides, a method of determining if a subject is at risk for a disorder related to a lesion in or the misexpression of a gene which encodes 21509 or 33770.

Such disorders include, e.g., a disorder associated with the misexpression of 21509 or 33770 gene; a disorder of the metabolism, e.g., steroid hormon, retinoid, or fatty acid metabolism.

The method includes one or more of the following:

detecting, in a tissue of the subject, the presence or absence of a mutation which affects the expression of the 21509 or 33770 gene, or detecting the presence or absence of a mutation in a region which controls the expression of the gene, e.g., a mutation in the 5′ control region;

detecting, in a tissue of the subject, the presence or absence of a mutation which alters the structure of the 21509 or 33770 gene;

detecting, in a tissue of the subject, the misexpression of the 21509 or 33770 gene, at the mRNA level, e.g., detecting a non-wild type level of a mRNA;

detecting, in a tissue of the subject, the misexpression of the gene, at the protein level, e.g., detecting a non-wild type level of a 21509 or 33770 polypeptide.

In preferred embodiments the method includes: ascertaining the existence of at least one of: a deletion of one or more nucleotides from the 21509 or 33770 gene; an insertion of one or more nucleotides into the gene, a point mutation, e.g., a substitution of one or more nucleotides of the gene, a gross chromosomal rearrangement of the gene, e.g., a translocation, inversion, or deletion.

For example, detecting the genetic lesion can include: (i) providing a probe/primer including an oligonucleotide containing a region of nucleotide sequence which hybridizes to a sense or antisense sequence from SEQ ID NO: 13, or naturally occurring mutants thereof or 5′ or 3′ flanking sequences naturally associated with the 21509 or 33770 gene; (ii) exposing the probe/primer to nucleic acid of the tissue; and detecting, by hybridization, e.g., in situ hybridization, of the probe/primer to the nucleic acid, the presence or absence of the genetic lesion.

In preferred embodiments detecting the misexpression includes ascertaining the existence of at least one of: an alteration in the level of a messenger RNA transcript of the 21509 or 33770 gene; the presence of a non-wild type splicing pattern of a messenger RNA transcript of the gene; or a non-wild type level of 21509 or 33770.

Methods of the invention can be used prenatally or to determine if a subject's offspring will be at risk for a disorder.

In preferred embodiments the method includes determining the structure of a 21509 or 33770 gene, an abnormal structure being indicative of risk for the disorder.

In preferred embodiments the method includes contacting a sample from the subject with an antibody to the 21509 or 33770 protein or a nucleic acid, which hybridizes specifically with the gene. These and other embodiments are discussed below.

Diagnostic and Prognostic Assays of 21509 and 33770

Diagnostic and prognostic assays of the invention include method for assessing the expression level of 21509 or 33770 molecules and for identifying variations and mutations in the sequence of 21509 or 33770 molecules.

Expression Monitoring and Profiling:

The presence, level, or absence of 21509 or 33770 protein or nucleic acid in a biological sample can be evaluated by obtaining a biological sample from a test subject and contacting the biological sample with a compound or an agent capable of detecting 21509 or 33770 protein or nucleic acid (e.g., mRNA, genomic DNA) that encodes 21509 or 33770 protein such that the presence of 21509 or 33770 protein or nucleic acid is detected in the biological sample. The term “biological sample” includes tissues, cells and biological fluids isolated from a subject, as well as tissues, cells and fluids present within a subject. A preferred biological sample is serum.

The level of expression of the 21509 or 33770 gene can be measured in a number of ways, including, but not limited to: measuring the mRNA encoded by the 21509 or 33770 genes; measuring the amount of protein encoded by the 21509 or 33770 genes; or measuring the activity of the protein encoded by the 21509 or 33770 genes.

The level of mRNA corresponding to the 21509 or 33770 gene in a cell can be determined both by in situ and by in vitro formats.

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length 21509 or 33770 nucleic acid, such as the nucleic acid of SEQ ID NO: 13, or a portion thereof, such as an oligonucleotide of at least 7, 15, 30, 50, 100, 250 or 500 nucleotides in length and sufficient to specifically hybridize under stringent conditions to 21509 or 33770 mRNA or genomic DNA. The probe can be disposed on an address of an array, e.g., an array described below. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array described below. A skilled artisan can adapt known mRNA detection methods for use in detecting the level of mRNA encoded by the 21509 or 33770 genes.

The level of mRNA in a sample that is encoded by one of 21509 or 33770 can be evaluated with nucleic acid amplification, e.g., by rtPCR (Mullis (1987) U.S. Pat. No. 4,683,202), ligase chain reaction (Barany (1991) Proc. Natl. Acad. Sci. USA 88:189-193), self sustained sequence replication (Guatelli et al., (1990) Proc. Natl. Acad. Sci. USA 87:1874-1878), transcriptional amplification system (Kwoh et al., (1989), Proc. Natl. Acad. Sci. USA 86:1173-1177), Q-Beta Replicase (Lizardi et al., (1988) Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to “mRNA that encodes the 21509 or 33770 gene being analyzed.

In another embodiment, the methods further contacting a control sample with a compound or agent capable of detecting 21509 or 33770 mRNA, or genomic DNA, and comparing the presence of 21509 or 33770 mRNA or genomic DNA in the control sample with the presence of 21509 or 33770 mRNA or genomic DNA in the test sample. In still another embodiment, serial analysis of gene expression, as described in U.S. Pat. No. 5,695,937, is used to detect 21509 or 33770 transcript levels.

A variety of methods can be used to determine the level of protein encoded by 21509 or 33770. In general, these methods include contacting an agent that selectively binds to the protein, such as an antibody with a sample, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect 21509 or 33770 protein in a biological sample in vitro as well as in vivo. In vitro techniques for detection of 21509 or 33770 protein include enzyme linked immunosorbent assays (ELISAs), immunoprecipitations, immunofluorescence, enzyme immunoassay (EIA), radioimmunoassay (RIA), and Western blot analysis. In vivo techniques for detection of 21509 or 33770 protein include introducing into a subject a labeled anti-21509 or 33770 antibody. For example, the antibody can be labeled with a radioactive marker whose presence and location in a subject can be detected by standard imaging techniques. In another embodiment, the sample is labeled, e.g., biotinylated and then contacted to the antibody, e.g., an anti-21509 or 33770 antibody positioned on an antibody array (as described below). The sample can be detected, e.g., with avidin coupled to a fluorescent label.

In another embodiment, the methods further include contacting the control sample with a compound or agent capable of detecting 21509 or 33770 protein, and comparing the presence of 21509 or 33770 protein in the control sample with the presence of 21509 or 33770 protein in the test sample.

The invention also includes kits for detecting the presence of 21509 or 33770 in a biological sample. For example, the kit can include a compound or agent capable of detecting 21509 or 33770 protein or mRNA in a biological sample; and a standard. The compound or agent can be packaged in a suitable container. The kit can further comprise instructions for using the kit to detect 21509 or 33770 protein or nucleic acid.

For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can include: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also includes a buffering agent, a preservative, or a protein stabilizing agent. The kit can also includes components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, along with instructions for interpreting the results of the assays performed using the kit.

The diagnostic methods described herein can identify subjects having, or at risk of developing, a disease or disorder associated with misexpressed or aberrant or unwanted 21509 or 33770 expression or activity. As used herein, the term “unwanted” includes an unwanted phenomenon involved in a biological response such as cardiovascular disease, hormonal imbalance, neurodegenerative disease, or deregulated cell proliferation.

In one embodiment, a disease or disorder associated with aberrant or unwanted 21509 or 33770 expression or activity is identified. A test sample is obtained from a subject and 21509 or 33770 protein or nucleic acid (e.g., mRNA or genomic DNA) is evaluated, wherein the level, e.g., the presence or absence, of 21509 or 33770 protein or nucleic acid is diagnostic for a subject having or at risk of developing a disease or disorder associated with aberrant or unwanted 21509 or 33770 expression or activity. As used herein, a “test sample” refers to a biological sample obtained from a subject of interest, including a biological fluid (e.g., serum), cell sample, or tissue.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted 21509 or 33770 expression or activity. For example, such methods can be used to determine whether a subject can be effectively treated with an agent for a cellular proliferation disorder, e.g., cancer, or a cardiovascular, neurodegenerative, or hormonal disorder.

In another aspect, the invention features a computer medium having a plurality of digitally encoded data records. Each data record includes a value representing the level of expression of 21509 or 33770 in a sample, and a descriptor of the sample. The descriptor of the sample can be an identifier of the sample, a subject from which the sample was derived (e.g., a patient), a diagnosis, or a treatment (e.g., a preferred treatment). In a preferred embodiment, the data record further includes values representing the level of expression of genes other than 21509 or 33770 (e.g., other genes associated with a 21509 or 33770-disorder, or other genes on an array). The data record can be structured as a table, e.g., a table that is part of a database such as a relational database (e.g., a SQL database of the Oracle or Sybase database environments).

Also featured is a method of evaluating a sample. The method includes providing a sample, e.g., from the subject, and determining a gene expression profile of the sample, wherein the profile includes a value representing the level of 21509 or 33770 expression. The method can further include comparing the value or the profile (i.e., multiple values) to a reference value or reference profile. The gene expression profile of the sample can be obtained by any of the methods described herein (e.g., by providing a nucleic acid from the sample and contacting the nucleic acid to an array). The method can be used to diagnose a cellular proliferative disorder in a subject wherein an increase in 21509 or 33770 expression is an indication that the subject has or is disposed to having a cellular proliferative disorder. The method can be used to monitor a treatment for abnormal cellular proliferation or differentiation in a subject. For example, the gene expression profile can be determined for a sample from a subject undergoing treatment. The profile can be compared to a reference profile or to a profile obtained from the subject prior to treatment or prior to onset of the disorder (see, e.g., Golub et al. (1999) Science 286:531).

In yet another aspect, the invention features a method of evaluating a test compound (see also, “Screening Assays”, above). The method includes providing a cell and a test compound; contacting the test compound to the cell; obtaining a subject expression profile for the contacted cell; and comparing the subject expression profile to one or more reference profiles. The profiles include a value representing the level of 21509 or 33770 expression. In a preferred embodiment, the subject expression profile is compared to a target profile, e.g., a profile for a normal cell or for desired condition of a cell. The test compound is evaluated favorably if the subject expression profile is more similar to the target profile than an expression profile obtained from an uncontacted cell.

In another aspect, the invention features, a method of evaluating a subject. The method includes: a) obtaining a sample from a subject, e.g., from a caregiver, e.g., a caregiver who obtains the sample from the subject; b) determining a subject expression profile for the sample. Optionally, the method further includes either or both of steps: c) comparing the subject expression profile to one or more reference expression profiles; and d) selecting the reference profile most similar to the subject reference profile. The subject expression profile and the reference profiles include a value representing the level of 21509 or 33770 expression. A variety of routine statistical measures can be used to compare two reference profiles. One possible metric is the length of the distance vector that is the difference between the two profiles. Each of the subject and reference profile is represented as a multi-dimensional vector, wherein each dimension is a value in the profile.

The method can further include transmitting a result to a caregiver. The result can be the subject expression profile, a result of a comparison of the subject expression profile with another profile, a most similar reference profile, or a descriptor of any of the aforementioned. The result can be transmitted across a computer network, e.g., the result can be in the form of a computer transmission, e.g., a computer data signal embedded in a carrier wave.

Also featured is a computer medium having executable code for effecting the following steps: receive a subject expression profile; access a database of reference expression profiles; and either i) select a matching reference profile most similar to the subject expression profile or ii) determine at least one comparison score for the similarity of the subject expression profile to at least one reference profile. The subject expression profile, and the reference expression profiles each include a value representing the level of 21509 or 33770 expression.

21509 and 33770 Arrays and Uses Thereof

In another aspect, the invention features an array that includes a substrate having a plurality of addresses. At least one address of the plurality includes a capture probe that binds specifically to a 21509 or 33770 molecule (e.g., a 21509 or 33770 nucleic acid or a 21509 or 33770 polypeptide). The array can have a density of at least than 10, 50, 100, 200, 500, 1,000, 2,000, or 10,000 or more addresses/cm², and ranges between. In a preferred embodiment, the plurality of addresses includes at least 10, 100, 500, 1,000, 5,000, 10,000, 50,000 addresses. In a preferred embodiment, the plurality of addresses includes equal to or less than 10, 100, 500, 1,000, 5,000, 10,000, or 50,000 addresses. The substrate can be a two-dimensional substrate such as a glass slide, a wafer (e.g., silica or plastic), a mass spectroscopy plate, or a three-dimensional substrate such as a gel pad. Addresses in addition to address of the plurality can be disposed on the array.

In a preferred embodiment, at least one address of the plurality includes a nucleic acid capture probe that hybridizes specifically to a 21509 or 33770 nucleic acid, e.g., the sense or anti-sense strand. In one preferred embodiment, a subset of addresses of the plurality of addresses has a nucleic acid capture probe for 21509 or 33770. Each address of the subset can include a capture probe that hybridizes to a different region of a 21509 or 33770 nucleic acid. In another preferred embodiment, addresses of the subset include a capture probe for a 21509 or 33770 nucleic acid. Each address of the subset is unique, overlapping, and complementary to a different variant of 21509 or 33770 (e.g., an allelic variant, or all possible hypothetical variants). The array can be used to sequence 21509 or 33770 by hybridization (see, e.g., U.S. Pat. No. 5,695,940).

An array can be generated by various methods, e.g., by photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145).

In another preferred embodiment, at least one address of the plurality includes a polypeptide capture probe that binds specifically to a 21509 or 33770 polypeptide or fragment thereof. The polypeptide can be a naturally-occurring interaction partner of 21509 or 33770 polypeptide. Preferably, the polypeptide is an antibody, e.g., an antibody described herein (see “Anti-21509 or 33770 Antibodies,” above), such as a monoclonal antibody or a single-chain antibody.

In another aspect, the invention features a method of analyzing the expression of 21509 or 33770. The method includes providing an array as described above; contacting the array with a sample and detecting binding of a 21509 or 33770-molecule (e.g., nucleic acid or polypeptide) to the array. In a preferred embodiment, the array is a nucleic acid array. Optionally the method further includes amplifying nucleic acid from the sample prior or during contact with the array.

In another embodiment, the array can be used to assay gene expression in a tissue to ascertain tissue specificity of genes in the array, particularly the expression of 21509 or 33770. If a sufficient number of diverse samples is analyzed, clustering (e.g., hierarchical clustering, k-means clustering, Bayesian clustering and the like) can be used to identify other genes which are co-regulated with 21509 or 33770. For example, the array can be used for the quantitation of the expression of multiple genes. Thus, not only tissue specificity, but also the level of expression of a battery of genes in the tissue is ascertained. Quantitative data can be used to group (e.g., cluster) genes on the basis of their tissue expression per se and level of expression in that tissue.

For example, array analysis of gene expression can be used to assess the effect of cell-cell interactions on 21509 or 33770 expression. A first tissue can be perturbed and nucleic acid from a second tissue that interacts with the first tissue can be analyzed. In this context, the effect of one cell type on another cell type in response to a biological stimulus can be determined, e.g., to monitor the effect of cell-cell interaction at the level of gene expression.

In another embodiment, cells are contacted with a therapeutic agent. The expression profile of the cells is determined using the array, and the expression profile is compared to the profile of like cells not contacted with the agent. For example, the assay can be used to determine or analyze the molecular basis of an undesirable effect of the therapeutic agent. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor expression of one or more genes in the array with respect to time. For example, samples obtained from different time points can be probed with the array. Such analysis can identify and/or characterize the development of a 21509 or 33770-associated disease or disorder; and processes, such as a cellular transformation associated with a 21509 or 33770-associated disease or disorder. The method can also evaluate the treatment and/or progression of a 21509 or 33770-associated disease or disorder

The array is also useful for ascertaining differential expression patterns of one or more genes in normal and abnormal cells. This provides a battery of genes (e.g., including 21509 or 33770) that could serve as a molecular target for diagnosis or therapeutic intervention.

In another aspect, the invention features an array having a plurality of addresses. Each address of the plurality includes a unique polypeptide. At least one address of the plurality has disposed thereon a 21509 or 33770 polypeptide or fragment thereof. Methods of producing polypeptide arrays are described in the art, e.g., in De Wildt et al. (2000). Nature Biotech. 18, 989-994; Lueking et al. (1999). Anal. Biochem. 270, 103-111; Ge, H. (2000). Nucleic Acids Res. 28, e3, I-VII; MacBeath, G., and Schreiber, S. L. (2000). Science 289, 1760-1763; and WO 99/51773A1. In a preferred embodiment, each addresses of the plurality has disposed thereon a polypeptide at least 60, 70, 80,85, 90, 95 or 99% identical to a 21509 or 33770 polypeptide or fragment thereof. For example, multiple variants of a 21509 or 33770 polypeptide (e.g., encoded by allelic variants, site-directed mutants, random mutants, or combinatorial mutants) can be disposed at individual addresses of the plurality. Addresses in addition to the address of the plurality can be disposed on the array.

The polypeptide array can be used to detect a 21509 or 33770 binding compound, e.g., an antibody in a sample from a subject with specificity for a 21509 or 33770 polypeptide or the presence of a 21509 or 33770-binding protein or ligand.

The array is also useful for ascertaining the effect of the expression of a gene on the expression of other genes in the same cell or in different cells (e.g., ascertaining the effect of 21509 or 33770 expression on the expression of other genes). This provides, for example, for a selection of alternate molecular targets for therapeutic intervention if the ultimate or downstream target cannot be regulated.

In another aspect, the invention features a method of analyzing a plurality of probes. The method is useful, e.g., for analyzing gene expression. The method includes: providing a two dimensional array having a plurality of addresses, each address of the plurality being positionally distinguishable from each other address of the plurality having a unique capture probe, e.g., wherein the capture probes are from a cell or subject which express 21509 or 33770 or from a cell or subject in which a 21509 or 33770 mediated response has been elicited, e.g., by contact of the cell with 21509 or 33770 nucleic acid or protein, or administration to the cell or subject 21509 or 33770 nucleic acid or protein; providing a two dimensional array having a plurality of addresses, each address of the plurality being positionally distinguishable from each other address of the plurality, and each address of the plurality having a unique capture probe, e.g., wherein the capture probes are from a cell or subject which does not express 21509 or 33770 (or does not express as highly as in the case of the 21509 or 33770 positive plurality of capture probes) or from a cell or subject which in which a 21509 or 33770 mediated response has not been elicited (or has been elicited to a lesser extent than in the first sample); contacting the array with one or more inquiry probes (which is preferably other than a 21509 or 33770 nucleic acid, polypeptide, or antibody), and thereby evaluating the plurality of capture probes. Binding, e.g., in the case of a nucleic acid, hybridization with a capture probe at an address of the plurality, is detected, e.g., by signal generated from a label attached to the nucleic acid, polypeptide, or antibody.

In another aspect, the invention features a method of analyzing a plurality of probes or a sample. The method is useful, e.g., for analyzing gene expression. The method includes: providing a two dimensional array having a plurality of addresses, each address of the plurality being positionally distinguishable from each other address of the plurality having a unique capture probe, contacting the array with a first sample from a cell or subject which express or mis-express 21509 or 33770 or from a cell or subject in which a 21509 or 33770-mediated response has been elicited, e.g., by contact of the cell with 21509 or 33770 nucleic acid or protein, or administration to the cell or subject 21509 or 33770 nucleic acid or protein; providing a two dimensional array having a plurality of addresses, each address of the plurality being positionally distinguishable from each other address of the plurality, and each address of the plurality having a unique capture probe, and contacting the array with a second sample from a cell or subject which does not express 21509 or 33770 (or does not express as highly as in the case of the 21509 or 33770 positive plurality of capture probes) or from a cell or subject which in which a 21509 or 33770 mediated response has not been elicited (or has been elicited to a lesser extent than in the first sample); and comparing the binding of the first sample with the binding of the second sample. Binding, e.g., in the case of a nucleic acid, hybridization with a capture probe at an address of the plurality, is detected, e.g., by signal generated from a label attached to the nucleic acid, polypeptide, or antibody. The same array can be used for both samples or different arrays can be used. If different arrays are used the plurality of addresses with capture probes should be present on both arrays.

In another aspect, the invention features a method of analyzing 21509 or 33770, e.g., analyzing structure, function, or relatedness to other nucleic acid or amino acid sequences. The method includes: providing a 21509 or 33770 nucleic acid or amino acid sequence; comparing the 21509 or 33770 sequence with one or more preferably a plurality of sequences from a collection of sequences, e.g., a nucleic acid or protein sequence database; to thereby analyze 21509 or 33770.

Detection of 21509 and 33770 Variations or Mutations

The methods of the invention can also be used to detect genetic alterations in a 21509 or 33770 gene, thereby determining if a subject with the altered gene is at risk for a disorder characterized by misregulation in 21509 or 33770 protein activity or nucleic acid expression, such as a cellular proliferative disorder, e.g., cancer, or a cardiovascular, neurodegenerative, or hormonal disorder. In preferred embodiments, the methods include detecting, in a sample from the subject, the presence or absence of a genetic alteration characterized by at least one of an alteration affecting the integrity of a gene encoding a 21509 or 33770-protein, or the mis-expression of the 21509 or 33770 gene. For example, such genetic alterations can be detected by ascertaining the existence of at least one of 1) a deletion of one or more nucleotides from a 21509 or 33770 gene; 2) an addition of one or more nucleotides to a 21509 or 33770 gene; 3) a substitution of one or more nucleotides of a 21509 or 33770 gene, 4) a chromosomal rearrangement of a 21509 or 33770 gene; 5) an alteration in the level of a messenger RNA transcript of a 21509 or 33770 gene, 6) aberrant modification of a 21509 or 33770 gene, such as of the methylation pattern of the genomic DNA, 7) the presence of a non-wild type splicing pattern of a messenger RNA transcript of a 21509 or 33770 gene, 8) a non-wild type level of a 21509 or 33770-protein, 9) allelic loss of a 21509 or 33770 gene, and 10) inappropriate post-translational modification of a 21509 or 33770-protein.

An alteration can be detected without a probe/primer in a polymerase chain reaction, such as anchor PCR or RACE PCR, or, alternatively, in a ligation chain reaction (LCR), the latter of which can be particularly useful for detecting point mutations in the 21509 or 33770-gene. This method can include the steps of collecting a sample of cells from a subject, isolating nucleic acid (e.g., genomic, mRNA or both) from the sample, contacting the nucleic acid sample with one or more primers which specifically hybridize to a 21509 or 33770 gene under conditions such that hybridization and amplification of the 21509 or 33770-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein. Alternatively, other amplification methods described herein or known in the art can be used.

In another embodiment, mutations in a 21509 or 33770 gene from a sample cell can be identified by detecting alterations in restriction enzyme cleavage patterns. For example, sample and control DNA is isolated, amplified (optionally), digested with one or more restriction endonucleases, and fragment length sizes are determined, e.g., by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 21509 or 33770 can be identified by hybridizing a sample and control nucleic acids, e.g., DNA or RNA, two-dimensional arrays, e.g., chip based arrays. Such arrays include a plurality of addresses, each of which is positionally distinguishable from the other. A different probe is located at each address of the plurality. A probe can be complementary to a region of a 21509 or 33770 nucleic acid or a putative variant (e.g., allelic variant) thereof. A probe can have one or more mismatches to a region of a 21509 or 33770 nucleic acid (e.g., a destabilizing mismatch). The arrays can have a high density of addresses, e.g., can contain hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in 21509 or 33770 can be identified in two-dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 21509 or 33770 gene and detect mutations by comparing the sequence of the sample 21509 or 33770 with the corresponding wild-type (control) sequence. Automated sequencing procedures can be utilized when performing the diagnostic assays ((1995) Biotechniques 19:448), including sequencing by mass spectrometry.

Other methods for detecting mutations in the 21509 or 33770 gene include methods in which protection from cleavage agents is used to detect mismatched bases in RNA/RNA or RNA/DNA heteroduplexes (Myers et al. (1985) Science 230:1242; Cotton et al. (1988) Proc. Natl. Acad Sci USA 85:4397; Saleeba et al. (1992) Methods Enzymol. 217:286-295).

In still another embodiment, the mismatch cleavage reaction employs one or more proteins that recognize mismatched base pairs in double-stranded DNA (so called “DNA mismatch repair” enzymes) in defined systems for detecting and mapping point mutations in 21509 or 33770 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662; U.S. Pat. No. 5,459,039).

In other embodiments, alterations in electrophoretic mobility will be used to identify mutations in 21509 or 33770 genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control 21509 or 33770 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension (Saiki et al (1986) Nature 324:163); Saiki et al. (1989) Proc. Natl Acad. Sci USA 86:6230). A further method of detecting point mutations is the chemical ligation of oligonucleotides as described in Xu et al. ((2001) Nature Biotechnol. 19:148). Adjacent oligonucleotides, one of which selectively anneals to the query site, are ligated together if the nucleotide at the query site of the sample nucleic acid is complementary to the query oligonucleotide; ligation can be monitored, e.g., by fluorescent dyes coupled to the oligonucleotides.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

In another aspect, the invention features a set of oligonucleotides. The set includes a plurality of oligonucleotides, each of which is at least partially complementary (e.g., at least 50%, 60%, 70%, 80%, 90%, 92%, 95%, 97%, 98%, or 99% complementary) to a 21509 or 33770 nucleic acid.

In a preferred embodiment the set includes a first and a second oligonucleotide. The first and second oligonucleotide can hybridize to the same or to different locations of SEQ ID NO: 13 or the complement of SEQ ID NO: 13. Different locations can be different but overlapping, or non-overlapping on the same strand. The first and second oligonucleotide can hybridize to sites on the same or on different strands.

The set can be useful, e.g., for identifying SNP's, or identifying specific alleles of 21509 or 33770. In a preferred embodiment, each oligonucleotide of the set has a different nucleotide at an interrogation position. In one embodiment, the set includes two oligonucleotides, each complementary to a different allele at a locus, e.g., a biallelic or polymorphic locus.

In another embodiment, the set includes four oligonucleotides, each having a different nucleotide (e.g., adenine, guanine, cytosine, or thymidine) at the interrogation position. The interrogation position can be a SNP or the site of a mutation. In another preferred embodiment, the oligonucleotides of the plurality are identical in sequence to one another (except for differences in length). The oligonucleotides can be provided with differential labels, such that an oligonucleotide that hybridizes to one allele provides a signal that is distinguishable from an oligonucleotide that hybridizes to a second allele. In still another embodiment, at least one of the oligonucleotides of the set has a nucleotide change at a position in addition to a query position, e.g., a destabilizing mutation to decrease the Tm of the oligonucleotide. In another embodiment, at least one oligonucleotide of the set has a non-natural nucleotide, e.g., inosine. In a preferred embodiment, the oligonucleotides are attached to a solid support, e.g., to different addresses of an array or to different beads or nanoparticles.

In a preferred embodiment the set of oligo nucleotides can be used to specifically amplify, e.g., by PCR, or detect, a 21509 or 33770 nucleic acid.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a 21509 or 33770 gene.

Use of 21509 or 33770 Molecules as Surrogate Markers

The 21509 or 33770 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the 21509 or 33770 molecules of the invention may be detected, and may be correlated with one or more biological states in vivo. For example, the 21509 or 33770 molecules of the invention may serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The 21509 or 33770 molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., a 21509 or 33770 marker) transcription or expression, the amplified marker may be in a quantity which is more readily detectable than the drug itself. Also, the marker may be more easily detected due to the nature of the marker itself; for example, using the methods described herein, anti-21509 or 33770 antibodies may be employed in an immune-based detection system for a 21509 or 33770 protein marker, or 21509 or 33770-specific radiolabeled probes may be used to detect a 21509 or 33770 mRNA marker. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am, J. Health-Syst. Pharm. 56 Suppl. 3: S16-S20.

The 21509 or 33770 molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35:1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or protein (e.g., 21509 or 33770 protein or RNA) for specific tumor markers in a subject, a drug or course of treatment may be selected that is optimized for the treatment of the specific tumor likely to be present in the subject. Similarly, the presence or absence of a specific sequence mutation in 21509 or 33770 DNA may correlate 21509 or 33770 drug response. The use of pharmacogenomic markers therefore permits the application of the most appropriate treatment for each subject without having to administer the therapy.

Pharmaceutical Compositions of 21509 and 33770

The nucleic acid and polypeptides, fragments thereof, as well as anti-21509 or 33770 antibodies (also referred to herein as “active compounds”) of the invention can be incorporated into pharmaceutical compositions. Such compositions typically include the nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

For antibodies, the preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The present invention encompasses agents which modulate expression or activity. An agent may, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e.,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

An antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Methods of Treatment for 21509 and 33770

The present invention provides for both prophylactic and therapeutic methods of treating a subject at risk of (or susceptible to) a disorder or having a disorder associated with aberrant or unwanted 21509 or 33770 expression or activity. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides.

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the 21509 or 33770 molecules of the present invention or 21509 or 33770 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

In one aspect, the invention provides a method for preventing in a subject, a disease or condition associated with an aberrant or unwanted 21509 or 33770 expression or activity, by administering to the subject a 21509 or 33770 or an agent which modulates 21509 or 33770 expression or at least one 21509 or 33770 activity. Subjects at risk for a disease which is caused or contributed to by aberrant or unwanted 21509 or 33770 expression or activity can be identified by, for example, any or a combination of diagnostic or prognostic assays as described herein. Administration of a prophylactic agent can occur prior to the manifestation of symptoms characteristic of the 21509 or 33770 aberrance, such that a disease or disorder is prevented or, alternatively, delayed in its progression. Depending on the type of 21509 or 33770 aberrance, for example, a 21509 or 33770, 21509 or 33770 agonist or 21509 or 33770 antagonist agent can be used for treating the subject. The appropriate agent can be determined based on screening assays described herein.

It is possible that some 21509 or 33770 disorders can be caused, at least in part, by an abnormal level of gene product, or by the presence of a gene product exhibiting abnormal activity. As such, the reduction in the level and/or activity of such gene products would bring about the amelioration of disorder symptoms.

The 21509 or 33770 molecules can act as novel diagnostic targets and therapeutic agents for controlling one or more of cellular proliferative and/or differentiative disorders, disorders associated with abnormal fatty acid biosynthesis or metabolism, hormonal imbalances, cardiovascular disease, and neural degeneration, all of which have been described above, as well as disorders associated with the kidneys, skeletal muscle, breast, lung, colon, liver, bone metabolism, and the immune system.

Disorders involving the kidney include, but are not limited to, congenital anomalies including, but not limited to, cystic diseases of the kidney, that include but are not limited to, cystic renal dysplasia, autosomal dominant (adult) polycystic kidney disease, autosomal recessive (childhood) polycystic kidney disease, and cystic diseases of renal medulla, which include, but are not limited to, medullary sponge kidney, and nephronophthisis-uremic medullary cystic disease complex, acquired (dialysis-associated) cystic disease, such as simple cysts; glomerular diseases including pathologies of glomerular injury that include, but are not limited to, in situ immune complex deposition, that includes, but is not limited to, anti-GBM nephritis, Heymann nephritis, and antibodies against planted antigens, circulating immune complex nephritis, antibodies to glomerular cells, cell-mediated immunity in glomerulonephritis, activation of alternative complement pathway, epithelial cell injury, and pathologies involving mediators of glomerular injury including cellular and soluble mediators, acute glomerulonephritis, such as acute proliferative (poststreptococcal, postinfectious) glomerulonephritis, including but not limited to, poststreptococcal glomerulonepbritis and nonstreptococcal acute glomerulonephritis, rapidly progressive (crescentic) glomerulonephritis, nephrotic syndrome, membranous glomerulonephritis (membranous nephropathy), minimal change disease (lipoid nephrosis), focal segmental glomerulosclerosis, membranoproliferative glomerulonephritis, IgA nephropathy (Berger disease), focal proliferative and necrotizing glomerulonephritis (focal glomerulonephritis), hereditary nephritis, including but not limited to, Alport syndrome and thin membrane disease (benign familial hematuria), chronic glomerulonephritis, glomerular lesions associated with systemic disease, including but not limited to, systemic lupus erythematosus, Henoch-Schonlein purpura, bacterial endocarditis, diabetic glomerulosclerosis, amyloidosis, fibrillary and immunotactoid glomerulonephritis, and other systemic disorders; diseases affecting tubules and interstitium, including acute tubular necrosis and tubulointerstitial nephritis, including but not limited to, pyelonephritis and urinary tract infection, acute pyelonephritis, chronic pyelonephritis and reflux nephropathy, and tubulointerstitial nephritis induced by drugs and toxins, including but not limited to, acute drug-induced interstitial nephritis, analgesic abuse nephropathy, nephropathy associated with nonsteroidal anti-inflammatory drugs, and other tubulointerstitial diseases including, but not limited to, urate nephropathy, hypercalcemia and nephrocalcinosis, and multiple myeloma; diseases of blood vessels including benign nephrosclerosis, malignant hypertension and accelerated nephrosclerosis, renal artery stenosis, and thrombotic microangiopathies including, but not limited to, classic (childhood) hemolytic-uremic syndrome, adult hemolytic-uremic syndrome/thrombotic thrombocytopenic purpura, idiopathic HUS/TTP, and other vascular disorders including, but not limited to, atherosclerotic ischemic renal disease, atheroembolic renal disease, sickle cell disease nephropathy, diffuse cortical necrosis, and renal infarcts; urinary tract obstruction (obstructive uropathy); urolithiasis (renal calculi, stones); and tumors of the kidney including, but not limited to, benign tumors, such as renal papillary adenoma, renal fibroma or hamartoma (renomedullary interstitial cell tumor), angiomyolipoma, and oncocytoma, and malignant tumors, including renal cell carcinoma (hypemephroma, adenocarcinoma of kidney), which includes urothelial carcinomas of renal pelvis.

Disorders involving the skeletal muscle include tumors such as rhabdomyosarcoma.

Disorders of the breast include, but are not limited to, disorders of development; inflammations, including but not limited to, acute mastitis, periductal mastitis, periductal mastitis (recurrent subareolar abscess, squamous metaplasia of lactiferous ducts), mammary duct ectasia, fat necrosis, granulomatous mastitis, and pathologies associated with silicone breast implants; fibrocystic changes; proliferative breast disease including, but not limited to, epithelial hyperplasia, sclerosing adenosis, and small duct papillomas; tumors including, but not limited to, stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, no special type, invasive lobular carcinoma, medullary carcinoma, colloid (mucinous) carcinoma, tubular carcinoma, and invasive papillary carcinoma, and miscellaneous malignant neoplasms.

Disorders in the male breast include, but are not limited to, gynecomastia and carcinoma.

Examples of disorders of the lung include, but are not limited to, congenital anomalies; atelectasis; diseases of vascular origin, such as pulmonary congestion and edema, including hemodynamic pulmonary edema and edema caused by microvascular injury, adult respiratory distress syndrome (diffuse alveolar damage), pulmonary embolism, hemorrhage, and infarction, and pulmonary hypertension and vascular sclerosis; chronic obstructive pulmonary disease, such as emphysema, chronic bronchitis, bronchial asthma, and bronchiectasis; diffuse interstitial (infiltrative, restrictive) diseases, such as pneumoconioses, sarcoidosis, idiopathic pulmonary fibrosis, desquamative interstitial pneumonitis, hypersensitivity pneumonitis, pulmonary eosinophilia (pulmonary infiltration with eosinophilia), Bronchiolitis obliterans-organizing pneumonia, diffuse pulmonary hemorrhage syndromes, including Goodpasture syndrome, idiopathic pulmonary hemosiderosis and other hemorrhagic syndromes, pulmonary involvement in collagen vascular disorders, and pulmonary alveolar proteinosis; complications of therapies, such as drug-induced lung disease, radiation-induced lung disease, and lung transplantation; tumors, such as bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflanunatory pleural effusions, noninflammatory pleural effusions, pneumothorax, and pleural tumors, including solitary fibrous tumors (pleural fibroma) and malignant mesothelioma.

Disorders involving the colon include, but are not limited to, congenital anomalies, such as atresia and stenosis, Meckel diverticulum, congenital aganglionic megacolon-Hirschsprung disease; enterocolitis, such as diarrhea and dysentery, infectious enterocolitis, including viral gastroenteritis, bacterial enterocolitis, necrotizing enterocolitis, antibiotic-associated colitis (pseudomembranous colitis), and collagenous and lymphocytic colitis, miscellaneous intestinal inflammatory disorders, including parasites and protozoa, acquired immunodeficiency syndrome, transplantation, drug-induced intestinal injury, radiation enterocolitis, neutropenic colitis (typhlitis), and diversion colitis; idiopathic inflammatory bowel disease, such as Crohn disease and ulcerative colitis; tumors of the colon, such as non-neoplastic polyps, adenomas, familial syndromes, colorectal carcinogenesis, colorectal carcinoma, and carcinoid tumors.

Disorders involving the liver include, but are not limited to, hepatic injury; jaundice and cholestasis, such as bilirubin and bile formation; hepatic failure and cirrhosis, such as cirrhosis, portal hypertension, including ascites, portosystemic shunts, and splenomegaly; infectious disorders, such as viral hepatitis, including hepatitis A-E infection and infection by other hepatitis viruses, clinicopathologic syndromes, such as the carrier state, asymptomatic infection, acute viral hepatitis, chronic viral hepatitis, and fulminant hepatitis; autoimmune hepatitis; drug- and toxin-induced liver disease, such as alcoholic liver disease; inborn errors of metabolism and pediatric liver disease, such as hemochromatosis, Wilson disease, a1-antitrypsin deficiency, and neonatal hepatitis; intrahepatic biliary tract disease, such as secondary biliary cirrhosis, primary biliary cirrhosis, primary sclerosing cholangitis, and anomalies of the biliary tree; circulatory disorders, such as impaired blood flow into the liver, including hepatic artery compromise and portal vein obstruction and thrombosis, impaired blood flow through the liver, including passive congestion and centrilobular necrosis and peliosis hepatis, hepatic vein outflow obstruction, including hepatic vein thrombosis (Budd-Chiari syndrome) and veno-occlusive disease; hepatic disease associated with pregnancy, such as preeclampsia and eclampsia, acute fatty liver of pregnancy, and intrehepatic cholestasis of pregnancy; hepatic complications of organ or bone marrow transplantation, such as drug toxicity after bone marrow transplantation, graft-versus-host disease and liver rejection, and nonimmunologic damage to liver allografts; tumors and tumorous conditions, such as nodular hyperplasias, adenomas, and malignant tumors, including primary carcinoma of the liver and metastatic tumors.

The 21509 or 33770 nucleic acid and protein of the invention can be used to treat and/or diagnose a variety of immune disorders. Examples of immune disorders or diseases include, but are not limited to, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjogren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy such as, atopic allergy.

Aberrant expression and/or activity of 21509 or 33770 molecules may mediate disorders associated with bone metabolism. “Bone metabolism” refers to direct or indirect effects in the formation or degeneration of bone structures, e.g., bone formation, bone resorption, etc., which may ultimately affect the concentrations in serum of calcium and phosphate. This term also includes activities mediated by 21509 or 33770 molecules effects in bone cells, e.g. osteoclasts and osteoblasts, that may in turn result in bone formation and degeneration. For example, 21509 or 33770 molecules may support different activities of bone resorbing osteoclasts such as the stimulation of differentiation of monocytes and mononuclear phagocytes into osteoclasts. Accordingly, 21509 or 33770 molecules that modulate the production of bone cells can influence bone formation and degeneration, and thus may be used to treat bone disorders. Examples of such disorders include, but are not limited to, osteoporosis, osteodystrophy, osteomalacia, rickets, osteitis fibrosa cystica, renal osteodystrophy, osteosclerosis, anti-convulsant treatment, osteopenia, fibrogenesis-imperfecta ossium, secondary hyperparathyrodism, hypoparathyroidism, hyperparathyroidism, cirrhosis, obstructive jaundice, drug induced metabolism, medullary carcinoma, chronic renal disease, rickets, sarcoidosis, glucocorticoid antagonism, malabsorption syndrome, steatorrhea, tropical sprue, idiopathic hypercalcemia and milk fever.

Disorders involving the brain include, but are not limited to, disorders involving neurons, and disorders involving glia, such as astrocytes, oligodendrocytes, ependymal cells, and microglia; cerebral edema, raised intracranial pressure and herniation, and hydrocephalus; malformations and developmental diseases, such as neural tube defects, forebrain anomalies, posterior fossa anomalies, and syringomyelia and hydromyelia; perinatal brain injury; cerebrovascular diseases, such as those related to hypoxia, ischemia, and infarction, including hypotension, hypoperfusion, and low-flow states—global cerebral ischemia and focal cerebral ischemia—infarction from obstruction of local blood supply, intracranial hemorrhage, including intracerebral (intraparenchymal) hemorrhage, subarachnoid hemorrhage and ruptured berry aneurysms, and vascular malformations, hypertensive cerebrovascular disease, including lacunar infarcts, slit hemorrhages, and hypertensive encephalopathy; infections, such as acute meningitis, including acute pyogenic (bacterial) meningitis and acute aseptic (viral) meningitis, acute focal suppurative infections, including brain abscess, subdural empyema, and extradural abscess, chronic bacterial meningoencephalitis, including tuberculosis and mycobacterioses, neurosyphilis, and neuroborreliosis (Lyme disease), viral meningoencephalitis, including arthropod-borne (Arbo) viral encephalitis, Herpes simplex virus Type 1, Herpes simplex virus Type 2, Varicalla-zoster virus (Herpes zoster), cytomegalovirus, poliomyelitis, rabies, and human immunodeficiency virus 1, including HIV-1 meningoencephalitis (subacute encephalitis), vacuolar myelopathy, AIDS-associated myopathy, peripheral neuropathy, and AIDS in children, progressive multifocal leukoencephalopathy, subacute sclerosing panencephalitis, fungal meningoencephalitis, other infectious diseases of the nervous system; transmissible spongiform encephalopathies (prion diseases); demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, including Alzheimer disease and Pick disease, degenerative diseases of basal ganglia and brain stem, including Parkinsonism, idiopathic Parkinson disease (paralysis agitans), progressive supranuclear palsy, corticobasal degenration, multiple system atrophy, including striatonigral degenration, Shy-Drager syndrome, and olivopontocerebellar atrophy, and Huntington disease; spinocerebellar degenerations, including spinocerebellar ataxias, including Friedreich ataxia, and ataxia-telanglectasia, degenerative diseases affecting motor neurons, including amyotrophic lateral sclerosis (motor neuron disease), bulbospinal atrophy (Kennedy syndrome), and spinal muscular atrophy; inborn errors of metabolism, such as leukodystrophies, including Krabbe disease, metachromatic leukodystrophy, adrenoleukodystrophy, Pelizaeus-Merzbacher disease, and Canavan disease, mitochondrial encephalomyopathies, including Leigh disease and other mitochondrial encephalomyopathies; toxic and acquired metabolic diseases, including vitamin deficiencies such as thiamine (vitamin B₁) deficiency and vitamin B₁₂ deficiency, neurologic sequelae of metabolic disturbances, including hypoglycemia, hyperglycemia, and hepatic encephatopathy, toxic disorders, including carbon monoxide, methanol, ethanol, and radiation, including combined methotrexate and radiation-induced injury; tumors, such as gliomas, including astrocytoma, including fibrillary (diffuse) astrocytoma and glioblastoma multiforme, pilocytic astrocytoma, pleomorphic xanthoastrocytoma, and brain stem glioma, oligodendroglioma, and ependymoma and related paraventricular mass lesions, neuronal tumors, poorly differentiated neoplasms, including medulloblastoma, other parenchymal tumors, including primary brain lymphoma, germ cell tumors, and pineal parenchymal tumors, meningiomas, metastatic tumors, paraneoplastic syndromes, peripheral nerve sheath tumors, including schwannoma, neurofibroma, and malignant peripheral nerve sheath tumor (malignant schwannoma), and neurocutaneous syndromes (phakomatoses), including neurofibromotosis, including Type 1 neurofibromatosis (NF1) and TYPE 2 neurofibromatosis (NF2), tuberous sclerosis, and Von Hippel-Lindau disease.

Additionally, 21509 or 33770 molecules may play an important role in the regulation of metabolism or pain disorders. Diseases of metabolic imbalance include, but are not limited to, obesity, anorexia nervosa, cachexia, lipid disorders, and diabetes. Examples of pain disorders include, but are not limited to, pain response elicited during various forms of tissue injury, e.g., inflammation, infection, and ischemia, usually referred to as hyperalgesia (described in, for example, Fields, H. L. (1987) Pain, New York:McGraw-Hill); pain associated with musculoskeletal disorders, e.g., joint pain; tooth pain; headaches; pain associated with surgery; pain related to irritable bowel syndrome; or chest pain.

As discussed, successful treatment of 21509 or 33770 disorders can be brought about by techniques that serve to inhibit the expression or activity of target gene products. For example, compounds, e.g., an agent identified using an assays described above, that proves to exhibit negative modulatory activity, can be used in accordance with the invention to prevent and/or ameliorate symptoms of 21509 or 33770 disorders. Such molecules can include, but are not limited to peptides, phosphopeptides, small organic or inorganic molecules, or antibodies (including, for example, polyclonal, monoclonal, humanized, anti-idiotypic, chimeric or single chain antibodies, and Fab, F(ab′)₂ and Fab expression library fragments, scFV molecules, and epitope-binding fragments thereof).

Further, antisense and ribozyme molecules that inhibit expression of the target gene can also be used in accordance with the invention to reduce the level of target gene expression, thus effectively reducing the level of target gene activity. Still further, triple helix molecules can be utilized in reducing the level of target gene activity. Antisense, ribozyme and triple helix molecules are discussed above.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances in that the target gene encodes an extracellular protein, it can be preferable to co-administer normal target gene protein into the cell or tissue in order to maintain the requisite level of cellular or tissue target gene activity.

Another method by which nucleic acid molecules may be utilized in treating or preventing a disease characterized by 21509 or 33770 expression is through the use of aptamer molecules specific for 21509 or 33770 protein. Aptamers are nucleic acid molecules having a tertiary structure which permits them to specifically bind to protein ligands (see, e.g., Osborne, et al. (1997) Curr. Opin. Chem Biol. 1: 5-9; and Patel, D. J. (1997) Curr Opin Chem Biol 1:32-46). Since nucleic acid molecules may in many cases be more conveniently introduced into target cells than therapeutic protein molecules may be, aptamers offer a method by which 21509 or 33770 protein activity may be specifically decreased without the introduction of drugs or other molecules which may have pluripotent effects.

Antibodies can be generated that are both specific for target gene product and that reduce target gene product activity. Such antibodies may, therefore, by administered in instances whereby negative modulatory techniques are appropriate for the treatment of 21509 or 33770 disorders. For a description of antibodies, see the Antibody section above.

In circumstances wherein injection of an animal or a human subject with a 21509 or 33770 protein or epitope for stimulating antibody production is harmful to the subject, it is possible to generate an immune response against 21509 or 33770 through the use of anti-idiotypic antibodies (see, for example, Herlyn, D. (1999) Ann Med 31:66-78; and Bhattacharya-Chatterjee, M., and Foon, K. A. (1998) Cancer Treat Res. 94:51-68). If an anti-idiotypic antibody is introduced into a mammal or human subject, it should stimulate the production of anti-anti-idiotypic antibodies, which should be specific to the 21509 or 33770 protein. Vaccines directed to a disease characterized by 21509 or 33770 expression may also be generated in this fashion.

In instances where the target antigen is intracellular and whole antibodies are used, internalizing antibodies may be preferred. Lipofectin or liposomes can be used to deliver the antibody or a fragment of the Fab region that binds to the target antigen into cells. Where fragments of the antibody are used, the smallest inhibitory fragment that binds to the target antigen is preferred. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al. (1993) Proc. Natl. Acad. Sci. USA 90:7889-7893).

The identified compounds that inhibit target gene expression, synthesis and/or activity can be administered to a patient at therapeutically effective doses to prevent, treat or ameliorate 21509 or 33770 disorders. A therapeutically effective dose refers to that amount of the compound sufficient to result in amelioration of symptoms of the disorders. Toxicity and therapeutic efficacy of such compounds can be determined by standard pharmaceutical procedures as described above.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays may utilize antibody mimics and/or “biosensors” that have been created through molecular imprinting techniques. The compound which is able to modulate 21509 or 33770 activity is used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell, R. J. et al (1996) Current Opinion in Biotechnology 7:89-94 and in Shea, K. J. (1994) Trends in Polymer Science 2:166-173. Such “imprinted” affinity matrixes are amenable to ligand-binding assays, whereby the immobilized monoclonal antibody component is replaced by an appropriately imprinted matrix. An example of the use of such matrixes in this way can be seen in Vlatakis, G. et al (1993) Nature 361:645-647. Through the use of isotope-labeling, the “free” concentration of compound which modulates the expression or activity of 21509 or 33770 can be readily monitored and used in calculations of IC₅₀.

Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. An rudimentary example of such a “biosensor” is discussed in Kriz, D. et al (1995) Analytical Chemistry 67:2142-2144.

Another aspect of the invention pertains to methods of modulating 21509 or 33770 expression or activity for therapeutic purposes. Accordingly, in an exemplary embodiment, the modulatory method of the invention involves contacting a cell with a 21509 or 33770 or agent that modulates one or more of the activities of 21509 or 33770 protein activity associated with the cell. An agent that modulates 21509 or 33770 protein activity can be an agent as described herein, such as a nucleic acid or a protein, a naturally-occurring target molecule of a 21509 or 33770 protein (e.g., a 21509 or 33770 substrate or receptor), a 21509 or 33770 antibody, a 21509 or 33770 agonist or antagonist, a peptidomimetic of a 21509 or 33770 agonist or antagonist, or other small molecule.

In one embodiment, the agent stimulates one or 21509 or 33770 activities. Examples of such stimulatory agents include active 21509 or 33770 protein and a nucleic acid molecule encoding 21509 or 33770. In another embodiment, the agent inhibits one or more 21509 or 33770 activities. Examples of such inhibitory agents include antisense 21509 or 33770 nucleic acid molecules, anti-21509 or 33770 antibodies, and 21509 or 33770 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 21509 or 33770 protein or nucleic acid molecule. In one embodiment, the method involves administering an agent (e.g., an agent identified by a screening assay described herein), or combination of agents that modulates (e.g., up regulates or down regulates) 21509 or 33770 expression or activity. In another embodiment, the method involves administering a 21509 or 33770 protein or nucleic acid molecule as therapy to compensate for reduced, aberrant, or unwanted 21509 or 33770 expression or activity.

Stimulation of 21509 or 33770 activity is desirable in situations in which 21509 or 33770 is abnormally downregulated and/or in which increased 21509 or 33770 activity is likely to have a beneficial effect. For example, stimulation of 21509 or 33770 activity is desirable in situations in which a 21509 or 33770 is downregulated and/or in which increased 21509 or 33770 activity is likely to have a beneficial effect. Likewise, inhibition of 21509 or 33770 activity is desirable in situations in which 21509 or 33770 is abnormally upregulated and/or in which decreased 21509 or 33770 activity is likely to have a beneficial effect.

21509 and 33770 Pharmacogenomics

The 21509 or 33770 molecules of the present invention, as well as agents, or modulators which have a stimulatory or inhibitory effect on 21509 or 33770 activity (e.g., 21509 or 33770 gene expression) as identified by a screening assay described herein can be administered to individuals to treat (prophylactically or therapeutically) 21509 or 33770 associated disorders (e.g., cellular proliferative disorders, e.g., cancer) involving aberrant or unwanted 21509 or 33770 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a 21509 or 33770 molecule or 21509 or 33770 modulator as well as tailoring the dosage and/or therapeutic regimen of treatment with a 21509 or 33770 molecule or 21509 or 33770 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23:983-985 and Linder, M. W. et al. (1997) Clin. Chem. 43:254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach,” can be utilized to identify genes that predict drug response. According to this method, if a gene that encodes a drug's target is known (e.g., a 21509 or 33770 protein of the present invention), all common variants of that gene can be fairly easily identified in the population and it can be determined if having one version of the gene versus another is associated with a particular drug response.

Alternatively, a method termed the “gene expression profiling,” can be utilized to identify genes that predict drug response. For example, the gene expression of an animal dosed with a drug (e.g., a 21509 or 33770 molecule or 21509 or 33770 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a 21509 or 33770 molecule or 21509 or 33770 modulator, such as a modulator identified by one of the exemplary screening assays described herein.

The present invention further provides methods for identifying new agents, or combinations, that are based on identifying agents that modulate the activity of one or more of the gene products encoded by one or more of the 21509 or 33770 genes of the present invention, wherein these products may be associated with resistance of the cells to a therapeutic agent. Specifically, the activity of the proteins encoded by the 21509 or 33770 genes of the present invention can be used as a basis for identifying agents for overcoming agent resistance. By blocking the activity of one or more of the resistance proteins, target cells, e.g., human cells, will become sensitive to treatment with an agent that the unmodified target cells were resistant to.

Monitoring the influence of agents (e.g., drugs) on the expression or activity of a 21509 or 33770 protein can be applied in clinical trials. For example, the effectiveness of an agent determined by a screening assay as described herein to increase 21509 or 33770 gene expression, protein levels, or upregulate 21509 or 33770 activity, can be monitored in clinical trials of subjects exhibiting decreased 21509 or 33770 gene expression, protein levels, or downregulated 21509 or 33770 activity. Alternatively, the effectiveness of an agent determined by a screening assay to decrease 21509 or 33770 gene expression, protein levels, or downregulate 21509 or 33770 activity, can be monitored in clinical trials of subjects exhibiting increased 21509 or 33770 gene expression, protein levels, or upregulated 21509 or 33770 activity. In such clinical trials, the expression or activity of a 21509 or 33770 gene, and preferably, other genes that have been implicated in, for example, a 21509 or 33770-associated disorder can be used as a “read out” or markers of the phenotype of a particular cell.

21509 or 33770 Informatics

The sequence of a 21509 or 33770 molecule is provided in a variety of media to facilitate use thereof. A sequence can be provided as a manufacture, other than an isolated nucleic acid or amino acid molecule, which contains a 21509 or 33770. Such a manufacture can provide a nucleotide or amino acid sequence, e.g., an open reading frame, in a form which allows examination of the manufacture using means not directly applicable to examining the nucleotide or amino acid sequences, or a subset thereof, as they exists in nature or in purified form. The sequence information can include, but is not limited to, 21509 or 33770 full-length nucleotide and/or amino acid sequences, partial nucleotide and/or amino acid sequences, polymorphic sequences including single nucleotide polymorphisms (SNPs), epitope sequence, and the like. In a preferred embodiment, the manufacture is a machine-readable medium, e.g., a magnetic, optical, chemical or mechanical information storage device.

As used herein, “machine-readable media” refers to any medium that can be read and accessed directly by a machine, e.g., a digital computer or analogue computer. Non-limiting examples of a computer include a desktop PC, laptop, mainframe, server (e.g., a web server, network server, or server farm), handheld digital assistant, pager, mobile telephone, and the like. The computer can be stand-alone or connected to a communications network, e.g., a local area network (such as a VPN or intranet), a wide area network (e.g., an Extranet or the Internet), or a telephone network (e.g., a wireless, DSL, or ISDN network). Machine-readable media include, but are not limited to: magnetic storage media, such as floppy discs, hard disc storage medium, and magnetic tape; optical storage media such as CD-ROM; electrical storage media such as RAM, ROM, EPROM, EEPROM, flash memory, and the like; and hybrids of these categories such as magnetic/optical storage media.

A variety of data storage structures are available to a skilled artisan for creating a machine-readable medium having recorded thereon a nucleotide or amino acid sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. The skilled artisan can readily adapt any number of data processor structuring formats (e.g., text file or database) in order to obtain computer readable medium having recorded thereon the nucleotide sequence information of the present invention.

In a preferred embodiment, the sequence information is stored in a relational database (such as Sybase or Oracle). The database can have a first table for storing sequence (nucleic acid and/or amino acid sequence) information. The sequence information can be stored in one field (e.g., a first column) of a table row and an identifier for the sequence can be store in another field (e.g., a second column) of the table row. The database can have a second table, e.g., storing annotations. The second table can have a field for the sequence identifier, a field for a descriptor or annotation text (e.g., the descriptor can refer to a functionality of the sequence, a field for the initial position in the sequence to which the annotation refers, and a field for the ultimate position in the sequence to which the annotation refers. Non-limiting examples for annotation to nucleic acid sequences include polymorphisms (e.g., SNP's) translational regulatory sites and splice junctions. Non-limiting examples for annotations to amino acid sequence include polypeptide domains, e.g., a domain described herein; active sites and other functional amino acids; and modification sites.

By providing the nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or target structural motif with the sequence information stored within the data storage means. A search is used to identify fragments or regions of the sequences of the invention which match a particular target sequence or target motif. The search can be a BLAST search or other routine sequence comparison, e.g., a search described herein.

Thus, in one aspect, the invention features a method of analyzing 21509 or 33770, e.g., analyzing structure, function, or relatedness to one or more other nucleic acid or amino acid sequences. The method includes: providing a 21509 or 33770 nucleic acid or amino acid sequence; comparing the 21509 or 33770 sequence with a second sequence, e.g., one or more preferably a plurality of sequences from a collection of sequences, e.g., a nucleic acid or protein sequence database to thereby analyze 21509 or 33770. The method can be performed in a machine, e.g., a computer, or manually by a skilled artisan.

The method can include evaluating the sequence identity between a 21509 or 33770 sequence and a database sequence. The method can be performed by accessing the database at a second site, e.g., over the Internet.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. Typical sequence lengths of a target sequence are from about 10 to 100 amino acids or from about 30 to 300 nucleotide residues. However, it is well recognized that commercially important fragments, such as sequence fragments involved in gene expression and protein processing, may be of shorter length.

Computer software is publicly available which allows a skilled artisan to access sequence information provided in a computer readable medium for analysis and comparison to other sequences. A variety of known algorithms are disclosed publicly and a variety of commercially available software for conducting search means are and can be used in the computer-based systems of the present invention. Examples of such software include, but are not limited to, MacPattern (EMBL), BLASTN and BLASTX (NCBI).

Thus, the invention features a method of making a computer readable record of a sequence of a 21509 or 33770 sequence which includes recording the sequence on a computer readable matrix. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′ end of the translated region.

In another aspect, the invention features, a method of analyzing a sequence. The method includes: providing a 21509 or 33770 sequence, or record, in machine-readable form; comparing a second sequence to the 21509 or 33770 sequence; thereby analyzing a sequence. Comparison can include comparing to sequences for sequence identity or determining if one sequence is included within the other, e.g., determining if the 21509 or 33770 sequence includes a sequence being compared. In a preferred embodiment the 21509 or 33770 or second sequence is stored on a first computer, e.g., at a first site and the comparison is performed, read, or recorded on a second computer, e.g., at a second site. E.g., the 21509 or 33770 or second sequence can be stored in a public or proprietary database in one computer, and the results of the comparison performed, read, or recorded on a second computer. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′ end of the translated region.

In another aspect, the invention provides a machine-readable medium for holding instructions for performing a method for determining whether a subject has a 21509 or 33770-associated disease or disorder or a pre-disposition to a 21509 or 33770-associated disease or disorder, wherein the method comprises the steps of determining 21509 or 33770 sequence information associated with the subject and based on the 21509 or 33770 sequence information, determining whether the subject has a 21509 or 33770-associated disease or disorder or a pre-disposition to a 21509 or 33770-associated disease or disorder and/or recommending a particular treatment for the disease, disorder or pre-disease condition.

The invention further provides in an electronic system and/or in a network, a method for determining whether a subject has a 21509 or 33770-associated disease or disorder or a pre-disposition to a disease associated with a 21509 or 33770 wherein the method comprises the steps of determining 21509 or 33770 sequence information associated with the subject, and based on the 21509 or 33770 sequence information, determining whether the subject has a 21509 or 33770-associated disease or disorder or a pre-disposition to a 21509 or 33770-associated disease or disorder, and/or recommending a particular treatment for the disease, disorder or pre-disease condition. In a preferred embodiment, the method further includes the step of receiving information, e.g., phenotypic or genotypic information, associated with the subject and/or acquiring from a network phenotypic information associated with the subject. The information can be stored in a database, e.g., a relational database. In another embodiment, the method further includes accessing the database, e.g., for records relating to other subjects, comparing the 21509 or 33770 sequence of the subject to the 21509 or 33770 sequences in the database to thereby determine whether the subject as a 21509 or 33770-associated disease or disorder, or a pre-disposition for such.

The present invention also provides in a network, a method for determining whether a subject has a 21509 or 33770 associated disease or disorder or a pre-disposition to a 21509 or 33770-associated disease or disorder associated with 21509 or 33770, said method comprising the steps of receiving 21509 or 33770 sequence information from the subject and/or information related thereto, receiving phenotypic information associated with the subject, acquiring information from the network corresponding to 21509 or 33770 and/or corresponding to a 21509 or 33770-associated disease or disorder (e.g., cellular proliferative disorders, e.g., cancer, or disorders arising from abnormal fatty acid or hormone biosynthesis or metabolism) and based on one or more of the phenotypic information, the 21509 or 33770 information (e.g., sequence information and/or information related thereto), and the acquired information, determining whether the subject has a 21509 or 33770-associated disease or disorder or a pre-disposition to a 21509 or 33770-associated disease or disorder. The method may further comprise the step of recommending a particular treatment for the disease, disorder or pre-disease condition.

The present invention also provides a method for determining whether a subject has a 21509 or 33770-associated disease or disorder or a pre-disposition to a 21509 or 33770-associated disease or disorder, said method comprising the steps of receiving information related to 21509 or 33770 (e.g., sequence information and/or information related thereto), receiving phenotypic information associated with the subject, acquiring information from the network related to 21509 or 33770 and/or related to a 21509 or 33770-associated disease or disorder, and based on one or more of the phenotypic information, the 21509 or 33770 information, and the acquired information, determining whether the subject has a 21509 or 33770-associated disease or disorder or a pre-disposition to a 21509 or 33770-associated disease or disorder. The method may further comprise the step of recommending a particular treatment for the disease, disorder or pre-disease condition.

This invention is further illustrated by the following examples that should not be construed as limiting. The contents of all references, patents and published patent applications cited throughout this application are incorporated herein by reference.

### Background of the 46638 Invention

Lipoxygenases are iron-containing dioxygenases that catalyze the hydroperoxidation of polyunsaturated fatty acids containing a cis,cis-1,4-pentadiene structure to yield a 1-hydroperoxy-2,4-trans, cis-pentadiene product. These enzymes are common in plants, where they are involved in diverse aspects of plant physiology, such as growth and development, pest resistance and senescence, as well as responses to wounding (Vick B. A., Zimmerman D. C. (1987) (In) Biochemistry of plants: A comprehensive treatise, Stumpf P. K., Ed., Vol. 9, pp.53-90, Academic Press, New-York). In mammals, a number of lipoxygenase isozymes are involved in the metabolism of prostaglandins and leukotrienes (Needleman P. et al. (1986) Annu. Rev. Biochem. 55:69-102).

Plant and mammalian lipoxygenases form a closely related family with no significant similarities to other known sequences. Crystal structures have been reported for several of these enzymes (Steczko J.et al. (1992) Biochemistry 31:4053-4057; Boyington J. C. et al. (1993) Science 260:1482-1486). Structurally, lipoxygenases contain a nonheme iron atom, which is bound by four ligands. The iron atom which is essential for enzymatic activity, exists in two oxidation states: Fe⁺² and Fe⁺³. Spectroscopic data show that the metal is bound to nitrogen- and oxygen-containing groups in the protein. The sequences of lipoxygenases share a highly conserved region of about 38 amino acids, five of which being histidine residues. These five histidines are typically clustered in a stretch of about forty amino acids (Peng Y. L. et al. (1994) J. Biol. Chem. 269:3755-3761). In addition, another conserved histidine occurs at a distance of about 149 to 170 residues from the last amino acid in the conserved region. These six histidines have been suggested as possible iron ligands (Boyington J. C. et al. (1993) supra).

Mammalian lipoxygenases are involved in the metabolism of prostaglandins and leukotrienes (Needleman P. et al. (1986) supra). For example, the hydroperoxidation of arachidonic acid by lipoxygenases leads to the synthesis of leukotrienes and lipoxins. These compounds are potent biological activators of cellular responses in inflammation and immunity (B. Samuelsson (1983) Science 220:568). Leukotrienes are synthesized by way of a 5-lypoxygenase pathway in neutrophils, eosinophils, monocytes, mast cells, and keratinocytes, as well as lung, spleen, brain, and heart (reviewed in Needleman P. et al. (1986) supra). Similarly, lipoxygenases, e.g., 12-lipoxygenase and 15-lipoxygenase, may catalyze the conversion of arachidonates12-hydroperoxy-eicosa-5,8,10,14-tetraenoic acid (HPETE) in platelets and 15-1HPETE in neutrophils, respectively (Needleman P. et al. (1986) supra). Deficiencies in 12-lipoxygenase have been found in patients with myeloproliferative disorders. These patients have also a marked increased incidence of hemorrhagic events (Needleman P. et al. (1986) supra). Moreover, modified forms of 12-HPETE have been shown to modulate the migration of smooth muscle cells in vitro (Schafer (1982) N. Eng. J. Med. 306:381-86). Similarly, 15-lipoxygenase products have been shown to modulate neutrophil migration and function (Serhan, C. N. et al. (1984) Biochem. Biophys. Res. Comm. 118:943-49). Thus, lipoxygenase products are known regulators of inflammatory responses, as well as immune and smooth muscle cell activity.

### Summary of the 46638 Invention

The present invention is based, in part, on the discovery of a novel lipoxygenase family member, referred to herein as “46638”. The nucleotide sequence of a cDNA encoding 46638 is shown in SEQ ID NO:22, and the amino acid sequence of a 46638 polypeptide is shown in SEQ ID NO:23. In addition, the nucleotide sequences of the coding region are depicted in SEQ ID NO:24.

Accordingly, in one aspect, the invention features a nucleic acid molecule that encodes a 46638 protein or polypeptide, e.g., a biologically active portion of the 46638 protein. In a preferred embodiment the isolated nucleic acid molecule encodes a polypeptide having the amino acid sequence of SEQ ID NO:23. In other embodiments, the invention provides isolated 46638 nucleic acid molecules having the nucleotide sequence shown in SEQ ID NO:22, SEQ ID NO:24, or the sequence of the DNA insert of the plasmid deposited with ATCC Accession Number ______. In still other embodiments, the invention provides nucleic acid molecules that are substantially identical (e.g., naturally occurring allelic variants) to the nucleotide sequence shown in SEQ ID NO:22, SEQ ID NO:24, or the sequence of the DNA insert of the plasmid deposited with ATCC Accession Number ______. In other embodiments, the invention provides a nucleic acid molecule which hybridizes under a stringency condition described herein to a nucleic acid molecule comprising the nucleotide sequence of SEQ ID NO:22, SEQ ID NO:24, or the sequence of the DNA insert of the plasmid deposited with ATCC Accession Number ______, wherein the nucleic acid encodes a full length 46638 protein or an active fragment thereof.

In a related aspect, the invention further provides nucleic acid constructs that include a 46638 nucleic acid molecule described herein. In certain embodiments, the nucleic acid molecules of the invention are operatively linked to native or heterologous regulatory sequences. Also included, are vectors and host cells containing the 46638 nucleic acid molecules of the invention e.g., vectors and host cells suitable for producing 46638 nucleic acid molecules and polypeptides.

In another related aspect, the invention provides nucleic acid fragments suitable as primers or hybridization probes for the detection of 46638-encoding nucleic acids.

In still another related aspect, isolated nucleic acid molecules that are antisense to a 46638 encoding nucleic acid molecule are provided.

In another aspect, the invention features, 46638 polypeptides, and biologically active or antigenic fragments thereof that are useful, e.g., as reagents or targets in assays applicable to treatment and diagnosis of 46638-mediated or -related disorders. In another embodiment, the invention provides 46638 polypeptides having a 46638 activity. Preferred polypeptides are 46638 proteins including at least one lipoxygenase domain, and, preferably, having a 46638 activity, e.g., a 46638 activity as described herein.

In other embodiments, the invention provides 46638 polypeptides, e.g., a 46638 polypeptide having the amino acid sequence shown in SEQ ID NO:23 or the amino acid sequence encoded by the cDNA insert of the plasmid deposited with ATCC Accession Number ______; an amino acid sequence that is substantially identical to the amino acid sequence shown in SEQ ID NO:23 or the amino acid sequence encoded by the cDNA insert of the plasmid deposited with ATCC Accession Number ______; or an amino acid sequence encoded by a nucleic acid molecule having a nucleotide sequence which hybridizes under a stringency condition described herein to a nucleic acid molecule comprising the nucleotide sequence of SEQ ID NO:22, SEQ ID NO:24, or the sequence of the DNA insert of the plasmid deposited with ATCC Accession Number ______, wherein the nucleic acid encodes a full length 46638 protein or an active fragment thereof.

In a related aspect, the invention further provides nucleic acid constructs which include a 46638 nucleic acid molecule described herein.

In a related aspect, the invention provides 46638 polypeptides or fragments operatively linked to non-46638 polypeptides to form fusion proteins.

In another aspect, the invention features antibodies and antigen-binding fragments thereof, that react with, or more preferably specifically bind 46638 polypeptides or fragments thereof, e.g., a lypoxygenase domain, a PLAT/LH2 domain, a transmembrane domain, a non-transmembrane domain of a 46638 polypeptide. In one embodiment, the antibodies or antigen-binding fragment thereof competitively inhibit the binding of a second antibody to a 46638 polypeptide or a fragment thereof, e.g., a lypoxygenase domain, a PLAT/LH2 domain, a transmembrane domain, a non-transmembrane domain of a 46638 polypeptide.

In another aspect, the invention provides methods of screening for compounds that modulate the expression or activity of the 46638 polypeptides or nucleic acids.

In still another aspect, the invention provides a process for modulating 46638 polypeptide or nucleic acid expression or activity, e.g. using the screened compounds. In certain embodiments, the methods involve treatment or prevention of conditions related to aberrant activity or expression of the 46638 polypeptides or nucleic acids, such as conditions involving aberrant or deficient cellular proliferation or differentiation (e.g., cancerous or pre-cancerous conditions); or conditions involving cells expressing the 46638 polypeptides, e.g., neural or prostate cells. Examples of the conditions that can be treated or prevented with the compounds of the invention include neurological disorders or reproductive, e.g., prostatic disorders.

In yet another aspect, the invention provides methods for inhibiting the proliferation or inducing the differentiation or killing, of a 46638-expressing cell, e.g., a hyperproliferative 46638-expressing cell. The method includes contacting the cell with an agent, e.g., a compound (e.g., a compound identified using the methods described herein) that modulates the activity, or expression, of the 46638 polypeptide or nucleic acid. In a preferred embodiment, the contacting step is effective in vitro or ex vivo. In other embodiments, the contacting step is effected in vivo, e.g., in a subject (e.g., a mammal, e.g., a human), as part of a therapeutic or prophylactic protocol.

In a preferred embodiment, the cell is a hyperproliferative cell, e.g., a cell found in a solid tumor, a soft tissue tumor, or a metastatic lesion, e.g. a tumor of the liver, ovary, breast, colon or lung.

In a preferred embodiment, the agent, e.g., the compound, is an inhibitor of a 46638 polypeptide. Preferably, the inhibitor is chosen from a peptide, a phosphopeptide, a small organic molecule, a small inorganic molecule and an antibody (e.g., an antibody conjugated to a therapeutic moiety selected from a cytotoxin, a cytotoxic agent and a radioactive metal ion). In another preferred embodiment, the agent, e.g., the compound, is an inhibitor of a 46638 nucleic acid, e.g., an antisense, a ribozyme, or a triple helix molecule.

In a preferred embodiment, the agent, e.g., the compound, is administered in combination with a cytotoxic agent. Examples of cytotoxic agents include anti-microtubule agent, a topoisomerase I inhibitor, a topoisomerase II inhibitor, an anti-metabolite, a mitotic inhibitor, an alkylating agent, an intercalating agent, an agent capable of interfering with a signal transduction pathway, an agent that promotes apoptosis or necrosis, and radiation.

In another aspect, the invention features methods for treating or preventing, in a subject, a disorder characterized by aberrant activity of a 46638-expressing cell. Preferably, the method includes comprising administering to the subject (e.g., a mammal, e.g., a human) an effective amount of a compound (e.g., a compound identified using the methods described herein) that modulates the activity, or expression, of the 46638 polypeptide or nucleic acid.

In a preferred embodiment, the disorder is a cancerous or pre-cancerous condition, e.g., a solid tumor, a soft tissue tumor, or a metastatic lesion. In a preferred embodiment, the tumor or metastatic lesion originates from a colon (e.g., a colon tumor or colonic liver metastasis), liver, lung, or ovary cell.

In other embodiments, the disorder is a neurological (e.g., a brain) disorder, or a reproductive disorder (e.g., a prostatic disorder).

In a further aspect, the invention provides methods for evaluating the efficacy of a treatment of a disorder, e.g., a proliferative disorder. The method includes: treating a subject, e.g., a patient or an animal, with a protocol under evaluation (e.g., treating a subject with one or more of: chemotherapy, radiation, and/or a compound identified using the methods described herein); and evaluating the expression of a 46638 nucleic acid or polypeptide before and after treatment. A change, e.g., a decrease or increase, in the level of a 46638 nucleic acid (e.g., mRNA) or polypeptide after treatment, relative to the level of expression before treatment, is indicative of the efficacy of the treatment of the disorder. The level of 46638 nucleic acid or polypeptide expression can be detected by any method described herein.

In a preferred embodiment, the evaluating step includes obtaining a sample (e.g., a tissue sample, e.g., a biopsy, or a fluid sample) from the subject, before and after treatment and comparing the level of expressing of a 46638 nucleic acid (e.g., mRNA) or polypeptide before and after treatment.

In another aspect, the invention provides methods for evaluating the efficacy of a therapeutic or prophylactic agent (e.g., an anti-neoplastic agent). The method includes: contacting a sample with an agent (e.g., a compound identified using the methods described herein, a cytotoxic agent) and, evaluating the expression of 46638 nucleic acid or polypeptide in the sample before and after the contacting step. A change, e.g., a decrease or increase, in the level of 46638 nucleic acid (e.g., mRNA) or polypeptide in the sample obtained after the contacting step, relative to the level of expression in the sample before the contacting step, is indicative of the efficacy of the agent. The level of 46638 nucleic acid or polypeptide expression can be detected by any method described herein. In a preferred embodiment, the sample includes cells obtained from a cancerous tissue or, e.g., liver, ovary, breast, colon or lung tissue.

In further aspect, the invention provides assays for determining the presence or absence of a genetic alteration in a 46638 polypeptide or nucleic acid molecule, including for disease diagnosis.

In another aspect, the invention features a two dimensional array having a plurality of addresses, each address of the plurality being positionally distinguishable from each other address of the plurality, and each address of the plurality having a unique capture probe, e.g., a nucleic acid or peptide sequence. At least one address of the plurality has a capture probe that recognizes a 46638 molecule. In one embodiment, the capture probe is a nucleic acid, e.g., a probe complementary to a 46638 nucleic acid sequence. In another embodiment, the capture probe is a polypeptide, e.g., an antibody specific for 46638 polypeptides. Also featured is a method of analyzing a sample by contacting the sample to the aforementioned array and detecting binding of the sample to the array.

Other features and advantages of the invention will be apparent from the following detailed description, and from the claims.

### Detailed Description of 46638

The human 46638 sequence (see SEQ ID NO:22, as recited in Example 7), which is approximately 3320 nucleotides long including untranslated regions, contains a predicted methionine-initiated coding sequence of about 2136 nucleotides, including the termination codon. The coding sequence encodes a 711 amino acid protein (see SEQ ID NO:23, as recited in Example 7).

Human 46638 contains the following regions or other structural features:

a predicted lipoxygenase domain (PFAM Accession PF00305) located at about amino acid 267 to 703 of SEQ ID NO:23;

a predicted PLAT/LH2 domain located at about amino acids 2 to 116 of SEQ ID NO:23;

a predicted transmembrane region located at about amino acids 345 to 366 of SEQ ID NO:23;

two predicted non-transmembrane regions located at about amino acids 1 to about 344 (N-terminal non-transmembrane region), and from about amino acids 367 to 711 (C-terminal non-transmembrane region);

four predicted N-glycosylation sites (PS00001) located from about amino acids 21 to 24, 405 to 408, 583 to 586, and 633 to 636 of SEQ ID NO:23;

two predicted cAMP/cGMP phosphorylation sites located at about amino acids 78 to 81 of SEQ ID NO:23, and 239 to 242 of SEQ ID NO:23;

nine predicted protein kinase C phosphorylation sites (PS00005) located at about amino acids 33 to 35, 117 to 119, 167 to 169, 242 to 244, 260 to 262, 423 to 425, 494 to 496, 608 to 610, and 621 to 623 of SEQ ID NO:23;

eleven predicted casein kinase II phosphorylation sites (PS00006) located at about amino acids 29 to 32, 90 to 93, 161 to 164, 178 to 181, 316 to 319, 382 to 385, 569 to 572, 624 to 627, 628 to 631, 657 to 660, and 698 to 701 of SEQ ID NO:23;

three predicted N-myristoylation sites (PS00008) located at about amino acids 17 to 22, 116 to 121 and 309 to 314 OfSEQIDNO:23; and

a predicted immunoglobulin/major histocompatibility complex protein signature located at about amino acids 585 to 588 of SEQ ID NO:23.

For general information regarding PFAM identifiers, PS prefix and PF prefix domain identification numbers, refer to Sonnhammer et al. (1997) Protein 28:405-420 and http://www.psc.edu/general/ software/packages/pfam/pfam.html.

A plasmid containing the nucleotide sequence encoding human 46638 (clone “Fbh46638FL”) was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

The 46638 protein contains a significant number of structural characteristics in common with members of the lipoxygenase family. The term “family” when referring to the protein and nucleic acid molecules of the invention means two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin, or alternatively, can contain homologues of non-human origin, e.g., rat or mouse proteins. Members of a family can also have common functional characteristics.

Lipoxygenase family members share a highly conserved region, which includes five histidines clustered in a stretch of about forty amino acids (Peng Y. L. et al. (1994) J. Biol. Chem. 269:3755-3761). In addition, another conserved histidine occurs at a distance of about 149 to 170 residues from the last amino acid of the conserved region. These six histidines have been suggested as possible iron ligands (Boyington J. C. et al. (1993) supra). When enzymatically active, lipoxygenase family member include a nonheme iron atom, Fe⁺² and Fe⁺³, which is bound by four ligands. Lipoxygenase family members catalyze the hydroperoxidation of polyunsaturated fatty acids containing a cis,cis-1,4-pentadiene structure to yield a 1-hydroperoxy-2,4-trans, cis-pentadiene product. Examples of lipoxygenase products include prostaglandins and leukotrienes (Needleman P. et al. (1986) supra). For example, the hydroperoxidation of arachidonic acid by lipoxygenases leads to the synthesis of leukotrienes and lipoxins. These compounds are potent biological activators of cellular responses in inflammation and immunity (B. Samuelsson (1983) Science 220:568). Accordingly, lipoxygenase family members are modulators of a variety of cellular processes, including inflammation and immunity.

A 46638 polypeptide can include at least one “lipoxygenase domain” or at least one region homologous with a “lipoxygenase domain”. A 46638 polypeptide can include at least one “PLAT/LH2” domain. A 46638 can optionally further include at least one transmembrane domain, at least one, preferably two, non-transmembrane domains; at least one, two, three, preferably four, N-glycosylation sites; at least one, preferably two, cAMP/cGMP phosphorylation sites; at least one, two, three, four, five, six, seven, eight, preferably nine, protein kinase C sites; at least one, two, three, four, five, six, seven, eight, nine, ten, preferably eleven, casein kinase II sites; at least one, two, preferably three N-myristoylation sites; and at least one immunoglobulin/major histocompatibility complex protein signature site.

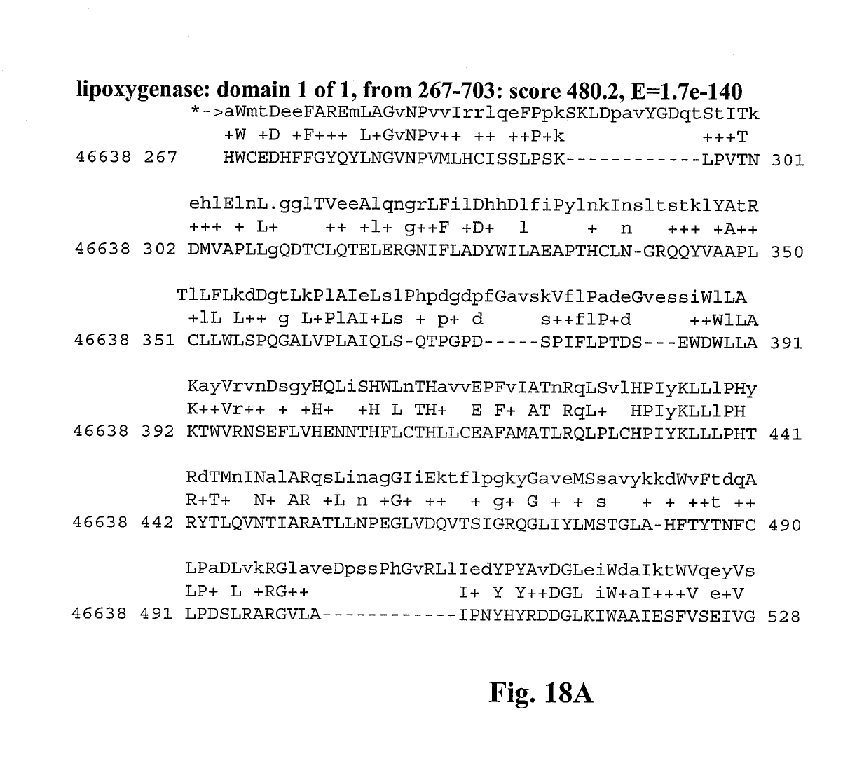

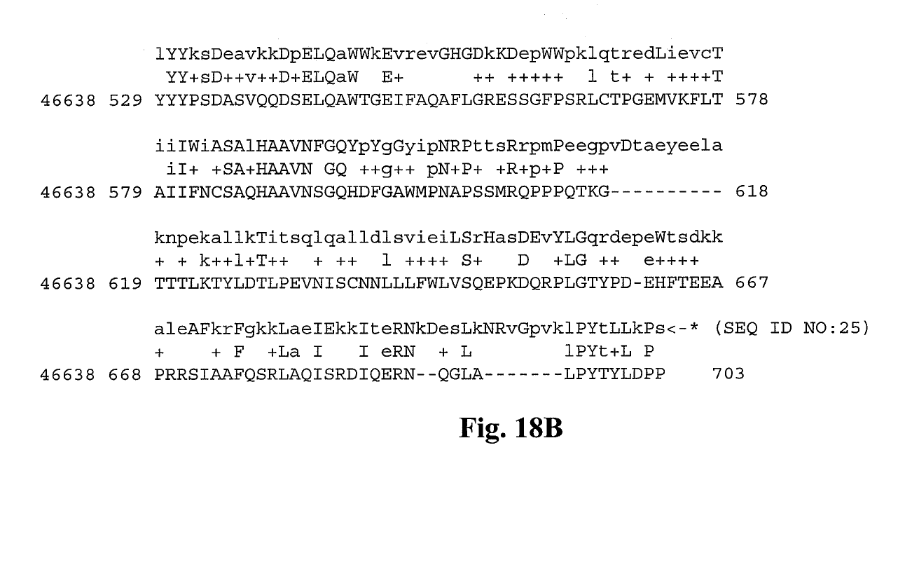

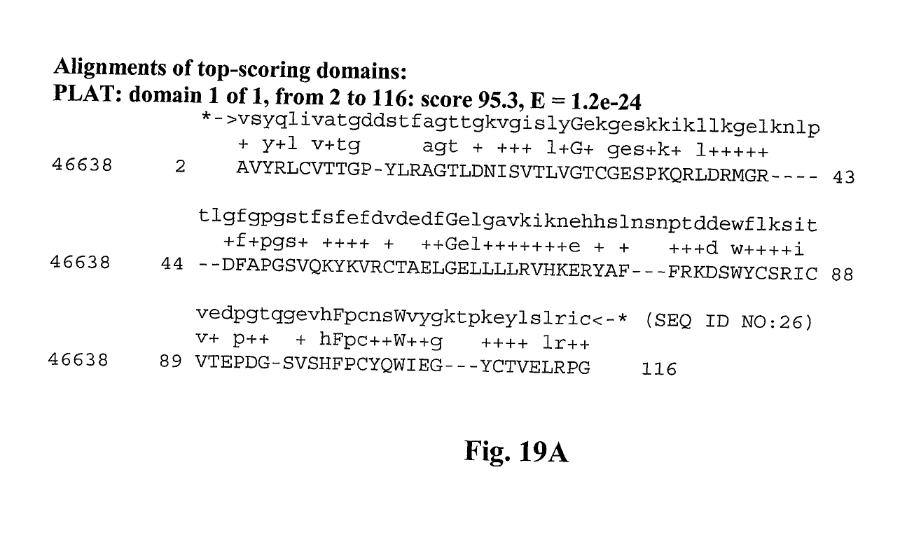

As used herein, the term “lipoxygenase domain” refers to a protein domain which is includes one, two, three, four, and preferably five histidine residues, clustered in a stretch of about forty amino acids. Preferably, the lipoxygenase domain further includes another histidine residue located at a distance of about 140 to 170 and preferably 149 to 160 residues from the last amino acid in the five histidine stretch. For example, the lipoxygenase domain of 46638 shows a cluster of five histidine residues located at amino acids 403, 408, 413, 432 and 440 of SEQ ID NO:23 (FIG. 18) and another histidine residue at position 589 of SEQ ID NO:23 (FIG. 18). Preferably, the lipoxygenase domain has an amino acid sequence of about 300 to about 600 amino acid residues and having a bit score for the alignment of the sequence to the lipoxygenase domain (HMM) of at least 100. Preferably, a lipoxygenase domain includes at least about 350 to about 550 amino acids, more preferably about 400 to about 500 amino acid residues, about 425 to 450, or about 436 amino acids and has a bit score for the alignment of the sequence to the lipoxygenase domain (HMM) of at least 200, preferably 300, more preferably 400 or greater. The lipoxygenase domain (HMM) has been assigned the PFAM Accession (PF00305) (http://genome.wustl.edu/Pfam/html). An alignment of the lipoxygenase domain (from about amino acids 267 to about 703 of SEQ ID NO:23) of human 46638 with a consensus amino acid sequence derived from a hidden Markov model (PFAM) is depicted in FIG. 18.

In a preferred embodiment, 46638 polypeptide or protein has a “lipoxygenase domain” or a region which includes at least about 350 to about 550 amino acids, more preferably about 400 to about 500 amino acid residues, about 425 to 450, or about 436 amino acid residues and has at least about 60%, 70% 80% 90% 95%, 99%, or 100% homology with a “lipoxygenase domain,” e.g., the lipoxygenase domain of human 46638 (e.g., residues 267 to 703 of SEQ ID NO:23).

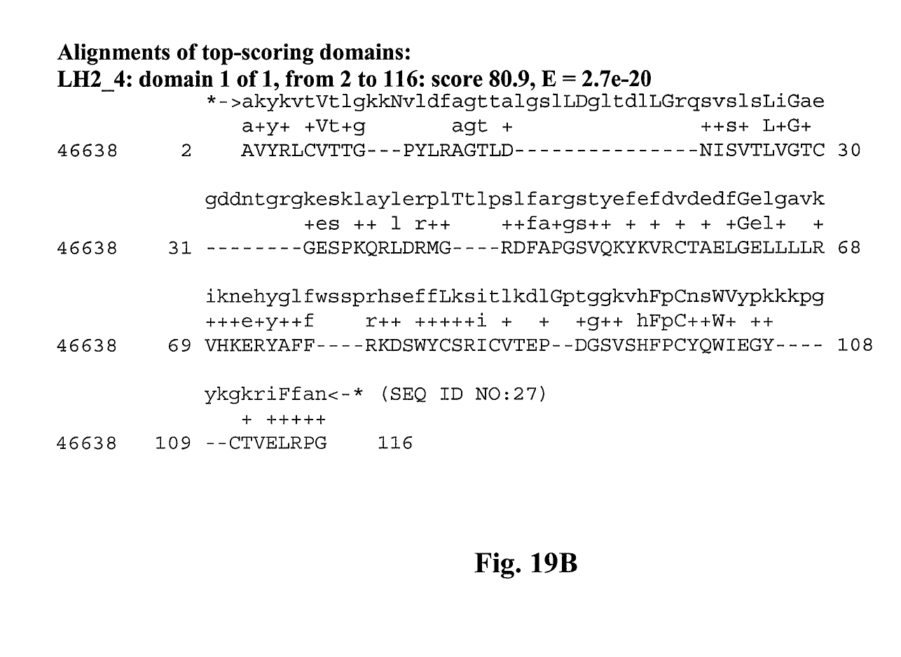

As used herein, the term “PLAT/LH2 domain”, also called Polycystin-1, Lipoxygenase Alpha-Toxin and Lipoxygenase Homology domains, respectively, refers to a protein domain found in a variety of membrane- or lipid-associated proteins. Preferably, this domain mediates membrane attachment. Preferably, the PLAT/LH2 domain has an amino acid sequence of about 25 to about 300 amino acid residues and having a bit score for the alignment of the sequence to the PLAT/LH2 domain (HMM) of at least 20. Preferably, a PLAT/LH2 domain includes at least about 50 to about 200 amino acids, more preferably about 100 to about 150 amino acid residues, about 105 to 120, or about 114 amino acids and has a bit score for the alignment of the sequence to the PLAT/LH2 domain (HMM) of at least 200, preferably 300, more preferably 400 or greater. The PLAT/LH2 domain (HMM) has been assigned the PFAM Accession (PF01477) (http://genome.wustl.edu/Pfam/html). An alignment of the lipoxygenase domain (from about amino acids 2 to about 116 of SEQ ID NO:23) of human 46638 with a consensus amino acid sequence derived from a hidden Markov model (PFAM and SMART) is depicted in FIGS. 19A and 19B.

To identify the presence of a “lipoxygenase” domain or a “PLAT/LH2 domain” in a 46638 protein sequence, and make the determination that a polypeptide or protein of interest has a particular profile, the amino acid sequence of the protein can be searched against the Pfam database of HMMs (e.g., the Pfam database, release 2.1) using the default parameters (http://www.sanger.ac.uk/Software/Pfam/HMM_search). For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al.(1987) Proc. Natl. Acad. Sci. USA 84:4355-4358; Krogh et al.(1994) J. Mol. Biol. 235:1501-1531; and Stultz et al.(l 993) Protein Sci. 2:305-314, the contents of which are incorporated herein by reference. A search was performed against the HMM database resulting in the identification of a “lipoxygenase” and a “PLAT/LH2 domain” in the amino acid sequence of human 46638 at about residues 267 to about 703, and about 2 to about 116, respectively, of SEQ ID NO:23 (see Example 7 and FIGS. 18 and 19A-19B).

A 46638 family member can include at least one lipoxygenase domain; and at least one PLAT/LH2 domain. Furthermore, a 46638 family member can include at least one, two, three, four, five, six, seven, eight, preferably nine protein kinase C phosphorylation sites (PS00005); at least one, two, three, four, five, six, seven, eight, nine, ten and preferably eleven predicted casein kinase II phosphorylation sites (PS00006); and at least one, two, preferably three predicted N-myristylation sites (PS00008).

In one embodiment, a 46638 protein includes at least one transmembrane domain. As used herein, the term “transmembrane domain” includes an amino acid sequence of about 15 amino acid residues in length that spans a phospholipid membrane. More preferably, a transmembrane domain includes about at least 16, 18, 20, 21. 22, 25, 30, 35 or 40 amino acid residues and spans a phospholipid membrane. Transmembrane domains are rich in hydrophobic residues, and typically have an α-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta W. N. et al, (1996) Annual Rev. Neuronsci. 19: 235-63, the contents of which are incorporated herein by reference.

In a preferred embodiment, a 46638 polypeptide or protein has at least one transmembrane domain or a region which includes at least 16, 18, 20, 21. 22, 25, 30, 35 or 40 amino acid residues and has at least about 60%, 70% 80% 90% 95%, 99%, or 100% homology with a “transmembrane domain,” e.g., at least one transmembrane domain of human 46638 (e.g., from about amino acid residues 345 to about 366 of SEQ ID NO:23).

In another embodiment, a 46638 protein includes at least one, preferably two “non-transmembrane domain”. As used herein, “non-transmembrane domains” are domains that reside outside of the membrane. When referring to plasma membranes, non-transmembrane domains include extracellular domains (i.e., outside of the cell) and intracellular domains (i.e., within the cell). When referring to membrane-bound proteins found in intracellular organelles (e.g., mitochondria, endoplasmic reticulum, peroxisomes and microsomes), non-transmembrane domains include those domains of the protein that reside in the cytosol (i.e., the cytoplasm), the lumen of the organelle, or the matrix or the intermembrane space (the latter two relate specifically to mitochondria organelles). The C-terminal amino acid residue of a non-transmembrane domain is adjacent to an N-terminal amino acid residue of a transmembrane domain in a naturally-occurring 46638, or 46638-like protein.

In a preferred embodiment, a 46638 polypeptide or protein has a “non-transmembrane domain” or a region which includes at least about 1-500, preferably about 100-400, more preferably about 200-350, and even more preferably about 300-350 amino acid residues, and has at least about 60%, 70% 80% 90% 95%, 99% or 100% homology with a “non-transmembrane domain”, e.g., a non-transmembrane domain of human 46638 (e.g., from about amino acid residues 1 to about 344 (N-terminal non-transmembrane domain), and from about amino acids 367 to about 711 (C-terminal non-transmembrane domain) of SEQ ID NO:23).

A non-transmembrane domain located at the N-terminus of a 46638 protein or polypeptide is referred to herein as an “N-terminal non-transmembrane domain”, or an “N-terminal non-transmembrane loop”. As used herein, an “N-terminal non-transmembrane domain” includes an amino acid sequence having about 1-500, preferably about 100-400, more preferably about 200-350, and even more preferably about 300-350 amino acid residues in length and is located outside the boundaries of a membrane. For example, an N-terminal non-transmembrane domain is located at about amino acid residues 1-344 of SEQ ID NO:23.

Similarly, a non-transmembrane domain located at the C-terminus of a 46638 protein or polypeptide is referred to herein as a “C-terminal non-transmembrane domain”, or a “C-terminal non-transmembrane loop”. As used herein, an “C-terminal non-transmembrane domain” includes an amino acid sequence having about 1-500, preferably about 100-400, more preferably about 200-350, and even more preferably about 300-350 amino acid residues in length and is located outside the boundaries of a membrane. For example, an C-terminal non-transmembrane domain is located at about amino acid residues 367 to about 711 of SEQ ID NO:23.

As the 46638 polypeptides of the invention may modulate 46638-mediated activities, they may be useful as of for developing novel diagnostic and therapeutic agents for 46638-mediated or related disorders, as described below.

As used herein, a “46638 activity”, “biological activity of 46638” or “functional activity of 46638”, refers to an activity exerted by a 46638 protein, polypeptide or nucleic acid molecule. For example, a 46638 activity can be an activity exerted by 46638 in a physiological milieu on, e.g., a 46638-responsive cell or on a 46638 substrate, e.g., a protein substrate. A 46638 activity can be determined in vivo or in vitro. In one embodiment, a 46638 activity is a direct activity, such as an association with a 46638 target molecule. A “target molecule” or “binding partner” is a molecule with which a 46638 protein binds or interacts in nature. In another embodiment, 46638 activity can also be an indirect activity, e.g., a cellular signaling activity mediated by interaction of the 46638 protein with a 46638 receptor.

The features of the 46638 molecules of the present invention can provide similar biological activities as lipoxygenase family members. For example, the 46638 proteins of the present invention can have one or more of the following activities: (1) ability to catalyze the hydroperoxidation of a substrate, e.g., a fatty acid substrate (e.g., arachidonic acid); (2) the ability to synthesize or metabolize leukotrienes, lipoxins and/or prostaglandins; (3) ability to bind an iron atom; (4) ability to associate or attach to a cell membrane; (5) the ability to modulate an inflammatory response; (6) the ability to modulate immune cell activity (e.g., migration, proliferation, differentiation of an immune cell); (7) the ability to modulate smooth muscle cell activity (e.g., migration, proliferation, differentiation of a smooth muscle cell); (8) the ability to modulate cellular proliferation, differentiation, tumorigenesis; or (9) the ability to modulate the activity of the cells or tissues in which a 46638 protein is expressed, e.g., prostate or neural cells. 46638 mRNA demonstrates increased expression in, for example, normal bronchial epithelial cells, normal prostate epithelial cells, and in normal brain tissues (cortex and hypothalamus). Lower levels of expression were also detected in normal or tumor cells of the breast; colon; lung; heart; placenta; skin; prostate; and ovary. Thus, the 46638 molecules can act, for example, as novel diagnostic targets and therapeutic agents for controlling inflammatory disorders, immune disorders, blood vessel disorders, cardiovascular disorders, disorders involving prostate or neural cells, cellular differentiation disorders, neurodegenerative disorders, liver disorders, ovarian disorders, lung disorders, colon disorders, breast disorders, skin disorders and disorders involving the placenta, as described in more detail below.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol/Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Stemberg disease.

46638 mRNA was found to be expressed in brain tissue, including normal cortex and hypothalamus. Accordingly, the molecules of the invention may mediate disorders involving aberrant activities of brain cells, for example neurodegenerative disorders. Disorders involving the brain include, but are not limited to, disorders involving neurons, and disorders involving glia, such as astrocytes, oligodendrocytes, ependymal cells, and microglia; cerebral edema, raised intracranial pressure and herniation, and hydrocephalus; malformations and developmental diseases, such as neural tube defects, forebrain anomalies, posterior fossa anomalies, and syringomyelia and hydromyelia; perinatal brain injury; cerebrovascular diseases, such as those related to hypoxia, ischemia, and infarction, including hypotension, hypoperfusion, and low-flow states—global cerebral ischemia and focal cerebral ischemia—infarction from obstruction of local blood supply, intracranial hemorrhage, including intracerebral (intraparenchymal) hemorrhage, subarachnoid hemorrhage and ruptured berry aneurysms, and vascular malformations, hypertensive cerebrovascular disease, including lacunar infarcts, slit hemorrhages, and hypertensive encephalopathy; infections, such as acute meningitis, including acute pyogenic (bacterial) meningitis and acute aseptic (viral) meningitis, acute focal suppurative infections, including brain abscess, subdural empyema, and extradural abscess, chronic bacterial meningoencephalitis, including tuberculosis and mycobacterioses, neurosyphilis, and neuroborreliosis (Lyme disease), viral meningoencephalitis, including arthropod-borne (Arbo) viral encephalitis, Herpes simplex virus Type 1, Herpes simplex virus Type 2, Varicalla-zoster virus (Herpes zoster), cytomegalovirus, poliomyelitis, rabies, and human immunodeficiency virus 1, including HIV-1 meningoencephalitis (subacute encephalitis), vacuolar myelopathy, AIDS-associated myopathy, peripheral neuropathy, and AIDS in children, progressive multifocal leukoencephalopathy, subacute sclerosing panencephalitis, fungal meningoencephalitis, other infectious diseases of the nervous system; transmissible spongiform encephalopathies (prion diseases); demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, including Alzheimer disease and Pick disease, degenerative diseases of basal ganglia and brain stem, including Parkinsonism, idiopathic Parkinson disease (paralysis agitans), progressive supranuclear palsy, corticobasal degenration, multiple system atrophy, including striatonigral degenration, Shy-Drager syndrome, and olivopontocerebellar atrophy, and Huntington disease; spinocerebellar degenerations, including spinocerebellar ataxias, including Friedreich ataxia, and ataxia-telanglectasia, degenerative diseases affecting motor neurons, including amyotrophic lateral sclerosis (motor neuron disease), bulbospinal atrophy (Kennedy syndrome), and spinal muscular atrophy; inborn errors of metabolism, such as leukodystrophies, including Krabbe disease, metachromatic leukodystrophy, adrenoleukodystrophy, Pelizaeus-Merzbacher disease, and Canavan disease, mitochondrial encephalomyopathies, including Leigh disease and other mitochondrial encephalomyopathies; toxic and acquired metabolic diseases, including vitamin deficiencies such as thiamine (vitamin B₁) deficiency and vitamin B₁₂ deficiency, neurologic sequelae of metabolic disturbances, including hypoglycemia, hyperglycemia, and hepatic encephatopathy, toxic disorders, including carbon monoxide, methanol, ethanol, and radiation, including combined methotrexate and radiation-induced injury; tumors, such as gliomas, including astrocytoma, including fibrillary (diffuse) astrocytoma and glioblastoma multiforme, pilocytic astrocytoma, pleomorphic xanthoastrocytoma, and brain stem glioma, oligodendroglioma, and ependymoma and related paraventricular mass lesions, neuronal tumors, poorly differentiated neoplasms, including medulloblastoma, other parenchymal tumors, including primary brain lymphoma, germ cell tumors, and pineal parenchymal tumors, meningiomas, metastatic tumors, paraneoplastic syndromes, peripheral nerve sheath tumors, including schwannoma, neurofibroma, and malignant peripheral nerve sheath tumor (malignant schwannoma), and neurocutaneous syndromes (phakomatoses), including neurofibromotosis, including Type 1 neurofibromatosis (NF1) and TYPE 2 neurofibromatosis (NF2), tuberous sclerosis, and Von Hippel-Lindau disease.

46638 mRNA was found to exhibit increased expression in prostate epithelial cells. Thus, the molecules of the invention may mediate disorders involving aberrant activities of these cells, for example prostate disorders. Disorders involving the prostate include, but are not limited to, inflammations, benign enlargement, for example, nodular hyperplasia (benign prostatic hypertrophy or hyperplasia), and tumors such as carcinoma. A “prostate disorder” can also include an abnormal condition occurring in the male pelvic region characterized by, e.g., male sexual dysfunction and/or urinary symptoms. This disorder may be manifested in the form of genitourinary inflammation (e.g., inflammation of smooth muscle cells) as in several common diseases of the http://164.195. 100.11/netacgi/nph-Parser?Sect1=PTO2&Sect2=HITOFF&u=/netahtm1/—h5http://164.195.100.11/netacgi/nph-Parser?Sect1=PTO2&Sect2=HITOFF&u=/netahtml/—h7prostate including prostatitis, benign prostatic hyperplasia and cancer, e.g., adenocarcinoma or carcinoma, of the http://164.195.100.11/netacei/nph-Parser?Sect1=PTO2&Sect2=HITOFF&u=/netahtm1/—h6http://164.195.100.11/netacgi/nph-Parser?Sect1=PTO2&Sect2=HITOFF&u=/netahtml/—h8prostate.

46638 mRNA was also found to be expressed in normal and tumor ovary cells. Thus, the molecules of the invention may mediate disorders involving aberrant activities of these cells, for example ovarian disorders. Disorders involving the ovary include, for example, polycystic ovarian disease, Stein-leventhal syndrome, Pseudomyxoma peritonei and stromal hyperthecosis; ovarian tumors such as, tumors of coelomic epithelium, serous tumors, mucinous tumors, endometeriod tumors, clear cell adenocarcinoma, cystadenofibroma, brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecoma-fibromas, androblastomas, hill cell tumors, and gonadoblastoma; and metastatic tumors such as Krukenberg tumors.

46638 mRNA was also found to be expressed in normal skin cells, and thus, the molecules of the invention may mediate disorders involving aberrant activities of these cells, for example diseases of the skin. Diseases of the skin include but are not limited to, disorders of pigmentation and melanocytes, including but not limited to, vitiligo, freckle, melasma, lentigo, nevocellular nevus, dysplastic nevi, and malignant melanoma; benign epithelial tumors, including but not limited to, seborrheic keratoses, acanthosis nigricans, fibroepithelial polyp, epithelial cyst, keratoacanthoma, and adnexal (appendage) tumors; premalignant and malignant epidermal tumors, including but not limited to, actinic keratosis, squamous cell carcinoma, basal cell carcinoma, and merkel cell carcinoma; tumors of the dermis, including but not limited to, benign fibrous histiocytoma, dermatofibrosarcoma protuberans, xanthomas, and dermal vascular tumors; tumors of cellular immigrants to the skin, including but not limited to, histiocytosis X, mycosis fingoides (cutaneous T-cell lymphoma), and mastocytosis; disorders of epidermal maturation, including but not limited to, ichthyosis; acute inflammatory dermatoses, including but not limited to, urticaria, acute eczematous dermatitis, and erythema multiforme; chronic inflammatory dermatoses, including but not limited to, psoriasis, lichen planus, and lupus erythematosus; blistering (bullous) diseases, including but not limited to, pemphigus, bullous pemphigoid, dermatitis herpetiformis, and noninflammatory blistering diseases: epidermolysis bullosa and porphyria; disorders of epidermal appendages, including but not limited to, acne vulgaris; panniculitis, including but not limited to, erythema nodosum and erythema induratum; and infection and infestation, such as verrucae, molluscum contagiosum, impetigo, superficial fungal infections, and arthropod bites, stings, and infestations.

46638 mRNA was also found to be expressed in normal and tumorous colon cells, and thus, the molecules of the invention may mediate disorders involving aberrant activities of these cells, for example diseases of the colon. Disorders involving the colon include, but are not limited to, congenital anomalies, such as atresia and stenosis, Meckel diverticulum, congenital aganglionic megacolon-Hirschsprung disease; enterocolitis, such as diarrhea and dysentery, infectious enterocolitis, including viral gastroenteritis, bacterial enterocolitis, necrotizing enterocolitis, antibiotic-associated colitis (pseudomembranous colitis), and collagenous and lymphocytic colitis, miscellaneous intestinal inflammatory disorders, including parasites and protozoa, acquired immunodeficiency syndrome, transplantation, drug-induced intestinal injury, radiation enterocolitis, neutropenic colitis (typhlitis), and diversion colitis; idiopathic inflammatory bowel disease, such as Crohn disease and ulcerative colitis; tumors of the colon, such as non-neoplastic polyps, adenomas, familial syndromes, colorectal carcinogenesis, colorectal carcinoma, and carcinoid tumors.

46638 mRNA was also found to be expressed in breast tumor cells, and thus, the molecules of the invention may mediate disorders involving aberrant activities of breast cells, for example diseases of the breast. Disorders of the breast include, but are not limited to, disorders of development; inflammations, including but not limited to, acute mastitis, periductal mastitis, periductal mastitis (recurrent subareolar abscess, squamous metaplasia of lactiferous ducts), mammary duct ectasia, fat necrosis, granulomatous mastitis, and pathologies associated with silicone breast implants; fibrocystic changes; proliferative breast disease including, but not limited to, epithelial hyperplasia, sclerosing adenosis, and small duct papillomas; tumors including, but not limited to, stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, no special type, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Expression of 46638 mRNA was found to be elevated in normal bronchial epithelial cells, and was also found in lung tumor cells. Thus, the molecules of the invention may mediate disorders involving aberrant activities of these cells, for example diseases of the lung. Examples of disorders of the lung include, but are not limited to, congenital anomalies; atelectasis; diseases of vascular origin, such as pulmonary congestion and edema, including hemodynamic pulmonary edema and edema caused by microvascular injury, adult respiratory distress syndrome (diffuse alveolar damage), pulmonary embolism, hemorrhage, and infarction, and pulmonary hypertension and vascular sclerosis; chronic obstructive pulmonary disease, such as emphysema, chronic bronchitis, bronchial asthma, and bronchiectasis; diffuse interstitial (infiltrative, restrictive) diseases, such as pneumoconioses, sarcoidosis, idiopathic pulmonary fibrosis, desquamative interstitial pneumonitis, hypersensitivity pneumonitis, pulmonary eosinophilia (pulmonary infiltration with eosinophilia), Bronchiolitis obliterans-organizing pneumonia, diffuse pulmonary hemorrhage syndromes, including Goodpasture syndrome, idiopathic pulmonary hemosiderosis and other hemorrhagic syndromes, pulmonary involvement in collagen vascular disorders, and pulmonary alveolar proteinosis; complications of therapies, such as drug-induced lung disease, radiation-induced lung disease, and lung transplantation; tumors, such as bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

The 46638 nucleic acid and protein of the invention can be used to treat and/or diagnose a variety of immune disorders. Examples of immune disorders or diseases include, but are not limited to, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjogren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy such as, atopic allergy.

The 46638 protein, fragments thereof, and derivatives and other variants of the sequence in SEQ ID NO:23 thereof are collectively referred to as “polypeptides or proteins of the invention” or “46638 polypeptides or proteins”. Nucleic acid molecules encoding such polypeptides or proteins are collectively referred to as “nucleic acids of the invention” or “46638 nucleic acids.” 46638 molecules refer to 46638 nucleic acids, polypeptides, and antibodies.

As used herein, the term “nucleic acid molecule” includes DNA molecules (e.g., a cDNA or genomic DNA), RNA molecules (e.g., an mRNA) and analogs of the DNA or RNA. A DNA or RNA analog can be synthesized from nucleotide analogs. The nucleic acid molecule can be single-stranded or double-stranded, but preferably is double-stranded DNA.

The term “isolated nucleic acid molecule” or “purified nucleic acid molecule” includes nucleic acid molecules that are separated from other nucleic acid molecules present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2× SSC, 0.1% SDS at least at 50° C. (the temperature of the washes can be increased to 55° C. for low stringency conditions); 2) medium stringency hybridization conditions in 6× SSC at about 45° C., followed by one or more washes in 0.2× SSC, 0.1% SDS at 60° C.; 3) high stringency hybridization conditions in 6× SSC at about 45° C., followed by one or more washes in 0.2× SSC, 0.1% SDS at 65° C.; and preferably 4) very high stringency hybridization conditions are 0.5M sodium phosphate, 7% SDS at 65° C., followed by one or more washes at 0.2× SSC, 1% SDS at 65° C. Very high stringency conditions (4) are the preferred conditions and the ones that should be used unless otherwise specified.

Preferably, an isolated nucleic acid molecule of the invention that hybridizes under a stringency condition described herein to the sequence of SEQ ID NO:22 or SEQ ID NO:24, corresponds to a naturally-occurring nucleic acid molecule.

As used herein, a “naturally-occurring” nucleic acid molecule refers to an RNA or DNA molecule having a nucleotide sequence that occurs in nature. For example a naturally occurring nucleic acid molecule can encode a natural protein.

As used herein, the terms “gene” and “recombinant gene” refer to nucleic acid molecules which include at least an open reading frame encoding a 46638 protein. The gene can optionally further include non-coding sequences, e.g., regulatory sequences and introns. Preferably, a gene encodes a mammalian 46638 protein or derivative thereof.

An “isolated” or “purified” polypeptide or protein is substantially free of cellular material or other contaminating proteins from the cell or tissue source from which the protein is derived, or substantially free from chemical precursors or other chemicals when chemically synthesized. “Substantially free” means that a preparation of 46638 protein is at least 10% pure. In a preferred embodiment, the preparation of 46638 protein has less than about 30%, 20%, 10% and more preferably 5% (by dry weight), of non-46638 protein (also referred to herein as a “contaminating protein”), or of chemical precursors or non-46638 chemicals. When the 46638 protein or biologically active portion thereof is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight.

A “non-essential” amino acid residue is a residue that can be altered from the wild-type sequence of 46638 without abolishing or substantially altering a 46638 activity. Preferably the alteration does not substantially alter the 46638 activity, e.g., the activity is at least 20%, 40%, 60%, 70% or 80% of wild-type. An “essential” amino acid residue is a residue that, when altered from the wild-type sequence of 46638, results in abolishing a 46638 activity such that less than 20% of the wild-type activity is present. For example, conserved amino acid residues in 46638 are predicted to be particularly unamenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 46638 protein is preferably replaced with another amino acid residue from the same side chain family. Alternatively, in another embodiment, mutations can be introduced randomly along all or part of a 46638 coding sequence, such as by saturation mutagenesis, and the resultant mutants can be screened for 46638 biological activity to identify mutants that retain activity. Following mutagenesis of SEQ ID NO:22 or SEQ ID NO:24, the encoded protein can be expressed recombinantly and the activity of the protein can be determined.

As used herein, a “biologically active portion” of a 46638 protein includes a fragment of a 46638 protein which participates in an interaction, e.g., an intramolecular or an inter-molecular interaction. An inter-molecular interaction can be a specific binding interaction or an enzymatic interaction (e.g., the interaction can be transient and a covalent bond is formed or broken). An inter-molecular interaction can be between a 46638 molecule and a non-46638 molecule or between a first 46638 molecule and a second 46638 molecule (e.g., a dimerization interaction). Biologically active portions of a 46638 protein include peptides comprising amino acid sequences sufficiently homologous to or derived from the amino acid sequence of the 46638 protein, e.g., the amino acid sequence shown in SEQ ID NO:23, which include less amino acids than the full length 46638 proteins, and exhibit at least one activity of a 46638 protein. Typically, biologically active portions comprise a domain or motif with at least one activity of the 46638 protein, e.g., the ability to catalyzed the hydroperoxidation of a substrate, e.g., a fatty acid substrate (e.g., arachidonic acid); the ability to synthesize or metabolize leukotrienes, lipoxins and /or prostaglandins; the ability to bind an iron atom; and /or the ability to associate or attach to a cell membrane. A biologically active portion of a 46638 protein can be a polypeptide which is, for example, 10, 25, 50, 100, 200, 300, 400 or more amino acids in length. Biologically active portions of a 46638 protein can be used as targets for developing agents which modulate a 46638 mediated activity, e.g., protease activity.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to 46638 nucleic acid molecules of the invention. BLAST protein searches can be performed with the XBLAST program, score=50, wordlength=3 to obtain amino acid sequences homologous to 46638 protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

Particular 46638 polypeptides of the present invention have an amino acid sequence substantially identical to the amino acid sequence of SEQ ID NO:23. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 60%, or 65% identity, likely 75% identity, more likely 85%, 90%. 91%, 92%, 93%, 94%, 95%, 96%, 97%, 98% or 99% identity to SEQ ID NO:23 are termed substantially identical.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 60%, or 65% identity, likely 75% identity, more likely 85%, 90%. 91%, 92%, 93%, 94%, 95%, 96%, 97%, 98% or 99% identity to SEQ ID NO:22 or 24 are termed substantially identical.

“Misexpression or aberrant expression”, as used herein, refers to a non-wildtype pattern of gene expression at the RNA or protein level. It includes: expression at non-wild type levels, i.e., over- or under-expression; a pattern of expression that differs from wild type in terms of the time or stage at which the gene is expressed, e.g., increased or decreased expression (as compared with wild type) at a predetermined developmental period or stage; a pattern of expression that differs from wild type in terms of altered, e.g., increased or decreased, expression (as compared with wild type) in a predetermined cell type or tissue type; a pattern of expression that differs from wild type in terms of the splicing size, translated amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

“Subject,” as used herein, refers to human and non-human animals. The term “non-human animals” of the invention includes all vertebrates, e.g., mammals, such as non-human primates (particularly higher primates), sheep, dog, rodent (e.g., mouse or rat), guinea pig, goat, pig, cat, rabbits, cow, and non-mammals, such as chickens, amphibians, reptiles, etc. In a preferred embodiment, the subject is a human. In another embodiment, the subject is an experimental animal or animal suitable as a disease model.

A “purified preparation of cells”, as used herein, refers to an in vitro preparation of cells. In the case cells from multicellular organisms (e.g., plants and animals), a purified preparation of cells is a subset of cells obtained from the organism, not the entire intact organism. In the case of unicellular microorganisms (e.g., cultured cells and microbial cells), it consists of a preparation of at least 10% and more preferably 50% of the subject cells.

Various aspects of the invention are described in further detail below.

Isolated Nucleic Acid Molecules of 46638

In one aspect, the invention provides, an isolated or purified, nucleic acid molecule that encodes a 46638 polypeptide described herein, e.g., a full-length 46638 protein or a fragment thereof, e.g., a biologically active portion of 46638 protein. Also included is a nucleic acid fragment suitable for use as a hybridization probe, which can be used, e.g., to identify a nucleic acid molecule encoding a polypeptide of the invention, 46638 mRNA, and fragments suitable for use as primers, e.g., PCR primers for the amplification or mutation of nucleic acid molecules.

In one embodiment, an isolated nucleic acid molecule of the invention includes the nucleotide sequence shown in SEQ ID NO:22, or a portion of any of these nucleotide sequences. In one embodiment, the nucleic acid molecule includes sequences encoding the human 46638 protein (i.e., “the coding region” of SEQ ID NO:22, as shown in SEQ ID NO:24), as well as 5′ untranslated sequences. Alternatively, the nucleic acid molecule can include only the coding region of SEQ ID NO:22 (e.g., SEQ ID NO:24) and, e.g., no flanking sequences which normally accompany the subject sequence. In another embodiment, the nucleic acid molecule encodes a sequence corresponding to a fragment of the protein from about amino acid 267 to 703 of SEQ ID NO:23.

In another embodiment, an isolated nucleic acid molecule of the invention includes a nucleic acid molecule which is a complement of the nucleotide sequence shown in SEQ ID NO:22 or SEQ ID NO:24, or a portion of any of these nucleotide sequences. In other embodiments, the nucleic acid molecule of the invention is sufficiently complementary to the nucleotide sequence shown in SEQ ID NO:22 or SEQ ID NO:24, such that it can hybridize (e.g., under a stringency condition described herein) to the nucleotide sequence shown in SEQ ID NO:22 or 24, thereby forming a stable duplex.

In one embodiment, an isolated nucleic acid molecule of the present invention includes a nucleotide sequence which is at least about 60%, 65%, 70%, 75%, 80%, 85%, 90%, 91%, 92%, 93%, 94%, 95%, 96%, 97%, 98%, 99%, or more homologous to the entire length of the nucleotide sequence shown in SEQ ID NO:22 or SEQ ID NO:24, or a portion, preferably of the same length, of any of these nucleotide sequences.

46638 Nucleic Acid Fragments

A nucleic acid molecule of the invention can include only a portion of the nucleic acid sequence of SEQ ID NO:22 or 24. For example, such a nucleic acid molecule can include a fragment which can be used as a probe or primer or a fragment encoding a portion of a 46638 protein, e.g., an immunogenic or biologically active portion of a 46638 protein. A fragment can comprise those nucleotides of SEQ ID NO:22, which encode a lipoxygenase domain of human 46638. The nucleotide sequence determined from the cloning of the 46638 gene allows for the generation of probes and primers designed for use in identifying and/or cloning other 46638 family members, or fragments thereof, as well as 46638 homologues, or fragments thereof, from other species.

In another embodiment, a nucleic acid includes a nucleotide sequence that includes part, or all, of the coding region and extends into either (or both) the 5′ or 3′ noncoding region. Other embodiments include a fragment which includes a nucleotide sequence encoding an amino acid fragment described herein. Nucleic acid fragments can encode a specific domain or site described herein or fragments thereof, particularly fragments thereof which are at least 50, 100, 150, 200, 250, 300, 350, 400, 450, 500, 550, 600, 650, 700, 712, 750 amino acids in length. Fragments also include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a domain, region, or functional site described herein. A nucleic acid fragment can also include one or more domain, region, or functional site described herein. Thus, for example, a 46638 nucleic acid fragment can include a sequence corresponding to a lipoxygenase domain or a PLAT/LH2 domain, at locations in the translated 46638 polypeptide described herein.

46638 probes and primers are provided. Typically a probe/primer is an isolated or purified oligonucleotide. The oligonucleotide typically includes a region of nucleotide sequence that hybridizes under a stringency condition described herein to at least about 7, 12 or 15, preferably about 20 or 25, more preferably about 30, 35, 40, 45, 50, 55, 60, 65, or 75 consecutive nucleotides of a sense or antisense sequence of SEQ ID NO:22 or SEQ ID NO:24, or of a naturally occurring allelic variant or mutant of SEQ ID NO:22 or SEQ ID NO:24.

In a preferred embodiment the nucleic acid is a probe which is at least 5 or 10, and less than 200, more preferably less than 100, or less than 50, base pairs in length. It should be identical, or differ by 1, or less than in 5 or 10 bases, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid which encodes, e.g., a lipoxygenase domain from about amino acid 267 to 703 of SEQ ID NO:23, and a PLAT/LH2 domain located from about amino acid 2 to about 116 of SEQ ID NO:23.

In another embodiment a set of primers is provided, e.g., primers suitable for use in a PCR, which can be used to amplify a selected region of a 46638 sequence, e.g., a domain, region, site or other sequence described herein. The primers should be at least 5, 10, or 50 base pairs in length and less than 100, or less than 200, base pairs in length. The primers should be identical, or differs by one base from a sequence disclosed herein or from a naturally occurring variant. For example, primers suitable for amplifying all or a portion of any of the following regions are provided: a lipoxygenase domain from about amino acid 267 to 703 of SEQ ID NO:23; and a PLAT/LH2 domain located from about amino acid 2 to 116 of SEQ ID NO:23.

A nucleic acid fragment can encode an epitope bearing region of a polypeptide described herein.

A nucleic acid fragment encoding a “biologically active portion of a 46638 polypeptide” can be prepared by isolating a portion of the nucleotide sequence of SEQ ID NO:22 or 24, which encodes a polypeptide having a 46638 biological activity (e.g., the biological activities of the 46638 proteins are described herein), expressing the encoded portion of the 46638 protein (e.g., by recombinant expression in vitro) and assessing the activity of the encoded portion of the 46638 protein. For example, a nucleic acid fragment encoding a biologically active portion of 46638 includes a lipoxygenase domain, e.g., amino acid residues about 267 to 703 of SEQ ID NO:23. A nucleic acid fragment encoding a biologically active portion of a 46638 polypeptide, may comprise a nucleotide sequence which is greater than 300 or more nucleotides in length.

In preferred embodiments, the nucleic acid fragment includes a nucleotide sequence that is other than the sequence of AW300461.

In preferred embodiments, the fragment includes at least one, and preferably at least 5, 10, 15, 25, 50, 100, 120, 130, 140, 141 nucleotides from nucleotides 1 to 141 of SEQ ID NO:22.

In preferred embodiments, the fragment comprises the coding region of 46638, e.g., the nucleotide sequence of SEQ ID NO:24.

In preferred embodiments, a nucleic acid includes a nucleotide sequence which is about 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000 or more nucleotides in length and hybridizes under a stringency condition described herein to a nucleic acid molecule of SEQ ID NO:22, or SEQ ID NO:24.

46638 Nucleic Acid Variants

The invention further encompasses nucleic acid molecules that differ from the nucleotide sequence shown in SEQ ID NO:22 or SEQ ID NO:24. Such differences can be due to degeneracy of the genetic code (and result in a nucleic acid which encodes the same 46638 proteins as those encoded by the nucleotide sequence disclosed herein. In another embodiment, an isolated nucleic acid molecule of the invention has a nucleotide sequence encoding a protein having an amino acid sequence which differs, by at least 1, but less than 5, 10, 20, 50, or 100 amino acid residues that shown in SEQ ID NO:23. If alignment is needed for this comparison the sequences should be aligned for maximum homology. “Looped” out sequences from deletions or insertions, or mismatches, are considered differences.

Nucleic acids of the inventor can be chosen for having codons, which are preferred, or non-preferred, for a particular expression system. E.g., the nucleic acid can be one in which at least one codon, at preferably at least 10%, or 20% of the codons has been altered such that the sequence is optimized for expression in E. coli, yeast, human, insect, or CHO cells.

Nucleic acid variants can be naturally occurring, such as allelic variants (same locus), homologs (different locus), and orthologs (different organism) or can be non naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

In a preferred embodiment, the nucleic acid differs from that of SEQ ID NO:22 or 24, e.g., as follows: by at least one but less than 10, 20, 30, or 40 nucleotides; at least one but less than 1%, 5%, 10% or 20% of the nucleotides in the subject nucleic acid. If necessary for this analysis the sequences should be aligned for maximum homology. “Looped” out sequences from deletions or insertions, or mismatches, are considered differences.

Orthologs, homologs, and allelic variants can be identified using methods known in the art. These variants comprise a nucleotide sequence encoding a polypeptide that is 50%, at least about 55%, typically at least about 70-75%, more typically at least about 80-85%, and most typically at least about 90-95% or more identical to the nucleotide sequence shown in SEQ ID NO:23 or a fragment of this sequence. Such nucleic acid molecules can readily be identified as being able to hybridize under a stringency condition described herein, to the nucleotide sequence shown in SEQ ID NO:23 or a fragment of the sequence. Nucleic acid molecules corresponding to orthologs, homologs, and allelic variants of the 46638 cDNAs of the invention can further be isolated by mapping to the same chromosome or locus as the 46638 gene.

Preferred variants include those that are correlated with modulating (stimulating and /or enhancing or inhibiting) cellular proliferation, differentiation, or tumorogenesis; modulating an immune response; modulating inflammation; modulating smooth muscle cell activity; modulating prostate or neural cell activities.

Allelic variants of 46638, e.g., human 46638, include both functional and non-functional proteins. Functional allelic variants are naturally occurring amino acid sequence variants of the 46638 protein within a population that maintain the ability to bind fatty acid substrates, and to catalyze the hydroperoxidation of a substrate, e.g., arachidonic acid Functional allelic variants will typically contain only conservative substitution of one or more amino acids of SEQ ID NO:23, or substitution, deletion or insertion of non-critical residues in non-critical regions of the protein. Non-functional allelic variants are naturally-occurring amino acid sequence variants of the 46638, e.g., human 46638, protein within a population that do not have the ability to bind fatty acid substrates, and to catalyze the hydroperoxidation of a substrate, e.g., arachidonic acid Non-functional allelic variants will typically contain a non-conservative substitution, a deletion, or insertion, or premature truncation of the amino acid sequence of SEQ ID NO:23, or a substitution, insertion, or deletion in critical residues or critical regions of the protein.

Moreover, nucleic acid molecules encoding other 46638 family members and, thus, which have a nucleotide sequence which differs from the 46638 sequences of SEQ ID NO:22 or SEQ ID NO:24 are intended to be within the scope of the invention.

Antisense Nucleic Acid Molecules, Ribozymes and Modified 46638 Nucleic Acid Molecules

In another aspect, the invention features, an isolated nucleic acid molecule which is antisense to 46638. An “antisense” nucleic acid can include a nucleotide sequence which is complementary to a “sense” nucleic acid encoding a protein, e.g., complementary to the coding strand of a double-stranded cDNA molecule or complementary to an mRNA sequence. The antisense nucleic acid can be complementary to an entire 46638 coding strand, or to only a portion thereof (e.g., the coding region of human 46638 corresponding to SEQ ID NO:24). In another embodiment, the antisense nucleic acid molecule is antisense to a “noncoding region” of the coding strand of a nucleotide sequence encoding 46638 (e.g., the 5′ and 3′ untranslated regions).

An antisense nucleic acid can be designed such that it is complementary to the entire coding region of 46638 mRNA, but more preferably is an oligonucleotide which is antisense to only a portion of the coding or noncoding region of46638 mRNA. For example, the antisense oligonucleotide can be complementary to the region surrounding the translation start site of 46638 mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject (e.g., by direct injection at a tissue site), or generated in situ such that they hybridize with or bind to cellular mRNA and/or genomic DNA encoding a 46638 protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. A ribozyme having specificity for a 46638-encoding nucleic acid can include one or more sequences complementary to the nucleotide sequence of a 46638 cDNA disclosed herein (i.e., SEQ ID NO:22 or SEQ ID NO:24), and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a 46638-encoding mRNA. See, e.g., Cech et al. U.S. Pat. No. 4,987,071; and Cech et al. U.S. Pat. No. 5,116,742. Alternatively, 46638 mRNA can be used to select a catalytic RNA having a specific ribonuclease activity from a pool of RNA molecules. See, e.g., Bartel, D. and Szostak, J. W. (1993) Science 261:1411-1418.

46638 gene expression can be inhibited by targeting nucleotide sequences complementary to the regulatory region of the 46638 (e.g., the 46638 promoter and/or enhancers) to form triple helical structures that prevent transcription of the 46638 gene in target cells. See generally, Helene, C. (1991) Anticancer Drug Des. 6:569-84; Helene, C. i (1992) Ann. N.Y. Acad. Sci. 660:27-36; and Maher, L. J. (1992) Bioassays 14:807-15. The potential sequences that can be targeted for triple helix formation can be increased by creating a so-called switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

The invention also provides detectably labeled oligonucleotide primer and probe molecules. Typically, such labels are chemiluminescent, fluorescent, radioactive, or colorimetric.

A 46638 nucleic acid molecule can be modified at the base moiety, sugar moiety or phosphate backbone to improve, e.g., the stability, hybridization, or solubility of the molecule. For non-limiting examples of synthetic oligonucleotides with modifications see Toulme (2001) Nature Biotech. 19:17 and Faria et al. (2001) Nature Biotech. 19:40-44. Such phosphoramidite oligonucleotides can be effective antisense agents.

For example, the deoxyribose phosphate backbone of the nucleic acid molecules can be modified to generate peptide nucleic acids (see Hyrup B. et al. (1996) Bioorganic & Medicinal Chemistry 4: 5-23). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup B. et al. (1996) supra and Perry-O'Keefe et al. Proc. Natl. Acad. Sci. 93: 14670-675.

PNAs of 46638 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of 46638 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al. (1996) supra)); or as probes or primers for DNA sequencing or hybridization (Hyrup B. et al. (1996) supra; Perry-O'Keefe supra).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (see, e.g., Krol et al. (1988) Bio-Techniques 6:958-976) or intercalating agents. (see, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

The invention also includes molecular beacon oligonucleotide primer and probe molecules having at least one region which is complementary to a 46638 nucleic acid of the invention, two complementary regions one having a fluorophore and one a quencher such that the molecular beacon is useful for quantitating the presence of the 46638 nucleic acid of the invention in a sample. Molecular beacon nucleic acids are described, for example, in Lizardi et al., U.S. Pat. No. 5,854,033; Nazarenko et a., U.S. Pat. No. 5,866,336, and Livak et a., U.S. Pat. No. 5,876,930.

Isolated 46638 Polypeptides

In another aspect, the invention features, an isolated 46638 protein, or fragment, e.g., a biologically active portion, for use as immunogens or antigens to raise or test (or more generally to bind) anti-46638 antibodies. 46638 protein can be isolated from cells or tissue sources using standard protein purification techniques. 46638 protein or fragments thereof can be produced by recombinant DNA techniques or synthesized chemically.

Polypeptides of the invention include those which arise as a result of the existence of multiple genes, alternative transcription events, alternative RNA splicing events, and alternative translational and post-translational events. The polypeptide can be expressed in systems, e.g., cultured cells, which result in substantially the same post-translational modifications present when expressed the polypeptide is expressed in a native cell, or in systems which result in the alteration or omission of post-translational modifications, e.g., glycosylation or cleavage, present when expressed in a native cell.

In a preferred embodiment, a 46638 polypeptide has one or more of the following characteristics:

(i) it has the ability to catalyze the hydroperoxidation of a substrate, e.g., a fatty acid substrate (e.g. arachidonic acid);

(ii) it synthesizes or metabolizes leukotrienes lipoxins and /or prostaglandins;

(iii) it binds to an iron atom;

(iv) it associates or attaches to a cell membrane;

(v) it has an amino acid composition of a 46638 polypeptide, e.g., a polypeptide of SEQ ID NO:23;

(vi) it has an overall sequence similarity of at least 60%, preferably at least 70, more preferably at least 80, 90, or 95%, with a polypeptide of SEQ ID NO:23;

(vii) it can be found in human tissue, e.g., prostate or neural tissue;

(viii) it has a lipoxygenase domain with a sequence similarity which is preferably about 70%, 80%, 90%, or 95%, with amino acid residues about 267 to about 703 of SEQ ID NO:23;

(x) it has at least three, preferably at least 4, more preferably at least 5, most preferably at least six histidines found in the amino acid sequence of the protein of SEQ ID NO:23; or

(xi) it has at least 10, preferably at least 12, and most preferably at least 15 of the 20 cysteines found in the amino acid sequence of the native protein.

In a preferred embodiment the 46638 protein, or fragment thereof, differs from the corresponding sequence in SEQ ID:2. In one embodiment it differs by at least one but by less than 15, 10 or 5 amino acid residues. In another it differs from the corresponding sequence in SEQ ID NO:23 by at least one residue but less than 20%, 15%, 10% or 5% of the residues in it differ from the corresponding sequence in SEQ ID NO:23. (If this comparison requires alignment the sequences should be aligned for maximum homology. “Looped” out sequences from deletions or insertions, or mismatches, are considered differences.) The differences are, preferably, differences or changes at a non essential residue or a conservative substitution. In a preferred embodiment the differences are not in the lipoxygenase domain. In another preferred embodiment one or more differences are in transmembrane domains or non-transmembrane domains.

Other embodiments include a protein that contain one or more changes in amino acid sequence, e.g., a change in an amino acid residue which is not essential for activity. Such 46638 proteins differ in amino acid sequence from SEQ ID NO:23, yet retain biological activity.

In one embodiment, the protein includes an amino acid sequence at least about 60%, 65%, 70%, 75%, 80%, 85%, 90%, 95%, 98% or more homologous to SEQ ID NO:23.

A 46638 protein or fragment is provided which varies from the sequence of SEQ ID NO:23 in regions defined by amino acids about 117 to 266 of SEQ ID NO:23 by at least one but by less than 15, 10 or 5 amino acid residues in the protein or fragment but which does not differ from SEQ ID NO:23 in regions defined by amino acids about 267 to about 703 of SEQ ID NO:23. (If this comparison requires alignment the sequences should be aligned for maximum homology. “Looped” out sequences from deletions or insertions, or mismatches, are considered differences.) In some embodiments the difference is at a non-essential residue or is a conservative substitution, while in others the difference is at an essential residue or is a non-conservative substitution.

In one embodiment, a biologically active portion of a 46638 protein includes a lipoxygenase domain. Moreover, other biologically active portions, in which other regions of the protein are deleted, can be prepared by recombinant techniques and evaluated for one or more of the functional activities of a native 46638 protein.

In a preferred embodiment, the 46638 protein has an amino acid sequence shown in SEQ ID NO:23. In other embodiments, the 46638 protein is substantially identical to SEQ ID NO:23. In yet another embodiment, the 46638 protein is substantially identical to SEQ ID NO:23 and retains the functional activity of the protein of SEQ ID NO:23, as described in detail in the subsections above.

46638 Chimeric or Fusion Proteins

In another aspect, the invention provides 46638 chimeric or fusion proteins. As used herein, a 46638 “chimeric protein” or “fusion protein” includes a 46638 polypeptide linked to a non-46638 polypeptide. A “non-46638 polypeptide” refers to a polypeptide having an amino acid sequence corresponding to a protein which is not substantially homologous to the 46638 protein, e.g., a protein which is different from the 46638 protein and which is derived from the same or a different organism. The 46638 polypeptide of the fusion protein can correspond to all or a portion e.g., a fragment described herein of a 46638 amino acid sequence. In a preferred embodiment, a 46638 fusion protein includes at least one (or two) biologically active portion of a 46638 protein. The non-46638 polypeptide can be fused to the N-terminus or C-terminus of the 46638 polypeptide.

The fusion protein can include a moiety which has a high affinity for a ligand. For example, the fusion protein can be a GST-46638 fusion protein in which the 46638 sequences are fused to the C-terminus of the GST sequences. Such fusion proteins can facilitate the purification of recombinant 46638. Alternatively, the fusion protein can be a 46638 protein containing a heterologous signal sequence at its N-terminus. In certain host cells (e.g., mammalian host cells), expression and/or secretion of 46638 can be increased through use of a heterologous signal sequence.

Fusion proteins can include all or a part of a serum protein, e.g., an IgG constant region, or human serum albumin.

The 46638 fusion proteins of the invention can be incorporated into pharmaceutical compositions and administered to a subject in vivo. The 46638 fusion proteins can be used to affect the bioavailability of a 46638 substrate. 46638 fusion proteins maybe useful therapeutically for the treatment of disorders caused by, for example, (i) aberrant modification or mutation of a gene encoding a 46638 protein; (ii) mis-regulation of the 46638 gene; and (iii) aberrant post-translational modification of a 46638 protein.

Moreover, the 46638-fusion proteins of the invention can be used as immunogens to produce anti-46638 antibodies in a subject, to purify 46638 ligands and in screening assays to identify molecules which inhibit the interaction of 46638 with a 46638 substrate.

Expression vectors are commercially available that already encode a fusion moiety (e.g., a GST polypeptide). A 46638-encoding nucleic acid can be cloned into such an expression vector such that the fusion moiety is linked in-frame to the 46638 protein.

Variants of 46638 Proteins

In another aspect, the invention also features a variant of a 46638 polypeptide, e.g., which functions as an agonist (mimetics) or as an antagonist. Variants of the 46638 proteins can be generated by mutagenesis, e.g., discrete point mutation, the insertion or deletion of sequences or the truncation of a 46638 protein. An agonist of the 46638 proteins can retain substantially the same, or a subset, of the biological activities of the naturally occurring form of a 46638 protein. An antagonist of a 46638 protein can inhibit one or more of the activities of the naturally occurring form of the 46638 protein by, for example, competitively modulating a 46638-mediated activity of a 46638 protein. Thus, specific biological effects can be elicited by treatment with a variant of limited function. Preferably, treatment of a subject with a variant having a subset of the biological activities of the naturally occurring form of the protein has fewer side effects in a subject relative to treatment with the naturally occurring form of the 46638 protein.

Variants of a 46638 protein can be identified by screening combinatorial libraries of mutants, e.g., truncation mutants, of a 46638 protein for agonist or antagonist activity.

Libraries of fragments e.g., N terminal, C terminal, or internal fragments, of a 46638 protein coding sequence can be used to generate a variegated population of fragments for screening and subsequent selection of variants of a 46638 protein. Variants in which a cysteine residues is added or deleted or in which a residue which is glycosylated is added or deleted are particularly preferred.

Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of 46638 proteins. Recursive ensemble mutagenesis (REM), a new technique which enhances the frequency of functional mutants in the libraries, can be used in combination with the screening assays to identify 46638 variants (Arkin and Yourvan (1992) Proc. Natl. Acad. Sci. USA 89:7811-7815; Delgrave et al. (1993) Protein Engineering 6:327-331).

Cell based assays can be exploited to analyze a variegated 46638 library. For example, a library of expression vectors can be transfected into a cell line, e.g., a cell line, which ordinarily responds to 46638 in a substrate-dependent manner. The transfected cells are then contacted with 46638 and the effect of the expression of the mutant on signaling by the 46638 substrate can be detected. Plasmid DNA can then be recovered from the cells which score for inhibition, or alternatively, potentiation of signaling by the 46638 substrate, and the individual clones further characterized.

In another aspect, the invention features a method of making a 46638 polypeptide, e.g., a peptide having a non-wild type activity, e.g., an antagonist, agonist, or super agonist of a naturally occurring 46638 polypeptide, e.g., a naturally occurring 46638 polypeptide. The method includes: altering the sequence of a 46638 polypeptide, e.g., altering the sequence, e.g., by substitution or deletion of one or more residues of a non-conserved region, a domain or residue disclosed herein, and testing the altered polypeptide for the desired activity.

In another aspect, the invention features a method of making a fragment or analog of a 46638 polypeptide a biological activity of a naturally occurring 46638 polypeptide. The method includes: altering the sequence, e.g., by substitution or deletion of one or more residues, of a 46638 polypeptide, e.g., altering the sequence of a non-conserved region, or a domain or residue described herein, and testing the altered polypeptide for the desired activity.

Anti-46638 Antibodies

In another aspect, the invention provides an anti-46638 antibody, or a fragment thereof (e.g., an antigen-binding fragment thereof). The term “antibody” as used herein refers to an immunoglobulin molecule or immunologically active portion thereof, i.e., an antigen-binding portion. As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4.

The anti-46638 antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

As used herein, the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 KDa or 214 amino acids) are encoded by a variable region gene at the NH2-terminus (about 110 amino acids) and a kappa or lambda constant region gene at the COOH—terminus. Full-length immunoglobulin “heavy chains” (about 50 KDa or 446 amino acids), are similarly encoded by a variable region gene (about 116 amino acids) and one of the other aforementioned constant region genes, e.g., gamma (encoding about 330 amino acids).

The term “antigen-binding fragment” of an antibody (or simply “antibody portion,” or “fragment”), as used herein, refers to one or more fragments of a full-length antibody that retain the ability to specifically bind to the antigen, e.g., 46638 polypeptide or fragment thereof. Examples of antigen-binding fragments of the anti-46638 antibody include, but are not limited to: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “antigen-binding fragment” of an antibody. These antibody fragments are obtained using conventional techniques known to those with skill in the art, and the fragments are screened for utility in the same manner as are intact antibodies.

The anti-46638 antibody can be a polyclonal or a monoclonal antibody. In other embodiments, the antibody can be recombinantly produced, e.g., produced by phage display or by combinatorial methods.

Phage display and combinatorial methods for generating anti-46638 antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J. 12:725-734; Hawkins et al. (1992) J. Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the anti-46638 antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Method of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An anti-46638 antibody can be one in which the variable region, or a portion thereof, e.g., the CDR's, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/IJS86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDR's (of heavy and or light immuoglobulin chains) replaced with a donor CDR. CDR's of the antibody may be replaced with at least a portion of a non-human CDR or only some of the CDR's may be replaced with non-human CDR's. It is only necessary to replace the number of CDR's required for binding of the humanized antibody to a 46638 or a fragment thereof. Preferably, the donor will be a rodent antibody, e.g., a rat or mouse antibody, and the recipient will be a human framework or a human consensus framework. Typically, the immunoglobulin providing the CDR's is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art. Humanized antibodies can be generated by replacing sequences of the Fv variable region which are not directly involved in antigen binding with equivalent sequences from human Fv variable regions. General methods for generating humanized antibodies are provided by Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a 46638 polypeptide or fragment thereof. The recombinant DNA encoding the humanized antibody, or fragment thereof, can then be cloned into an appropriate expression vector.

Humanized or CDR-grafted antibodies can be produced by CDR-grafting or CDR substitution, wherein one, two, or all CDR's of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Preferred humanized antibodies have amino acid substitutions in the framework region, such as to improve binding to the antigen. For example, a humanized antibody will have framework residues identical to the donor framework residue or to another amino acid other than the recipient framework residue. To generate such antibodies, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

In preferred embodiments an antibody can be made by immunizing with purified 46638 antigen, or a fragment thereof, e.g., a fragment described herein, membrane associated antigen, tissue, e.g., crude tissue preparations, whole cells, preferably living cells, lysed cells, or cell fractions, e.g., membrane fractions.

A full-length 46638 protein or, antigenic peptide fragment of 46638 can be used as an immunogen or can be used to identify anti-46638 antibodies made with other immunogens, e.g., cells, membrane preparations, and the like. The antigenic peptide of 46638 should include at least 8 amino acid residues of the amino acid sequence shown in SEQ ID NO:23 and encompasses an epitope of 46638. Preferably, the antigenic peptide includes at least 10 amino acid residues, more preferably at least 15 amino acid residues, even more preferably at least 20 amino acid residues, and most preferably at least 30 amino acid residues.

Fragments of 46638 which include residues about 508 to 510 and from 603 to 621 of SEQ ID NO:23 can be used to make, e.g., used as immunogens or used to characterize the specificity of an antibody, antibodies against hydrophilic regions of the 46638 protein. Similarly, fragments of 46638 which include residues from about 20 to 30, from 580 to 583, and from 643 to 645 of can be used to make an antibody against a hydrophobic region of the 46638 protein; and a fragment of 46638 which includes residues about 281 to 321, about 441 to 471, or about 481 to 521 of SEQ ID NO:23 can be used to make an antibody against the lipoxygenase region of the 46638 protein. Moreover, fragments of 46638 which include residues about 1-344 or a portion thereof, or 367-711 or a portion thereof of SEQ ID NO:23 can be used to make antibodies against the non-transmembrane domain (e.g., extracellular or intraluminal domain, or cytoplasmic domain) of a 46638 polypeptide.

In a preferred embodiment the antibody can bind to the extracellular portion of the 46638 protein, e.g., it can bind to a whole cell which expresses the 46638 protein. In another embodiment, the antibody binds an intracellular portion of the 46638 protein.

Antibodies reactive with, or specific for, any of these regions, or other regions or domains described herein are provided.

Antibodies which bind only native 46638 protein, only denatured or otherwise non-native 46638 protein, or which bind both, are with in the invention. Antibodies with linear or conformational epitopes are within the invention. Conformational epitopes can sometimes be identified by identifying antibodies which bind to native but not denatured 46638 protein.

Preferred epitopes encompassed by the antigenic peptide are regions of 46638 are located on the surface of the protein, e.g., hydrophilic regions, as well as regions with high antigenicity. For example, an Emini surface probability analysis of the human 46638 protein sequence can be used to indicate the regions that have a particularly high probability of being localized to the surface of the 46638 protein and are thus likely to constitute surface residues useful for targeting antibody production.

The anti-46638 antibody can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target 46638 protein.

In a preferred embodiment the antibody has: effector function; and can fix complement. In other embodiments the antibody does not; recruit effector cells; or fix complement.

In a preferred embodiment, the antibody has reduced or no ability to bind an Fc receptor. For example., it is a isotype or subtype, fragment or other mutant, which does not support binding to an Fc receptor, e.g., it has a mutagenized or deleted Fc receptor binding region.

In a preferred embodiment, an anti-46638 antibody alters (e.g., increases or decreases) the lipoxygenase activity of a 46638 polypeptide.

The antibody can be coupled to a toxin, e.g., a polypeptide toxin, e,g, ricin or diphtheria toxin or active fragment hereof, or a radioactive nucleus, or imaging agent, e.g. a radioactive, enzymatic, or other, e.g., imaging agent, e.g., a NMR contrast agent. Labels which produce detectable radioactive emissions or fluorescence are preferred.

An anti-46638 antibody (e.g., monoclonal antibody) can be used to isolate 46638 by standard techniques, such as affinity chromatography or immunoprecipitation. Moreover, an anti-46638 antibody can be used to detect 46638 protein (e.g., in a cellular lysate or cell supernatant) in order to evaluate the abundance and pattern of expression of the protein. Anti-46638 antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labelling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

The invention also includes a nucleic acids which encodes an anti-46638 antibody, e.g., an anti-46638 antibody described herein. Also included are vectors which include the nucleic acid and sells transformed with the nucleic acid, particularly cells which are useful for producing an antibody, e.g., mammalian cells, e.g. CHO or lymphatic cells.

The invention also includes cell lines, e.g., hybridomas, which make an anti-46638 antibody, e.g., and antibody described herein, and method of using said cells to make a 46638 antibody.

46638 Recombinant Expression Vectors Host Cells and Genetically Engineered Cells

In another aspect, the invention includes, vectors, preferably expression vectors, containing a nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a 46638 nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., 46638 proteins, mutant forms of 46638 proteins, fusion proteins, and the like).

The recombinant expression vectors of the invention can be designed for expression of 46638 proteins in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in 46638 activity assays, (e.g., direct assays or competitive assays described in detail below), or to generate antibodies specific for 46638 proteins. In a preferred embodiment, a fusion protein expressed in a retroviral expression vector of the present invention can be used to infect bone marrow cells which are subsequently transplanted into irradiated recipients. The pathology of the subject recipient is then examined after sufficient time has passed (e.g., six weeks).

To maximize recombinant protein expression in E. coli is to express the protein in a host bacteria with an impaired capacity to proteolytically cleave the recombinant protein (Gottesman, S., (1990) Gene Expression Technology: Methods in Enzymology 185, Academic Press, San Diego, Calif. 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The 46638 expression vector can be a yeast expression vector, a vector for expression in insect cells, e.g., a baculovirus expression vector or a vector suitable for expression in mammalian cells.

When used in mammalian cells, the expression vector's control functions can be provided by viral regulatory elements. For example, commonly used promoters are derived from polyoma, Adenovirus 2, cytomegalovirus and Simian Virus 40.

In another embodiment, the promoter is an inducible promoter, e.g., a promoter regulated by a steroid hormone, by a polypeptide hormone (e.g., by means of a signal transduction pathway), or by a heterologous polypeptide (e.g., the tetracycline-inducible systems, “Tet-On” and “Tet-Off”; see, e.g., Clontech Inc., CA, Gossen and Bujard (1992) Proc. Natl. Acad. Sci. USA 89:5547, and Paillard (1989) Human Gene Therapy 9:983).

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus.

Another aspect the invention provides a host cell which includes a nucleic acid molecule described herein, e.g., a 46638 nucleic acid molecule within a recombinant expression vector or a 46638 nucleic acid molecule containing sequences which allow it to homologously recombine into a specific site of the host cell's genome. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a 46638 protein can be expressed in bacterial cells (such as E. coli), insect cells, yeast or mammalian cells (such as Chinese hamster ovary cells (CHO) or COS cells). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell of the invention can be used to produce (i.e., express) a 46638 protein. Accordingly, the invention further provides methods for producing a 46638 protein using the host cells of the invention. In one embodiment, the method includes culturing the host cell of the invention (into which a recombinant expression vector encoding a 46638 protein has been introduced) in a suitable medium such that a 46638 protein is produced. In another embodiment, the method further includes isolating a 46638 protein from the medium or the host cell.

In another aspect, the invention features, a cell or purified preparation of cells which include a 46638 transgene, or which otherwise misexpress 46638. The cell preparation can consist of human or non-human cells, e.g., rodent cells, e.g., mouse or rat cells, rabbit cells, or pig cells. In preferred embodiments, the cell or cells include a 46638 transgene, e.g., a heterologous form of a 46638, e.g., a gene derived from humans (in the case of a non-human cell). The 46638 transgene can be misexpressed, e.g., overexpressed or underexpressed. In other preferred embodiments, the cell or cells include a gene that mis-expresses an endogenous 46638, e.g., a gene the expression of which is disrupted, e.g., a knockout. Such cells can serve as a model for studying disorders that are related to mutated or mis-expressed 46638 alleles or for use in drug screening.

In another aspect, the invention features, a human cell, e.g., a hematopoietic stem cell, transformed with nucleic acid which encodes a subject 46638 polypeptide.

Also provided are cells, preferably human cells, e.g., human hematopoietic or fibroblast cells, in which an endogenous 46638 is under the control of a regulatory sequence that does not normally control the expression of the endogenous 46638 gene. The expression characteristics of an endogenous gene within a cell, e.g., a cell line or microorganism, can be modified by inserting a heterologous DNA regulatory element into the genome of the cell such that the inserted regulatory element is operably linked to the endogenous 46638 gene. For example, an endogenous 46638 gene which is “transcriptionally silent,” e.g., not normally expressed, or expressed only at very low levels, may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published in May 16, 1991.

In a preferred embodiment, recombinant cells described herein can be used for replacement therapy in a subject. For example, a nucleic acid encoding a 46638 polypeptide operably linked to an inducible promoter (e.g., a steroid hormone receptor-regulated promoter) is introduced into a human or nonhuman, e.g., mammalian, e.g., porcine recombinant cell. The cell is cultivated and encapsulated in a biocompatible material, such as poly-lysine alginate, and subsequently implanted into the subject. See, e.g., Lanza (1996) Nat. Biotechnol. 14:1107; Joki et al. (2001) Nat. Biotechnol. 19:35; and U.S. Pat. No. 5,876,742. Production of a 46638 polypeptide can be regulated in the subject by administering an agent (e.g., a steroid hormone) to the subject. In another preferred embodiment, the implanted recombinant cells express and secrete an antibody specific for a 46638 polypeptide. The antibody can be any antibody or any antibody derivative described herein.

46638 Transgenic Animals

The invention provides non-human transgenic animals. Such animals are useful for studying the function and/or activity of a 46638 protein and for identifying and/or evaluating modulators of 46638 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous 46638 gene has been altered by, e.g., by homologous recombination between the endogenous gene and an exogenous DNA molecule introduced into a cell of the animal, e.g., an embryonic cell of the animal, prior to development of the animal.

Intronic sequences and polyadenylation signals can also be included in the transgene to increase the efficiency of expression of the transgene. A tissue-specific regulatory sequence(s) can be operably linked to a transgene of the invention to direct expression of a 46638 protein to particular cells. A transgenic founder animal can be identified based upon the presence of a 46638 transgene in its genome and/or expression of 46638 mRNA in tissues or cells of the animals. A transgenic founder animal can then be used to breed additional animals carrying the transgene. Moreover, transgenic animals carrying a transgene encoding a 46638 protein can further be bred to other transgenic animals carrying other transgenes.

46638 proteins or polypeptides can be expressed in transgenic animals or plants, e.g., a nucleic acid encoding the protein or polypeptide can be introduced into the genome of an animal. In preferred embodiments the nucleic acid is placed under the control of a tissue specific promoter, e.g., a milk or egg specific promoter, and recovered from the milk or eggs produced by the animal. Suitable animals are mice, pigs, cows, goats, and sheep.

The invention also includes a population of cells from a transgenic animal, as discussed, e.g., below.

Uses of 46638

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic).

The protein of the invention can be used in vitro, e.g., use in vitro to synthesize hydroperoxidated product compounds

The isolated nucleic acid molecules of the invention can be used, for example, to express a 46638 protein (e.g., via a recombinant expression vector in a host cell in gene therapy applications), to detect a 46638 mRNA (e.g., in a biological sample) or a genetic alteration in a 46638 gene, and to modulate 46638 activity, as described further below. The 46638 proteins can be used to treat disorders characterized by insufficient or excessive production of a 46638 substrate or production of 46638 inhibitors. In addition, the 46638 proteins can be used to screen for naturally occurring 46638 substrates, to screen for drugs or compounds which modulate 46638 activity, as well as to treat disorders characterized by insufficient or excessive production of 46638 protein or production of 46638 protein forms which have decreased, aberrant or unwanted activity compared to 46638 wild type protein Moreover, the anti-46638 antibodies of the invention can be used to detect and isolate 46638 proteins, regulate the bioavailability of 46638 proteins, and modulate 46638 activity.

A method of evaluating a compound for the ability to interact with, e.g., bind, a subject 46638 polypeptide is provided. The method includes: contacting the compound with the subject 46638 polypeptide; and evaluating ability of the compound to interact with, e.g., to bind or form a complex with the subject 46638 polypeptide. This method can be performed in vitro, e.g., in a cell free system, or in vivo, e.g., in a two-hybrid interaction trap assay. This method can be used to identify naturally occurring molecules that interact with subject 46638 polypeptide. It can also be used to find natural or synthetic inhibitors of subject 46638 polypeptide. Screening methods are discussed in more detail below.

46638 Screening Assays

The invention provides methods (also referred to herein as “screening assays”) for identifying modulators, i.e., candidate or test compounds or agents (e.g., proteins, peptides, peptidomimetics, peptoids, small molecules or other drugs) which bind to 46638 proteins, have a stimulatory or inhibitory effect on, for example, 46638 expression or 46638 activity, or have a stimulatory or inhibitory effect on, for example, the expression or activity of a 46638 substrate. Compounds thus identified can be used to modulate the activity of target gene products (e.g., 46638 genes) in a therapeutic protocol, to elaborate the biological function of the target gene product, or to identify compounds that disrupt normal target gene interactions.

In one embodiment, the invention provides assays for screening candidate or test compounds which are substrates of a 46638 protein or polypeptide or a biologically active portion thereof. In another embodiment, the invention provides assays for screening candidate or test compounds that bind to or modulate an activity of a 46638 protein or polypeptide or a biologically active portion thereof.

The test compounds of the present invention can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Nat?. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladnersupra.).

In one embodiment, an assay is a cell-based assay in which a cell which expresses a 46638 protein or biologically active portion thereof is contacted with a test compound, and the ability of the test compound to modulate 46638 activity is determined. Determining the ability of the test compound to modulate 46638 activity can be accomplished by monitoring, for example, lipoxygenase activity. The cell, for example, can be of mammalian origin, e.g., human.

The ability of the test compound to modulate 46638 binding to a compound, e.g., a 46638 substrate, or to bind to 46638 can also be evaluated. This can be accomplished, for example, by coupling the compound, e.g., the substrate, with a radioisotope or enzymatic label such that binding of the compound, e.g., the substrate, to 46638 can be determined by detecting the labeled compound, e.g., substrate, in a complex. Alternatively, 46638 could be coupled with a radioisotope or enzymatic label to monitor the ability of a test compound to modulate 46638 binding to a 46638 substrate in a complex. For example, compounds (e.g., 46638 substrates) can be labeled with ¹²⁵, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a 46638 substrate) to interact with 46638 with or without the labeling of any of the interactants can be evaluated. For example, a microphysiometer can be used to detect the interaction of a compound with 46638 without the labeling of either the compound or the 46638. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 46638.

In yet another embodiment, a cell-free assay is provided in which a 46638 protein or biologically active portion thereof is contacted with a test compound and the ability of the test compound to bind to the 46638 protein or biologically active portion thereof is evaluated. Preferred biologically active portions of the 46638 proteins to be used in assays of the present invention include fragments which participate in interactions with non-46638 molecules, e.g., fragments with high surface probability scores.

Soluble and/or membrane-bound forms of isolated proteins (e.g., 46638 proteins or biologically active portions thereof) can be used in the cell-free assays of the invention. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPS O), or N-dodecyl=N,N-dimethyl-3-ammonio-1-propane sulfonate.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of the 46638 protein to bind to a target molecule can be accomplished using real-time Biomolecular Interaction Analysis (BIA) (see, e.g., Sjolander, S. and Urbaniczky, C. (1991) Anal Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BLAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize either 46638, an anti-46638 antibody or its target molecule to facilitate separation of complexed from uncomplexed forms of one or both of the proteins, as well as to accommodate automation of the assay. Binding of a test compound to a 46638 protein, or interaction of a 46638 protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/46638 fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or 46638 protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of 46638 binding or activity determined using standard techniques.

Other techniques for immobilizing either a 46638 protein or a target molecule on matrices include using conjugation of biotin and streptavidin. Biotinylated 46638 protein or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with 46638 protein or target molecules but which do not interfere with binding of the 46638 protein to its target molecule. Such antibodies can be derivatized to the wells of the plate, and unbound target or 46638 protein trapped in the wells by antibody conjugation. Methods for detecting such complexes, in addition to those described above for the GST-immobilized complexes, include immunodetection of complexes using antibodies reactive with the 46638 protein or target molecule, as well as enzyme-linked assays which rely on detecting an enzymatic activity associated with the 46638 protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., (1993) Trends Biochem Sci 18:284-7); chromatography (gel filtration chromatography, ion-exchange chromatography); electrophoresis (see, e.g., Ausubel, F. et al., eds. Current Protocols in Molecular Biology 1999, J. Wiley: New York.); and immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N. H., (1998) J Mol Recognit 11:141-8; Hage, D. S., and Tweed, S. A. (1997) J Chromatogr B Biomed Sci Appl. 699:499-525). Further, fluorescence energy transfer may also be conveniently utilized, as described herein, to detect binding without further purification of the complex from solution.

In a preferred embodiment, the assay includes contacting the 46638 protein or biologically active portion thereof with a known compound which binds 46638 to form an assay mixture, contacting the assay mixture with a test compound, and determining the ability of the test compound to interact with a 46638 protein, wherein determining the ability of the test compound to interact with a 46638 protein includes determining the ability of the test compound to preferentially bind to 46638 or biologically active portion thereof, or to modulate the activity of a target molecule, as compared to the known compound.

The target gene products of the invention can, in vivo, interact with one or more cellular or extracellular macromolecules, such as proteins. For the purposes of this discussion, such cellular and extracellular macromolecules are referred to herein as “binding partners.”Compounds that disrupt such interactions can be useful in regulating the activity of the target gene product. Such compounds can include, but are not limited to molecules such as antibodies, peptides, and small molecules. The preferred target genes/products for use in this embodiment are the 46638 genes herein identified. In an alternative embodiment, the invention provides methods for determining the ability of the test compound to modulate the activity of a 46638 protein through modulation of the activity of a downstream effector of a 46638 target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the 46638 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with 46638 (“46638-binding proteins” or “46638-bp”) and are involved in 46638 activity. Such 46638-bps can be activators or inhibitors of signals by the 46638 proteins or 46638 targets as, for example, downstream elements of a 46638-mediated signaling pathway.

The two-hybrid system is based on the modular nature of most transcription factors, which consist of separable DNA-binding and activation domains. Briefly, the assay utilizes two different DNA constructs. In one construct, the gene that codes for a 46638 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively the: 46638 protein can be the fused to the activator domain.) If the “bait” and the “prey” proteins are able to interact, in vivo, forming a 46638-dependent complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 46638 protein.

In another embodiment, modulators of 46638 expression are identified. For example, a cell or cell free mixture is contacted with a candidate compound and the expression of 46638 mRNA or protein evaluated relative to the level of expression of 46638 mRNA or protein in the absence of the candidate compound. When expression of 46638 mRNA or protein is greater in the presence of the candidate compound than in its absence, the candidate compound is identified as a stimulator of 46638 mRNA or protein expression. Alternatively, when expression of 46638 mRNA or protein is less (statistically significantly less) in the presence of the candidate compound than in its absence, the candidate compound is identified as an inhibitor of 46638 mRNA or protein expression. The level of 46638 mRNA or protein expression can be determined by methods described herein for detecting 46638 mRNA or protein.

In another aspect, the invention pertains to a combination of two or more of the assays described herein. For example, a modulating agent can be identified using a cell-based or a cell free assay, and the ability of the agent to modulate the activity of a 46638 protein can be confirmed in vivo, e.g., in an animal such as an animal model for a disorder as described herein.

This invention further pertains to novel agents identified by the above-described screening assays. Accordingly, it is within the scope of this invention to further use an agent identified as described herein (e.g., a 46638 modulating agent, an antisense 46638 nucleic acid molecule, a 46638-specific antibody, or a 46638-binding partner) in an appropriate animal model to determine the efficacy, toxicity, side effects, or mechanism of action, of treatment with such an agent. Furthermore, novel agents identified by the above-described screening assays can be used for treatments as described herein.

46638 Detection Assays

Portions or fragments of the nucleic acid sequences identified herein can be used as polynucleotide reagents. For example, these sequences can be used to: (i) map their respective genes on a chromosome e.g., to locate gene regions associated with genetic disease or to associate 46638 with a disease; (ii) identify an individual from a minute biological sample (tissue typing); and (iii) aid in forensic identification of a biological sample. These applications are described in the subsections below.

46638 Chromosome Mapping

The 46638 nucleotide sequences or portions thereof can be used to map the location of the 46638 genes on a chromosome. This process is called chromosome mapping. Chromosome mapping is useful in correlating the 46638 sequences with genes associated with disease.

Briefly, 46638 genes can be mapped to chromosomes by preparing PCR primers (preferably 15-25 bp in length) from the 46638 nucleotide sequences. These primers can then be used for PCR screening of somatic cell hybrids containing individual human chromosomes. Only those hybrids containing the human gene corresponding to the 46638 sequences will yield an amplified fragment.

A panel of somatic cell hybrids in which each cell line contains either a single human chromosome or a small number of human chromosomes, and a full set of mouse chromosomes, can allow easy mapping of individual genes to specific human chromosomes. (D'Eustachio P. et al. (1983) Science 220:919-924).

Other mapping strategies e.g., in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries can be used to map 46638 to a chromosomal location.

Fluorescence in situ hybridization (FISH) of a DNA sequence to a metaphase chromosomal spread can further be used to provide a precise chromosomal location in one step. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Venna et al., Human Chromosomes: A Manual of Basic Techniques ((1988) Pergamon Press, New York).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the 46638 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

46638 Tissue Typing

46638 sequences can be used to identify individuals from biological samples using, e.g., restriction fragment length polymorphism (RFLP). In this technique, an individual's genomic DNA is digested with one or more restriction enzymes, the fragments separated, e.g., in a Southern blot, and probed to yield bands for identification. The sequences of the present invention are useful as additional DNA markers for RFLP (described in U.S. Pat. No. 5,272,057).

Furthermore, the sequences of the present invention can also be used to determine the actual base-by-base DNA sequence of selected portions of an individual's genome. Thus, the 46638 nucleotide sequences described herein can be used to prepare two PCR primers from the 5′ and 3′ ends of the sequences. These primers can then be used to amplify an individual's DNA and subsequently sequence it. Panels of corresponding DNA sequences from individuals, prepared in this manner, can provide unique individual identifications, as each individual will have a unique set of such DNA sequences due to allelic differences.

Allelic variation occurs to some degree in the coding regions of these sequences, and to a greater degree in the noncoding regions. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:22 can provide positive individual identification with a panel of perhaps 10 to 1,000 primers which each yield a noncoding amplified sequence of 100 bases. If predicted coding sequences, such as those in SEQ ID NO:24 are used, a more appropriate number of primers for positive individual identification would be 500-2,000.

If a panel of reagents from 46638 nucleotide sequences described herein is used to generate a unique identification database for an individual, those same reagents can later be used to identify tissue from that individual. Using the unique identification database, positive identification of the individual, living or dead, can be made from extremely small tissue samples.

Use of Partial 46638 Sequences in Forensic Biology

DNA-based identification techniques can also be used in forensic biology. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:22 (e.g., fragments derived from the noncoding regions of SEQ ID NO:22 having a length of at least 20 bases, preferably at least 30 bases) are particularly appropriate for this use.

The 46638 nucleotide sequences described herein can further be used to provide polynucleotide reagents, e.g., labeled or labelable probes which can be used in, for example, an in situ hybridization technique, to identify a specific tissue. This can be very useful in cases where a forensic pathologist is presented with a tissue of unknown origin. Panels of such 46638 probes can be used to identify tissue by species and/or by organ type.

In a similar fashion, these reagents, e.g., 46638 primers or probes can be used to screen tissue culture for contamination (i.e. screen for the presence of a mixture of different types of cells in a culture).

Predictive Medicine of 46638

The present invention also pertains to the field of predictive medicine in which diagnostic assays, prognostic assays, and monitoring clinical trials are used for prognostic (predictive) purposes to thereby treat an individual.

Generally, the invention provides, a method of determining if a subject is at risk for a disorder related to a lesion in or the misexpression of a gene which encodes 46638.

Such disorders include, e.g., a disorder associated with the excessive O-methyltransferase activity or insufficient O-methyltransferase activity

The method includes one or more of the following:

detecting, in a tissue of the subject, the presence or absence of a mutation which affects the expression of the 46638 gene, or detecting the presence or absence of a mutation in a region which controls the expression of the gene, e.g., a mutation in the 5′ control region;

detecting, in a tissue of the subject, the presence or absence of a mutation which alters the structure of the 46638 gene;

detecting, in a tissue of the subject, the misexpression of the 46638 gene, at the mRNA level, e.g., detecting a non-wild type level of a mRNA;

detecting, in a tissue of the subject, the misexpression of the gene, at the protein level, e.g., detecting a non-wild type level of a 46638 polypeptide.

In preferred embodiments the method includes: ascertaining the existence of at least one of: a deletion of one or more nucleotides from the 46638 gene; an insertion of one or more nucleotides into the gene, a point mutation, e.g., a substitution of one or more nucleotides of the gene, a gross chromosomal rearrangement of the gene, e.g., a translocation, inversion, or deletion.

For example, detecting the genetic lesion can include: (i) providing a probe/primer including an oligonucleotide containing a region of nucleotide sequence which hybridizes to a sense or antisense sequence from SEQ ID NO:22, or naturally occurring mutants thereof or 5′ or 3′ flanking sequences naturally associated with the 46638 gene; (ii) exposing the probe/primer to nucleic acid of the tissue; and detecting, by hybridization, e.g., in situ hybridization, of the probe/primer to the nucleic acid, the presence or absence of the genetic lesion.

In preferred embodiments detecting the misexpression includes ascertaining the existence of at least one of: an alteration in the level of a messenger RNA transcript of the 46638 gene; the presence of a non-wild type splicing pattern of a messenger RNA transcript of the gene; or a non-wild type level of 46638.

Methods of the invention can be used prenatally or to determine if a subject's offspring will be at risk for a disorder.

In preferred embodiments the method includes determining the structure of a 46638 gene, an abnormal structure being indicative of risk for the disorder.

In preferred embodiments the method includes contacting a sample from the subject with an antibody to the 46638 protein or a nucleic acid, which hybridizes specifically with the gene. These and other embodiments are discussed below.

Diagnostic and Prognostic Assays of 46638

Diagnostic and prognostic assays of the invention include method for assessing the expression level of 46638 molecules and for identifying variations and mutations in the sequence of 46638 molecules.

Expression Monitoring and Profiling:

The presence, level, or absence of 46638 protein or nucleic acid in a biological sample can be evaluated by obtaining a biological sample from a test subject and contacting the biological sample with a compound or an agent capable of detecting 46638 protein or nucleic acid (e.g., mRNA, genomic DNA) that encodes 46638 protein such that the presence of 46638 protein or nucleic acid is detected in the biological sample. The term “biological sample” includes tissues, cells and biological fluids isolated from a subject, as well as tissues, cells and fluids present within a subject. A preferred biological sample is serum. The level of expression of the 46638 gene can be measured in a number of ways, including, but not limited to: measuring the mRNA encoded by the 46638 genes; measuring the amount of protein encoded by the 46638 genes; or measuring the activity of the protein encoded by the 46638 genes.

The level of mRNA corresponding to the 46638 gene in a cell can be determined both by in situ and by in vitro formats.

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length 46638 nucleic acid, such as the nucleic acid of SEQ ID NO:22, or a portion thereof, such as an oligonucleotide of at least 7, 15, 30, 50, 100, 250 or 500 nucleotides in length and sufficient to specifically hybridize under stringent conditions to 46638 mRNA or genomic DNA. The probe can be disposed on an address of an array, e.g., an array described below. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array described below. A skilled artisan can adapt known mRNA detection methods for use in detecting the level of mRNA encoded by the 46638 genes.

The level of mRNA in a sample that is encoded by one of 46638 can be evaluated with nucleic acid amplification, e.g., by rtPCR (Mullis (1987) U.S. Pat. No. 4,683,202), ligase chain reaction (Barany (1991) Proc. Natl. Acad. Sci. USA 88:189-193), self sustained sequence replication (Guatelli et al., (1990) Proc. Natl. Acad. Sci. USA 87:1874-1878), transcriptional amplification system (Kwoh et al., (1989), Proc. Natl. Acad. Sci. USA 86:1173-1177), Q-Beta Replicase (Lizardi et al., (1988) Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the 46638 gene being analyzed.

In another embodiment, the methods further contacting a control sample with a compound or agent capable of detecting 46638 mRNA, or genomic DNA, and comparing the presence of 46638 mRNA or genomic DNA in the control sample with the presence of 46638 mRNA or genomic DNA in the test sample. In still another embodiment, serial analysis of gene expression, as described in U.S. Pat. No. 5,695,937, is used to detect 46638 transcript levels.

A variety of methods can be used to determine the level of protein encoded by 46638. In general, these methods include contacting an agent that selectively binds to the protein, such as an antibody with a sample, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect 46638 protein in a biological sample in vitro as well as in vivo. In vitro techniques for detection of 46638 protein include enzyme linked immunosorbent assays (ELISAs), immunoprecipitations, immunofluorescence, enzyme immunoassay (EIA), radioimmunoassay (RIA), and Western blot analysis. In vivo techniques for detection of 46638 protein include introducing into a subject a labeled anti-46638 antibody. For example, the antibody can be labeled with a radioactive marker whose presence and location in a subject can be detected by standard imaging techniques. In another embodiment, the sample is labeled, e.g., biotinylated and then contacted to the antibody, e.g., an anti-46638 antibody positioned on an antibody array (as described below). The sample can be detected, e.g., with avidin coupled to a fluorescent label.

In another embodiment, the methods further include contacting the control sample with a compound or agent capable of detecting 46638 protein, and comparing the presence of 46638 protein in the control sample with the presence of 46638 protein in the test sample.

The invention also includes kits for detecting the presence of 46638 in a biological sample. For example, the kit can include a compound or agent capable of detecting 46638 protein or mRNA in a biological sample; and a standard. The compound or agent can be packaged in a suitable container. The kit can further comprise instructions for using the kit to detect 46638 protein or nucleic acid.

For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can include: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also includes a buffering agent, a preservative, or a protein stabilizing agent. The kit can also includes components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, 1 ! along with instructions for interpreting the results of the assays performed using the kit.

The diagnostic methods described herein can identify subjects having, or at risk of developing, a disease or disorder associated with misexpressed or aberrant or unwanted 46638 expression or activity. As used herein, the term “unwanted” includes an unwanted phenomenon involved in a biological response such as pain or deregulated cell proliferation.

In one embodiment, a disease or disorder associated with aberrant or unwanted 46638 expression or activity is identified. A test sample is obtained from a subject and 46638 protein or nucleic acid (e.g., mRNA or genomic DNA) is evaluated, wherein the level, e.g., the presence or absence, of 46638 protein or nucleic acid is diagnostic for a subject having or at risk of developing a disease or disorder associated with aberrant or unwanted 46638 expression or activity. As used herein, a “test sample” refers to a biological sample obtained from a subject of interest, including a biological fluid (e.g., serum), cell sample, or tissue.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted 46638 expression or activity. For example, such methods can be used to determine whether a subject can be effectively treated with an agent for a cell proliferative or differentiative disorder.

In another aspect, the invention features a computer medium having a plurality of digitally encoded data records. Each data record includes a value representing the level of expression of 46638 in a sample, and a descriptor of the sample. The descriptor of the sample can be an identifier of the sample, a subject from which the sample was derived (e.g., a patient), a diagnosis, or a treatment (e.g., a preferred treatment). In a preferred embodiment, the data record further includes values representing the level of expression of genes other than 46638 (e.g., other genes associated with a 46638-disorder, or other genes on an array). The data record can be structured as a table, e.g., a table that is part of a database such as a relational database (e.g., a SQL database of the Oracle or Sybase database environments).

Also featured is a method of evaluating a sample. The method includes providing a sample, e.g., from the subject, and determining a gene expression profile of the sample, wherein the profile includes a value representing the level of 46638 expression. The method can further include comparing the value or the profile (i.e., multiple values) to a reference value or reference profile. The gene expression profile of the sample can be obtained by any of the methods described herein (e.g., by providing a nucleic acid from the sample and contacting the nucleic acid to an array). The method can be used to diagnose a disorder, e.g., a disorder as described herein, in a subject wherein a change in 46638 expression is an indication that the subject has or is disposed to having a disorder. The method can be used to monitor a treatment for a disorder, e.g., a disorder as described herein, in a subject. For example, the gene expression profile can be determined for a sample from a subject undergoing treatment. The profile can be compared to a reference profile or to a profile obtained from the subject prior to treatment or prior to onset of the disorder (see, e.g., Golub et al. (1999) Science 286:531).

In yet another aspect, the invention features a method of evaluating a test compound (see also, “Screening Assays”, above). The method includes providing a cell and a test compound; contacting the test compound to the cell; obtaining a subject expression profile for the contacted cell; and comparing the subject expression profile to one or more reference profiles. The profiles include a value representing the level of 46638 expression. In a preferred embodiment, the subject expression profile is compared to a target profile, e.g., a profile for a normal cell or for desired condition of a cell. The test compound is evaluated favorably if the subject expression profile is more similar to the target profile than an expression profile obtained from an uncontacted cell.

In another aspect, the invention features, a method of evaluating a subject. The method includes: a) obtaining a sample from a subject, e.g., from a caregiver, e.g., a caregiver who obtains the sample from the subject; b) determining a subject expression profile for the sample. Optionally, the method further includes either or both of steps: c) comparing the subject expression profile to one or more reference expression profiles; and d) selecting the reference profile most similar to the subject reference profile. The subject expression profile and the reference profiles include a value representing the level of 46638 expression. A variety of routine statistical measures can be used to compare two reference profiles. One possible metric is the length of the distance vector that is the difference between the two profiles. Each of the subject and reference profile is represented as a multi-dimensional vector, wherein each dimension is a value in the profile.

The method can further include transmitting a result to a caregiver. The result can be the subject expression profile, a result of a comparison of the subject expression profile with another profile, a most similar reference profile, or a descriptor of any of the aforementioned. The result can be transmitted across a computer network, e.g., the result can be in the form of a computer transmission, e.g., a computer data signal embedded in a carrier wave.

Also featured is a computer medium having executable code for effecting the following steps: receive a subject expression profile; access a database of reference expression profiles; and either i) select a matching reference profile most similar to the subject expression profile or ii) determine at least one comparison score for the similarity of the subject expression profile to at least one reference profile. The subject expression profile, and the reference expression profiles each include a value representing the level of 46638 expression.

46638 Arrays and Uses Thereof

In another aspect, the invention features an array that includes a substrate having a plurality of addresses. At least one address of the plurality includes a capture probe that binds specifically to a 46638 molecule (e.g., a 46638 nucleic acid or a 46638 polypeptide). The array can have a density of at least than 10, 50, 100, 200, 500, 1,000, 2,000, or 10,000 or more addresses/cm², and ranges between. In a preferred embodiment, the plurality of addresses includes at least 10, 100, 500, 1,000, 5,000, 10,000, 50,000 addresses. In a preferred embodiment, the plurality of addresses includes equal to or less than 10, 100, 500, 1,000, 5,000, 10,000, or 50,000 addresses. The substrate can be a two-dimensional substrate such as a glass slide, a wafer (e.g., silica or plastic), a mass spectroscopy plate, or a three-dimensional substrate such as a gel pad. Addresses in addition to address of the plurality can be disposed on the array.

In a preferred embodiment, at least one address of the plurality includes a nucleic acid capture probe that hybridizes specifically to a 46638 nucleic acid, e.g., the sense or anti-sense strand. In one preferred embodiment, a subset of addresses of the plurality of addresses has a nucleic acid capture probe for 46638. Each address of the subset can include a capture probe that hybridizes to a different region of a 46638 nucleic acid. In another preferred embodiment, addresses of the subset include a capture probe for a 46638 nucleic acid. Each address of the subset is unique, overlapping, and complementary to a different variant of 46638 (e.g., an allelic variant, or all possible hypothetical variants). The array can be used to sequence 46638 by hybridization (see, e.g., U.S. Pat. No. 5,695,940).

An array can be generated by various methods, e.g., by photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145).

In another preferred embodiment, at least one address of the plurality includes a polypeptide capture probe that binds specifically to a 46638 polypeptide or fragment thereof. The polypeptide can be a naturally-occurring interaction partner of 46638 polypeptide. Preferably, the polypeptide is an antibody, e.g., an antibody described herein (see “Anti-46638 Antibodies,” above), such as a monoclonal antibody or a single-chain antibody.

In another aspect, the invention features a method of analyzing the expression of 46638. The method includes providing an array as described above; contacting the array with a sample and detecting binding of a 46638-molecule (e.g., nucleic acid or polypeptide) to the array. In a preferred embodiment, the array is a nucleic acid array. Optionally the method further includes amplifying nucleic acid from the sample prior or during contact with the array.

In another embodiment, the array can be used to assay gene expression in a tissue to ascertain tissue specificity of genes in the array, particularly the expression of 46638. If a sufficient number of diverse samples is analyzed, clustering (e.g., hierarchical clustering, k-means clustering, Bayesian clustering and the like) can be used to identify other genes which are co-regulated with 46638. For example, the array can be used for the quantitation of the expression of multiple genes. Thus, not only tissue specificity, but also the level of expression of a battery of genes in the tissue is ascertained. Quantitative data can be used to group (e.g., cluster) genes on the basis of their tissue expression per se and level of expression in that tissue.

For example, array analysis of gene expression can be used to assess the effect of cell-cell interactions on 46638 expression. A first tissue can be perturbed and nucleic acid from a second tissue that interacts with the first tissue can be analyzed. In this context, the effect of one cell type on another cell type in response to a biological stimulus can be determined, e.g., to monitor the effect of cell-cell interaction at the level of gene expression.

In another embodiment, cells are contacted with a therapeutic agent. The expression profile of the cells is determined using the array, and the expression profile is compared to the profile of like cells not contacted with the agent. For example, the assay can be used to determine or analyze the molecular basis of an undesirable effect of the therapeutic agent. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor expression of one or more genes in the array with respect to time. For example, samples obtained from different time points can be probed with the array. Such analysis can identify and/or characterize the development of a 46638-associated disease or disorder; and processes, such as a cellular transformation associated with a 46638-associated disease or disorder. The method can also evaluate the treatment and/or progression of a 46638-associated disease or disorder

The array is also useful for ascertaining differential expression patterns of one or more genes in normal and abnormal cells. This provides a battery of genes (e.g., including 46638) that could serve as a molecular target for diagnosis or therapeutic intervention.

In another aspect, the invention features an array having a plurality of addresses. Each address of the plurality includes a unique polypeptide. At least one address of the plurality has disposed thereon a 46638 polypeptide or fragment thereof. Methods of producing polypeptide arrays are described in the art, e.g., in De Wildt et al. (2000). Nature Biotech. 18, 989-994; Lueking et al. (1999). Anal. Biochem. 270, 103-111; Ge, H. (2000). Nucleic Acids Res. 28, e3, I-VII; MacBeath, G., and Schreiber, S. L. (2000). Science 289, 1760-1763; and WO 99/51773A1. In a preferred embodiment, each addresses of the plurality has disposed thereon a polypeptide at least 60, 70, 80,85, 90, 95 or 99% identical to a 46638 polypeptide or fragment thereof. For example, multiple variants of a 46638 polypeptide (e.g., encoded by allelic variants, site-directed mutants, random mutants, or combinatorial mutants) can be disposed at individual addresses of the plurality. Addresses in addition to the address of the plurality can be disposed on the array.

The polypeptide array can be used to detect a 46638 binding compound, e.g., an antibody in a sample from a subject with specificity for a 46638 polypeptide or the presence of a 46638-binding protein or ligand.

The array is also useful for ascertaining the effect of the expression of a gene on the expression of other genes in the same cell or in different cells (e.g., ascertaining the effect of 46638 expression on the expression of other genes). This provides, for example, for a selection of alternate molecular targets for therapeutic intervention if the ultimate or downstream target cannot be regulated.

In another aspect, the invention features a method of analyzing a plurality of probes. The method is useful, e.g., for analyzing gene expression. The method includes: providing a two dimensional array having a plurality of addresses, each address of the plurality being positionally distinguishable from each other address of the plurality having a unique capture probe, e.g., wherein the capture probes are from a cell or subject which express 46638 or from a cell or subject in which a 46638 mediated response has been elicited, e.g., by contact of the cell with 46638 nucleic acid or protein, or administration to the cell or subject 46638 nucleic acid or protein; providing a two dimensional array having a plurality of addresses, each address of the plurality being positionally distinguishable from each other address of the plurality, and each address of the plurality having a unique capture probe, e.g., wherein the capture probes are from a cell or subject which does not express 46638 (or does not express as highly as in the case of the 46638 positive plurality of capture probes) or from a cell or subject which in which a 46638 mediated response has not been elicited (or has been elicited to a lesser extent than in the first sample); contacting the array with one or more inquiry probes (which is preferably other than a 46638 nucleic acid, polypeptide, or antibody), and thereby evaluating the plurality of capture probes. Binding, e.g., in the case of a nucleic acid, hybridization with a capture probe at an address of the plurality, is detected, e.g., by signal generated from a label attached to the nucleic acid, polypeptide, or antibody.

In another aspect, the invention features a method of analyzing a plurality of probes or a sample. The method is useful, e.g., for analyzing gene expression. The method includes: providing a two dimensional array having a plurality of addresses, each address of the plurality being positionally distinguishable from each other address of the plurality having a unique capture probe, contacting the array with a first sample from a cell or subject which express or mis-express 46638 or from a cell or subject in which a 46638-mediated response has been elicited, e.g., by contact of the cell with 46638 nucleic acid or protein, or administration to the cell or subject 46638 nucleic acid or protein; providing a two dimensional array having a plurality of addresses, each address of the plurality being positionally distinguishable from each other address of the plurality, and each address of the plurality having a unique capture probe, and contacting the array with a second sample from a cell or subject which does not express 46638 (or does not express as highly as in the case of the 46638 positive plurality of capture probes) or from a cell or subject which in which a 46638 mediated response has not been elicited (or has been elicited to a lesser extent than in the first sample); and comparing the binding of the first sample with the binding of the second sample. Binding, e.g., in the case of a nucleic acid, hybridization with a capture probe at an address of the plurality, is detected, e.g., by signal generated from a label attached to the nucleic acid, polypeptide, or antibody. The same array can be used for both samples or different arrays can be used. If different arrays are used the plurality of addresses with capture probes should be present on both arrays.

In another aspect, the invention features a method of analyzing 46638, e.g., analyzing structure, function, or relatedness to other nucleic acid or amino acid sequences. The method includes: providing a 46638 nucleic acid or amino acid sequence; comparing the 46638 sequence with one or more preferably a plurality of sequences from a collection of sequences, e.g., a nucleic acid or protein sequence database; to thereby analyze 46638.

Detection of 46638 Variations or Mutations

The methods of the invention can also be used to detect genetic alterations in a 46638 gene, thereby determining if a subject with the altered gene is at risk for a disorder characterized by misregulation in 46638 protein activity or nucleic acid expression, such as a organogenetic, blood coagulative or immunological disorder. In preferred embodiments, the methods include detecting, in a sample from the subject, the presence or absence of a genetic alteration characterized by at least one of an alteration affecting the integrity of a gene encoding a 46638-protein, or the mis-expression of the 46638 gene. For example, such genetic alterations can be detected by ascertaining the existence of at least one of 1) a deletion of one or more nucleotides from a 46638 gene; 2) an addition of one or more nucleotides to a 46638 gene; 3) a substitution of one or more nucleotides of a 46638 gene, 4) a chromosomal rearrangement of a 46638 gene; 5) an alteration in the level of a messenger RNA transcript of a 46638 gene, 6) aberrant modification of a 46638 gene, such as of the methylation pattern of the genomic DNA, 7) the presence of a non-wild type splicing pattern of a messenger RNA transcript of a 46638 gene, 8) a non-wild type level of a 46638-protein, 9) allelic loss of a 46638 gene, and 10) inappropriate post-translational modification of a 46638-protein.

An alteration can be detected without a probe/primer in a polymerase chain reaction, such as anchor PCR or RACE PCR, or, alternatively, in a ligation chain reaction (LCR), the latter of which can be particularly useful for detecting point mutations in the 46638-gene. This method can include the steps of collecting a sample of cells from a subject, isolating nucleic acid (e.g., genomic, mRNA or both) from the sample, contacting the nucleic acid sample with one or more primers which specifically hybridize to a 46638 gene under conditions such that hybridization and amplification of the 46638-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein. Alternatively, other amplification methods described herein or known in the art can be used.

In another embodiment, mutations in a 46638 gene from a sample cell can be identified by detecting alterations in restriction enzyme cleavage patterns. For example, sample and control DNA is isolated, amplified (optionally), digested with one or more restriction endonucleases, and fragment length sizes are determined, e.g., by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 46638 can be identified by hybridizing a sample and control nucleic acids, e.g., DNA or RNA, two-dimensional arrays, e.g., chip based arrays. Such arrays include a plurality of addresses, each of which is positionally distinguishable from the other. A different probe is located at each address of the plurality. A probe can be complementary to a region of a 46638 nucleic acid or a putative variant (e.g., allelic variant) thereof. A probe can have one or more mismatches to a region of a 46638 nucleic acid (e.g., a destabilizing mismatch). The arrays can have a high density of addresses, e.g., can contain hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in 46638 can be identified in two-dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 46638 gene and detect mutations by comparing the sequence of the sample 46638 with the corresponding wild-type (control) sequence. Automated sequencing procedures can be utilized when performing the diagnostic assays ((1995) Biotechniques 19:448), including sequencing by mass spectrometry.

Other methods for detecting mutations in the 46638 gene include methods in which protection from cleavage agents is used to detect mismatched bases in RNA/RNA or RNA/DNA heteroduplexes (Myers et al. (1985) Science 230:1242; Cotton et al. (1988) Proc. NatlAcadSci USA 85:4397; Saleeba et al. (1992) Methods Enzymol 217:286-295).

In still another embodiment, the mismatch cleavage reaction employs one or more proteins that recognize mismatched base pairs in double-stranded DNA (so called “DNA mismatch repair” enzymes) in defined systems for detecting and mapping point mutations in 46638 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662; U.S. Pat. No. 5,459,039).

In other embodiments, alterations in electrophoretic mobility will be used to identify mutations in 46638 genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control 46638 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension (Saiki et al. (1986) Nature 324:163); Saiki et al. (1989) Proc. Natl Acad. Sci USA 86:6230). A further method of detecting point mutations is the chemical ligation of oligonucleotides as described in Xu et al. ((2001) Nature Biotechnol. 19:148). Adjacent oligonucleotides, one of which selectively anneals to the query site, are ligated together if the nucleotide at the query site of the sample nucleic acid is complementary to the query oligonucleotide; ligation can be monitored, e.g., by fluorescent dyes coupled to the oligonucleotides.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

In another aspect, the invention features a set of oligonucleotides. The set includes a plurality of oligonucleotides, each of which is at least partially complementary (e.g., at least 50%, 60%, 70%, 80%, 90%, 92%, 95%, 97%, 98%, or 99% complementary) to a 46638 nucleic acid.

In a preferred embodiment the set includes a first and a second oligonucleotide. The first and second oligonucleotide can hybridize to the same or to different locations of SEQ ID NO:22 or the complement of SEQ ID NO:22. Different locations can be different but overlapping, or non-overlapping on the same strand. The first and second oligonucleotide can hybridize to sites on the same or on different strands.

The set can be useful, e.g., for identifying SNP's, or identifying specific alleles of 46638. In a preferred embodiment, each oligonucleotide of the set has a different nucleotide at an interrogation position. In one embodiment, the set includes two oligonucleotides, each complementary to a different allele at a locus, e.g., a biallelic or polymorphic locus.

In another embodiment, the set includes four oligonucleotides, each having a different nucleotide (e.g., adenine, guanine, cytosine, or thymidine) at the interrogation position. The interrogation position can be a SNP or the site of a mutation. In another preferred embodiment, the oligonucleotides of the plurality are identical in sequence to one another (except for differences in length). The oligonucleotides can be provided with differential labels, such that an oligonucleotide that hybridizes to one allele provides a signal that is distinguishable from an oligonucleotide that hybridizes to a second allele. In still another embodiment, at least one of the oligonucleotides of the set has a nucleotide change at a position in addition to a query position, e.g., a destabilizing mutation to decrease the Tm of the oligonucleotide. In another embodiment, at least one oligonucleotide of the set has a non-natural nucleotide, e.g., inosine. In a preferred embodiment, the oligonucleotides are attached to a solid support, e.g., to different addresses of an array or to different beads or nanoparticles.

In a preferred embodiment the set of oligo nucleotides can be used to specifically amplify, e.g., by PCR, or detect, a 46638 nucleic acid.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a 46638 gene.

Use of 46638 Molecules as Surrogate Markers

The 46638 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the 46638 molecules of the invention may be detected, and may be correlated with one or more biological states in vivo. For example, the 46638 molecules of the invention may serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The 46638 molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., a 46638 marker) transcription or expression, the amplified marker may be in a quantity which is more readily detectable than the drug itself. Also, the marker may be more easily detected due to the nature of the marker itself; for example, using the methods described herein, anti-46638 antibodies may be employed in an immune-based detection system for a 46638 protein marker, or 46638-specific radiolabeled probes may be used to detect a 46638 mRNA marker. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am, J Health-Syst. Pharm. 56 Suppl. 3: S16-S20.

The 46638 molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35:1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or protein (e.g., 46638 protein or RNA) for specific tumor markers in a subject, a drug or course of treatment may be selected that is optimized for the treatment of the specific tumor likely to be present in the subject. Similarly, the presence or absence of a specific sequence mutation in 46638 DNA may correlate 46638 drug response. The use of pharmacogenomic markers therefore permits the application of the most appropriate treatment for each subject without having to administer the therapy.

Pharmaceutical Compositions of 46638

The nucleic acid and polypeptides, fragments thereof, as well as anti-46638 antibodies (also referred to herein as “active compounds”) of the invention can be incorporated into pharmaceutical compositions. Such compositions typically include the nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELT™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

For antibodies, the preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The present invention encompasses agents which modulate expression or activity. An agent may, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e.,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

An antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Methods of Treatment for 46638

The present invention provides for both prophylactic and therapeutic methods of treating a subject at risk of (or susceptible to) a disorder or having a disorder associated with aberrant or unwanted 46638 expression or activity. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides.

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the 46638 molecules of the present invention or 46638 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

In one aspect, the invention provides a method for preventing in a subject, a disease or condition associated with an aberrant or unwanted 46638 expression or activity, by administering to the subject a 46638 or an agent which modulates 46638 expression or at least one 46638 activity. Subjects at risk for a disease which is caused or contributed to by aberrant or unwanted 46638 expression or activity can be identified by, for example, any or a combination of diagnostic or prognostic assays as described herein. Administration of a prophylactic agent can occur prior to the manifestation of symptoms characteristic of the 46638 aberrance, such that a disease or disorder is prevented or, alternatively, delayed in its progression. Depending on the type of 46638 aberrance, for example, a 46638, 46638 agonist or 46638 antagonist agent can be used for treating the subject. The appropriate agent can be determined based on screening assays described herein.

It is possible that some 46638 disorders can be caused, at least in part, by an abnormal level of gene product, or by the presence of a gene product exhibiting abnormal activity. As such, the reduction in the level and/or activity of such gene products would bring about the amelioration of disorder symptoms.

The 46638 molecules can act as novel diagnostic targets and therapeutic agents for controlling one or more of cellular proliferative and/or differentiative disorders, immune disorders and neurological disorders as described above, as well as disorders associated with bone metabolism, cardiovascular disorders, liver disorders, viral diseases, pain or metabolic disorders.

Aberrant expression and/or activity of 46638 molecules may mediate disorders associated with bone metabolism. “Bone metabolism” refers to direct or indirect effects in the formation or degeneration of bone structures, e.g., bone formation, bone resorption, etc., which may ultimately affect the concentrations in serum of calcium and phosphate. This term also includes activities mediated by 46638 molecules effects in bone cells, e.g. osteoclasts and osteoblasts, that may in turn result in bone formation and degeneration. For example, 46638 molecules may support different activities of bone resorbing osteoclasts such as the stimulation of differentiation of monocytes and mononuclear phagocytes into osteoclasts. Accordingly, 46638 molecules that modulate the production of bone cells can influence bone formation and degeneration, and thus may be used to treat bone disorders. Examples of such disorders include, but are not limited to, osteoporosis, osteodystrophy, osteomalacia, rickets, osteitis fibrosa cystica, renal osteodystrophy, osteosclerosis, anti-convulsant treatment, osteopenia, fibrogenesis-imperfecta ossium, secondary hyperparathyrodism, hypoparathyroidism, hyperparathyroidism, cirrhosis, obstructive jaundice, drug induced metabolism, medullary carcinoma, chronic renal disease, rickets, sarcoidosis, glucocorticoid antagonism, malabsorption syndrome, steatorrhea, tropical sprue, idiopathic hypercalcemia and milk fever.

Additionally, 46638 molecules may play an important role in the etiology of certain viral diseases, including but not limited to Hepatitis B, Hepatitis C and Herpes Simplex Virus (HSV). Modulators of 46638 activity could be used to control viral diseases. The modulators can be used in the treatment and/or diagnosis of viral infected tissue or virus-associated tissue fibrosis, especially liver and liver fibrosis. Also, 46638 modulators can be used in the treatment and/or diagnosis of virus-associated carcinoma, especially hepatocellular cancer.

Additionally, 46638 may play an important role in the regulation of metabolism or pain disorders. Diseases of metabolic imbalance include, but are not limited to, obesity, anorexia nervosa, cachexia, lipid disorders, and diabetes. Examples of pain disorders include, but are not limited to, pain response elicited during various forms of tissue injury, e.g., inflammation, infection, and ischemia, usually referred to as hyperalgesia (described in, for example, Fields, H. L. (1987) Pain, New York:McGraw-Hill); pain associated with musculoskeletal disorders, e.g., joint pain; tooth pain; headaches; pain associated with surgery; pain related to irritable bowel syndrome; or chest pain.

Examples of disorders involving the heart or “cardiovascular disorder” include, but are not limited to, a disease, disorder, or state involving the cardiovascular system, e.g., the heart, the blood vessels, and/or the blood. A cardiovascular disorder can be caused by an imbalance in arterial pressure, a malfunction of the heart, or an occlusion of a blood vessel, e.g., by a thrombus. Examples of such disorders include hypertension, atherosclerosis, coronary artery spasm, congestive heart failure, coronary artery disease, valvular disease, arrhythmias, and cardiomyopathies.

Disorders which may be treated or diagnosed by methods described herein include, but are not limited to, disorders associated with an accumulation in the liver of fibrous tissue, such as that resulting from an imbalance between production and degradation of the extracellular matrix accompanied by the collapse and condensation of preexisting fibers. The methods described herein can be used to diagnose or treat hepatocellular necrosis or injury induced by a wide variety of agents including processes which disturb homeostasis, such as an inflammatory process, tissue damage resulting from toxic injury or altered hepatic blood flow, and infections (e.g., bacterial, viral and parasitic). For example, the methods can be used for the early detection of hepatic injury, such as portal hypertension or hepatic fibrosis. In addition, the methods can be employed to detect liver fibrosis attributed to inborn errors of metabolism, for example, fibrosis resulting from a storage disorder such as Gaucher's disease (lipid abnormalities) or a glycogen storage disease, Al-antitrypsin deficiency; a disorder mediating the accumulation (e.g., storage) of an exogenous substance, for example, hemochromatosis (iron-overload syndrome) and copper storage diseases (Wilson's disease), disorders resulting in the accumulation of a toxic metabolite (e.g., tyrosinemia, fructosemia and galactosemia) and peroxisomal disorders (e.g., Zellweger syndrome). Additionally, the methods described herein may be useful for the early detection and treatment of liver injury associated with the administration of various chemicals or drugs, such as for example, methotrexate, isonizaid, oxyphenisatin, methyldopa, chlorpromazine, tolbutamide or alcohol, or which represents a hepatic manifestation of a vascular disorder such as obstruction of either the intrahepatic or extrahepatic bile flow or an alteration in hepatic circulation resulting, for example, from chronic heart failure, veno-occlusive disease, portal vein thrombosis or Budd-Chiari syndrome.

As discussed, successful treatment of 46638 disorders can be brought about by techniques that serve to inhibit the expression or activity of target gene products. For example, compounds, e.g., an agent identified using an assays described above, that proves to exhibit negative modulatory activity, can be used in accordance with the invention to prevent and/or ameliorate symptoms of 46638 disorders. Such molecules can include, but are not limited to peptides, phosphopeptides, small organic or inorganic molecules, or antibodies (including, for example, polyclonal, monoclonal, humanized, anti-idiotypic, chimeric or single chain antibodies, and Fab, F(ab′)₂ and Fab expression library fragments, scFV molecules, and epitope-binding fragments thereof).

Further, antisense and ribozyme molecules that inhibit expression of the target gene can also be used in accordance with the invention to reduce the level of target gene expression, thus effectively reducing the level of target gene activity. Still further, triple helix molecules can be utilized in reducing the level of target gene activity. Antisense, ribozyme and triple helix molecules are discussed above.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances in that the target gene encodes an extracellular protein, it can be preferable to co-administer normal target gene protein into the cell or tissue in order to maintain the requisite level of cellular or tissue target gene activity.

Another method by which nucleic acid molecules may be utilized in treating or preventing a disease characterized by 46638 expression is through the use of aptamer molecules specific for 46638 protein. Aptamers are nucleic acid molecules having a tertiary structure which permits them to specifically bind to protein ligands (see, e.g., Osborne, et al. (1997) Curr. Opin. Chem Biol. 1: 5-9; and Patel, D. J. (1997) Curr Opin Chem Biol 1:32-46). Since nucleic acid molecules may in many cases be more conveniently introduced into target cells than therapeutic protein molecules may be, aptamers offer a method by which 46638 protein activity may be specifically decreased without the introduction of drugs or other molecules which may have pluripotent effects.

Antibodies can be generated that are both specific for target gene product and that reduce target gene product activity. Such antibodies may, therefore, by administered in instances whereby negative modulatory techniques are appropriate for the treatment of 46638 disorders. For a description of antibodies, see the Antibody section above.

In circumstances wherein injection of an animal or a human subject with a 46638 protein or epitope for stimulating antibody production is harmful to the subject, it is possible to generate an immune response against 46638 through the use of anti-idiotypic antibodies (see, for example, Herlyn, D. (1999) Ann Med 31:66-78; and Bhattacharya-Chatterjee, M., and Foon, K. A. (1998) Cancer Treat Res. 94:51-68). If an anti-idiotypic antibody is introduced into a mammal or human subject, it should stimulate the production of anti-anti-idiotypic antibodies, which should be specific to the 46638 protein. Vaccines directed to a disease characterized by 46638 expression may also be generated in this fashion.

In instances where the target antigen is intracellular and whole antibodies are used, internalizing antibodies may be preferred. Lipofectin or liposomes can be used to deliver the antibody or a fragment of the Fab region that binds to the target antigen into cells. Where fragments of the antibody are used, the smallest inhibitory fragment that binds to the target antigen is preferred. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al. (1993) Proc. Natl. Acad. Sci. USA 90:7889-7893).

The identified compounds that inhibit target gene expression, synthesis and/or activity can be administered to a patient at therapeutically effective doses to prevent, treat or ameliorate 46638 disorders. A therapeutically effective dose refers to that amount of the compound sufficient to result in amelioration of symptoms of the disorders. Toxicity and therapeutic efficacy of such compounds can be determined by standard pharmaceutical procedures as described above.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays may utilize antibody mimics and/or “biosensors” that have been created through molecular imprinting techniques. The compound which is able to modulate 46638 activity is used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell, R. J. et al (1996) Current Opinion in Biotechnology 7:89-94 and in Shea, K. J. (1994) Trends in Polymer Science 2:166-173. Such “imprinted” affinity matrixes are amenable to ligand-binding assays, whereby the immobilized monoclonal antibody component is replaced by an appropriately imprinted matrix. An example of the use of such matrixes in this way can be seen in Vlatakis, G. et al (1993) Nature 361:645-647. Through the use of isotope-labeling, the “free” concentration of compound which modulates the expression or activity of 46638 can be readily monitored and used in calculations of IC₅₀.

Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. An rudimentary example of such a “biosensor” is discussed in Kriz, D. et al (1995) Analytical Chemistry 67:2142-2144.

Another aspect of the invention pertains to methods of modulating 46638 expression or activity for therapeutic purposes. Accordingly, in an exemplary embodiment, the modulatory method of the invention involves contacting a cell with a 46638 or agent that modulates one or more of the activities of 46638 protein activity associated with the cell. An agent that modulates 46638 protein activity can be an agent as described herein, such as a nucleic acid or a protein, a naturally-occurring target molecule of a 46638 protein (e.g., a 46638 substrate or receptor), a 46638 antibody, a 46638 agonist or antagonist, a peptidomimetic of a 46638 agonist or antagonist, or other small molecule.

In one embodiment, the agent stimulates one or 46638 activities. Examples of such stimulatory agents include active 46638 protein and a nucleic acid molecule encoding 46638. In another embodiment, the agent inhibits one or more 46638 activities. Examples of such inhibitory agents include antisense 46638 nucleic acid molecules, anti-46638 antibodies, and 46638 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 46638 protein or nucleic acid molecule. In one embodiment, the method involves administering an agent (e.g., an agent identified by a screening assay described herein), or combination of agents that modulates (e.g., up regulates or down regulates) 46638 expression or activity. In another embodiment, the method involves administering a 46638 protein or nucleic acid molecule as therapy to compensate for reduced, aberrant, or unwanted 46638 expression or activity.

Stimulation of 46638 activity is desirable in situations in which 46638 is abnormally downregulated and/or in which increased 46638 activity is likely to have a beneficial effect. For example, stimulation of 46638 activity is desirable in situations in which a 46638 is downregulated and/or in which increased 46638 activity is likely to have a beneficial effect. Likewise, inhibition of 46638 activity is desirable in situations in which 46638 is abnormally upregulated and/or in which decreased 46638 activity is likely to have a beneficial effect.

46638 Pharmacogenomics

The 46638 molecules of the present invention, as well as agents, or modulators which have a stimulatory or inhibitory effect on 46638 activity (e.g., 46638 gene expression) as identified by a screening assay described herein can be administered to individuals to treat (prophylactically or therapeutically) 46638 associated disorders associated with aberrant or unwanted 46638 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a 46638 molecule or 46638 modulator as well as tailoring the dosage and/or therapeutic regimen of treatment with a 46638 molecule or 46638 modulator.

Pharmacogenomics deals with clinically significant hereditary variations in the response to drugs due to altered drug disposition and abnormal action in affected persons. See, for example, Eichelbaum, M. et al. (1996) Clin. Exp. PharmacoL Physiol. 23:983-985 and Linder, M. W. et al. (1997) Clin. Chem. 43:254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach,” can be utilized to identify genes that predict drug response. According to this method, if a gene that encodes a drug's target is known (e.g., a 46638 protein of the present invention), all common variants of that gene can be fairly easily identified in the population and it can be determined if having one version of the gene versus another is associated with a particular drug response.

Alternatively, a method termed the “gene expression profiling,” can be utilized to identify genes that predict drug response. For example, the gene expression of an animal dosed with a drug (e.g., a 46638 molecule or 46638 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a 46638 molecule or 46638 modulator, such as a modulator identified by one of the exemplary screening assays described herein.

The present invention further provides methods for identifying new agents, or combinations, that are based on identifying agents that modulate the activity of one or more of the gene products encoded by one or more of the 46638 genes of the present invention, wherein these products may be associated with resistance of the cells to a therapeutic agent. Specifically, the activity of the proteins encoded by the 46638 genes of the present invention can be used as a basis for identifying agents for overcoming agent resistance. By blocking the activity of one or more of the resistance proteins, target cells, e.g., human cells, will become sensitive to treatment with an agent that the unmodified target cells were resistant to.

Monitoring the influence of agents (e.g., drugs) on the expression or activity of a 46638 protein can be applied in clinical trials. For example, the effectiveness of an agent determined by a screening assay as described herein to increase 46638 gene expression, protein levels, or upregulate 46638 activity, can be monitored in clinical trials of subjects exhibiting decreased 46638 gene expression, protein levels, or downregulated 46638 activity. Alternatively, the effectiveness of an agent determined by a screening assay to decrease 46638 gene expression, protein levels, or downregulate 46638 activity, can be monitored in clinical trials of subjects exhibiting increased 46638 gene expression, protein levels, or upregulated 46638 activity. In such clinical trials, the expression or activity of a 46638 gene, and preferably, other genes that have been implicated in, for example, a 46638-associated disorder can be used as a “read out” or markers of the phenotype of a particular cell.

46638 Informatics

The sequence of a 46638 molecule is provided in a variety of media to facilitate use thereof. A sequence can be provided as a manufacture, other than an isolated nucleic acid or amino acid molecule, which contains a 46638. Such a manufacture can provide a nucleotide or amino acid sequence, e.g., an open reading frame, in a form which allows examination of the manufacture using means not directly applicable to examining the nucleotide or amino acid sequences, or a subset thereof, as they exists in nature or in purified form. The sequence information can include, but is not limited to, 46638 full-length nucleotide and/or amino acid sequences, partial nucleotide and/or amino acid sequences, polymorphic sequences including single nucleotide polymorphisms (SNPs), epitope sequence, and the like. In a preferred embodiment, the manufacture is a machine-readable medium, e.g., a magnetic, optical, chemical or mechanical information storage device.

As used herein, “machine-readable media” refers to any medium that can be read and accessed directly by a machine, e.g., a digital computer or analogue computer. Non-limiting examples of a computer include a desktop PC, laptop, mainframe, server (e.g., a web server, network server, or server farm), handheld digital assistant, pager, mobile telephone, and the like. The computer can be stand-alone or connected to a communications network, e.g., a local area network (such as a VPN or intranet), a wide area network (e.g., an Extranet or the Internet), or a telephone network (e.g., a wireless, DSL, or ISDN network). Machine-readable media include, but are not limited to: magnetic storage media, such as floppy discs, hard disc storage medium, and magnetic tape; optical storage media such as CD-ROM; electrical storage media such as RAM, ROM, EPROM, EEPROM, flash memory, and the like; and hybrids of these categories such as magnetic/optical storage media.

A variety of data storage structures are available to a skilled artisan for creating a machine-readable medium having recorded thereon a nucleotide or amino acid sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. The skilled artisan can readily adapt any number of data processor structuring formats (e.g., text file or database) in order to obtain computer readable medium having recorded thereon the nucleotide sequence information of the present invention.

In a preferred embodiment, the sequence information is stored in a relational database (such as Sybase or Oracle). The database can have a first table for storing sequence (nucleic acid and/or amino acid sequence) information. The sequence information can be stored in one field (e.g., a first column) of a table row and an identifier for the sequence can be store in another field (e.g., a second column) of the table row. The database can have a second table, e.g., storing annotations. The second table can have a field for the sequence identifier, a field for a descriptor or annotation text (e.g., the descriptor can refer to a functionality of the sequence, a field for the initial position in the sequence to which the annotation refers, and a field for the ultimate position in the sequence to which the annotation refers. Non-limiting examples for annotation to nucleic acid sequences include polymorphisms (e.g., SNP's) translational regulatory sites and splice junctions. Non-limiting examples for annotations to amino acid sequence include polypeptide domains, e.g., a domain described herein; active sites and other functional amino acids; and modification sites.

By providing the nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or target structural motif with the sequence information stored within the data storage means. A search is used to identify fragments or regions of the sequences of the invention which match a particular target sequence or target motif. The search can be a BLAST search or other routine sequence comparison, e.g., a search described herein.

Thus, in one aspect, the invention features a method of analyzing 46638, e.g., analyzing structure, function, or relatedness to one or more other nucleic acid or amino acid sequences. The method includes: providing a 46638 nucleic acid or amino acid sequence; comparing the 46638 sequence with a second sequence, e.g., one or more preferably a plurality of sequences from a collection of sequences, e.g., a nucleic acid or protein sequence database to thereby analyze 46638. The method can be performed in a machine, e.g., a computer, or manually by a skilled artisan.

The method can include evaluating the sequence identity between a 46638 sequence and a database sequence. The method can be performed by accessing the database at a second site, e.g., over the Internet.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. Typical sequence lengths of a target sequence are from about 10 to 100 amino acids or from about 30 to 300 nucleotide residues. However, it is well recognized that commercially important fragments, such as sequence fragments involved in gene expression and protein processing, may be of shorter length.

Computer software is publicly available which allows a skilled artisan to access sequence information provided in a computer readable medium for analysis and comparison to other sequences. A variety of known algorithms are disclosed publicly and a variety of commercially available software for conducting search means are and can be used in the computer-based systems of the present invention. Examples of such software include, but are not limited to, MacPattern (EMBL), BLASTN and BLASTX (NCBI).

Thus, the invention features a method of making a computer readable record of a sequence of a 46638 sequence which includes recording the sequence on a computer readable matrix. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′ end of the translated region.

In another aspect, the invention features, a method of analyzing a sequence. The method includes: providing a 46638 sequence, or record, in machine-readable form; comparing a second sequence to the 46638 sequence; thereby analyzing a sequence. Comparison can include comparing to sequences for sequence identity or determining if one sequence is included within the other, e.g., determining if the 46638 sequence includes a sequence being compared. In a preferred embodiment the 46638 or second sequence is stored on a first computer, e.g., at a first site and the comparison is performed, read, or recorded on a second computer, e.g., at a second site. E.g., the 46638 or second sequence can be stored in a public or proprietary database in one computer, and the results of the comparison performed, read, or recorded on a second computer. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′ end of the translated region.

In another aspect, the invention provides a machine-readable medium for holding instructions for performing a method for determining whether a subject has a 46638-associated disease or disorder or a pre-disposition to a 46638-associated disease or disorder, wherein the method comprises the steps of determining 46638 sequence information associated with the subject and based on the 46638 sequence information, determining whether the subject has a 46638-associated disease or disorder or a pre-disposition to a 46638-associated disease or disorder and/or recommending a particular treatment for the disease, disorder or pre-disease condition.

The invention further provides in an electronic system and/or in a network, a method for determining whether a subject has a 46638-associated disease or disorder or a pre-disposition to a disease associated with a 46638 wherein the method comprises the steps of determining 46638 sequence information associated with the subject, and based on the 46638 sequence information, determining whether the subject has a 46638-associated disease or disorder or a pre-disposition to a 46638-associated disease or disorder, and/or recommending a particular treatment for the disease, disorder or pre-disease condition. In a preferred embodiment, the method further includes the step of receiving information, e.g., phenotypic or genotypic information, associated with the subject and/or acquiring from a network phenotypic information associated with the subject. The information can be stored in a database, e.g., a relational database. In another embodiment, the method further includes accessing the database, e.g., for records relating to other subjects, comparing the 46638 sequence of the subject to the 46638 sequences in the database to thereby determine whether the subject as a 46638-associated disease or disorder, or a pre-disposition for such.

The present invention also provides in a network, a method for determining whether a subject has a 46638 associated disease or disorder or a pre-disposition to a 46638-associated disease or disorder associated with 46638, said method comprising the steps of receiving 46638 sequence information from the subject and/or information related thereto, receiving phenotypic information associated with the subject, acquiring information from the network corresponding to 46638 and/or corresponding to a 46638-associated disease or disorder (e.g., cellular proliferative and/or differentiative disorders), and based on one or more of the phenotypic information, the 46638 information (e.g., sequence information and/or information related thereto), and the acquired information, determining whether the subject has a 46638-associated disease or disorder or a pre-disposition to a 46638-associated disease or disorder. The method may further comprise the step of recommending a particular treatment for the disease, disorder or pre-disease condition.

The present invention also provides a method for determining whether a subject has a 46638-associated disease or disorder or a pre-disposition to a 46638-associated disease or disorder, said method comprising the steps of receiving information related to 46638 (e.g., sequence information and/or information related thereto), receiving phenotypic information associated with the subject, acquiring information from the network related to 46638 and/or related to a 46638-associated disease or disorder, and based on one or more of the phenotypic information, the 46638 information, and the acquired information, determining whether the subject has a 46638-associated disease or disorder or a pre-disposition to a 46638-associated disease or disorder. The method may further comprise the step of recommending a particular treatment for the disease, disorder or pre-disease condition.

This invention is further illustrated by the following examples that should not be construed as limiting. The contents of all references, patents and published patent applications cited throughout this application are incorporated herein by reference.

### Background of the 50090 Invention

Mitochondrial and peroxisomal β-oxidation enzymes degrade saturated and unsaturated fatty acids by sequentially removing two-carbon units from Coenzyme A (CoA)-activated fatty acids. The peroxisomal pathway oxidizes long and very long chain fatty acids and branched chain acyl-CoAs, while mitochondria oxidize short-, medium-, and long-chain fatty acids to produce energy for cells. Mitochondrial β-oxidation is a major energy source for cardiac and skeletal muscle. In liver, β-oxidation provides ketone bodies to the peripheral circulation when glucose levels are low, for example, during starvation, endurance exercise, and diabetes. See, for example, Eaton et al. (1996) Biochem. J. 320:345-357. The chief roles of peroxisomal β-oxidation are to shorten toxic lipophilic carboxylic acids to facilitate their excretion and to shorten very-long-chain fatty acids prior to mitochondrial β-oxidation.

Enzymes in the peroxisomal and mitochondrial pathways include long-chain specific and membrane bound acyl-CoA dehydrogenase, enoyl-CoA hydratase, L-3-hydroxyacyl-CoA dehydrogenase, and 3-ketoacyl-CoA thiolase. After shortening of long-chain fatty acyl-CoAs by one or more rounds of β-oxidation, soluble matrix enzymes having affinity for short- and medium-chain fatty acids complete the degradation of the acyl-CoA. Yao and Schulz (1996) J. Biol. Chem. 271(30):17816-17820. Inherited deficiencies in mitochondrial and peroxisomal beta-oxidation enzymes are associated with severe diseases, some of which manifest themselves soon after birth and lead to death within a few years.

### Summary of the 50090 Invention

The present invention is based, in part, on the discovery of a novel human hydratase, referred to herein as “50090”. In one embodiment, the present invention provides nucleic acids encoding a human hydratase. The nucleotide sequence of a cDNA encoding 50090 is shown as SEQ ID NO:28 and the amino acid sequence of a 50090 polypeptide is shown as SEQ ID NO:29 in Example 12. In addition, the nucleotide sequences of the coding region are depicted in Example 12 as SEQ ID NO:30.

Accordingly, in one aspect, the invention features a nucleic acid molecule that encodes a 50090 protein or polypeptide, e.g., a biologically active portion of the 50090 protein. In a preferred embodiment the isolated nucleic acid molecule encodes a polypeptide having the amino acid sequence of SEQ ID NO:29. In other embodiments, the invention provides isolated 50090 nucleic acid molecules having the nucleotide sequence shown in SEQ ID NO:28, SEQ ID NO:30, or the sequence of the DNA insert of the plasmid deposited with ATCC Accession Number ______. In still other embodiments, the invention provides nucleic acid molecules that are substantially identical (e.g., naturally occurring allelic variants) to the nucleotide sequence shown in SEQ ID NO:28, SEQ ID NO:30, or the sequence of the DNA insert of the plasmid deposited with ATCC Accession Number ______. In other embodiments, the invention provides a nucleic acid molecule which hybridizes under stringent hybridization conditions to a nucleic acid molecule comprising the nucleotide sequence of SEQ ID NO:28 or 30, or the sequence of the DNA insert of the plasmid deposited with ATCC Accession Number ______, wherein the nucleic acid encodes a full length 50090 protein or an active fragment thereof.

In a related aspect, the invention further provides nucleic acid constructs that include a 50090 nucleic acid molecule described herein. In certain embodiments, the nucleic acid molecules of the invention are operatively linked to native or heterologous regulatory sequences. Also included, are vectors and host cells containing the 50090 nucleic acid molecules of the invention e.g., vectors and host cells suitable for producing 50090 nucleic acid molecules and polypeptides.

In another related aspect, the invention provides nucleic acid fragments suitable as primers or hybridization probes for the detection of 50090-encoding nucleic acids.

In still another related aspect, isolated nucleic acid molecules that are antisense to a 50090-encoding nucleic acid molecule are provided.

In another aspect, the invention features 50090 polypeptides, and biologically active or antigenic fragments thereof that are useful, e.g., as reagents or targets in assays applicable to treatment and diagnosis of 50090-mediated or -related disorders, e.g., a hydratase associated disorder (e.g., genetic disorders, neuronal disorders, cancer, infectious diseases, liver disorders, and cardiac and skeletal muscle disorders).

In another embodiment, the invention provides 50090 polypeptides having a 50090 activity. Preferred polypeptides are 50090 proteins including an enoyl-CoA hydratase/isomerase domain, and, preferably, having a 50090 activity, e.g., a 50090 activity as described herein (e.g., a hydratase mediated activity, including, e.g., catalysis of the hydration of 2-trans-enoyl-CoA into 3-hydroxylacyl-CoA.

In other embodiments, the invention provides 50090 polypeptides, e.g., a 50090 polypeptide having the amino acid sequence shown in SEQ ID NO:29; the amino acid sequence encoded by the cDNA insert of the plasmid deposited with ATCC Accession Number ______; an amino acid sequence that is substantially identical to the amino acid sequence shown in SEQ ID NO:29; or an amino acid sequence encoded by a nucleic acid molecule having a nucleotide sequence that hybridizes under stringent hybridization conditions to a nucleic acid molecule comprising the nucleotide sequence of SEQ ID NO:28 or SEQ ID NO:30, or the sequence of the DNA insert of the plasmid deposited with ATCC Accession Number ______, wherein the nucleic acid encodes a full length 50090 protein or an active fragment thereof.

In a related aspect, the invention further provides nucleic acid constructs that include a 50090 nucleic acid molecule described herein.

In a related aspect, the invention provides 50090 polypeptides or fragments operatively linked to non-50090 polypeptides to form fusion proteins.

In another aspect, the invention features antibodies and antigen-binding fragments thereof, that react with, or more preferably, specifically bind 50090 polypeptides.

In another aspect, the invention provides methods of screening for compounds that modulate the expression or activity of the 50090 polypeptides or nucleic acids.

In still another aspect, the invention provides a process for modulating 50090 polypeptide or nucleic acid expression or activity, e.g. using the screened compounds. For example, the screened compounds can be used to modulate a hydratase mediated activity, including, fatty acid oxidation. In certain embodiments, the methods involve treatment of conditions related to aberrant activity or expression of the 50090 polypeptides or nucleic acids, such as conditions involving aberrant hydratase activity, e.g., a proliferative or muscular condition.

The invention also provides assays for determining the activity of or the presence or absence of 50090 polypeptides or nucleic acid molecules in a biological sample, including for disease diagnosis.

In yet another aspect, the invention provides methods for inhibiting the proliferation, or inducing the killing, of a 50090-expressing cell, e.g., a hyper-proliferative 50090-expressing cell. The method includes contacting the cell with a compound (e.g., a compound identified using the methods described herein) that modulates the activity, or expression, of the 50090 polypeptide or nucleic acid. In a preferred embodiment, the contacting step is effective in vitro or ex vivo. In other embodiments, the contacting step is effected in vivo, e.g., in a subject (e.g., a mammal, e.g., a human), as part of a therapeutic or prophylactic protocol. In a preferred embodiment, the cell is a hyperproliferative cell, e.g., a cell found in a solid tumor, a soft tissue tumor, or a metastatic lesion.

In a preferred embodiment, the compound is an inhibitor of a 50090 polypeptide. Preferably, the inhibitor is chosen from a peptide, a phosphopeptide, a small organic molecule, a small inorganic molecule and an antibody (e.g., an antibody conjugated to a therapeutic moiety selected from a cytotoxin, a cytotoxic agent and a radioactive metal ion). In another preferred embodiment, the compound is an inhibitor of a 50090 nucleic acid, e.g., an antisense, i a ribozyme, or a triple helix molecule.

In a preferred embodiment, the compound is administered in combination with a cytotoxic agent. Examples of cytotoxic agents include anti-microtubule agent, a topoisomerase I inhibitor, a topoisomerase II inhibitor, an anti-metabolite, a mitotic inhibitor, an alkylating agent, an intercalating agent, an agent capable of interfering with a signal transduction pathway, an agent that promotes apoptosis or necrosis, and radiation.

In another aspect, the invention features methods for treating or preventing a disorder characterized by aberrant cellular proliferation or differentiation of a 50090-expressing cell, in a subject. Preferably, the method includes comprising administering to the subject (e.g., a mammal, e.g., a human) an effective amount of a compound (e.g., a compound identified using the methods described herein) that modulates the activity, or expression, of the 50090 polypeptide or nucleic acid. In a preferred embodiment, the disorder is a cancerous or pre-cancerous condition.

In a further aspect, the invention provides methods for evaluating the efficacy of a treatment of a disorder, e.g., proliferative disorder or a muscular disorder. The method includes: treating a subject, e.g., a patient or an animal, with a protocol under evaluation (e.g., treating a subject with one or more of: chemotherapy, radiation, and/or a compound identified using the methods described herein); and evaluating the expression of a 50090 nucleic acid or polypeptide before and after treatment. A change, e.g., a decrease or increase, in the level of a 50090 nucleic acid (e.g., mRNA) or polypeptide after treatment, relative to the level of expression before treatment, is indicative of the efficacy of the treatment of the disorder. The level of 50090 nucleic acid or polypeptide expression can be detected by any method described herein.

In a preferred embodiment, the evaluating step includes obtaining a sample (e.g., a tissue sample such as a biopsy, or a fluid sample) from the subject, before and after treatment and comparing the level of expressing of a 50090 nucleic acid (e.g., mRNA) or polypeptide before and after treatment.

In another aspect, the invention provides methods for evaluating the efficacy of a therapeutic or prophylactic agent (e.g., an anti-neoplastic agent). The method includes: contacting a sample with an agent (e.g., a compound identified using the methods described herein, a cytotoxic agent) and, evaluating the expression of 50090 nucleic acid or polypeptide in the sample before and after the contacting step. A change, e.g., a decrease or increase, in the level of 50090 nucleic acid (e.g., mRNA) or polypeptide in the sample obtained after the contacting step, relative to the level of expression in the sample before the contacting step, is indicative of the efficacy of the agent. The level of 50090 nucleic acid or polypeptide expression can be detected by any method described herein. In a preferred embodiment, the sample includes cells obtained from a cancerous tissue or a neuronal tissue.

In further aspect the invention provides assays for determining the presence or absence of a genetic alteration in a 50090 polypeptide or nucleic acid molecule, including for disease diagnosis.

In another aspect, the invention features a two dimensional array having a plurality of addresses, each address of the plurality being positionally distinguishable from each other address of the plurality, and each address of the plurality having a unique capture probe, e.g., a nucleic acid or peptide sequence. At least one address of the plurality has a capture probe that recognizes a 50090 molecule. In one embodiment, the capture probe is a nucleic acid, e.g., a probe complementary to a 50090 nucleic acid sequence. In another embodiment, the capture probe is a polypeptide, e.g., an antibody specific for 50090 polypeptides. Also featured is a method of analyzing a sample by contacting the sample to the aforementioned array and detecting binding of the sample to the array.

Other features and advantages of the invention will be apparent from the following detailed description, and from the claims.

### Detailed Description of 50090

The human 50090 sequence (SEQ ID NO:28, Example 12), which is approximately 1639 nucleotides in length, including untranslated regions, contains a predicted methionine-initiated coding sequence of about 912 nucleotides, including the termination codon (nucleotides indicated as “coding” of SEQ ID NO:28 in Example 12; SEQ ID NO:30). The coding sequence encodes a 303 amino acid protein (SEQ ID NO:29).

Human 50090 contains one or more of the following regions or other structural features:

a predicted signal peptide located at amino acid 1 to about amino acid 21 of SEQ ID NO:29;

two predicted cAMP/cGMP protein kinase phosphorylation sites (PS00004) located at about amino acids 40 to 43 and 66 to 69 of SEQ ID NO:29;

three predicted protein kinase C phosphorylation sites (PS00005) located at about amino acids 49 to 51, 167 to 169 and 233 to 235 of SEQ ID NO:29;

two predicted casein kinase II phosphorylation sites (PS00006) located at about amino acids 105 to 108 and 210 to 213 of SEQ ID NO:29;

three predicted N-myristoylation sites (PS00008) located at about amino acids 148 to 153, 176 to 181, and 188 to 192 of SEQ ID NO:29; and

one predicted amidation site (PS00009) at about amino acid residues 38 to 41 of SEQ ID NO:29.

For general information regarding PFAM identifiers, PS prefix and PF prefix domain identification numbers, refer to Sonnhammer et al. (1997) Protein 28:405-420 and http://www.psc.edu/general/software/packages/pfam/pfam.html.

A plasmid containing the nucleotide sequence encoding human 50090 was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is requiredunder 35 U.S.C. §112.

The 50090 protein contains a significant number of structural characteristics in common with members of the enoyl-CoA hydratase/isomerase family. The term “family” when referring to the protein and nucleic acid molecules of the invention means two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin, or alternatively, can contain homologues of non-human origin, e.g., rat or mouse proteins. Members of a family can also have common functional characteristics. Members of the enoyl-CoA hydratase/isomerase family include enoyl-CoA hydratase, napthoate synthase, carnitate racemase, 3-hydroxybutyryl-CoA dehydratase, and dodecanoyl-CoA delta-isomerase.

As used herein, “hydratase/isomerase” includes a protein or polypeptide that is involved in fatty acid metabolism. Enoyl-CoA hydratase (E.C. 4.2.1.17) catalyzes the hydration of 2-trans-enoyl-CoA into 3-hydroxyacyl-CoA and 3-2trans-enoyl-CoA isomerase shifts the 3-double bond of the intermediates of unsaturated fatty acid oxidation to the 2-trans position. As the 50090 molecules of the present invention may modulate hydratase mediated activities, these molecules may be useful for developing novel diagnostic and therapeutic agents for hydratase associated disorders.

A 50090 polypeptide can include an “enoyl-CoA hydratase/isomerase domain” or regions homologous with a “enoyl-CoA hydratase/isomerase domain”.

As used herein, the term “enoyl-CoA hydratase/isomerase domain” includes an amino acid sequence of about 100 to 200 amino acid residues in length and having a bit score for the alignment of the sequence to the enoyl-CoA hydratase/isomerase domain (HMM) of at least 90. Preferably, an enoyl-CoA hydratase/isomerase domain includes at least about 125-185 amino acids, more preferably about 140-175 amino acid residues, or about 150-170 amino acids and has a bit score for the alignment of the sequence to the enoyl-CoA hydratase/isomerase domain (HMM) of at least 140 or greater. The enoyl-CoA hydratase/isomerase domain (HMM) has been assigned the PFAM Accession PF00378 (http;//genome.wustl.edu/Pfam/.html). Preferably, the enoyl-CoA hydratase/isomerase domain is rich in glycine and hydrophobic residues, and includes an active site containing at least two glutamic acid residues and at least five, ten, preferably fifteen, and more preferably seventeen highly conserved amino acids. See, Wu et al. (1997) Biochemistry 36:2211-2220.

In a preferred embodiment, a 50090 polypeptide or protein has an “enoyl-CoA hydratase/isomerase domain” or a region that includes at least about 125-185, and more preferably about 140-170 amino acid residues and has at least about 50%, 60%, 70% 80% 90% 95%, 99%, or 100% homology with a “enoyl-CoA hydratase/isomerase domain,” e.g., the enoyl-CoA hydratase/isomerase domain of human 50090 (e.g., residues 57-225 of SEQ ID NO:29).

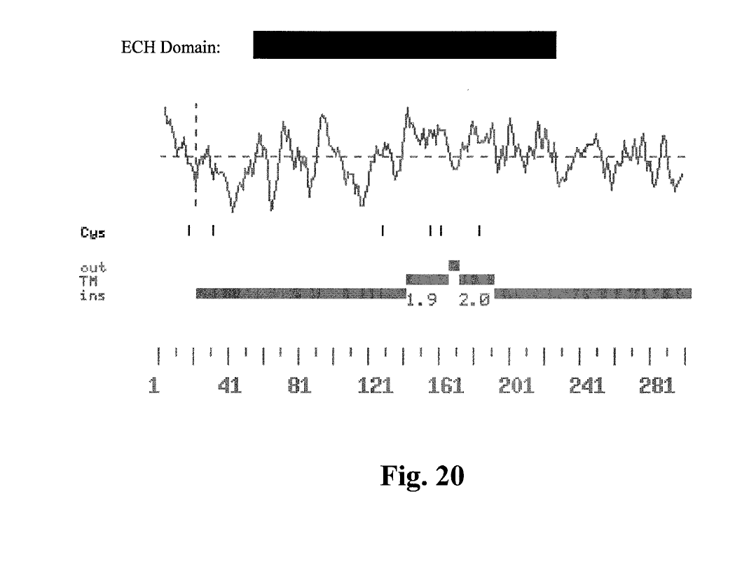

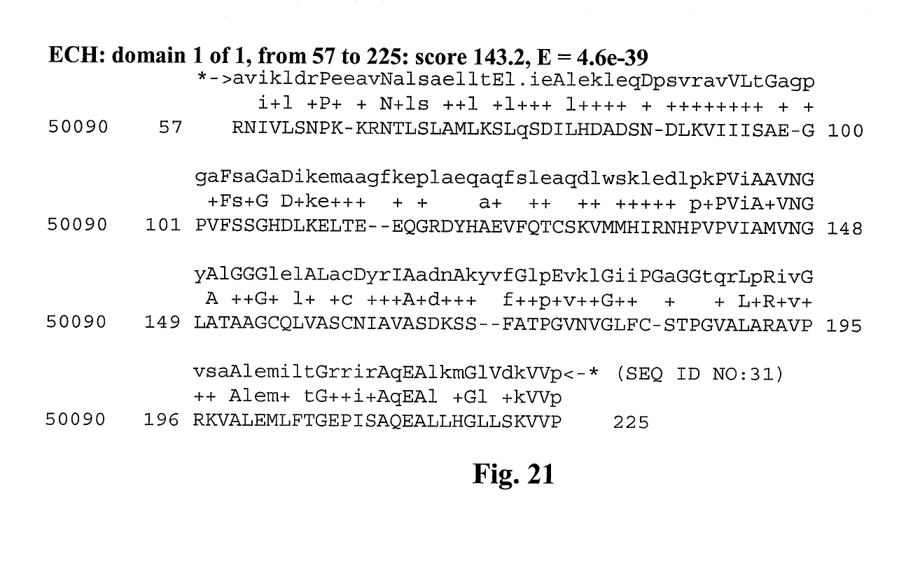

To identify the presence of a “enoyl-CoA hydratase/isomerase” domain in a 50090 protein sequence, and make the determination that a polypeptide or protein of interest has a particular profile, the amino acid sequence of the protein can be searched against a database of HMMs (e.g., the Pfam database, release 2.1) using the default parameters (http://www.sanger.ac.uk/Software/Pfam/HMM_search). For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al (1987) Proc. Natl. Acad. Sci. USA 84:4355-4358; Krogh et al.(1994) J. Mol. Biol. 235:1501-1531; and Stultz et al.(1993) Protein Sci. 2:305-314, the contents of which are incorporated herein by reference. A search was performed against the HMM database resulting in the identification of a “enoyl-CoA hydratase/isomerase domain” in the amino acid sequence of human 50090 at about residues 57-225 of SEQ ID NO:29 (see FIG. 20).

The 50090 molecule further can include a signal sequence. As used herein, a “signal sequence” refers to a peptide of about 20-30 amino acid residues in length that occurs at the N-terminus of secretory and integral membrane proteins and that contains a majority of hydrophobic amino acid residues. For example, a signal sequence contains at least about 15-45 amino acid residues, preferably about 20-40 amino acid residues, more preferably about 21-33 amino acid residues, and more preferably about 23-31 amino acid residues, and has at least about 40-70%, preferably about 50-65%, and more preferably about 55-60% hydrophobic amino acid residues (e.g., alanine, valine, leucine, isoleucine, phenylalanine, tyrosine, tryptophan, or proline). Such a “signal sequence”, also referred to in the art as a “signal peptide”, serves to direct a protein containing such a sequence to a lipid bilayer.

In one embodiment, a 50090 protein contains amino acids 1-21 of SEQ ID NO:29. In other embodiments the 50090 protein does not include amino acids 1-21 of SEQ ID NO:29, and can, e.g., correspond to amino acids 22 to 303 of SEQ ID NO:29. A 50090 protein can be located within the cytoplasm or mitochondria of a cell.

As the 50090 polypeptides of the invention may modulate 50090-mediated activities, they may be useful as of for developing novel diagnostic and therapeutic agents for 50090-mediated or -related disorders, as described below.

As used herein, a “50090 activity”, “biological activity of 50090” or “functional activity of 50090”, refers to an activity exerted by a 50090 protein, polypeptide or nucleic acid molecule on e.g., a 50090-responsive cell or on a 50090 substrate, e.g., a protein substrate, as determined in vivo or in vitro, according to standard assay techniques. In one embodiment, a 50090 activity is a direct activity, such as an association with a 50090 target molecule, or an enzymatic activity on a second protein. A “target molecule” or “binding partner” is a molecule that a 50090 protein binds or interacts with in nature. In another embodiment, a 50090 activity is an indirect activity, such as a cellular signaling activity mediated by interaction of the 50090 protein with a second protein.

Based on the above-described sequence similarities, the 50090 molecules are predicted to have similar biological activities as other hydratase/isomerase family members. For example, the 50090 proteins of the present invention is predicted to have one or more of the following activities: (1) catalyze the hydration of 2-trans-enoyl-CoA into 3-hydroxyacyl-CoA; (2) catalyze the shift of the 3-double bond of the intermediates of unsaturated fatty acid oxidation to the 2-trans position; (3) oxidation of fatty acids; (4) modulation of fatty acid accumulation; (5) modulation of signal transduction, (6) modulation of gene expression; or (7) modulation of cell proliferation, differentiation, or morphogenesis.

As used herein, a “hydratase mediated activity” includes an activity that involves a hydratase, e.g., a hydratase in a cardiac or a muscle cell, associated with fatty acid oxidation. Hydratase mediated activities include hydration of 2-trans-enoyl-CoA into 3-hydroxylacyl-CoA and the shift of the 3-double bond of the intermediates of unsaturated fatty acid oxidation to the 2-trans position.

As the 50090 molecules of the present invention may modulate hydratase mediated activities, these molecules may be useful for developing novel diagnostic and therapeutic agents for hydratase associated disorders. As used herein, a “hydratase associated disorder” includes a disorder, disease or condition that is characterized by a misregulation of hydratase mediated activity. Hydratase associated disorders include genetic disorders, neuronal disorders, cancer, infectious diseases, liver disorders, and cardiac and skeletal muscle disorders, and other disorders associated with defects in fatty acid oxidation. For example, patients deficient in mitochondrial trifunctional protein (which includes enoyl-CoA hydratase) have reduced long-chain enoyl-CoA hydratase activities and suffer from non-ketotic hypoglycemia, sudden infant death syndrome, cardiomyopathy, hepatic dysfunction, and muscle weakness, and may die at an early age. Inherited conditions associated with peroxisomal beta-oxidation include Zellweger syndrome, neonatal adrenoleukodystrophy, infantile Refsum's disease, acyl-CoA oxidase deficiency, peroxisomal thiolase deficiency, and bifunctional protein deficiency. Suzuki et al. (1994) Am. J. Hum. Genet. 54:36-43. Patients with peroxisomal bifunctional enzyme, including enoyl-CoA hydratase, deficiency suffer from hypotonia, seizures, psychomotor defects, and defective neuronal migration; accumulate very-long-chain fatty acids; and typically die within a few years of birth. See, Watkins et al. (1989) J. Clin. Invest. 83:771-777.

Neuronal disorders include cognitive and neurodegenerative disorders, examples of which include, but are not limited to, Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, progressive supranuclear palsy, epilepsy, Jakob-Creutzfieldt disease, or AIDS related dementia; autonomic function disorders such as hypertension and sleep disorders, and neuropsychiatric disorders, such as depression, schizophrenia, schizoaffective disorder, korsakoff's psychosis, mania, anxiety disorders, or phobic disorders; learning or memory disorders, e.g., amnesia or age-related memory loss, attention deficit disorder, psychoactive substance use disorders, anxiety, phobias, panic disorder, as well as bipolar affective disorder, e.g., severe bipolar affective (mood) disorder (BP-1), and bipolar affective neurological disorders, e.g., migraine and obesity. Further CNS-related disorders include, for example, those listed in the American Psychiatric Association's Diagnostic and Statistical manual of Mental Disorders (DSM), the most current version of which is incorporated herein by reference in its entirety.

Further examples of hydratase-associated disorders include muscular disorders such as muscular dystrophy (e.g., Duchenne muscular dystrophy or myotonic dystrophy), spinal muscular atrophy, congenital myopathies, central core disease, rod myopathy, central nuclear myopathy, Lambert-Eaton syndrome, denervation, paralysis, and muscle weakness (e.g., ataxia, myotonia, and myokymia) and infantile spinal muscular atrophy (Werdnig-Hoffman disease).

Hydratase disorders also include cellular proliferation, growth, differentiation, or migration disorders. Cellular proliferation, growth, differentiation, or migration disorders include those disorders that affect cell proliferation, growth, differentiation, or migration processes. As used herein, a “cellular proliferation, growth, differentiation, or migration process” is a process by which a cell increases in number, size or content, by which a cell develops a specialized set of characteristics which differ from that of other cells, or by which a cell moves closer to or further from a particular location or stimulus. The 50090 molecules of the present invention can be involved with proliferation and transcriptional activation mechanisms, which are known to be involved in cellular growth, differentiation, and migration processes. Thus, the 50090 molecules may modulate cellular growth, differentiation, or migration, and may play a role in disorders characterized by aberrantly regulated growth, differentiation, or migration. Such disorders include cancer, e.g., carcinoma, sarcoma, or leukemia; tumor angiogenesis and metastasis; skeletal dysplasia; neuronal deficiencies resulting from impaired neural induction and patterning; hepatic disorders; cardiovascular disorders; and hematopoietic and/or myeloproliferative disorders.

The 50090 protein, fragments thereof, and derivatives and other variants of the sequence in SEQ ID NO:29 thereof are collectively referred to as “polypeptides or proteins of the invention” or “50090 polypeptides or proteins”. Nucleic acid molecules encoding such polypeptides or proteins are collectively referred to as “nucleic acids of the invention” or “50090 nucleic acids.” 50090 molecules refer to 50090 nucleic acids, polypeptides, and antibodies.

As used herein, the term “nucleic acid molecule” includes DNA molecules (e.g., a cDNA or genomic DNA) and RNA molecules (e.g., mRNA) and analogs of the DNA or RNA generated, e.g., by the use of nucleotide analogs. The nucleic acid molecule can be single-stranded or double-stranded, but preferably is double-stranded DNA.

The term “isolated or purified nucleic acid molecule” includes nucleic acid molecules that are separated from other nucleic acid molecules that are present in the natural source of the nucleic acid. For example, with regard to genomic DNA, the term “isolated” includes nucleic acid molecules that are separated from the chromosome with which the genomic DNA is naturally associated. Preferably, an “isolated” nucleic acid is free of sequences that naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6×sodium chloride/sodium citrate (SSC) at about 45° C., followed by two washes in 0.2× SSC, 0. 1% SDS at least at 50° C. (the temperature of the washes can be increased to 55° C. for low stringency conditions); 2) medium stringency hybridization conditions in 6× SSC at about 45° C., followed by one or more washes in 0.2× SSC, 0. 1% SDS at 60° C.; 3) high stringency hybridization conditions in 6× SSC at about 45° C., followed by one or more washes in 0.2× SSC, 0.1% SDS at 65° C.; and preferably 4) very high stringency hybridization conditions are 0.5M sodium phosphate, 7% SDS at 65° C., followed by one or more washes at 0.2× SSC, 1% SDS at 65° C. Very high stringency conditions (4) are the preferred conditions and the ones that should be used unless otherwise specified. Preferably, an isolated nucleic acid molecule of the invention that hybridizes under stringent conditions to the sequence of SEQ ID NO:28 or SEQ ID NO:30, corresponds to a naturally-occurring nucleic acid molecule.

As used herein, a “naturally-occurring” nucleic acid molecule refers to an RNA or DNA molecule having a nucleotide sequence that occurs in nature (e.g., encodes a natural protein).

As used herein, the terms “gene” and “recombinant gene” refer to nucleic acid molecules that include an open reading frame encoding a 50090 protein, preferably a mammalian 50090 protein, and can further include non-coding regulatory sequences, and introns.

An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, “substantially free” means a preparation of 50090 protein having less than about 30%, 20%, 10% and more preferably 5% (by dry weight) of non-50090 protein (also referred to herein as a “contaminating protein”), or of chemical precursors or non-50090 chemicals. When the 50090 protein or biologically active portion thereof is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight.

A “non-essential” amino acid residue is a residue that can be altered from the wild-type sequence of 50090 (e.g., the sequence of SEQ ID NO:28 or SEQ ID NO:30, or the nucleotide sequence of the DNA insert of the plasmid deposited with ATCC as Accession Number ______) without abolishing or more preferably, without substantially altering a biological activity, whereas an “essential” amino acid residue results in such a change. For example, amino acid residues that are conserved among the polypeptides of the present invention, e.g., a number of those present in the enoyl-CoA hydratase/isomerase domain, are predicted to be particularly unamenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 50090 protein can be preferably replaced with another amino acid residue from the same side chain family. Alternatively, in another embodiment, mutations can be introduced randomly along all or part of a 50090 coding sequence, such as by saturation mutagenesis, and the resultant mutants can be screened for 50090 biological activity to identify mutants that retain activity. Following mutagenesis of SEQ ID NO:28 or SEQ ID NO:30, or the nucleotide sequence of the DNA insert of the plasmid deposited with ATCC as Accession Number ______, the encoded protein can be expressed recombinantly and the activity of the protein can be determined.

As used herein, a “biologically active portion” of a 50090 protein includes a fragment of a 50090 protein that participates in an interaction between a 50090 molecule and a non-50090 molecule. Biologically active portions of a 50090 protein include peptides comprising amino acid sequences sufficiently homologous to or derived from the amino acid sequence of the 50090 protein, e.g., the amino acid sequence shown in SEQ ID NO:29, which include fewer amino acids than the full length 50090 proteins, and exhibit at least one activity of a 50090 protein. Typically, biologically active portions comprise an enoyl-CoA hydratase/isomerase domain or motif with at least one activity of the 50090 protein, e.g., the ability to catalyze the hydration of 2-trans-enoyl-CoA into 3-hydroxyacyl-CoA. A biologically active portion of a 50090 protein can be a polypeptide that is, for example, 10, 25, 50, 100, 200, or 300 amino acids in length. Biologically active portions of a 50090 protein can be used as targets for developing agents that modulate a 50090-mediated activity, e.g., a hydratase mediated activity as described herein.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least about 30%, preferably at least about 40%, more preferably at least about 50%, even more preferably at least about 60%, and even more preferably at least about 70%, 80%, 90%, or 100% of the length of the reference sequence (e.g., when aligning a second sequence to the 50090 amino acid sequence of SEQ ID NO:29 having 304 amino acid residues, at least 91, preferably at least 142, more preferably at least 172, even more preferably at least 182, and even more preferably at least 213, 243, or 274 amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to 50090 nucleic acid molecules of the invention. BLAST protein searches can be performed with the XBLAST program, score=50, C 10 wordlength=3 to obtain amino acid sequences homologous to 50090 protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

Particular 50090 polypeptides of the present invention have an amino acid sequence substantially identical to the amino acid sequence of SEQ ID NO:29. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 60%, or 65% identity, likely 75% identity, more likely 85%, 90%. 91%, 92%, 93%, 94%, 95%, 96%, 97%, 98% or 99% identity to SEQ ID NO:29 are termed substantially identical.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 60%, or 65% identity, likely 75% identity, more likely 85%, 90%. 91%, 92%, 93%, 94%, 95%, 96%, 97%, 98% or 99% identity to SEQ ID NO:28 or 30 are termed substantially identical.

“Misexpression or aberrant expression”, as used herein, refers to a non-wild type pattern of gene expression, at the RNA or protein level. It includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

“Subject”, as used herein, can refer to a mammal, e.g., a human, or to an experimental or animal or disease model. The subject can also be a non-human animal, e.g., a horse, cow, goat, or other domestic animal.

A “purified preparation of cells”, as used herein, refers to, in the case of plant or animal cells, an in vitro preparation of cells and not an entire intact plant or animal. In the case of cultured cells or microbial cells, it consists of a preparation of at least 10% and more preferably 50% of the subject cells.

Various aspects of the invention are described in further detail below.

Isolated Nucleic Acid Molecules of 50090

In one aspect, the invention provides, an isolated or purified, nucleic acid molecule that encodes a 50090 polypeptide described herein, e.g., a full length 50090 protein or a fragment thereof, e.g., a biologically active portion of 50090 protein. Also included is a nucleic acid fragment suitable for use as a hybridization probe, which can be used, e.g., to a identify nucleic acid molecule encoding a polypeptide of the invention, 50090 mRNA, and fragments suitable for use as primers, e.g., PCR primers for the amplification or mutation of nucleic acid molecules.

In one embodiment, an isolated nucleic acid molecule of the invention includes the nucleotide sequence shown in SEQ ID NO:28, or the nucleotide sequence of the DNA insert of the plasmid deposited with ATCC as Accession Number ______, or a portion of any of these nucleotide sequences. In one embodiment, the nucleic acid molecule includes sequences encoding the human 50090 protein (i.e., “the coding region” as shown in SEQ ID NO:30), as well as 5′ untranslated sequences (as shown in FIG. 20). Alternatively, the nucleic acid molecule can include only the coding region of SEQ ID NO:28 (i.e., SEQ ID NO:30) and, e.g., no flanking sequences that normally accompany the subject sequence.

In another embodiment, an isolated nucleic acid molecule of the invention includes a nucleic acid molecule that is a complement of the nucleotide sequence shown in SEQ ID NO:28 or SEQ ID NO:30, or the nucleotide sequence of the DNA insert of the plasmid deposited with ATCC as Accession Number ______, or a portion of any of these nucleotide sequences. In other embodiments, the nucleic acid molecule of the invention is sufficiently complementary to the nucleotide sequence shown in SEQ ID NO:28 or SEQ ID NO:30, or the nucleotide sequence of the DNA insert of the plasmid deposited with ATCC as Accession Number such that it can hybridize to the nucleotide sequence shown in SEQ ID NO:28 or SEQ ID NO:30, or the nucleotide sequence of the DNA insert of the plasmid deposited with ATCC as Accession Number ______, thereby forming a stable duplex.

In one embodiment, an isolated nucleic acid molecule of the present invention includes a nucleotide sequence that is at least about 60%, 65%, 70%, 75%, 80%, 85%, 90%, 91%, 92%, 93%, 94%, 95%, 96%, 97%, 98%, 99%, or more homologous to the entire length of the nucleotide sequence shown in SEQ ID NO:28 or SEQ ID NO:30, or the entire length of the nucleotide sequence of the DNA insert of the plasmid deposited with ATCC as Accession Number ______, or a portion, preferably of the same length, of any of these nucleotide sequences.

50090 Nucleic Acid Fragments

A nucleic acid molecule of the invention can include only a portion of the nucleic acid sequence of SEQ ID NO:28 or SEQ ID NO:30, or the nucleotide sequence of the DNA insert of the plasmid deposited with ATCC as Accession Number ______. For example, such a nucleic acid molecule can include a fragment that can be used as a probe or primer or a fragment encoding a portion of a 50090 protein, e.g., an immunogenic or biologically active portion of a 50090 protein. A fragment can comprise nucleotides of SEQ ID NO:28 encoding amino acids 57 to 225 of SEQ ID NO:29, which encodes a enoyl-CoA/hydratase/isomerase domain of human 50090, as well as any other domain or region described herein. In preferred embodiments, the nucleic acid fragment is at least 30, 500, 100, 150, 200, 250, 300, 350, 400, 450, or 500 nucleotides in length and less than 900, 850, 800, 750, 700, 650, or 600 nucleotides in length. The nucleotide sequence determined from the cloning of the 50090 gene allows for the generation of probes and primers designed for use in identifying and/or cloning other 50090 family members, or fragments thereof, as well as 50090 homologues, or fragments thereof, from other species.

In another embodiment, a nucleic acid includes a nucleotide sequence that includes part, or all, of the coding region and extends into either (or both) the 5′ or 3′ noncoding region. Other embodiments include a fragment that includes a nucleotide sequence encoding an amino acid fragment described herein. Nucleic acid fragments can encode a specific domain or site described herein or fragments thereof, particularly fragments thereof that are at least 30 amino acids in length. Fragments also can include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a domain, region, or functional site described herein. Thus, for example, a 50090 nucleic acid fragment can include a sequence corresponding to an enoyl-CoA hydratase/isomerase domain at locations in the translated 50090 polypeptide described herein.

50090 probes and primers are provided. Typically a probe/primer is an isolated or purified oligonucleotide. The oligonucleotide typically includes a region of nucleotide sequence that hybridizes under stringent conditions to at least about 7, 12 or 15, preferably about 20 or 25, more preferably about 30, 35, 40, 45, 50, 55, 60, 65, or 75 consecutive nucleotides of a sense or antisense sequence of SEQ ID NO:28 or SEQ ID NO:30, or the nucleotide sequence of the DNA insert of the plasmid deposited with ATCC as Accession Number ______, or of a naturally occurring allelic variant or mutant of SEQ ID NO:28 or SEQ ID NO:30, or the nucleotide sequence of the DNA insert of the plasmid deposited with ATCC as Accession Number ______.

In a preferred embodiment the nucleic acid is a probe that is at least 5 or 10, and less than 200, more preferably less than 100, or less than 50, base pairs in length. It should be identical, or differ by 1, or less than in 5 or 10 bases, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid which encodes, e.g., an enoyl-CoA hydratase/isomerase domain located from about amino acids 57 to 225 of SEQ ID NO:29 or any other domain or region described herein.

In another embodiment a set of primers is provided, e.g., primers suitable for use in a PCR, which can be used to amplify a selected region of a 50090 sequence, e.g., a domain, region, site or other sequence described herein. The primers should be at least 5, 10, or 50 base pairs in length and less than 100 or 200 base pairs in length. The primers should be identical, or differs by one base from a sequence disclosed herein or from a naturally occurring variant. For example, primers suitable for amplifying all or a portion of any of the following regions are provided: an enoyl-CoA hydratase/isomerase domain located from about amino acids 57 to 225 of SEQ ID NO:29.

A nucleic acid fragment can encode an epitope-bearing region of a polypeptide described herein.

A nucleic acid fragment encoding a “biologically active portion of a 50090 polypeptide” can be prepared by isolating a portion of the nucleotide sequence of SEQ ID NO:28 or 30, or the nucleotide sequence of the DNA insert of the plasmid deposited with ATCC as Accession Number ______, which encodes a polypeptide having a 50090 biological activity (e.g., the biological activities of the 50090 proteins are described herein), expressing the encoded portion of the 50090 protein (e.g., by recombinant expression in vitro) and assessing the activity of the encoded portion of the 50090 protein. For example, a nucleic acid fragment encoding a biologically active portion of 50090 includes an enoyl-CoA hydratase/isomerase domain located from about amino acids 57-225 of SEQ ID NO:29. A nucleic acid fragment encoding a biologically active portion of a 50090 polypeptide may comprise a nucleotide sequence that is greater than 300 or more nucleotides in length.

In preferred embodiments, nucleic acids include a nucleotide sequence that is about or more than 300, 400, 500, 600, 700, 800, 900, 1000, 1100, 1200, 1300, 1400, 1500, or 1600 nucleotides in length. The nucleic acid can hybridizes under stringent hybridization conditions to a nucleic acid molecule of SEQ ID NO:28, or SEQ ID NO:30, or the nucleotide sequence of the DNA insert of the plasmid deposited with ATCC as Accession Number

50090 Nucleic Acid Variants

The invention further encompasses nucleic acid molecules that differ from the nucleotide sequence shown in SEQ ID NO:28 or SEQ ID NO:30, or the nucleotide sequence of the DNA insert of the plasmid deposited with ATCC as Accession Number ______. Such differences can be due to degeneracy of the genetic code (and result in a nucleic acid that encodes the same 50090 proteins as those encoded by the nucleotide sequence disclosed herein. In another embodiment, an isolated nucleic acid molecule of the invention has a nucleotide sequence encoding a protein having an amino acid sequence which differs, by at least 1, but less than 5, 10, 20, 50, or 100 amino acid residues than that shown in SEQ ID NO:29. Ifalignment is needed for this comparison the sequences should be aligned for maximum homology. “Looped” out sequences from deletions or insertions, or mismatches, are considered differences.

Nucleic acids of the invention can be chosen for having codons that are preferred or non-preferred for a particular expression system. For example, the nucleic acid can be one in which at least one codon, preferably at least 10%, or 20% of the codons, has been altered such that the sequence is optimized for expression in E. coli, yeast, human, insect, or Chinese hamster ovary (CHO) cells.

Nucleic acid variants can be naturally occurring, such as allelic variants (same locus), homologs (different locus), and orthologs (different organism) or can be non-naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

In a preferred embodiment, the nucleic acid differs from that of SEQ ID NO:28 or SEQ ID NO:30, or the sequence in ATCC Accession Number ______, e.g., as follows: by at least one but less than 10, 20, 30, or 40 nucleotides; at least one but less than 1%, 5%, 10% or 20% of the in the subject nucleic acid. If necessary for this analysis, the sequences should be aligned for maximum homology. “Looped” out sequences from deletions or insertions, or mismatches, are considered differences.

Orthologs, homologs, and allelic variants can be identified using methods known in the art. These variants comprise a nucleotide sequence encoding a polypeptide that is at least about 60%, typically at least about 70-75%, more typically at least about 80-85%, and most typically at least about 90-95% or more (e.g., 99%) identical to the nucleotide sequence shown in SEQ ID NO:29 or a fragment of this sequence. Such nucleic acid molecules can readily be identified as being able to hybridize under stringent conditions to the nucleotide sequence shown in SEQ ID NO:29 or a fragment of the sequence. Nucleic acid molecules corresponding to orthologs, homologs, and allelic variants of the 50090 cDNAs of the invention can further be isolated by mapping to the same chromosome or locus as the 50090 gene.

Preferred variants include those that are correlated with modulating cell proliferation, differentiation, or morphogenesis, fatty acid β-oxidation, modulating signal transduction, and modulating gene expression.

Allelic variants of 50090, e.g., human 50090, include both functional and non-functional proteins. Functional allelic variants are naturally occurring amino acid sequence variants of the 50090 protein within a population that maintain the ability to hydrate 2-trans enoyl-CoA into 3-hydroxylacyl-CoA. Functional allelic variants will typically contain only conservative substitution of one or more amino acids of SEQ ID NO:29, or substitution, deletion or insertion of non-critical residues in non-critical regions of the protein. Non-functional allelic variants are naturally-occurring amino acid sequence variants of the 50090, e.g., human 50090, protein within a population that do not have the ability to hydrate 2-trans-enoyl-CoA. Non-functional allelic variants will typically contain a non-conservative substitution, a deletion, or insertion, or premature truncation of the amino acid sequence of SEQ ID NO:29, or a substitution, insertion, or deletion in critical residues or critical regions of the protein.

Moreover, nucleic acid molecules encoding other 50090 family members and, thus, which have a nucleotide sequence which differs from the 50090 sequences of SEQ ID NO:28 or SEQ ID NO:30, or the nucleotide sequence of the DNA insert of the plasmid deposited with ATCC as Accession Number are intended to be within the scope of the invention.

Antisense Nucleic Acid Molecules, Ribozymes and Modified 50090 Nucleic Acid Molecules

In another aspect, the invention features an isolated nucleic acid molecule that is antisense to 50090. An “antisense” nucleic acid can include a nucleotide sequence that is complementary to a “sense” nucleic acid encoding a protein, e.g., complementary to the coding strand of a double-stranded cDNA molecule or complementary to an mRNA sequence. The antisense nucleic acid can be complementary to an entire 50090 coding strand, or to only a portion thereof (e.g., the coding region of 50090 corresponding to SEQ ID NO:30). In another embodiment, the antisense nucleic acid molecule is antisense to a “noncoding region” of the coding strand of a nucleotide sequence encoding 50090 (e.g., the 5′ and 3′ untranslated regions).

An antisense nucleic acid can be designed such that it is complementary to the entire coding region of 50090 mRNA, but more preferably is an oligonucleotide that is antisense to only a portion of the coding or noncoding region of 50090 mRNA. For example, the antisense oligonucleotide can be complementary to the region surrounding the translation start site of 50090 mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in antisense orientation (i.e., RNA transcribed from the inserted nucleic acid will be of an antisense orientation to a target nucleic acid of interest, described further in the following subsection).

The antisense nucleic acid molecules of the invention are typically administered to a subject (e.g., by direct injection at a tissue site), or generated in situ such that they hybridize with or bind to cellular mRNA and/or genomic DNA encoding a 50090 protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. A ribozyme having specificity for a 50090-encoding nucleic acid can include one or more sequences complementary to the nucleotide sequence of a 50090 cDNA disclosed herein (i.e., SEQ ID NO:28 or SEQ ID NO:30), and a sequence having known catalytic sequences responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a 50090-encoding mRNA. See, e.g., U.S. Pat. No. 4,987,071 and 5,116,742. Alternatively, 50090 mRNA can be used to select a catalytic RNA having a specific ribonuclease activity from a pool of RNA molecules. See, e.g., Bartel, D. and Szostak, J. W. (1993) Science 261:1411-1418.

50090 gene expression can be inhibited by targeting nucleotide sequences complementary to the regulatory region of the 50090 (e.g., the 50090 promoter and/or enhancers) to form triple helical structures that prevent transcription of the 50090 gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15. The potential sequences that can be targeted for triple helix formation can be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

The invention also provides detectably labeled oligonucleotide primer and probe molecules. Typically, such labels are chemiluminescent, fluorescent, radioactive, or calorimetric.

A 50090 nucleic acid molecule can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. (1996) Proc. Natl. Acad. Sci. USA 93: 14670-675.

PNAs of 50090 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of 50090 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

The invention also includes molecular beacon oligonucleotide primer and probe molecules having at least one region which is complementary to a 50090 nucleic acid of the invention, two complementary regions one having a fluorophore and one a quencher such that the molecular beacon is useful for quantitating the presence of the 50090 nucleic acid of the invention in a sample. Molecular beacon nucleic acids are described, for example, in U.S. Pat. Nos. 5,854,033, 5,866,336, and 5,876,930.

Isolated 50090 Polypeptides

In another aspect, the invention features an isolated 50090 protein or fragment, e.g., a biologically active portion, for use as immunogens or antigens to raise or test (or more generally to bind) anti-50090 antibodies. 50090 protein can be isolated from cells or tissue sources using standard protein purification techniques. 50090 protein or fragments thereof can be produced by recombinant DNA techniques or synthesized chemically. 50090 fragments are at least 10, 20, 40, 80, 100, or 150 amino acids in length and less than 303, 2750, 250, 225, or 200 amino acids in length.

Polypeptides of the invention include those that arise as a result of the existence of multiple genes, alternative transcription events, alternative RNA splicing events, and alternative translational and postranslational events. The polypeptide can be expressed in systems, e.g., cultured cells, which result in substantially the same postranslational modifications present when expressed the polypeptide is expressed in a native cell, or in systems which result in the alteration or omission of postranslational modifications, e.g., glycosylation or cleavage, present when expressed in a native cell.

In a preferred embodiment, a 50090 polypeptide has one or more of the following characteristics:

(i) it has a signal peptide;

(ii) it associates or attaches to a cell membrane;

(iii) it catalyzes the hydration of 2-trans-enoyl-CoA into 3-hydroxylacyl-CoA;

(iv) it catalyzes the shift of the 3-double bond of the intermediates of unsaturated fatty acid oxidation to the 2-trans position;

(v) it has an amino acid composition of SEQ ID NO:29;

(vi) it has an overall sequence similarity of at least 60%, preferably at least 70%, more preferably at least 80%, 90%, 95%, 96%, 97%, 98%, or 99% with a polypeptide of SEQ ID NO:29;

(vii) it can be found in human tissue;

(viii) or

it has at least two, preferably at least three, and most preferably at least four of the six cysteines found in the amino acid sequence of the native protein.

In a preferred embodiment the 50090 protein or fragment thereof differs from the corresponding sequence in SEQ ID NO:29. In one embodiment, it differs by at least one but by less than 15, 10 or 5 amino acid residues. In another embodiment, it differs from the corresponding sequence in SEQ ID NO:29 by at least one residue but less than 20%, 15%, 10% or 5% of the residues in it differ from the corresponding sequence in SEQ ID NO:29. (If this comparison requires alignment the sequences should be aligned for maximum homology. “Looped” out sequences from deletions or insertions, or mismatches, are considered differences.) The differences are, preferably, differences or changes at a non-essential residue or a conservative substitution. In another preferred embodiment one or more differences are in the enoyl-CoA hydratase/isomerase domain of amino acid residues 57 to 225 of SEQ ID NO:29.

Other embodiments include a protein that contains one or more changes in amino acid sequence, e.g., a change in an amino acid residue that is not essential for activity. Such 50090 proteins differ in amino acid sequence from SEQ ID NO:29, yet retain biological activity.

In one embodiment, the protein includes an amino acid sequence at least about 60%, 65%, 70%, 75%, 80%, 85%, 90%, 95%, 98% or more homologous to SEQ ID NO:29.

A 50090 protein or fragment is provided that varies from the sequence of SEQ ID NO:29 in non-active site residues by at least one but by less than 15, 10 or 5 amino acid residues in the protein or fragment, but which does not differ from SEQ ID NO:29 in the enoyl-CoA hydratase/isomerase domain of amino acid residues 57 to 225 (If this comparison requires alignment the sequences should be aligned for maximum homology. “Looped” out sequences from deletions or insertions, or mismatches, are considered differences.) In some embodiments the difference is at a non-essential residue or is a conservative substitution, while in others the difference is at an essential residue or is a non conservative substitution.

In a preferred embodiment, the 50090 protein has an amino acid sequence shown in SEQ ID NO:29. In other embodiments, the 50090 protein is substantially identical to SEQ ID NO:29. In yet another embodiment, the 50090 protein is substantially identical to SEQ ID NO:29 and retains the functional activity of the protein of SEQ ID NO:29, as described in detail in the subsections above.

0090 Chimeric or Fusion Proteins

In another aspect, the invention provides 50090 chimeric or fusion proteins. As used herein, a 50090 “chimeric protein” or “fusion protein” includes a 50090 polypeptide linked to a non-50090 polypeptide. A “non-50090 polypeptide” refers to a polypeptide having an amino acid sequence corresponding to a protein which is not substantially homologous to the 50090 protein, e.g., a protein that is different from the 50090 protein and that is derived from the same or a different organism. The 50090 polypeptide of the fusion protein can correspond to all or a portion e.g., a fragment described herein of a 50090 amino acid sequence. In a preferred embodiment, a 50090 fusion protein includes at least one (or two) biologically active portion of a 50090 protein. The non-50090 polypeptide can be fused to the N-terminus or C-terminus of the 50090 polypeptide.

The fusion protein can include a moiety that has a high affinity for a ligand. For example, the fusion protein can be a GST-50090 fusion protein in which the 50090 sequences are fused to the C-terminus of the GST sequences. Such fusion proteins can facilitate the purification of recombinant 50090. Alternatively, the fusion protein can be a 50090 protein containing a heterologous signal sequence at its N-terminus. In certain host cells (e.g., mammalian host cells), expression and/or secretion of 50090 can be increased through use of a heterologous signal sequence.

Fusion proteins can include all or a part of a serum protein, e.g., an IgG constant region, or human serum albumin.

The 50090 fusion proteins of the invention can be incorporated into pharmaceutical compositions and administered to a subject in vivo. The 50090 fusion proteins can be used to affect the bioavailability of a 50090 substrate. 50090 fusion proteins may be useful therapeutically for the treatment of disorders caused by, for example, (i) aberrant modification or mutation of a gene encoding a 50090 protein; (ii) mis-regulation of the 50090 gene; and (iii) aberrant post-translational modification of a 50090 protein.

Moreover, the 50090-fusion proteins of the invention can be used as immunogens to produce anti-50090 antibodies in a subject, to purify 50090 ligands and in screening assays to identify molecules that inhibit the interaction of 50090 with a 50090 substrate.

Expression vectors are commercially available that already encode a fusion moiety (e.g., a GST polypeptide). A 50090-encoding nucleic acid can be cloned into such an expression vector such that the fusion moiety is linked in-frame to the 50090 protein.

Variants of 50090 Proteins

In another aspect, the invention also features a variant of a 50090 polypeptide, e.g., which functions as an agonist (mimetics) or as an antagonist. Variants of the 50090 proteins can be generated by mutagenesis, e.g., discrete point mutation, the insertion or deletion of sequences or the truncation of a 50090 protein. An agonist of the 50090 proteins can retain substantially the same, or a subset, of the biological activities of the naturally occurring form of a 50090 protein. An antagonist of a 50090 protein can inhibit one or more of the activities of the naturally occurring form of the 50090 protein by, for example, competitively modulating a 50090-mediated activity of a 50090 protein. Thus, specific biological effects can be elicited by treatment with a variant of limited function. Preferably, treatment of a subject with a variant having a subset of the biological activities of the naturally occurring form of the protein has fewer side effects in a subject relative to treatment with the naturally occurring form of the 50090 protein.

Variants of a 50090 protein can be identified by screening combinatorial libraries of mutants, e.g., truncation mutants, of a 50090 protein for agonist or antagonist activity.

Libraries of fragments e.g., N terminal, C terminal, or internal fragments, of a 50090 protein coding sequence can be used to generate a variegated population of fragments for screening and subsequent selection of variants of a 50090 protein.

Variants in which a cysteine residue is added or deleted or in which a residue that is glycosylated is added or deleted are particularly preferred.

Methods for screening gene products of combinatorial libraries made by point mutations or truncation, and for screening cDNA libraries for gene products having a selected property. Recursive ensemble mutagenesis (REM), a new technique which enhances the frequency of functional mutants in the libraries, can be used in combination with the screening assays to identify 50090 variants (Arkin and Yourvan (1992) Proc. Natl. Acad. Sci. USA 89:7811-7815; Delgrave et al. (1993) Protein Engineering 6(3):327-331).

Cell based assays can be exploited to analyze a variegated 50090 library. For example, a library of expression vectors can be transfected into a cell line, e.g., a cell line, which ordinarily responds to 50090 in a substrate-dependent manner. The transfected cells are then contacted with 50090 and the effect of the expression of the mutant on signaling by the 50090 substrate can be detected. Plasmid DNA can then be recovered from the cells that score for inhibition, or alternatively, potentiation of signalling by the 50090 substrate, and the individual clones further characterized.

In another aspect, the invention features a method of making a 50090 polypeptide, e.g., a peptide having a non-wild type activity, e.g., an antagonist, agonist, or super agonist of a naturally occurring 50090 polypeptide, e.g., a naturally occurring 50090 polypeptide. The method includes: altering the sequence of a 50090 polypeptide, e.g., altering the sequence such as by substitution or deletion of one or more residues of a non-conserved region, a domain or residue disclosed herein, and testing the altered polypeptide for the desired activity.

In another aspect, the invention features a method of making a fragment or analog of a 50090 polypeptide a biological activity of a naturally occurring 50090 polypeptide. The method includes: altering the sequence, e.g., by substitution or deletion of one or more residues, of a 50090 polypeptide, e.g., altering the sequence of a non-conserved region, or a domain or residue described herein, and testing the altered polypeptide for the desired activity.

Anti-50090 Antibodies

In another aspect, the invention provides an anti-50090 antibody, or a fragment thereof (e.g., an antigen-binding fragment thereof). The term “antibody” as used herein refers to an immunoglobulin molecule or immunologically active portion thereof, i.e., an antigen-binding portion. As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4.

The anti-50090 antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

As used herein, the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd or 214 amino acids) are encoded by a variable region gene at the NH2-terminus (about 110 amino acids) and a kappa or lambda constant region gene at the COOH—terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids).

The term “antigen-binding fragment” of an antibody (or simply “antibody portion,” or “fragment”), as used herein, refers to one or more fragments of a full-length antibody that retain the ability to specifically bind to the antigen, e.g., 50090 polypeptide or fragment thereof. Examples of antigen-binding fragments of the anti-50090 antibody include, but are not limited to: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “antigen-binding fragment” of an antibody. These antibody fragments are obtained using conventional techniques known to those with skill in the art, and the fragments are screened for utility in the same manner as are intact antibodies.

The anti-50090 antibody can be a polyclonal or a monoclonal antibody, or other preparation where all or substantially all of the antibodies in the preparation bind to a single epitope. In other embodiments, the antibody can be recombinantly produced, e.g., produced by phage display or by combinatorial methods.

Phage display and combinatorial methods for generating anti-50090 antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9: 1370-1372; Hay et al. (1992) Hum Antibod Hybridomas 3:81-85; Huse et al. (1989) Science 246:1275-1281; Griffths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the anti-50090 antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), or camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An anti-50090 antibody can be one in which the variable region, or a portion thereof, e.g., the CDR's, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559). Antibody maybe replaced with at least a portion of a non-human CDR or only some of the CDR's may be replaced with non-human CDR's. It is only necessary to replace the number of CDR's required for binding of the humanized antibody to a 50090 or a fragment thereof.

A humanized or CDR-grafted antibody will have at least one or two but generally all three recipient CDR's (of heavy and or light immuoglobulin chains) replaced with a donor CDR. Preferably, the donor will be a rodent antibody, e.g., a rat or mouse antibody, and the recipient will be a human framework or a human consensus framework. Typically, the immunoglobulin providing the CDR's is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art. Humanized antibodies can be generated by replacing sequences of the Fv variable region that are not directly involved in antigen binding with equivalent sequences from human Fv variable regions. General methods for generating humanized antibodies are provided by Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a 50090 polypeptide or fragment thereof. The recombinant DNA encoding the humanized antibody, or fragment thereof, can then be cloned into an appropriate expression vector.

Humanized or CDR-grafted antibodies can be produced by CDR-grafting or CDR substitution, wherein one, two, or all CDR's of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Preferred humanized antibodies have amino acid substitutions in the framework region, such as to improve binding to the antigen. For example, a humanized antibody will have framework residues identical to the donor framework residue or to another amino acid other than the recipient framework residue. To generate such antibodies, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

In preferred embodiments an antibody can be made by immunizing with purified 50090 antigen, or a fragment thereof, e.g., a fragment described herein, membrane associated antigen, tissue, e.g., crude tissue preparations, lysed cells, or cell fractions, e.g., membrane fractions.

A full-length 50090 protein or antigenic peptide fragment of 50090 can be used as an immunogen or can be used to identify anti-50090 antibodies made with other immunogens, e.g., cells, membrane preparations, and the like. The antigenic peptide of 50090 should include at least 8 amino acid residues of the amino acid sequence shown in SEQ ID NO:29 and encompasses an epitope of 50090. Preferably, the antigenic peptide includes at least 10 amino acid residues, more preferably at least 15 amino acid residues, even more preferably at least 20 amino acid residues, and most preferably at least 30 amino acid residues.

Fragments of 50090 can be used to make, e.g., used as immunogens or used to characterize the specificity of an antibody. Antibodies can be made against hydrophilic regions of the 50090 protein, e.g., about amino acid residues 31 to 55, amino acid residues 106 to 123, and amino acid residues 215 to 235 of SEQ ID NO:29. Similarly, a fragment of 50090 that includes from about amino acids 70 to 79, amino acid residue 91 to 105, and amino acid residue 235 to 251 of SEQ ID NO:29 can be used to make an antibody against a hydrophobic region of the 50090 protein.

Antibodies reactive with, or specific for, any of these regions, or other regions or domains described herein are provided.

Preferred epitopes encompassed by the antigenic peptide are regions of 50090 that are located on the surface of the protein, e.g., hydrophilic regions, as well as regions with high antigenicity. For example, an Emini surface probability analysis of the human 50090 protein sequence can be used to indicate the regions that have a particularly high probability of being localized to the surface of the 50090 protein and are thus likely to constitute surface residues useful for targeting antibody production.

In a preferred embodiment, the antibody binds an epitope on any domain or region on 50090 proteins described herein.

Chimeric, humanized, but most preferably, completely human antibodies are desirable for applications that include repeated administration, e.g., therapeutic treatment (and some diagnostic applications) of human patients.

The anti-50090 antibody can be a single chain antibody. A single-chain antibody (scFV) may be engineered (see, for example, Colcher, D., et al. (1999) Ann. NY Acad. Sci. 880:263-80; and Reiter, Y. (1996) Clin. Cancer Res. (2):245-52). The single chain antibody can be dimerized or multimerized to generate multivalent antibodies having specificities for different epitopes of the same target 50090 protein.

In a preferred embodiment the antibody has effector function and can fix complement. In other embodiments the antibody does not recruit effector cells or fix complement.

In a preferred embodiment, the antibody has reduced or no ability to bind an Fc receptor. For example, it is an isotype or subtype, fragment or other mutant, which does not support binding to an Fc receptor, e.g., it has a mutagenized or deleted Fc receptor binding region.

The antibody can be coupled to a toxin, e.g., a polypeptide toxin such as ricin or diptheria toxin or active fragments thereof, or a radionuclide or imaging agent, e.g. a radioactive, enzymatic, or other, e.g., imaging agent, e.g., a NMR contrast agent. Labels that produce detectable radioactive emissions or fluorescence are preferred.

An anti-50090 antibody (e.g., monoclonal antibody) can be used to isolate 50090 by standard techniques, such as affinity chromatography or immunoprecipitation. Moreover, an anti-50090 antibody can be used to detect 50090 protein (e.g., in a cellular lysate or cell supernatant) in order to evaluate the abundance and pattern of expression of the protein. Anti-50090 antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labelling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidinibiotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

The invention also includes a nucleic acid that encodes an anti-50090 antibody, e.g., an anti-50090 antibody described herein. Also included are vectors that include the nucleic acid and cells transformed with the nucleic acid, particularly cells which are useful for producing an antibody, e.g., mammalian cells such as CHO or lymphatic cells.

The invention also includes cell lines, e.g., hybridomas, which make an anti-50090 antibody, e.g., and antibody described herein, and method of using said cells to make a 50090 antibody.

50090 Recombinant Expression Vectors, Host Cells and Genetically Engineered Cells

In another aspect, the invention includes, vectors, preferably expression vectors, containing a nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a 50090 nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids described herein (e.g., 50090 proteins, mutant forms of 50090 proteins, fusion proteins, and the like).

The recombinant expression vectors of the invention can be designed for expression of 50090 proteins in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.), which fuse glutathione S-transferase (GST), maltose E binding protein, or protein A, respectively, to the target recombinant protein.

Purified fusion proteins can be used in 50090 activity assays, (e.g., direct assays or competitive assays described in detail below), or to generate antibodies specific for 50090 proteins. In a preferred embodiment, a fusion protein expressed in a retroviral expression vector of the present invention can be used to infect bone marrow cells that are subsequently transplanted into irradiated recipients. The pathology of the subject recipient is then examined after sufficient time has passed (e.g., six (6) weeks).

To maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The 50090 expression vector can be a yeast expression vector, a vector for expression in insect cells, e.g., a baculovirus expression vector, or a vector suitable for expression in mammalian cells.

When used in mammalian cells, the expression vector's control functions are often provided by viral regulatory elements. For example, commonly used promoters are derived from polyoma, Adenovirus 2, cytomegalovirus and Simian Virus 40.

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus. For a discussion of the regulation of gene expression using antisense genes see Weintraub, H. et al., Antisense RNA as a molecular tool for genetic analysis, Reviews—Trends in Genetics, Vol. 1(1) 1986.

Another aspect the invention provides a host cell that includes a nucleic acid molecule described herein, e.g., a 50090 nucleic acid molecule within a recombinant expression vector or a 50090 nucleic acid molecule containing sequences that allow it to homologously recombine into a specific site of the host cell's genome. The terms “host cell” and “recombinant host cell” are used interchangeably herein. Such terms refer not only to the particular subject cell, but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a 50090 protein can be expressed in bacterial cells such as E. coli, insect cells, yeast or mammalian cells (such as CHO or COS cells). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation

A host cell of the invention can be used to produce (i.e., express) a 50090 protein. Accordingly, the invention further provides methods for producing a 50090 protein using the host cells of the invention. In one embodiment, the method includes culturing the host cell of the invention (into which a recombinant expression vector encoding a 50090 protein has been introduced) in a suitable medium such that a 50090 protein is produced. In another embodiment, the method further includes isolating a 50090 protein from the medium or the host cell.

In another aspect, the invention features, a cell or purified preparation of cells which include a 50090 transgene, or which otherwise misexpress 50090. The cell preparation can consist of human or non human cells, e.g., rodent cells, e.g., mouse or rat cells, rabbit cells, or pig cells. In preferred embodiments, the cell or cells include a 50090 transgene, e.g., a heterologous form of a 50090, e.g., a gene derived from humans (in the case of a non-human cell). The 50090 transgene can be misexpressed, e.g., overexpressed or underexpressed. In other preferred embodiments, the cell or cells include a gene that misexpresses an endogenous 50090, e.g., a gene the expression of which is disrupted, e.g., a knockout. Such cells can serve as a model for studying disorders that are related to mutated or mis-expressed 50090 alleles or for use in drug screening.

In another aspect, the invention features, a human cell, e.g., a hematopoietic stem cell, transformed with nucleic acid that encodes a subject 50090 polypeptide.

Also provided are cells, preferably human cells, e.g., human hematopoietic or fibroblast cells, in which an endogenous 50090 is under the control of a regulatory sequence that does not normally control the expression of the endogenous 50090 gene. The expression characteristics of an endogenous gene within a cell, e.g., a cell line or microorganism, can be modified by inserting a heterologous DNA regulatory element into the genome of the cell such that the inserted regulatory element is operably linked to the endogenous 50090 gene. For example, an endogenous 50090 gene that is “transcriptionally silent,” e.g., not normally expressed, or expressed only at very low levels, may be activated by inserting a regulatory element that is capable of promoting the expression of a normally expressed gene product in that cell. Techniques such as targeted homologous recombination can be used to insert the heterologous DNA as described in, e.g., U.S. Pat. No. 5,272,071 and PCT Publication No. WO 91/06667.

50090 Transgenic Animals

The invention provides non-human transgenic animals. Such animals are useful for studying the function and/or activity of a 50090 protein and for identifying and/or evaluating modulators of 50090 activity. As used herein, a “transgenic animal” is a non-human animal, preferably a mammal, more preferably a rodent such as a rat or mouse, in which one or more of the cells of the animal include a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous 50090 gene has been altered by, e.g., by homologous recombination between the endogenous gene and an exogenous DNA molecule introduced into a cell of the animal, e.g., an embryonic cell of the animal, prior to development of the animal.

Intronic sequences and polyadenylation signals can also be included in the transgene to increase the efficiency of expression of the transgene. A tissue-specific regulatory sequence(s) can be operably linked to a transgene of the invention to direct expression of a 50090 protein to particular cells. A transgenic founder animal can be identified based upon the presence of a 50090 transgene in its genome and/or expression of 50090 mRNA in tissues or cells of the animals. A transgenic founder animal can then be used to breed additional animals carrying the transgene. Moreover, transgenic animals carrying a transgene encoding a 50090 protein can further be bred to other transgenic animals carrying other transgenes. 50090 proteins or polypeptides can be expressed in transgenic animals or plants, e.g., a nucleic acid encoding the protein or polypeptide can be introduced into the genome of an animal. In preferred embodiments the nucleic acid is placed under the control of a tissue specific promoter, e.g., a milk or egg specific promoter, and recovered from the milk or eggs produced by the animal. Suitable animals are mice, pigs, cows, goats, and sheep.

The invention also includes a population of cells from a transgenic animal, as discussed, e.g., below.

Uses of 50090

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic).

The isolated nucleic acid molecules of the invention can be used, for example, to express a 50090 protein (e.g., via a recombinant expression vector in a host cell in gene therapy applications), to detect a 50090 mRNA (e.g., in a biological sample) or a genetic alteration in a 50090 gene, and to modulate 50090 activity, as described further below. The 50090 proteins can be used to treat disorders characterized by insufficient or excessive production of a 50090 substrate or production of 50090 inhibitors. In addition, the 50090 proteins can be used to screen for naturally occurring 50090 substrates, to screen for drugs or compounds which modulate 50090 activity, as well as to treat disorders characterized by insufficient or excessive production of 50090 protein or production of 50090 protein forms which have decreased, aberrant or unwanted activity compared to 50090 wild type protein (e.g., a liver or a muscular disorder). Moreover, the anti-50090 antibodies of the invention can be used to detect and isolate 50090 proteins, regulate the bioavailability of 50090 proteins, and modulate 50090 activities.

A method of evaluating a compound for the ability to interact with, e.g., bind, a subject 50090 polypeptide is provided. The method includes: contacting the compound with the subject 50090 polypeptide; and evaluating ability of the compound to interact with, e.g., to bind or form a complex with the subject 50090 polypeptide. This method can be performed in vitro, e.g., in a cell free system, or in vivo, e.g., in a two-hybrid interaction trap assay. This method can be used to identify naturally occurring molecules that interact with subject 50090 polypeptide. It can also be used to find natural or synthetic inhibitors of subject 50090 polypeptide. Screening methods are discussed in more detail below.

50090 Screening Assays:

The invention provides methods (also referred to herein as “screening assays”) for identifying modulators, i.e., candidate or test compounds or agents (e.g., proteins, peptides, peptidomimetics, peptoids, small molecules or other drugs) that bind to 50090 proteins, have a stimulatory or inhibitory effect on, for example, 50090 expression or 50090 activity, or have a stimulatory or inhibitory effect on, for example, the expression or activity of a 50090 substrate. Compounds thus identified can be used to modulate the activity of target gene products (e.g., 50090 genes) in a therapeutic protocol, to elaborate the biological function of the target gene product, or to identify compounds that disrupt normal target gene interactions.

In one embodiment, the invention provides assays for screening candidate or test compounds that are substrates of a 50090 protein or polypeptide or a biologically active portion thereof. In another embodiment, the invention provides assays for screening candidate or test compounds that bind to or modulate the activity of a 50090 protein or polypeptide or a biologically active portion thereof.

The test compounds of the present invention can be obtained using any of the numerous approaches in combinatorial library methods known in the art, including: biological libraries; peptoid libraries [libraries of molecules having the functionalities of peptides, but with a novel, non-peptide backbone which are resistant to enzymatic degradation but which nevertheless remain bioactive] (see, e.g., Zuckerman, R. N. et al. (1994) J. Med. Chem. 37: 2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, K. S. (1997) Anticancer Drug Des. 12:165).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria and spores (U.S. Pat. No. 5,223,409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell that expresses a 50090 protein or biologically active portion thereof is contacted with a test compound, and the ability of the test compound to modulate 50090 activity is determined. Determining the ability of the test compound to modulate 50090 activity can be accomplished by monitoring, for example, proteolytic activity. The cell, for example, can be of mammalian origin, e.g., mouse or human.

The ability of the test compound to modulate 50090 binding to a compound, e.g., a 50090 substrate, or to bind to 50090 can also be evaluated. This can be accomplished, for example, by coupling the compound, e.g., the substrate, with a radioisotope or enzymatic label such that binding of the compound, e.g., the substrate, to 50090 can be determined by detecting the labeled compound, e.g., substrate, in a complex. Alternatively, 50090 could be coupled with a radioisotope or enzymatic label to monitor the ability of a test compound to modulate 50090 binding to a 50090 substrate in a complex. For example, compounds (e.g., 50090 substrates) can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a 50090 substrate) to interact with 50090, with or without the labeling of any of the interactants can be evaluated. For example, a microphysiometer can be used to detect the interaction of a compound with 50090 without the labeling of either the compound or 50090. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 50090.

In yet another embodiment, a cell-free assay is provided in which a 50090 protein or biologically active portion thereof is contacted with a test compound and the ability of the test compound to bind to the 50090 protein or biologically active portion thereof is evaluated. Preferred biologically active portions of the 50090 proteins to be used in assays of the present invention include fragments that participate in interactions with non-50090 molecules, e.g., fragments with high surface probability scores.

Soluble and/or membrane-bound forms of isolated proteins (e.g., 50090 proteins or biologically active portions thereof) can be used in the cell-free assays of the invention. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, U.S. Pat. No. 5,631,169; U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of the 50090 protein to bind to a target molecule can be accomplished using real-time Biomolecular Interaction Analysis (BIA) (see, e.g., Sjolander, S. and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BLAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize either 50090, an anti-50090 antibody or its target molecule to facilitate separation of complexed from uncomplexed forms of one or both of the proteins, as well as to accommodate automation of the assay. Binding of a test compound to a 50090 protein, or interaction of a 50090 protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/50090 fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or 50090 protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of 50090 binding or activity determined using standard techniques.

Other techniques for immobilizing either a 50090 protein or a target molecule on matrices include using conjugation of biotin and streptavidin. Biotinylated 50090 protein or target molecules can be prepared from biotin-NHS(N-hydroxy-succinimide) using techniques known in the art (e.g., biotinylation kit, Pierce Chemicals, Rockford, ILL.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with 50090 protein or target molecules but which do not interfere with binding of the 50090 protein to its target molecule. Such antibodies can be derivatized to the wells of the plate, and unbound target or 50090 protein trapped in the wells by antibody conjugation. Methods for detecting such complexes, in addition to those described above for the GST-immobilized complexes, include immunodetection of complexes using antibodies reactive with the 50090 protein or target molecule, as well as enzyme-linked assays which rely on detecting an enzymatic activity associated with the 50090 protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., (1993) Trends Biochem Sci (8):284-7); chromatography (gel filtration chromatography, ion-exchange chromatography); electrophoresis (see, e.g., Ausubel, F. et al., eds. Current Protocols in Molecular Biology 1999, J. Wiley: New York.); and immunoprecipitation (see, for example, Ausubel, F. et al., eds. Current Protocols in Molecular Biology 1999, J. Wiley: New York). Such resins and chromatographic techniques are known to one skilled in the art (see, e.g., Heegaard, N. H., (1998) J Mol Recognit 11 (1-6):141-8; Hage, D. S., and Tweed, S. A. (1997) J Chromatogr B Biomed Sci Appl 699(1-2):499-525). Further, fluorescence energy transfer may also be conveniently utilized, as described herein, to detect binding without further purification of the complex from solution.

In a preferred embodiment, the assay includes contacting the 50090 protein or biologically active portion thereof with a known compound that binds 50090 to form an assay mixture, contacting the assay mixture with a test compound, and determining the ability of the test compound to interact with a 50090 protein, wherein determining the ability of the test compound to interact with a 50090 protein includes determining the ability of the test compound to preferentially bind to 50090 or biologically active portion thereof, or to modulate the activity of a target molecule, as compared to the known compound.

The target gene products of the invention can, in vivo, interact with one or more cellular or extracellular macromolecules, such as proteins. For the purposes of this discussion, such cellular and extracellular macromolecules are referred to herein as “binding partners.”

Compounds that disrupt such interactions can be useful in regulating the activity of the target gene product. Such compounds can include, but are not limited to, molecules such as antibodies, peptides, and small molecules. The preferred target genes/products for use in this embodiment are the 50090 genes herein identified. In an alternative embodiment, the invention provides methods for determining the ability of the test compound to modulate the activity of a 50090 protein through modulation of the activity of a downstream effector of a 50090 target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the target gene product and the binding partner is prepared under conditions and for a time sufficient to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product, or the interactive cellular or extracellular binding partner, is anchored onto a solid surface (e.g., a microtiter plate), while the non-anchored species is labeled either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays).

The addition of a test substance that competes with and displaces one of the species from the preformed complex will result in the generation of a signal above background. In this way, test substances that disrupt target gene product-binding partner interaction can be identified.

In yet another aspect, the 50090 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and WO94/10300), to identify other proteins, which bind to or interact with 50090 (“50090-binding proteins” or “50090-bp”) and are involved in 50090 activity. Such 50090-bps can be activators or inhibitors of signals by the 50090 proteins or 50090 targets as, for example, downstream elements of a 50090-mediated signaling pathway.

The two-hybrid system is based on the modular nature of most transcription factors, which consist of separable DNA-binding and activation domains. Briefly, the assay utilizes two different DNA constructs. In one construct, the gene that codes for a 50090 protein is fused to a gene encoding the DNA binding domain of a known transcription factor (e.g., GAL-4). In the 30 other construct, a DNA sequence, from a library of DNA sequences, that encodes an unidentified protein (“prey” or “sample”) is fused to a gene that codes for the activation domain of the known transcription factor. (Alternatively, the 50090 protein can be fused to the activator domain.) If the “bait” and the “prey” proteins are able to interact in vivo forming a 50090-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein that interacts with the 50090 protein.

In another embodiment, modulators of 50090 expression are identified. For example, a cell or cell free mixture is contacted with a candidate compound and the expression of 50090 mRNA or protein evaluated relative to the level of expression of 50090 mRNA or protein in the absence of the candidate compound. When expression of 50090 mRNA or protein is greater in the presence of the candidate compound than in its absence, the candidate compound is identified as a stimulator of 50090 mRNA or protein expression. Alternatively, when expression of 50090 mRNA or protein is less (statistically significantly less) in the presence of the candidate compound than in its absence, the candidate compound is identified as an inhibitor of 50090 mRNA or protein expression. The level of 50090 mRNA or protein expression can be determined by methods described herein for detecting 50090 mRNA or protein.

In another aspect, the invention pertains to a combination of two or more of the assays described herein. For example, a modulating agent can be identified using a cell-based or a cell free assay, and the ability of the agent to modulate the activity of a 50090 protein can be confirmed in vivo, e.g., in an animal such as an animal model for cancer.

This invention further pertains to novel agents identified by the above-described screening assays. Accordingly, it is within the scope of this invention to further use an agent identified as described herein (e.g., a 50090 modulating agent, an antisense 50090 nucleic acid molecule, a 50090-specific antibody, or a 50090-binding partner) in an appropriate animal model to determine the efficacy, toxicity, side effects, or mechanism of action, of treatment with such an agent. Furthermore, novel agents identified by the above-described screening assays can be used for treatments as described herein.

50090 Detection Assays

Portions or fragments of the nucleic acid sequences identified herein can be used as polynucleotide reagents. For example, these sequences can be used to: (i) map their respective genes on a chromosome e.g., to locate gene regions associated with genetic disease or to associate 50090 with a disease; (ii) identify an individual from a minute biological sample (tissue typing); and (iii) aid in forensic identification of a biological sample. These applications are described in the subsections below.

50090 Chromosome Mapping

The 50090 nucleotide sequences or portions thereof can be used to map the location of the 50090 genes on a chromosome. This process is called chromosome mapping. Chromosome mapping is useful in correlating the 50090 sequences with genes associated with disease.

Briefly, 50090 genes can be mapped to chromosomes by preparing PCR primers (preferably 15-25 bp in length) from the 50090 nucleotide sequences. These primers can then be used for PCR screening of somatic cell hybrids containing individual human chromosomes. Only those hybrids containing the human gene corresponding to the 50090 sequences will yield an amplified fragment.

A panel of somatic cell hybrids in which each cell line contains either a single human chromosome or a small number of human chromosomes, and a full set of mouse chromosomes, can allow easy mapping of individual genes to specific human chromosomes. (D'Eustachio P. et al., (1983) Science 220:919-924).

Other mapping strategies e.g., in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries can be used to map 50090 to a chromosomal location.

Fluorescence in situ hybridization (FISH) of a DNA sequence to a metaphase chromosomal spread can further be used to provide a precise chromosomal location in one step. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the 50090 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

50090 Tissue Typing

50090 sequences can be used to identify individuals from biological samples using, e.g., restriction fragment length polymorphism (RFLP). In this technique, an individual's genomic DNA is digested with one or more restriction enzymes, the fragments separated, e.g., in a Southern blot, and probed to yield bands for identification. The sequences of the present invention are useful as additional DNA markers for RFLP (described in U.S. Pat. No. 5,272,057).

Furthermore, the sequences of the present invention can also be used to determine the actual base-by-base DNA sequence of selected portions of an individual's genome. Thus, the 50090 nucleotide sequences described herein can be used to prepare two PCR primers from the 5′ and 3′ ends of the sequences. These primers can then be used to amplify an individual's DNA and subsequently sequence it. Panels of corresponding DNA sequences from individuals, prepared in this manner, can provide unique individual identifications, as each individual will have a unique set of such DNA sequences due to allelic differences.

Allelic variation occurs to some degree in the coding regions of these sequences, and to a greater degree in the noncoding regions. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:28 can provide positive individual identification with a panel of perhaps 10 to 1,000 primers that each yield a noncoding amplified sequence of 100 bases. If predicted coding sequences, such as those in SEQ ID NO:30 are used, a more appropriate number of primers for positive individual identification would be 500-2,000.

If a panel of reagents from 50090 nucleotide sequences described herein is used to generate a unique identification database for an individual, those same reagents can later be used to identify tissue from that individual. Using the unique identification database, positive identification of the individual, living or dead, can be made from extremely small tissue samples.

Use of Partial 50090 Sequences in Forensic Biology

DNA-based identification techniques can also be used in forensic biology. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:28 (e.g., fragments derived from the noncoding regions of SEQ ID NO:28 having a length of at least 20 bases, preferably at least 30 bases) are particularly appropriate for this use.

The 50090 nucleotide sequences described herein can further be used to provide polynucleotide reagents, e.g., labeled or labelable probes which can be used in, for example, an in situ hybridization technique, to identify a specific tissue. This can be very useful in cases where a forensic pathologist is presented with a tissue of unknown origin. Panels of such 50090 probes can be used to identify tissue by species and/or by organ type.

In a similar fashion, these reagents, e.g., 50090 primers or probes can be used to screen tissue culture for contamination (i.e. screen for the presence of a mixture of different types of cells in a culture).

Predictive Medicine of 50090

The present invention also pertains to the field of predictive medicine in which diagnostic assays, prognostic assays, and monitoring clinical trials are used for prognostic (predictive) purposes to thereby treat an individual.

Generally, the invention provides, a method of determining if a subject is at risk for a disorder related to a lesion in or the misexpression of a gene that encodes a 50090 polypeptide. Such disorders include, e.g., a disorder associated with the misexpression of a 50090 molecule, a proliferation disorder, or a cardiac or muscle cell disorder.

The method includes one or more of the following:

detecting, in a tissue of the subject, the presence or absence of a mutation that affects the expression of the 50090 gene, or detecting the presence or absence of a mutation in a region which controls the expression of the gene, e.g., a mutation in the 5′ control region;

detecting, in a tissue of the subject, the presence or absence of a mutation that alters the structure of the 50090 gene;

detecting, in a tissue of the subject, the misexpression of the 50090 gene, at the mRNA level, e.g., detecting a non-wild type level of a mRNA;

detecting, in a tissue of the subject, the misexpression of the gene, at the protein level, e.g., detecting a non-wild type level of a 50090 polypeptide.

In preferred embodiments, the method includes ascertaining the existence of at least one of: a deletion of one or more nucleotides from the 50090 gene; an insertion of one or more nucleotides into the gene, a point mutation, e.g., a substitution of one or more nucleotides of the gene, a gross chromosomal rearrangement of the gene, e.g., a translocation, inversion, or deletion.

For example, detecting the genetic lesion can include: (i) providing a probe/primer including an oligonucleotide containing a region of nucleotide sequence which hybridizes to a sense or antisense sequence from SEQ ID NO:28, or naturally occurring mutants thereof or 5′ or 3′ flanking sequences naturally associated with the 50090 gene; (ii) exposing the probe/primer to nucleic acid of the tissue; and detecting, by hybridization, e.g., in situ hybridization, of the probe/primer to the nucleic acid, the presence or absence of the genetic lesion.

In preferred embodiments, detecting the misexpression includes ascertaining the existence of at least one of: an alteration in the level of a messenger RNA transcript of the 50090 gene; the presence of a non-wild type splicing pattern of a messenger RNA transcript of the gene; or a non-wild type level of 50090.

Methods of the invention can be used prenatally or to determine if a subject's offspring will be at risk for a disorder.

In preferred embodiments the method includes determining the structure of a 50090 gene, an abnormal structure being indicative of risk for the disorder.

In preferred embodiments the method includes contacting a sample form the subject with an antibody to the 50090 protein or a nucleic acid that hybridizes specifically with the gene. These and other embodiments are discussed below.

Diagnostic and Prognostic Assays of 50090

Diagnostic and prognostic assays of the invention include method for assessing the expression level of 50090 molecules and for identifying variations and mutations in the sequence of 50090 molecules.

Expression Monitoring and Profilling:

The presence, level, or absence of 50090 protein or nucleic acid in a biological sample can be evaluated by obtaining a biological sample from a test subject and contacting the biological sample with a compound or an agent capable of detecting 50090 protein or nucleic acid (e.g., mRNA, genomic DNA) that encodes 50090 protein such that the presence of 50090 protein or nucleic acid is detected in the biological sample. The term “biological sample” includes tissues, cells and biological fluids isolated from a subject, as well as tissues, cells and fluids present within a subject. A preferred biological sample is serum. The level of expression of the 50090 gene can be measured in a number of ways, including, but not limited to: measuring the mRNA encoded by the 50090 genes; measuring the amount of protein encoded by the 50090 genes; or measuring the activity of the protein encoded by the 50090 genes.

The level of mRNA corresponding to the 50090 gene in a cell can be determined both by in situ and by in vitro formats.

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length 50090 nucleic acid, such as the nucleic acid of SEQ ID NO:28, or a portion thereof, such as an oligonucleotide of at least 7, 15, 30, 50, 100, 250 or 500 nucleotides in length and sufficient to specifically hybridize under stringent conditions to 50090 mRNA or genomic DNA. The probe can be disposed on an address of an array, e.g., an array described below. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array described below. A skilled artisan can adapt known mRNA detection methods for use in detecting the level of mRNA encoded by the 50090 genes.

The level of mRNA in a sample that is encoded by one of 50090 can be evaluated with nucleic acid amplification, e.g., by rtPCR (Mullis (1987) U.S. Pat. No. 4,683,202), ligase chain reaction (Barany (1991) Proc. Natl. Acad. Sci. USA 88:189-193), self sustained sequence replication (Guatelli et al., (1990) Proc. Natl. Acad. Sci. USA 87:1874-1878), transcriptional amplification system (Kwoh et al., (1989), Proc. Natl. Acad. Sci. USA 86:1173-1177), Q-Beta Replicase (Lizardi et al., (1988) Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the 50090 gene being analyzed.

In another embodiment, the methods further contacting a control sample with a compound or agent capable of detecting 50090 mRNA, or genomic DNA, and comparing the presence of 50090 mRNA or genomic DNA in the control sample with the presence of 50090 mRNA or genomic DNA in the test sample. In still another embodiment, serial analysis of gene expression, as described in U.S. Pat. No. 5,695,937, is used to detect 50090 transcript levels.

A variety of methods can be used to determine the level of protein encoded by 50090. In general, these methods include contacting an agent that selectively binds to the protein, such as an antibody with a sample, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect 50090 protein in a biological sample in vitro as well as in vivo. In vitro techniques for detection of 50090 protein include enzyme linked immunosorbent assays (ELISAs), immunoprecipitations, immunofluorescence, enzyme immunoassay (EIA), radioimmunoassay (RIA), and Western blot analysis. In vivo techniques for detection of 50090 protein include introducing into a subject a labeled anti-50090 antibody. For example, the antibody can be labeled with a radioactive marker whose presence and location in a subject can be detected by standard imaging techniques. In another embodiment, the sample is labeled, e.g., biotinylated and then contacted to the antibody, e.g., an anti-50090 antibody positioned on an antibody array (as described below). The sample can be detected, e.g., with avidin coupled to a fluorescent label.

In another embodiment, the methods further include contacting the control sample with a compound or agent capable of detecting 50090 protein, and comparing the presence of 50090 protein in the control sample with the presence of 50090 protein in the test sample.

The invention also includes kits for detecting the presence of 50090 in a biological sample. For example, the kit can include a compound or agent capable of detecting 50090 protein or mRNA in a biological sample; and a standard. The compound or agent can be packaged in a suitable container. The kit can further comprise instructions for using the kit to detect 50090 protein or nucleic acid.

For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can include: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also includes a buffering agent, a preservative, or a protein stabilizing agent. The kit can also includes components necessary for detecting the detectable agent (e.g., an enzyme or a substrate). The kit can also contain a control sample or a series of control samples that can be assayed and compared to the test sample contained. Each component of the kit can be enclosed within an individual container and all of the various containers can be within a single package, along with instructions for interpreting the results of the assays performed using the kit.

The diagnostic methods described herein can identify subjects having, or at risk of developing, a disease or disorder associated with misexpressed or aberrant or unwanted 50090 expression or activity. As used herein, the term “unwanted” includes an unwanted phenomenon involved in a biological response such as pain or deregulated cell proliferation.

In one embodiment, a disease or disorder associated with aberrant or unwanted 50090 expression or activity is identified. A test sample is obtained from a subject and 50090 protein or nucleic acid (e.g., mRNA or genomic DNA) is evaluated, wherein the level, e.g., the presence or absence, of 50090 protein or nucleic acid is diagnostic for a subject having or at risk of developing a disease or disorder associated with aberrant or unwanted 50090 expression or activity. As used herein, a “test sample” refers to a biological sample obtained from a subject of interest, including a biological fluid (e.g., serum), cell sample, or tissue.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted 50090 expression or activity. For example, such methods can be used to determine whether a subject can be effectively treated with an agent for a genetic disorder, neuronal disorder, liver disorder, cardiac or skeletal muscle disorder, or cancer.

In another aspect, the invention features a computer medium having a plurality of digitally encoded data records. Each data record includes a value representing the level of expression of 50090 in a sample, and a descriptor of the sample. The descriptor of the sample can be an identifier of the sample, a subject from which the sample was derived (e.g., a patient), a diagnosis, or a treatment (e.g., a preferred treatment). In a preferred embodiment, the data record further includes values representing the level of expression of genes other than 50090 (e.g., other genes associated with a 50090-disorder, or other genes on an array). The data record can be structured as a table, e.g., a table that is part of a database such as a relational database (e.g., a SQL database of the Oracle or Sybase database environments).

Also featured is a method of evaluating a sample. The method includes providing a sample, e.g., from the subject, and determining a gene expression profile of the sample, wherein the profile includes a value representing the level of 50090 expression. The method can further include comparing the value or the profile (i.e., multiple values) to a reference value or reference profile. The gene expression profile of the sample can be obtained by any of the methods described herein (e.g., by providing a nucleic acid from the sample and contacting the nucleic acid to an array). The profile can be compared to a reference profile or to a profile obtained from the subject prior to treatment or prior to onset of the disorder (see, e.g., Golub et al. (1999) Science 286:531).

In yet another aspect, the invention features a method of evaluating a test compound (see also, “Screening Assays”, above). The method includes providing a cell and a test compound; contacting the test compound to the cell; obtaining a subject expression profile for the contacted cell; and comparing the subject expression profile to one or more reference profiles. The profiles include a value representing the level of 50090 expression. In a preferred embodiment, the subject expression profile is compared to a target profile, e.g., a profile for a normal cell or for desired condition of a cell. The test compound is evaluated favorably if the subject expression profile is more similar to the target profile than an expression profile obtained from an uncontacted cell.

In another aspect, the invention features, a method of evaluating a subject. The method includes: a) obtaining a sample from a subject, e.g., from a caregiver, e.g., a caregiver who obtains the sample from the subject; b) determining a subject expression profile for the sample. Optionally, the method further includes either or both of steps: c) comparing the subject expression profile to one or more reference expression profiles; and d) selecting the reference profile most similar to the subject reference profile. The subject expression profile and the reference profiles include a value representing the level of 50090 expression. A variety of routine statistical measures can be used to compare two reference profiles. One possible metric is the length of the distance vector that is the difference between the two profiles. Each of the subject and reference profile is represented as a multi-dimensional vector, wherein each dimension is a value in the profile.

The method can further include transmitting a result to a caregiver. The result can be the subject expression profile, a result of a comparison of the subject expression profile with another profile, a most similar reference profile, or a descriptor of any of the aforementioned. The result can be transmitted across a computer network, e.g., the result can be in the form of a computer transmission, e.g., a computer data signal embedded in a carrier wave.

Also featured is a computer medium having executable code for effecting the following steps: receive a subject expression profile; access a database of reference expression profiles; and either i) select a matching reference profile most similar to the subject expression profile or ii) determine at least one comparison score for the similarity of the subject expression profile to at least one reference profile. The subject expression profile, and the reference expression profiles each include a value representing the level of 50090 expression.

50090 Arrays and Uses Thereof

In another aspect, the invention features an array that includes a substrate having a plurality of addresses. At least one address of the plurality includes a capture probe that binds specifically to a 50090 molecule (e.g., a 50090 nucleic acid or a 50090 polypeptide). The array can have a density of at least than 10, 50, 100, 200, 500, 1,000, 2,000, or 10,000 or more addresses/cm², and ranges between. In a preferred embodiment, the plurality of addresses includes at least 10, 100, 500, 1,000, 5,000, 10,000, 50,000 addresses. In a preferred embodiment, the plurality of addresses includes equal to or less than 10, 100, 500, 1,000, 5,000, 10,000, or 50,000 addresses. The substrate can be a two-dimensional substrate such as a glass slide, a wafer (e.g., silica or plastic), a mass spectroscopy plate, or a three-dimensional substrate such as a gel pad. Addresses in addition to address of the plurality can be disposed on the array.

In a preferred embodiment, at least one address of the plurality includes a nucleic acid capture probe that hybridizes specifically to a 50090 nucleic acid, e.g., the sense or anti-sense strand. In one preferred embodiment, a subset of addresses of the plurality of addresses has a nucleic acid capture probe for 50090. Each address of the subset can include a capture probe that hybridizes to a different region of a 50090 nucleic acid. In another preferred embodiment, addresses of the subset include a capture probe for a 50090 nucleic acid. Each address of the subset is unique, overlapping, and complementary to a different variant of 50090 (e.g., an allelic variant, or all possible hypothetical variants). The array can be used to sequence 50090 by hybridization (see, e.g., U.S. Pat. No. 5,695,940).

An array can be generated by various methods, e.g., by photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145).

In another preferred embodiment, at least one address of the plurality includes a polypeptide capture probe that binds specifically to a 50090 polypeptide or fragment thereof. The polypeptide can be a naturally-occurring interaction partner of 50090 polypeptide. Preferably, the polypeptide is an antibody, e.g., an antibody described herein (see “Anti-50090 Antibodies,” above), such as a monoclonal antibody or a single-chain antibody.

In another aspect, the invention features a method of analyzing the expression of 50090. The method includes providing an array as described above; contacting the array with a sample and detecting binding of a 50090-molecule (e.g., nucleic acid or polypeptide) to the array. In a preferred embodiment, the array is a nucleic acid array. Optionally the method further includes amplifying nucleic acid from the sample prior or during contact with the array.

In another embodiment, the array can be used to assay gene expression in a tissue to ascertain tissue specificity of genes in the array, particularly the expression of 50090. If a sufficient number of diverse samples is analyzed, clustering (e.g., hierarchical clustering, k-means clustering, Bayesian clustering and the like) can be used to identify other genes which are co-regulated with 50090. For example, the array can be used for the quantitation of the expression of multiple genes. Thus, not only tissue specificity, but also the level of expression of a battery of genes in the tissue is ascertained. Quantitative data can be used to group (e.g., cluster) genes on the basis of their tissue expression per se and level of expression in that tissue.

For example, array analysis of gene expression can be used to assess the effect of cell-cell interactions on 50090 expression. A first tissue can be perturbed and nucleic acid from a second tissue that interacts with the first tissue can be analyzed. In this context, the effect of one cell type on another cell type in response to a biological stimulus can be determined, e.g., to monitor the effect of cell-cell interaction at the level of gene expression.

In another embodiment, cells are contacted with a therapeutic agent. The expression profile of the cells is determined using the array, and the expression profile is compared to the profile of like cells not contacted with the agent. For example, the assay can be used to determine or analyze the molecular basis of an undesirable effect of the therapeutic agent. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor expression of one or more genes in the array with respect to time. For example, samples obtained from different time points can be probed with the array. Such analysis can identify and/or characterize the development of a 50090-associated disease or disorder; and processes, such as a cellular transformation associated with a 50090-associated disease or disorder. The method can also evaluate the treatment and/or progression of a 50090-associated disease or disorder The array is also useful for ascertaining differential expression patterns of one or more genes in normal and abnormal cells. This provides a battery of genes (e.g., including 50090) that could serve as a molecular target for diagnosis or therapeutic intervention.

In another aspect, the invention features an array having a plurality of addresses. Each address of the plurality includes a unique polypeptide. At least one address of the plurality has disposed thereon a 50090 polypeptide or fragment thereof. Methods of producing polypeptide arrays are described in the art, e.g., in De Wildt et al. (2000). Nature Biotech. 18, 989-994; Lueking et al. (1999). Anal. Biochem. 270, 103-111; Ge, H. (2000). Nucleic Acids Res. 28, e3, 1-VII; MacBeath, G., and Schreiber, S. L. (2000). Science 289, 1760-1763; and WO 99/51773A1. In a preferred embodiment, each addresses of the plurality has disposed thereon a polypeptide at least 60, 70, 80,85, 90, 95 or 99% identical to a 50090 polypeptide or fragment thereof. For example, multiple variants of a 50090 polypeptide (e.g., encoded by allelic variants, site-directed mutants, random mutants, or combinatorial mutants) can be disposed at individual addresses of the plurality. Addresses in addition to the address of the plurality can be disposed on the array.

The polypeptide array can be used to detect a 50090 binding compound, e.g., an antibody in a sample from a subject with specificity for a 50090 polypeptide or the presence of a 50090-binding protein or ligand.

The array is also useful for ascertaining the effect of the expression of a gene on the expression of other genes in the same cell or in different cells (e.g., ascertaining the effect of 50090 expression on the expression of other genes). This provides, for example, for a selection of alternate molecular targets for therapeutic intervention if the ultimate or downstream target cannot be regulated.

In another aspect, the invention features a method of analyzing a plurality of probes. The method is useful, e.g., for analyzing gene expression. The method includes: providing a two dimensional array having a plurality of addresses, each address of the plurality being positionally distinguishable from each other address of the plurality having a unique capture probe, e.g., wherein the capture probes are from a cell or subject which express 50090 or from a cell or subject in which a 50090 mediated response has been elicited, e.g., by contact of the cell with 50090 nucleic acid or protein, or administration to the cell or subject 50090 nucleic acid or protein; providing a two dimensional array having a plurality of addresses, each address of the plurality being positionally distinguishable from each other address of the plurality, and each address of the plurality having a unique capture probe, e.g., wherein the capture probes are from a cell or subject which does not express 50090 (or does not express as highly as in the case of the 50090 positive plurality of capture probes) or from a cell or subject which in which a 50090 mediated response has not been elicited (or has been elicited to a lesser extent than in the first sample); contacting the array with one or more inquiry probes (which is preferably other than a 50090 nucleic acid, polypeptide, or antibody), and thereby evaluating the plurality of capture probes. Binding, e.g., in the case of a nucleic acid, hybridization with a capture probe at an address of the plurality, is detected, e.g., by signal generated from a label attached to the nucleic acid, polypeptide, or antibody.

In another aspect, the invention features a method of analyzing a plurality of probes or a sample. The method is useful, e.g., for analyzing gene expression. The method includes: providing a two dimensional array having a plurality of addresses, each address of the plurality being positionally distinguishable from each other address of the plurality having a unique capture probe, contacting the array with a first sample from a cell or subject which express or mis-express 50090 or from a cell or subject in which a 50090-mediated response has been elicited, e.g., by contact of the cell with 50090 nucleic acid or protein, or administration to the cell or subject 50090 nucleic acid or protein; providing a two dimensional array having a plurality of addresses, each address of the plurality being positionally distinguishable from each other address of the plurality, and each address of the plurality having a unique capture probe, and contacting the array with a second sample from a cell or subject which does not express 50090 (or does not express as highly as in the case of the 50090 positive plurality of capture probes) or from a cell or subject which in which a 50090 mediated response has not been elicited (or has been elicited to a lesser extent than in the first sample); and comparing the binding of the first sample with the binding of the second sample. Binding, e.g., in the case of a nucleic acid, hybridization with a capture probe at an address of the plurality, is detected, e.g., by signal generated from a label attached to the nucleic acid, polypeptide, or antibody. The same array can be used for both samples or different arrays can be used. If different arrays are used the plurality of addresses with capture probes should be present on both arrays.

In another aspect, the invention features a method of analyzing 50090, e.g., analyzing structure, function, or relatedness to other nucleic acid or amino acid sequences. The method includes: providing a 50090 nucleic acid or amino acid sequence; comparing the 50090 sequence with one or more preferably a plurality of sequences from a collection of sequences, e.g., a nucleic acid or protein sequence database; to thereby analyze 50090.

Detection of 50090 Variations or Mutations

The methods of the invention can also be used to detect genetic alterations in a 50090 gene, thereby determining if a subject with the altered gene is at risk for a disorder characterized by misregulation in 50090 protein activity or nucleic acid expression, such as a neuronal disorder, cancer, infectious diseases, liver disorders, and cardiac and skeletal muscle disorders. In preferred embodiments, the methods include detecting, in a sample from the subject, the presence or absence of a genetic alteration characterized by at least one of an alteration affecting the integrity of a gene encoding a 50090-protein, or the mis-expression of the 50090 gene. For example, such genetic alterations can be detected by ascertaining the existence of at least one of 1) a deletion of one or more nucleotides from a 50090 gene; 2) an addition of one or more nucleotides to a 50090 gene; 3) a substitution of one or more nucleotides of a 50090 gene, 4) a chromosomal rearrangement of a 50090 gene; 5) an alteration in the level of a messenger RNA transcript of a 50090 gene, 6) aberrant modification of a 50090 gene, such as of the methylation pattern of the genomic DNA, 7) the presence of a non-wild type splicing pattern of a messenger RNA transcript of a 50090 gene, 8) a non-wild type level of a 50090-protein, 9) allelic loss of a 50090 gene, and 10) inappropriate post-translational modification of a 50090-protein.

An alteration can be detected without a probe/primer in a polymerase chain reaction, such as anchor PCR or RACE PCR, or, alternatively, in a ligation chain reaction (LCR), the latter of which can be particularly useful for detecting point mutations in the 50090-gene. This method can include the steps of collecting a sample of cells from a subject, isolating nucleic acid (e.g., genomic, mRNA or both) from the sample, contacting the nucleic acid sample with one or more primers which specifically hybridize to a 50090 gene under conditions such that hybridization and amplification of the 50090-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein. Alternatively, other amplification methods described herein or known in the art can be used.

In another embodiment, mutations in a 50090 gene from a sample cell can be identified by detecting alterations in restriction enzyme cleavage patterns. For example, sample and control DNA is isolated, amplified (optionally), digested with one or more restriction endonucleases, and fragment length sizes are determined, e.g., by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 50090 can be identified by hybridizing a sample and control nucleic acids, e.g., DNA or RNA, two-dimensional arrays, e.g., chip based arrays. Such arrays include a plurality of addresses, each of which is positionally distinguishable from the other. A different probe is located at each address of the plurality. A probe can be complementary to a region of a 50090 nucleic acid or a putative variant (e.g., allelic variant) thereof. A probe can have one or more mismatches to a region of a 50090 nucleic acid (e.g., a destabilizing mismatch). The arrays can have a high density of addresses, e.g., can contain hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in 50090 can be identified in two-dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 50090 gene and detect mutations by comparing the sequence of the sample 50090 with the corresponding wild-type (control) sequence. Automated sequencing procedures can be utilized when performing the diagnostic assays ((1995) Biotechniques 19:448), including sequencing by mass spectrometry.

Other methods for detecting mutations in the 50090 gene include methods in which protection from cleavage agents is used to detect mismatched bases in RNA/RNA or RNA/DNA heteroduplexes (Myers et al. (1985) Science 230:1242; Cotton et al. (1988) Proc. Natl. Acad Sci USA 85:4397; Saleeba et al. (1992) Methods Enzymol. 217:286-295).

In still another embodiment, the mismatch cleavage reaction employs one or more proteins that recognize mismatched base pairs in double-stranded DNA (so called “DNA mismatch repair” enzymes) in defined systems for detecting and mapping point mutations in 50090 cDNAs obtained from samples of cells. For example, the mutY enzyme of E. coli cleaves A at G/A mismatches and the thymidine DNA glycosylase from HeLa cells cleaves T at G/T mismatches (Hsu et al (1994) Carcinogenesis 15:1657-1662; U.S. Pat. No. 5,459,039).

In other embodiments, alterations in electrophoretic mobility will be used to identify mutations in 50090 genes. For example, single strand conformation polymorphism (SSCP) may be used to detect differences in electrophoretic mobility between mutant and wild type nucleic acids (Orita et al (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control 50090 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension (Saiki et al. (1986) Nature 324:163); Saiki et al. (1989) Proc. Natl Acad. Sci USA 86:6230). A further method of detecting point mutations is the chemical ligation of oligonucleotides as described in Xu et al. ((2001) Nature Biotechnol. 19:148). Adjacent oligonucleotides, one of which selectively anneals to the query site, are ligated together if the nucleotide at the query site of the sample nucleic acid is complementary to the query oligonucleotide; ligation can be monitored, e.g., by fluorescent dyes coupled to the oligonucleotides.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6: 1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

In another aspect, the invention features a set of oligonucleotides. The set includes a plurality of oligonucleotides, each of which is at least partially complementary (e.g., at least 50%, 60%, 70%, 80%, 90%, 92%, 95%, 97%, 98%, or 99% complementary) to a 50090 nucleic acid.

In a preferred embodiment the set includes a first and a second oligonucleotide. The first and second oligonucleotide can hybridize to the same or to different locations of SEQ ID NO:28 or the complement of SEQ ID NO:28. Different locations can be different but overlapping or or nonoverlapping on the same strand. The first and second oligonucleotide can hybridize to sites on the same or on different strands.

The set can be useful, e.g., for identifying SNP's, or identifying specific alleles of 50090. In a preferred embodiment, each oligonucleotide of the set has a different nucleotide at an interrogation position. In one embodiment, the set includes two oligonucleotides, each complementary to a different allele at a locus, e.g., a biallelic or polymorphic locus.

In another embodiment, the set includes four oligonucleotides, each having a different nucleotide (e.g., adenine, guanine, cytosine, or thymidine) at the interrogation position. The interrogation position can be a SNP or the site of a mutation. In another preferred embodiment, the oligonucleotides of the plurality are identical in sequence to one another (except for differences in length). The oligonucleotides can be provided with differential labels, such that an oligonucleotide that hybridizes to one allele provides a signal that is distinguishable from an oligonucleotide that hybridizes to a second allele. In still another embodiment, at least one of the oligonucleotides of the set has a nucleotide change at a position in addition to a query position, e.g., a destabilizing mutation to decrease the Tm of the oligonucleotide. In another embodiment, at least one oligonucleotide of the set has a non-natural nucleotide, e.g., inosine. In a preferred embodiment, the oligonucleotides are attached to a solid support, e.g., to different addresses of an array or to different beads or nanoparticles.

In a preferred embodiment the set of oligo nucleotides can be used to specifically amplify, e.g., by PCR, or detect, a 50090 nucleic acid.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a 50090 gene.

Use of 50090 Molecules as Surrogate Markers

The 50090 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the 50090 molecules of the invention may be detected, and may be correlated with one or more biological states in vivo. For example, the 50090 molecules of the invention may serve as surrogate markers for one or more disorders or disease states or for conditions leading up to disease states. As used herein, a “surrogate marker” is an objective biochemical marker that correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The 50090 molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., a 50090 marker) transcription or expression, the amplified marker may be in a quantity which is more readily detectable than the drug itself. Also, the marker may be more easily detected due to the nature of the marker itself; for example, using the methods described herein, anti-50090 antibodies may be employed in an immune-based detection system for a 50090 protein marker, or 50090-specific radiolabeled probes may be used to detect a 50090 mRNA marker. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am, J Health-Syst. Pharm. 56 Suppl. 3: S16-S20.

The 50090 molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35:1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or protein (e.g., 50090 protein or RNA) for specific tumor markers in a subject, a drug or course of treatment may be selected that is optimized for the treatment of the specific tumor likely to be present in the subject. Similarly, the presence or absence of a specific sequence mutation in 50090 DNA may correlate 50090 drug response. The use of pharmacogenomic markers therefore permits the application of the most appropriate treatment for each subject without having to administer the therapy.

Pharmaceutical Compositions of 50090

The nucleic acid and polypeptides, fragments thereof, as well as anti-50090 antibodies (also referred to herein as “active compounds”) of the invention can be incorporated into pharmaceutical compositions. Such compositions typically include the nucleic acid molecule, protein, or antibody and a pharmaceutically acceptable carrier. As used herein, “pharmaceutically acceptable carrier” includes solvents, dispersion media, coatings, antibacterial and antifingal agents, isotonic and absorption delaying agents, and the like, which are compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

For antibodies, the preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The present invention encompasses agents that modulate expression or activity. An agent may, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 μg/kg to about 500 mg/kg, about 100 μg/kg to about 5 mg/kg, or about 1 μg/kg to about 50 μg/kg. It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

An antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response; the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Methods of Treatment for 50090

The present invention provides for both prophylactic and therapeutic methods of treating a subject at risk of (or susceptible to) a disorder or having a disorder associated with aberrant or unwanted 50090 expression or activity. With respect to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the 50090 molecules of the present invention or 50090 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

In one aspect, the invention provides a method for preventing in a subject, a disease or condition associated with an aberrant or unwanted 50090 expression or activity, by administering to the subject a 50090 or an agent which modulates 50090 expression or at least one 50090 activity. Subjects at risk for a disease which is caused or contributed to by aberrant or unwanted 50090 expression or activity can be identified by, for example, any or a combination of diagnostic or prognostic assays as described herein. Administration of a prophylactic agent can occur prior to the manifestation of symptoms characteristic of the 50090 aberrance, such that a disease or disorder is prevented or, alternatively, delayed in its progression. Depending on the type of 50090 aberrance, 50090, a 50090 agonist, or a 50090 antagonist can be used for treating the subject. The appropriate agent can be determined based on screening assays described herein.

It is possible that some 50090 disorders can be caused, at least in part, by an abnormal level of gene product, or by the presence of a gene product exhibiting abnormal activity. As such, the reduction in the level and/or activity of such gene products would bring about the amelioration of disorder symptoms.

The 50090 molecules can act as novel diagnostic targets and therapeutic agents for controlling one or more disorders associated with defects in fatty acid oxidation, or proliferation or muscular disorders.

As discussed above, successful treatment of 50090 disorders can be brought about by techniques that serve to inhibit the expression or activity of target gene products. For example, compounds, e.g., an agent identified using an assay described above, that prove to exhibit negative modulatory activity, can be used in accordance with the invention to prevent and/or ameliorate symptoms of 50090 disorders. Such molecules can include, but are not limited to peptides, phosphopeptides, small organic or inorganic molecules, or antibodies (including, for example, polyclonal, monoclonal, humanized, anti-idiotypic, chimeric or single chain antibodies, and Fab, F(ab)₂ and Fab expression library fragments, scFV molecules, and epitope-binding fragments thereof).

Further, antisense and ribozyme molecules that inhibit expression of the target gene can also be used in accordance with the invention to reduce the level of target gene expression, thus effectively reducing the level of target gene activity. Still further, triple helix molecules can be utilized in reducing the level of target gene activity. Antisense, ribozyme and triple helix molecules are discussed above.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances in that the target gene encodes an extracellular protein, it can be preferable to co-administer normal target gene protein into the cell or tissue in order to maintain the requisite level of cellular or tissue target gene activity.

Another method by which nucleic acid molecules may be utilized in treating or preventing a disease characterized by 50090 expression is through the use of aptamer molecules specific for 50090 protein. Aptamers are nucleic acid molecules having a tertiary structure that permits them to specifically bind to protein ligands (see, e.g., Osborne, et al. Curr. Opin. Chem Biol. 1997, 1(1): 5-9; and Patel, D. J. Curr Opin Chem Biol 1997 Jun;1(1):32-46). Since nucleic acid molecules may in many cases be more conveniently introduced into target cells than therapeutic protein molecules may be, aptamers offer a method by which 50090 protein activity may be specifically decreased without the introduction of drugs or other molecules which may have pluripotent effects.

Antibodies can be generated that are both specific for target gene product and that reduce target gene product activity. Such antibodies may, therefore, by administered in instances whereby negative modulatory techniques are appropriate for the treatment of 50090 disorders. For a description of antibodies, see the Antibody section above.

In circumstances wherein injection of an animal or a human subject with a 50090 protein or epitope for stimulating antibody production is harmful to the subject, it is possible to generate an immune response against 50090 through the use of anti-idiotypic antibodies (see, for example, Herlyn, D. Ann Med 1999;31(1):66-78; and Bhattacharya-Chatterjee, M., and Foon, K. A. Cancer Treat Res 1998;94:51-68). If an anti-idiotypic antibody is introduced into a mammal or human subject, it should stimulate the production of anti-anti-idiotypic antibodies, which should be specific to the 50090 protein. Vaccines directed to a disease characterized by 50090 expression may also be generated in this fashion.

In instances where the target antigen is intracellular and whole antibodies are used, internalizing antibodies may be preferred. Lipofectin or liposomes can be used to deliver the antibody or a fragment of the Fab region that binds to the target antigen into cells. Where fragments of the antibody are used, the smallest inhibitory fragment that binds to the target antigen is preferred. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al. (1993) Proc. Natl. Acad. Sci. USA 90:7889-7893).

The identified compounds that inhibit target gene expression, synthesis and/or activity can be administered to a patient at therapeutically effective doses to prevent, treat or ameliorate 50090 disorders. A therapeutically effective dose refers to that amount of the compound sufficient to result in amelioration of symptoms of the disorders.

Toxicity and therapeutic efficacy of such compounds can be determined by standard pharmaceutical procedures in cell cultures or experimental animals as described above for pharmaceutical compositions. The data obtained from the cell culture assays and animal studies can be used in formulating a range of dosage for use in humans. The dosage of such compounds lies preferably within a range of circulating concentrations that include the ED₅₀ with little or no toxicity, as described above.

Another example of determination of effective dose for an individual is the ability to directly assay levels of “free” and “bound” compound in the serum of the test subject. Such assays may utilize antibody mimics and/or “biosensors” that have been created through molecular imprinting techniques. The compound which is able to modulate 50090 activity is used as a template, or “imprinting molecule”, to spatially organize polymerizable monomers prior to their polymerization with catalytic reagents. The subsequent removal of the imprinted molecule leaves a polymer matrix that contains a repeated “negative image” of the compound and is able to selectively rebind the molecule under biological assay conditions. A detailed review of this technique can be seen in Ansell, R. J. et al (1996) Current Opinion in Biotechnology 7:89-94 and in Shea, K. J. (1994) Trends in Polymer Science 2:166-173. Such “imprinted” affinity matrixes are amenable to ligand-binding assays, whereby the immobilized monoclonal antibody component is replaced by an appropriately imprinted matrix. An example of the use of such matrixes in this way can be seen in Vlatakis, G. et al (1993) Nature 361:645-647. Through the use of isotope-labeling, the “free” concentration of compound which modulates the expression or activity of 50090 can be readily monitored and used in calculations of IC₅₀. Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. An rudimentary example of such a “biosensor” is discussed in Kriz, D. et al (1995) Analytical Chemistry 67:2142-2144.

Another aspect of the invention pertains to methods of modulating 50090 expression or activity for therapeutic purposes. Accordingly, in an exemplary embodiment, the modulatory method of the invention involves contacting a cell with a 50090 or agent that modulates one or more of the activities of 50090 protein activity associated with the cell. An agent that modulates 50090 protein activity can be an agent as described herein, such as a nucleic acid or a protein, a naturally-occurring target molecule of a 50090 protein (e.g., a 50090 substrate or receptor), a 50090 antibody, a 50090 agonist or antagonist, a peptidomimetic of a 50090 agonist or antagonist, or other small molecule.

In one embodiment, the agent stimulates one or more 50090 activities. Examples of such stimulatory agents include active 50090 protein and a nucleic acid molecule encoding 50090. In another embodiment, the agent inhibits one or more 50090 activities. Examples of such inhibitory agents include antisense 50090 nucleic acid molecules, anti-50090 antibodies, and 50090 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 50090 protein or nucleic acid molecule. In one embodiment, the method involves administering an agent (e.g., an agent identified by a screening assay described herein), or combination of agents that modulates (e.g., upregulates or downregulates) 50090 expression or activity. In another embodiment, the method involves administering a 50090 protein or nucleic acid molecule as therapy to compensate for reduced, aberrant, or unwanted 50090 expression or activity.

Stimulation of 50090 activity is desirable in situations in which 50090 is abnormally downregulated and/or in which increased 50090 activity is likely to have a beneficial effect. For example, stimulation of 50090 activity is desirable in situations in which a 50090 is downregulated and/or in which increased 50090 activity is likely to have a beneficial effect. Likewise, inhibition of 50090 activity is desirable in situations in which 50090 is abnormally upregulated and/or in which decreased 50090 activity is likely to have a beneficial effect.

The 50090 molecules can act as novel diagnostic targets and therapeutic agents for controlling one or more of cellular proliferative and/or differentiative disorders, cardiac disorders, and muscle disorders, as described above, as well as disorders associated with bone metabolism, immune disorders, liver disorders, viral diseases, or metabolic disorders.

Aberrant expression and/or activity of 50090 molecules may mediate disorders associated with bone metabolism. “Bone metabolism” refers to direct or indirect effects in the formation or degeneration of bone structures, e.g., bone formation, bone resorption, etc., which may ultimately affect the concentrations in serum of calcium and phosphate. This term also includes activities mediated by 50090 molecules effects in bone cells, e.g. osteoclasts and osteoblasts, that may in turn result in bone formation and degeneration. For example, 50090 molecules may support different activities of bone resorbing osteoclasts such as the stimulation of differentiation of monocytes and mononuclear phagocytes into osteoclasts. Accordingly, 50090 molecules that modulate the production of bone cells can influence bone formation and degeneration, and thus may be used to treat bone disorders. Examples of such disorders include, but are not limited to, osteoporosis, osteodystrophy, osteomalacia, rickets, osteitis fibrosa cystica, renal osteodystrophy, osteosclerosis, anti-convulsant treatment, osteopenia, fibrogenesis-imperfecta ossium, secondary hyperparathyrodism, hypoparathyroidism, hyperparathyroidism, cirrhosis, obstructive jaundice, drug induced metabolism, medullary carcinoma, chronic renal disease, rickets, sarcoidosis, glucocorticoid antagonism, malabsorption syndrome, steatorrhea, tropical sprue, idiopathic hypercalcemia and milk fever.

The 50090 nucleic acid and protein of the invention can be used to treat and/or diagnose a variety of immune disorders. Examples of hematopoieitic disorders or diseases include, but are not limited to, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjogren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions,leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy such as, atopic allergy.

Examples of disorders involving the heart or “cardiovascular disorder” include, but are not limited to, a disease, disorder, or state involving the cardiovascular system, e.g., the heart, the blood vessels, and/or the blood. A cardiovascular disorder can be caused by an imbalance in arterial pressure, a malfunction of the heart, or an occlusion of a blood vessel, e.g., by a thrombus. Examples of such disorders include hypertension, atherosclerosis, coronary artery spasm, congestive heart failure, coronary artery disease, valvular disease, arrhythmias, and cardiomyopathies.

Disorders which may be treated or diagnosed by methods described herein include, but are not limited to, disorders associated with an accumulation in the liver of fibrous tissue, such as that resulting from an imbalance between production and degradation of the extracellular matrix accompanied by the collapse and condensation of preexisting fibers. The methods described herein can be used to diagnose or treat hepatocellular necrosis or injury induced by a wide variety of agents including processes which disturb homeostasis, such as an inflammatory process, tissue damage resulting from toxic injury or altered hepatic blood flow, and infections (e.g., bacterial, viral and parasitic). For example, the methods can be used for the early detection of hepatic injury, such as portal hypertension or hepatic fibrosis. In addition, the methods can be employed to detect liver fibrosis attributed to inborn errors of metabolsim, for example, fibrosis resulting from a storage disorder such as Gaucher's disease (lipid abnormalities) or a glycogen storage disease, Al-antitrypsin deficiency; a disorder mediating the accumulation (e.g., storage) of an exogenous substance, for example, hemochromatosis (iron-overload syndrome) and copper storage diseases (Wilson's disease), disorders resulting in the accumulation of a toxic metabolite (e.g., tyrosinemia, fructosemia and galactosemia) and peroxisomal disorders (e.g., Zellweger syndrome). Additionally, the methods described herein may be useful for the early detection and treatment of liver injury associated with the administration of various chemicals or drugs, such as for example, methotrexate, isonizaid, oxyphenisatin, methyldopa, chlorpromazine, tolbutamide or alcohol, or which represents a hepatic manifestation of a vascular disorder such as obstruction of either the intrahepatic or extrahepatic bile flow or an alteration in hepatic circulation resulting, for example, from chronic heart failure, veno-occlusive disease, portal vein thrombosis or Budd-Chiari syndrome.

Additionally, 50090 molecules may play an important role in the etiology of certain viral diseases, inducing but not limited to Hepatitis B, Heptitis C and Herpes Simplex Virus (HSV). Modulators of 50090 activity could be used to control viral diseases. The modulators can be used in the treatment and/or diagnosis of viral infected tissue or virus-associated tissue fibrosis, especially liver and liver fibrosis. Also, 50090 modulators can be used in the treatment and/or diagnosis of virus-associated carcinoma, especially hepatocellular cancer.

Additionally, 50090 may play an important role in the regulation of metabolism disorders. Diseases of metabolic imbalance include, but are not limited to, obesity, anorexia nervosa, cachexia, lipid disorders diabetes.

50090 Pharmacogenomics

The 50090 molecules of the present invention, as well as agents or modulators that have a stimulatory or inhibitory effect on 50090 activity (e.g., 50090 gene expression) as identified by a screening assay described herein can be administered to individuals to treat (prophylactically or therapeutically) 50090 associated disorders (e.g., liver disorders, cardiac disorders, or muscular disorders) associated with aberrant or unwanted 50090 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a 50090 molecule or 50090 modulator as well as tailoring the dosage and/or therapeutic regimen of treatment with a 50090 molecule or 50090 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11):983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomic approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high-resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach” can be utilized to identify genes that predict drug response. According to this method, if a gene that encodes a drug's target is known (e.g., a 50090 protein of the present invention), all common variants of that gene can be fairly easily identified in the population and it can be determined if having one version of the gene versus another is associated with a particular drug response.

A method termed the “gene expression profiling” also can be utilized to identify genes that predict drug response. For example, the gene expression of an animal dosed with a drug (e.g., a 50090 molecule or 50090 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a 50090 molecule or 50090 modulator, such as a modulator identified by one of the exemplary screening assays described herein.

The present invention further provides methods for identifying new agents, or combinations, that are based on identifying agents that modulate the activity of one or more of the gene products encoded by one or more of the 50090 genes of the present invention, wherein these products may be associated with resistance of the cells to a therapeutic agent. Specifically, the activity of the proteins encoded by the 50090 genes of the present invention can be used as a basis for identifying agents for overcoming agent resistance. By blocking the activity of one or more of the resistance proteins, target cells, e.g., human cells, will become sensitive to treatment with an agent that the unmodified target cells were resistant to.

Monitoring the influence of agents (e.g., drugs) on the expression or activity of a 50090 protein can be applied in clinical trials. For example, the effectiveness of an agent determined by a screening assay as described herein to increase 50090 gene expression, protein levels, or upregulate 50090 activity, can be monitored in clinical trials of subjects exhibiting decreased 50090 gene expression, protein levels, or downregulated 50090 activity. Alternatively, the effectiveness of an agent determined by a screening assay to decrease 50090 gene expression, protein levels, or downregulate 50090 activity, can be monitored in clinical trials of subjects exhibiting increased 50090 gene expression, protein levels, or upregulated 50090 activity. In such clinical trials, the expression or activity of a 50090 gene, and preferably, other genes that have been implicated in, for example, a 50090-associated disorder can be used as a “read out” or markers of the phenotype of a particular cell.

50090 Informatics

The sequence of a 50090 molecule is provided in a variety of media to facilitate use thereof. A sequence can be provided as a manufacture, other than an isolated nucleic acid or amino acid molecule, which contains a 50090. Such a manufacture can provide a nucleotide or amino acid sequence, e.g., an open reading frame, in a form which allows examination of the manufacture using means not directly applicable to examining the nucleotide or amino acid sequences, or a subset thereof, as they exists in nature or in purified form. The sequence information can include, but is not limited to, 50090 full-length nucleotide and/or amino acid sequences, partial nucleotide and/or amino acid sequences, polymorphic sequences including single nucleotide polymorphisms (SNPs), epitope sequence, and the like. In a preferred embodiment, the manufacture is a machine-readable medium, e.g., a magnetic, optical, chemical or mechanical information storage device.

As used herein, “machine-readable media” refers to any medium that can be read and accessed directly by a machine, e.g., a digital computer or analogue computer. Non-limiting examples of a computer include a desktop PC, laptop, mainframe, server (e.g., a web server, network server, or server farm), handheld digital assistant, pager, mobile telephone, and the like. The computer can be stand-alone or connected to a communications network, e.g., a local area network (such as a VPN or intranet), a wide area network (e.g., an Extranet or the Internet), or a telephone network (e.g., a wireless, DSL, or ISDN network). Machine-readable media include, but are not limited to: magnetic storage media, such as floppy discs, hard disc storage medium, and magnetic tape; optical storage media such as CD-ROM; electrical storage media such as RAM, ROM, EPROM, EEPROM, flash memory, and the like; and hybrids of these categories such as magnetic/optical storage media.

A variety of data storage structures are available to a skilled artisan for creating a machine-readable medium having recorded thereon a nucleotide or amino acid sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. The skilled artisan can readily adapt any number of data processor structuring formats (e.g., text file or database) in order to obtain computer readable medium having recorded thereon the nucleotide sequence information of the present invention.

In a preferred embodiment, the sequence information is stored in a relational database (such as Sybase or Oracle). The database can have a first table for storing sequence (nucleic acid and/or amino acid sequence) information. The sequence information can be stored in one field (e.g., a first column) of a table row and an identifier for the sequence can be store in another field (e.g., a second column) of the table row. The database can have a second table, e.g., storing annotations. The second table can have a field for the sequence identifier, a field for a descriptor or annotation text (e.g., the descriptor can refer to a functionality of the sequence, a field for the initial position in the sequence to which the annotation refers, and a field for the ultimate position in the sequence to which the annotation refers. Non-limiting examples for annotation to nucleic acid sequences include polymorphisms (e.g., SNP's) translational regulatory sites and splice junctions. Non-limiting examples for annotations to amino acid sequence include polypeptide domains, e.g., a domain described herein; active sites and other functional amino acids; and modification sites.

By providing the nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or target structural motif with the sequence information stored within the data storage means. A search is used to identify fragments or regions of the sequences of the invention that match a particular target sequence or target motif. The search can be a BLAST search or other routine sequence comparison, e.g., a search described herein.

Thus, in one aspect, the invention features a method of analyzing 50090, e.g., analyzing structure, function, or relatedness to one or more other nucleic acid or amino acid sequences. The method includes: providing a 50090 nucleic acid or amino acid sequence; comparing the 50090 sequence with a second sequence, e.g., one or more preferably a plurality of sequences from a collection of sequences, e.g., a nucleic acid or protein sequence database to thereby analyze 50090. The method can be performed in a machine, e.g., a computer, or manually by a skilled artisan.

The method can include evaluating the sequence identity between a 50090 sequence and a database sequence. The method can be performed by accessing the database at a second site, e.g., over the Internet.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. Typical sequence lengths of a target sequence are from about 10 to 100 amino acids or from about 30 to 300 nucleotide residues. However, it is well recognized that commercially important fragments, such as sequence fragments involved in gene expression and protein processing, may be of shorter length.

Computer software is publicly available which allows a skilled artisan to access sequence information provided in a computer readable medium for analysis and comparison to other sequences. A variety of known algorithms are disclosed publicly and a variety of commercially available software for conducting search means are and can be used in the computer-based systems of the present invention. Examples of such software include, but are not limited to, MacPattern (EMBL), BLASTN and BLASTX (NCBI).

Thus, the invention features a method of making a computer readable record of a sequence of a 50090 sequence, which includes recording the sequence on a computer readable matrix. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′ end of the translated region.

In another aspect, the invention features, a method of analyzing a sequence. The method includes: providing a 50090 sequence, or record, in machine-readable form; comparing a second sequence to the 50090 sequence; thereby analyzing a sequence. Comparison can include comparing to sequences for sequence identity or determining if one sequence is included within the other, e.g., determining if the 50090 sequence includes a sequence being compared. In a preferred embodiment the 50090 or second sequence is stored on a first computer, e.g., at a first site and the comparison is performed, read, or recorded on a second computer, e.g., at a second site. E.g., the 50090 or second sequence can be stored in a public or proprietary database in one computer, and the results of the comparison performed, read, or recorded on a second computer. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′ end of the translated region.

In another aspect, the invention provides a machine-readable medium for holding instructions for performing a method for determining whether a subject has a 50090-associated disease or disorder or a pre-disposition to a 50090-associated disease or disorder, wherein the method comprises the steps of determining 50090 sequence information associated with the subject and based on the 50090 sequence information, determining whether the subject has a 50090-associated disease or disorder or a pre-disposition to a 50090-associated disease or disorder and/or recommending a particular treatment for the disease, disorder or pre-disease condition.

The invention further provides in an electronic system and/or in a network, a method for determining whether a subject has a 50090-associated disease or disorder or a pre-disposition to a disease associated with a 50090 wherein the method comprises the steps of determining 50090 sequence information associated with the subject, and based on the 50090 sequence information, determining whether the subject has a 50090-associated disease or disorder or a pre-disposition to a 50090-associated disease or disorder, and/or recommending a particular treatment for the disease, disorder or pre-disease condition. In a preferred embodiment, the method further includes the step of receiving information, e.g., phenotypic or genotypic information, associated with the subject and/or acquiring from a network phenotypic information associated with the subject. The information can be stored in a database, e.g., a relational database. In another embodiment, the method further includes accessing the database, e.g., for records relating to other subjects, comparing the 50090 sequence of the subject to the 50090 sequences in the database to thereby determine whether the subject as a 50090-associated disease or disorder, or a pre-disposition for such.

The present invention also provides in a network, a method for determining whether a subject has a 50090 associated disease or disorder or a pre-disposition to a 50090-associated disease or disorder associated with 50090, said method comprising the steps of receiving 50090 sequence information from the subject and/or information related thereto, receiving phenotypic information associated with the subject, acquiring information from the network corresponding to 50090 and/or corresponding to a 50090-associated disease or disorder (e.g., cancer, cardiac and skeletal muscle disorder, liver disorders, infectious disease, and neuronal disorders), and based on one or more of the phenotypic information, the 50090 information (e.g., sequence information and/or information related thereto), and the acquired information, determining whether the subject has a 50090-associated disease or disorder or a pre-disposition to a 50090-associated disease or disorder. The method may further comprise the step of recommending a particular treatment for the disease, disorder or pre-disease condition.

The present invention also provides a method for determining whether a subject has a 50090-associated disease or disorder or a pre-disposition to a 50090-associated disease or disorder, said method comprising the steps of receiving information related to 50090 (e.g., sequence information and/or information related thereto), receiving phenotypic information associated with the subject, acquiring information from the network related to 50090 and/or related to a 50090-associated disease or disorder, and based on one or more of the phenotypic information, the 50090 information, and the acquired information, determining whether the subject has a 50090-associated disease or disorder or a pre-disposition to a 50090-associated disease or disorder. The method may further comprise the step of recommending a particular treatment for the disease, disorder or pre-disease condition.

This invention is further illustrated by the following examples that should not be construed as limiting. The contents of all references, patents and published patent applications cited throughout this application are incorporated herein by reference.

### EXAMPLES

### Examples for 33312. 33303, and 32579

### Example 1

### Identification and Characterization of Human 33312, 33303, and 32579 cDNAs Human 33312

The human 33312 nucleic acid sequence is recited as follows:

(SEQ ID NO:1)

CCGGGCAGGTACGCGGGGAGAGCTCAGGACCTCTGAGAAGAATGGAGCCC
TCCTGGCTTCAGGAACTCATGGCTCACCCCTTCTTGCTGCTGATCCTCCT
CTGCATGTCTCTGCTGCTGTTTCAGGTAATCAGGTTGTACCAGAGGAGGA
GATGGATGATCAGAGCCCTGCACCTGTTTCCTGCACCCCCTGCCCACTGG
TTCTATGGCCACAAGGAGTTTTACCCCCTTGTGGGTTGGACCCTTTACGA
TGTTCTTCAGTGTCCATGACCCAGACTATGCCAAGATTCTCCTGAAAAGA
CAAGATCCCAAAAGTGCTGTTAGCCACAAAATCCTTGAATCCTGGGTTGG
TCGAGGACTTGTGACCCTGGATGGTTCTAAATGGAAAAAGCACCGCCAGA
TTGTGAAACCTGGCTTCAACATCAGCATTCTGAAAATATTCATCACCATG
ATGTCTGAGAGTGTTCGGATGATGCTGAACAAATGGGAGGAACACATTGC
CCAAAACTCACGTCTGGAGCTCTTTCAACATGTCTCCCTGATGACCCTGG
ACAGCATCATGAAGTGTGCCTTCAGCCACCAGGGCAGCATCCAGTTGGAC
AGTACCCTGGACTCATACCTGAAAGCAGTGTTCAACCTTAGCAAAATCTC
CAACCAGCGCATGAACAATTTTCTACATCACAACGACCTGGTTTTCAAAT
TCAGCTCTCAAGGCCAAATCTTTTCTAAATTTAACCAAGAACTTCATCAG
TTCACAGAGAAAGTAATCCAGGACCGGAAGGAGTCTCTTAAGGATAAGCT
AAAACAAGATACTACTCAGAAAAGGCGCTGGGATTTTCTGGACATACTTT
TGAGTGCCAAAAGCGAAAACACCAAAGATTTCTCTGAAGCAGATCTCCAG
GCTGAAGTGAAAACGTTCATGTTTGCAGGACATGACACCACATCCAGTGC
TATCTCCTGGATCCTTTACTGCTTGGCKAAGTACCCTGAGCATCAGCAGA
GATGCCGAGATGAAATCAGGGAACTCCTAGGGGATGGGTCTTCTATTACC
TGGGAACACCTGAGCCAGATGCCTTACACCACGATGTGCATCAAGGAATG
CCTCCGCCTCTACGCACCGGTAGTAAACATATCCCGGTTACTCGACAAAC
CCATCACCTTTCCAGATGGACGCTCCTTACCTGCAGGAATAACTGTGTTT
ATCAATATTTGGGCTCTTCACCACAACCCCTATTTCTGGGAAGACCCTCA
GGTCTTTAACCCCTTGAGATTCTCCAGGGAAAATTCTGAAAAAATACATC
CCTATGCCTTCATACCATTCTCAGCTGGATTAAGGAACTGCATTGGGCAG
CATTTTGCCATAATTGAGTGTAAAGTGGCAGTGGCATTAACTCTGCTCCG
CTTCAACAAGAATGGAATCCATGTGTTTGCAAAAAAAGTTTGCTAATTTT
AAGTCCTTTCGTATAAGAATTAATGAGACAATTTTCCTACCAAAGGAAGA
ACAAAAGGATAAATATAATACAAAATATATGTATATGGTTGTTTGACAAA
TTATATAACTTAGGATACTTCTGACACAAAAACACCTGAAAAAACTCAAG
CTGACTTCCACTGCGAAGGGAAATTATTGGTTTGTGTAACTAGTGGTAGA
GTGGCTTTCAAGCATAGTTTGATCAAAACTCCACTCAGTATCTGCATTAC
TTTTATCTCTGCAAATATCTGCATGATAGCTTTATTCTCAGTTATCTTTC
CCCATAATAAAAAATATCTGCCAAAAAAAAAAAAAAAAAAAAACGCTCGA
AAGGG.

The human 33312 sequence (SEQ ID NO:1) is approximately 1975 nucleotides long. The nucleic acid sequence includes an initiation codon (ATG) and a termination codon (TAA), which are bolded and underscored above. The region between and inclusive of the initiation codon and the termination codon is a methionine-initiated coding sequence of about 1518 nucleotides, including the termination codon (nucleotides indicated as “coding” of SEQ ID NO: 1; SEQ ID NO:3). The coding sequence encodes a 505 amino acid protein (SEQ ID NO:2), which is recited as follows:

(SEQ ID NO:2)

MEPSWLQELMAHPFLLLILLCMSLLLFQVIRLYQRRRWMIRALHLFPAPP
AHWFYGHKEFYPVKEFEVYHKLMEKYPCAVPLWVGPFTMFFSVHDPDYAK
ILLKRQDPKSAVSHKILESWVGRGLVTLDGSKWKKHRQIVKPGFNISILK
IFITMMSESVRMMLNKWEEHIAQNSRLELFQHVSLMTLDSIMKCAFSHQG
SIQLDSTLDSYLKAVFNLSKISNQRMNNFLHHNDLVFKFSSQGQIFSKFN
QELHQFTEKVIQDRKESLKDKLKQDTTQKRRWDFLDILLSAKSENTKDFS
EADLQAEVKTFMFAGHDTTSSAISWILYCLAKYPEHQQRCRDEIRELLGD
GSSITWEHLSQMPYTTMCIKECLRLYAPVVNISRLLDKPITFPDGRSLPA
GITVFINIWALHHNPYFWEDPQVFNPLRFSRENSEKIHPYAFIPFSAGLR
NCIGQHFAIIECKVAVALTLLRFKLAPDHSRPPQPVRQVVLKSKNGIHVF
AKKVC.

Human 33303

The human 33303 nucleic acid sequence is recited as follows:

(SEQ ID NO:4)

ATGGAGGCGACCGGCACCTGGGCGCTGCTGCTGGCGCTGGCGCTGCTCCT
GCTGCTGACGCTGGCGCTGTCCGGGACCAGGGCCCGAGGCCACCTGCCCC
CCGGGCCCACGCCGCTACCACTGCTGGGAAACCTCCTGCAGCTACGGCCC
GGGGCGCTGTATTCAGGGCTCATGCGGCTGAGTAAGAAGTACGGACCGGT
GTTCACCATCTACCTGGGACCGTGGCGGCCTGTGGTGGTCCTGGTTGGGC
AGGAGGCTGTGCGGGAGGCCCTGGGAGGTCAGGCTGAGGAGTTCAGCGGC
CGGGGAACCGTAGCGATGCTGGAAGGGACTTTTGATGGCCATGGGGTTTT
CTTCTCCAACGGGGAGCGGTGGAGGCAGCTGAGGAAGTTTACCATGCTTG
CTCTGCGGGACCTGGGCATGGGGAAGCGAGAAGGCGAGGAGCTGATCCAG
GCGGAGGCCCGGTGTCTGGTGGAGACATTCCAGGGGACAGAAGGACGCCC
ATTCGATCCCTCCCTGCTGCTGGCCCAGGCCACCTCCAACGTAGTCTGCT
CCCTCCTCTTTGGCCTCCGCTTCTCCTATGAGGATAAGGAGTTCCAGGCC
GTGGTCCGGGCAGCTGGTGGTACCCTGCTGGGAGTCAGCTCCCAGGGGGG
TCAGACCTACGAGATGTTCTCCTGGTTCCTGCGGCCCCTGCCAGGCCCCC
ACAAGCAGCTCCTCCACCACGTCAGCACCTTGGCTGCCTTCACAGTCCGG
CAGGTGCAGCAGCACCAGGGGAACCTGGATGCTTCGGGCCCCGCACGTGA
CCTTGTCGATGCCTTCCTGCTGAAGATGGCACAGGAGGAACAAAACCCAG
GCACAGAATTCACCAACAAGAACATGCTGATGACAGTCATTTATTTGCTG
TTTGCTGGGACGATGACGGTCAGCACCACGGTCGGCTATACCCTCCTGCT
CCTGATGAAATACCCTCATGTCCAAAAGTGGGTACGTGAGGAGCTGAATC
GGGAGCTGGGGGCTGGCCAGGCACCAAGCCTAGGGGACCGTACCCGCCTC
CCTTACACCGACGCGGTTCTGCATGAGGCGCAGCGGCTGCTGGCGCTGGT
GCCCATGGGAATACCCCGCACCCTCATGCGGACCACCCGCTTCCGAGGGT
ACACCCTGCCCCAGGGCACGGAGGTCTTCCCCCTCCTTGGCTCCATCCTG
CATGACCCCAACATCTTCAAGCACCCAGAAGAGTTCAACCCAGACCGTTT
CCTGGATGCAGATGGACGGTTCAGGAAGCATGAGGCGTTCCTGCCCTTCT
CCTTAGGGAAGCGTGTCTGCCTTGGAGAGGGCCTGGCAAAAGCGGAGCTC
TTCCTCTTCTTCACCACCATCCTACAAGCCTTCTCCCTGGAGAGCCCGTG
CCCGCCGGACACCCTGAGCCTCAAGCCCACCGTCAGTGGCCTTTTCAACA
TTCCCCCAGCCTTCCAGCTGCAAGTCCGTCCCACTGACCTTCACTCCACC
ACGCAGACCAGATGAAGGAAGGCAACTTGGAAGTGGTGGGTGCCCAGGAC
GGTGCCTCCAGCCTCAACAGTGGGCATGGACAGGGTTAATGTCTCCAGAG
TGTACACTGCAGGCAGCCACATTTACACGCCTGCAGTTGTTTTCCGGAGT
CTGTCCCACGGCCCACACGCTCACTTGACTCATGCTGCTAAGATGCACAA
CCGCACACCCATACACAACTACAAGGGCCACAAAGCAACTGCTGGGTTAG
CTTTCCACAGACATAAATATAGTCCATCTGCAATCACAAGCACATAGCCA
GGTAACCCACCAACTCCCCTGGATCTGCAGCCCACACGTGGGAGTCTGGC
TGTCACCTTCACAAGCCACAGAAACGGCCACACATGTTCACAGCTCACAC
GCCCTCTCCATTCATCGAACTTCTCAG.

The human 33303 sequence (SEQ ID NO:4) is approximately 1927 nucleotides long. The nucleic acid sequence includes an initiation codon (ATG) and a termination codon (TGA), which are bolded and underscored above. The region between and inclusive of the initiation codon and the termination codon is a methionine-initiated coding sequence of about 1515 nucleotides, including the termination codon (nucleotides indicated as “coding” of SEQ ID NO:4; SEQ ID NO:6). The coding sequence encodes a 504 amino acid protein (SEQ ID NO:5), which is recited as follows:

(SEQ ID NO:5)

MMEATGTWALLLALALLLLLTLALSGTRARGHLPPGPTPLPLLGNLLQLR
PGALYSGLMRLSKKYGPVFTIYLGPWRPVVVLVGQEAVREALGGQAEEFS
GRGTVAMLEGTFDGHGVFFSNGERWRQLRKFTMLALRDLGMGKREGEELI
QAEARCLVETFQGTEGRPFDPSLLLAQATSNVVCSLLFGLRFSYEDKEFQ
AVVRAAGGTLLGVSSQGGQTYEMFSWFLRPLPGPHKQLLHHVSTLAAFTV
RQVQQHQGNLDASGPARDLVDAFLLKMAQEEQNPGTEFTNKNMLMTVIYL
LFAGTMTVSTTVGYTLLLLMKYPHVQKWVREELNRELGAGQAPSLGDRTR
LPYTDAVLHEAQRLLALVPMGIPRTLMRTTRFRGYTLPQGTEVFPLLGSI
LHDPNKNMLMTVIYLLFAGTMTVSTTVGYTLLLLMKYPHVQKWVREELNR
ELGAGQAPSLGDRTRLPYTDAVLHEAQRLLALVPMGIPRTLMRTTRFRGY
TLPQGTEVFPLLGSILHDPNIFKHPEEFNPDRFLDADGRFRKHEAFLPFS
LGKRVCLGEGLAKAELFLFFTTILQAFSLESPCPPDTLSLKPTVSGLFNI
PPAFQLQVRPTDLHSTTQTR.

Human 32579

The human 32579 nucleic acid sequence is recited as follows:

(SEQ ID NO:7)

GGCGCCGCGGGTCAGGCAGCTGCGTGCGCGTCTCCTCCAGGCAGCAAGGG
GAACCCGAGGCCGCCGGCGCCCGGACCATGTCGTCTCCGGGGCCGTCGCA
GCCGCCGGCCGAGGACCCGCCCTGGCCCGCGCGCCTCCTGCGTGCGCCTC
TGGGGCTGCTGCGGCTGGACCCCAGCGGGGGCGCGCTGCTGCTATGCGGC
CTCGTAGCGCTGCTGGGCTGGAGCTGGCTGCGGAGGCGCCGGGCGCGGGG
CATCCCGCCCGGGCCCACGCCCTGGCCTCTGGTGGGCAACTTCGGTCACG
TGCTGCTGCCTCCCTTCCTCCGGCGGCGGAGCTGGCTGAGCAGCAGGACC
AGGGCCGCAGGGATTGATCCCTCGGTCATAGGCCCGCAGGTGCTCCTGGC
TCACCTAGCCCGCGTGTACGGCAGCATCTTCAGCTTCTTTATCGGCCACT
ACCTGGTGGTGGTCCTCAGCGACTTCCACAGCGTGCGCGAGGCGCTGGTG
CAGCAGGCCGAGGTCTTCAGCGACCGCCCGCGGGTGCCGCTCATCTCCAT
CGTGACCAAGGAGAAGGGGGTTGTGTTTGCACATTATGGTCCCGTCTGGA
GACAACAAAGGAAGTTCTCTCATTCAACTCTTCGTCATTTTGGGTTGGGA
AAACTTAGCTTGGAGCCCAAGATTATTGAGGAGTTCAAATATGTGAAAGC
AGAAATGCAAAAGCACGGAGAAGACCCCTTCTGCCCTTTCTCCATCATCA
GCAATGCCGTCTCTAACATCATTTGCTCCTTGTGCTTTGGCCAGCGCTTT
GATTACACTAATAGTGAGTTCAAGAAAATGCTTGGTTTTATGTCACGAGG
CCTAGAAATCTGTCTGAACAGTCAAGTCCTCCTGGTCAACATATGCCCTT
GGCTTTATTACCTTCCCTTTGGACCATTTAAGGAATTAAGACAAATTGAA
AAGGATATAACCAGTTTCCTTAAAAAAATCATCAAAGACCATCAAGAGTC
TCTGGATAGAGAGAACCCTCAGGACTTCATAGACATGTACCTTCTCCACA
TGGAAGAGGAGAGGAAAAATAATAGTAACAGCAGTTTTGATGAAGAGTAC
TTATTTTATATCATTGGGGATCTCTTTATTGCTGGGACTGATACCACAAC
TAACTCTTTGCTCTGGTGCCTGCTGTATATGTCGCTGAACCCCGATGTAC
AAGAAAAGGTTCATGAAGAAATTGAAAGAGTCATTGGCGCCAACCGAGCT
CCTTCCCTCACAGACAAGGCCCAGATGCCCTACACAGAAGCCACCATCAT
GGAAGTGCAGAGGCTAACTGTGGTGGTGCCGCTTGCCATTCCTCATATGA
CCTCAGAGAACACAGTGCTCCAAGGGTATACCATTCCTAAAGGCACATTG
ATCTTACCCAACCTGTGGTCAGTACATAGAGACCCAGCCATTTGGGAGAA
ACCGGAGGATTTCTACCCTAATCGATTTCTGGATGACCAAGGACAACTAA
TTAAAAAAGAAACCTTTATTCCTTTTGGGATAGGGAAGCGGGTGTGTATG
GGAGAACAACTGGCAAAGATGGAATTATTCCTAATGTTTGTGAGCCTAAT
GCAGAGTTTCGCATTTGCTTTACCTGAGGATTCTAAGAAGCCCCTCCTGA
CTGGAAGATTTGGTCTAACTTTAGCCCCACATCCATTTAATATAACTATT
TCAAGGAGATGAAGAGCATCTCCAAGAAGAGATGGTAAAAAGATATATAA
ATACATATCCTTCTAAGCAGATTCTTCCTACTGCAAAGGACAGTGAATCC
AGCAACTCAGTGGATCCAAGCTGGGCTCAGAGGTCGGAAGGAGGGTAGAG
CACACTGGGAGGTTTCATCTTGGAGGATTCCTCAGCAGGATACTTCAGCC
ATTTTAGTAATGCAGGTCTGTGATTTGGGGGATAGAAAACAAAGTACCTA
TGAAACGGGATATCTGGATTTTACTTGCAGTGGCTTCCACCGATGGGCCA
ATCTTCTCATTTCTTAGTGCCTCAGACATCCCATATGTAAAATGAGAGTA
ATAAAACTTGGCTTCTCTCTAAAAAAAARMAMTAAAAAAAAAAAAAAAA.

The human 32579 sequence (SEQ ID NO:7) is approximately 2099 nucleotides long. The nucleic acid sequence includes an initiation codon (ATG) and a termination codon (TGA), which are bolded and underscored above. The region between and inclusive of the initiation codon and the termination codon is a methionine-initiated coding sequence of about 1635 nucleotides, including the termination codon (nucleotides indicated as “coding” of SEQ ID NO:7; SEQ ID NO:9). The coding sequence encodes a 544 amino acid protein (SEQ ID NO:8), which is recited as follows:

(SEQ ID NO:8)

MSSPGPSQPPAEDPPWPARLLRAPLGLLRLDPSGGALLLCGLVALLGWSW
LRRRRARGIPPGPTPWPLVGNFGHVLLPPFLRRRSWLSSRTRAAGIDPSV
IGPQVLLAHLARVYGSIFSFFIGHYLVVVLSDFHSVREALVQQAEVFSDR
PRVPLISIVTKEKGVVFAHYGPVWRQQRKFSHSTLRHFGLGKLSLEPKII
EEFKYVKAEMQKHGEDPFCPFSIISNAVSNIICSLCFGQRFDYTNSEFKK
MLGFMSRGLEICLNSQVLLVNICPWLYYLPFGPFKELRQIEKDITSFLKK
IIKDHQESLDRENPQDFIDMYLLHMEEERKNNSNSSFDEEYLFYIIGDLF
IAGTDTTTNSLLWCLLYMSLNPDVQEKVHEEIERVIGANRAPSLTDKAQM
PYTEATIMEVQRLTVVVPLAIPHMTSENTVLQGYTIPKGTLILPNLWSVH
RDPAIWEKPEDFYPNRFLDDQGQLIKKETFIPFGIGKRVCMGEQLAKMEL
FLMFVSLMQSFAFALPEDSKKPLLTGRFGLTLAPHPFNITISRR.

### Examples for 21509 and 33770

### Example 2

### Characterization of Human 21509 and 33770 cDNA

The nucleotide sequence of 21509 and 33770 DNA shown as SEQ ID NOs:13 and 16, respectively, including 5′ and 3′ untranslated region, are approximately 1050 and 2060 nucleotides long, respectively. The amino acid sequence of 21509 and 33770 polypeptide shown as SEQ ID NOs:14 and 17, respectively, are 237 and 487 residues in length. The nucleotide coding sequences of 21509 and 33770 shown as SEQ ID NOs:15 and 18, respectively, are approximately 711 and 1461 nucleotides long.

### Example 3

### Tissue Distribution of 21509 and 33770 mRNA

Endogenous human 21509 gene expression was determined using the Perkin-Elmer/ABI 7700 Sequence Detection System which employs TaqMan technology. Briefly, TaqMan technology relies on standard RT-PCR with the addition of a third gene-specific oligonucleotide (referred to as a probe) which has a fluorescent dye coupled to its 5′ end (typically 6-FAM) and a quenching dye at the 3′ end (typically TAMRA). When the fluorescently tagged oligonucleotide is intact, the fluorescent signal from the 5′ dye is quenched. As PCR proceeds, the 5′ to 3′ nucleolytic activity of taq polymerase digest the labeled primer, producing a free nucleotide labeled with 6-FAM, which is now detected as a fluorescent signal. The PCR cycle where fluorescence is first released and detected is directly proportional to the starting amount of the gene of interest in the test sample, thus providing a way of quantitating the initial template concentration. Samples can be internally controlled by the addition of a second set of primers/probe specific for a housekeeping gene such as GAPDH which has been labeled with a different fluorophore on the 5′ end (typically VIC).

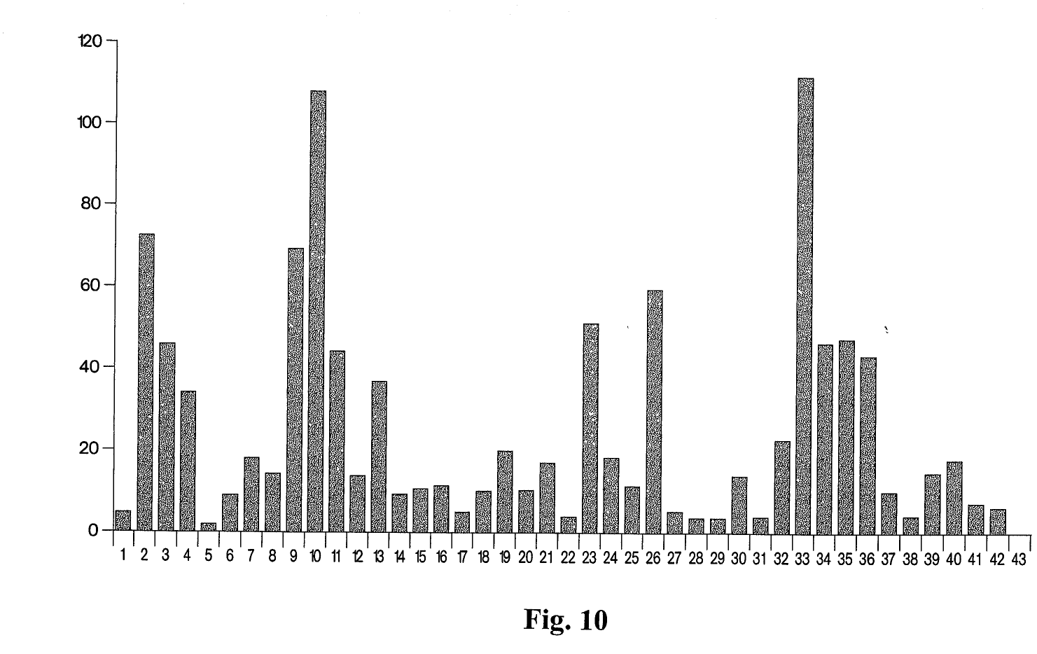

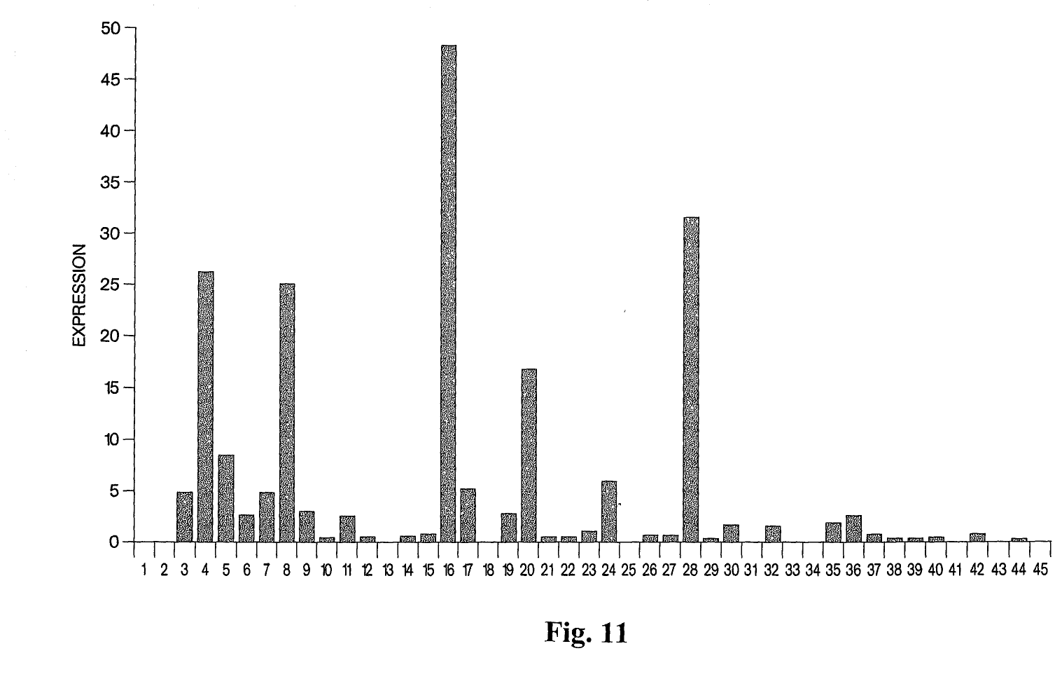

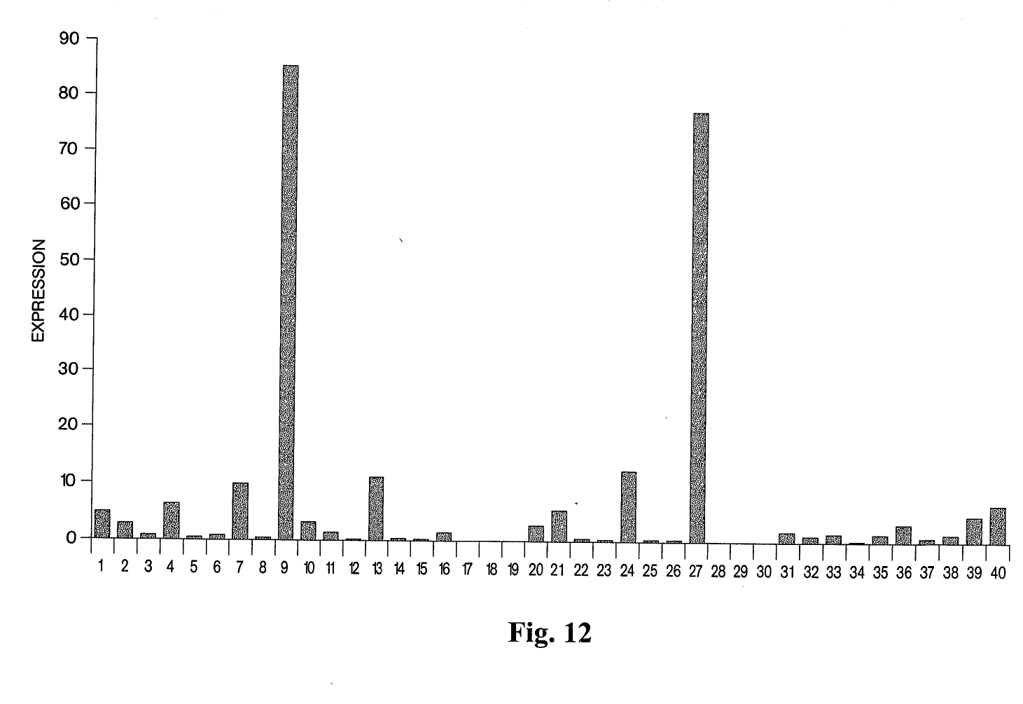

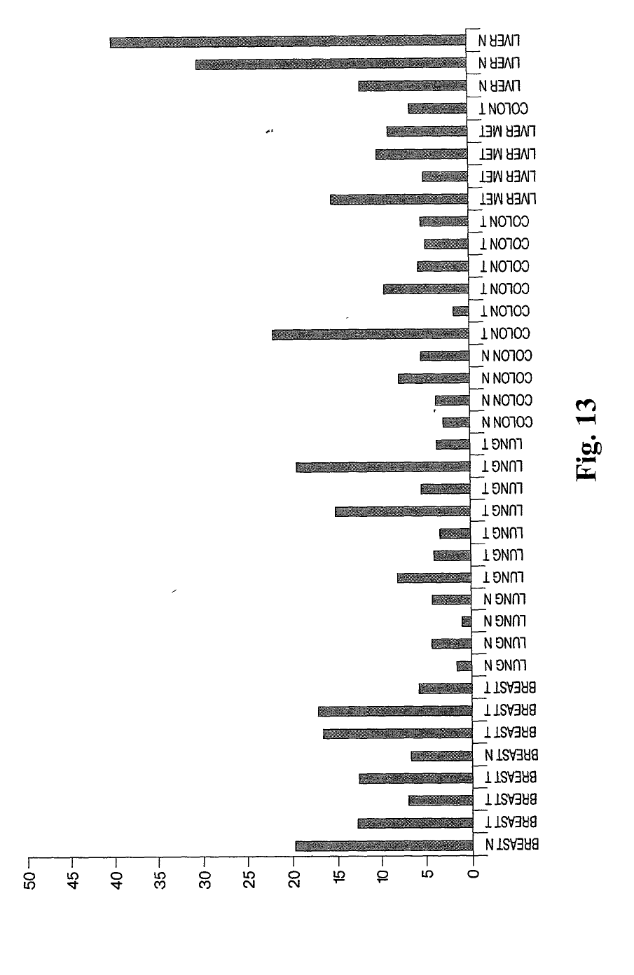

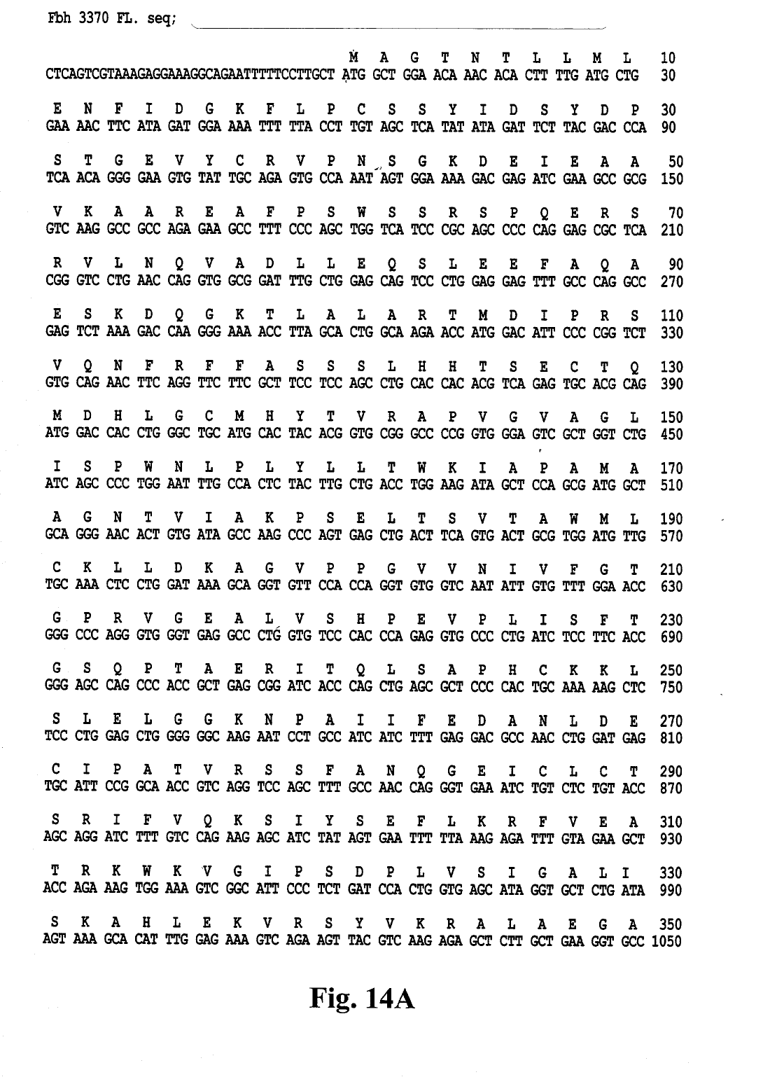

To determine the level of 21509 in various human tissues a primer/probe set was designed. Total RNA was prepared from a series of human tissues using an RNeasy kit from Qiagen. First strand cDNA was prepared from one ug total RNA using an oligo dT primer and Superscript II reverse transcriptase (Gibco/BRL). cDNA obtained from approximately 50 ng total RNA was used per TaqMan reaction. Tissues tested include normal and tumorous human tissues shown in FIGS. 10-13. Expression of 21509 RNA was detected in most of the tissues analyzed, with notable expression occurring, e.g., in epithelial cell (FIG. 10, column 33 and FIG. 11, column 28), nervous (FIG. 10, columns 7-12 and FIG. 11, columns 15-21), heart (FIG. 10, columns 2-4), liver (FIG. 10, columns 24-28), kidney (FIG. 10, column 23 and FIG. 11, column 8), endothelial cell (FIG. 10, column 34 and FIG. 11, columns 4-5), skeletal muscle (FIG. 10, column 35), and breast (FIG. 10, columns 13-14) tissues. In addition, increased expression of human 21509 RNA was detected in several tumor samples, as compared to tissue-matched normal tissue samples, from breast (FIG. 12, column 9), prostate (FIG. 10, column 19), colon (FIG. 10, column 21, FIG. 13, column 24), lung (FIG. 12, column 24, FIG. 13, columns 16 and 18), and ovary (FIG. 12, column 13) tumors. The incidence of tumor-associated expression of 21509 RNA in ovary, breast, colon, and lung tissues was further evaluated by in situ hybridization (see Table 2). Notable tumor-associated expression of 21509 is seen in ovarian, colon, and lung tumors. 21509 RNA is also expressed in both normal and malignant breast epithelium. This data suggests a role for 21509 in tumor development.

TABLE 2

| | | |
|---|---|---|
| Spectrum # | Tissue Diagnosis | Results |

OVARY: 0/3 normals; 2/2 borderline tumors; 3/3 invasive tumors

| | | |
|---|---|---|
| MDA 201 | Ovary Normal | (−) |
| MDA 202 | Ovary Normal | (−) |
| MDA 203 | Ovary Normal | (−) |
| CLN 350 | Ovary Tumor: LMP-mucinous | (+++/+) |
| MDA 206 | Ovary Tumor: LMP-mucinous | (+/−) |
| MDA 300 | Ovary Tumor: MD-AC [endometrioid] | (++/+) |
| CLN 5 | Ovary Tumor: MD-PS | (++/+) |
| MDA 205 | Ovary Tumor: PS | (+++) |

BREAST: 2/2 normals; 3/3 tumors

| | | |
|---|---|---|
| PIT 370 | Breast Normal | (+++/++) |
| PIT 35 | Breast Normal | (+++/++) |
| MDA 161 | Breast Tumor: MD/PD-IDC | (+++/++) |
| NDR 6 | Breast Tumor: MD/PD-IDC | (++/+) |
| CLN 172 | Breast Tumor: MD-AC [lobular] | (++/−) |

COLON: 0/2 normals; 3/3 tumors; 1/1 metastasis

| | | |
|---|---|---|
| PIT 337 | Colon Normal | (−) |
| CHT 521 | Colon Normal | (−) |
| CLN 609 | Colon Tumor | (++/+) |
| CHT 910 | Colon Tumor | (+++/+) |
| CHT 528 | Colon Tumor | (+++/+) |
| NDR 100 | Colon Metastasis | (+++/+) |

LUNG: 0/2 normals; 1/2 tumors

| | | |
|---|---|---|
| CHT 330 | Lung Normal | (−) |
| CHT 813* | Lung Normal | (−) |
| CHT 547 | Lung Tumor: WD/MD-AC | (−) |
| CHT 813* | Lung Tumor: MD-SCC | (++/+) |

MD-AC = moderately differentiated adenocarcinoma;
MD/PD-IDC = moderately/poorly differentiated invasive ductal carcinoma;
WD/MD-AC = well/moderately differentiated adenocarcinoma;
MD-SCC = moderately differentiated squamous cell carcinoma.

### Example 4

### Tissue Distribution of 21509 or 33770 mRNA by Northern Analysis

Northern blot hybridizations with various RNA samples can be performed under standard conditions and washed under stringent conditions, i.e., 0.2× SSC at 65° C. A DNA probe corresponding to all or a portion of the 21509 or 33770 cDNA (SEQ ID NO: 13 or SEQ ID NO: 16, respectively) can be used. The DNA was radioactively labeled with ³²P-dCTP using the Prime-It Kit (Stratagene, La Jolla, Calif.) according to the instructions of the supplier. Filters containing mRNA from mouse hematopoietic and endocrine tissues, and cancer cell lines (Clontech, Palo Alto, Calif.) can be probed in ExpressHyb hybridization solution (Clontech) and washed at high stringency according to manufacturer's recommendations.

### Example 5

### Recombinant Expression of 21509 or 33770 in Bacterial Cells

In this example, 21509 or 33770 is expressed as a recombinant glutathione-S-transferase (GST) fusion polypeptide in E. coli and the fusion polypeptide is isolated and characterized. Specifically, 21509 or 33770 is fused to GST and this fusion polypeptide is expressed in E. coli, e.g., strain PEB199. Expression of the GST-21509 or 33770 fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 6

### Expression of Recombinant 21509 or 33770 Protein in COS Cells

To express the 21509 or 33770 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire 21509 or 33770 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the 21509 or 33770 DNA sequence is amplified by PCR using two primers. The 5′ primer contains the restriction site of interest followed by approximately twenty nucleotides of the 21509 or 33770 coding sequence starting from the initiation codon; the 3′ end sequence contains complementary sequences to the other restriction site of interest, a translation stop codon, the HA tag or FLAG tag and the last 20 nucleotides of the 21509 or 33770 coding sequence. The PCR amplified fragment and the pcDNA/Amp vector are digested with the appropriate restriction enzymes and the vector is dephosphorylated using the CIAP enzyme (New England Biolabs, Beverly, Mass.). Preferably the two restriction sites chosen are different so that the 21509 or 33770_gene is inserted in the correct orientation. The ligation mixture is transformed into E. coli cells (strains HB 101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the 21509 or 33770-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. (1989) Molecular Cloning: A Laboratory Manual. 2nd, ed., Cold Spring Harbor Laboratory, Cold Spring Harbor Laboratory Press, Cold Spring Harbor, N.Y. The expression of the 21509 or 33770 polypeptide is detected by radiolabelling (³⁵S-methionine or ³⁵S-cysteine available from NEN, Boston, Mass., can be used) and immunoprecipitation (Harlow, E. and Lane, D. (1988) Antibodies: A Laboratory Manual, Cold Spring Harbor Laboratory Press, Cold Spring Harbor, N.Y.) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the 21509 or 33770 coding sequence is cloned directly into the polylinker of the pcDNA/Amp vector using the appropriate restriction sites. The resulting plasmid is transfected into COS cells in the manner described above, and the expression of the 21509 or 33770 polypeptide is detected by radiolabelling and immunoprecipitation using a 21509 or 33770 specific monoclonal antibody.

### Examples for 46638

### Example 7

### Identification and Characterization of Human 46638 cDNA

The human 46638 nucleic acid sequence is recited as follows:

(SEQ ID NO:22)

CCGGACACCTGGGCTCCCGCCCAGGATCCTGCAGGCCCAGGGCGGTCCTG
GAGCGGAAAGAATGCCACGCGGGGCATTCAGACCCTGTTTGCCGGCGCTG
TATTTCGCTTTCCTGACCTGCCCTACTCCAGAGCAGAGAATGCAGTGGAA
CCCAGGCTCCTGATATCCATCTGGGTGAGCCAGCCAGAGGGACCGGCTGT
GTCAGAGGCAAGCAAACAAGTATTAGAGTGCAAGACTGTGGGCGGAGAGA
GGAAGCCCGAGCCGCCAGCAGGGAGCTTCGGAGAGAGAAAGCCCAGGAAC
ATCCCAGAGAGAGCTGGGCCCATCCTCAGCCCTACCCAGCCCCGCAGCCC
CTAGCCCTCCGCCCAGAAACCCAGCCCTGTCCGGCGTGCCGCTCTTCTCC
TCCAGGCCGGCTGCTGCTGCGGCCAGCGTTGCCGGGGCATCCCTTCCTCC
TTCCCATCATGGCAGTGTACCGCCTGTGTGTGACCACTGGTCCCTACCTG
AGGGCCGGCACACTGGACAACATCTCTGTCACACTGGTGGGCACGTGTGG
TGAAAGCCCCAAGCAGCGGCTAGATCGAATGGGCAGGGACTTCGCCCCTG
GATCGGTACAGAAGTACAAGGTGCGTTGCACAGCGGAGCTGGGTGAGCTC
TTGCTGCTGCGTGTACACAAGGAGCGCTACGCTTTCTTCCGCAAGGACTC
TTGGTACTGTAGCCGCATCTGTGTCACCGAACCGGATGGTAGTGTATCCC
ACTTCCCCTGCTATCAGTGGATTGAAGGCTACTGCACCGTGGAGCTGAGG
CCAGGAACAGCAAGAACTATTTGTCAGGACTCTCTTCCCCTCCTCCTGGA
TCACAGGACACGGGAGCTCCGGGCCCGACAAGAATGCTACCGCTGGAAGA
TCTATGCCCCTGGCTTCCCCTGCATGGTAGACGTCAACAGCTTTCAGGAG
ATGGAGTCAGACAAGAAATTTGCCTTGACAAAGACGACAACTTGTGTAGA
CCAGGGTGACAGCAGTGGGAATCGGTACCTGCCCGGCTTCCCCATGAAAA
TTGACATCCCATCCCTGATGTACATGGAGCCCAATGTTCGATACTCAGCC
ACCAAGACGATCTCGCTGCTCTTCAATGCCATCCCTGCGTCCTTGGGAAT
GAAGCTTCGAGGGCTGTTGGATCGCAAGGGCTCCTGGAAGAAGCTGGATG
ACATGCAGAACATCTTCTGGTGCCATAAGACCTTCACGACAAAGTATGTC
ACAGAGCACTGGTGTGAAGATCACTTCTTTGGGTACCAGTACCTGAATGG
TGTCAATCCCGTCATGCTCCACTGCATCTCTAGCTTGCCCAGCAAGCTGC
CTGTCACCAATGACATGGTGGCCCCCTTGCTGGGACAGGACACATGCCTG
CAGACAGAGCTAGAGAGGGGGAACATCTTCCTAGCGGACTACTGGATCCT
GGCGGAGGCCCCCACCCACTGCCTAAACGGCCGCCAGCAGTACGTGGCCG
CCCCACTGTGCCTGCTGTGGCTCAGCCCCCAGGGGGCGCTGGTGCCCTTG
GCCATCCAGCTCAGCCAGACCCCCGGGCCTGACAGCCCCATCTTCCTGCC
CACTGACTCCGAATGGGACTGGCTGCTGGCCAAGACGTGGGTGCGCAACT
CTGAGTTCCTGGTGCACGAAAACAACACGCACTTTCTGTGCACGCATTTG
CTGTGCGAGGCCTTCGCCATGGCCACGCTGCGCCAGCTGCCGCTCTGCCA
CCCCATCTACAAGCTCCTACTCCCCCACACTCGATACACGCTGCAGGTGA
ACACCATCGCGAGGGCCACGCTGCTCAACCCCGAGGGCCTCGTGGACCAG
GTCACGTCCATCGGGAGGCAAGGCCTCATCTACCTCATGAGCACGGGCCT
GGCCCACTTCACCTACACCAATTTCTGCCTTCCGGACAGCCTGCGGGCCC
GCGGCGTCCTGGCTATCCCCAACTACCACTACCGAGACGACGGCCTGAAG
ATCTGGGCGGCCATTGAGAGCTTTGTCTCAGAAATCGTGGGCTACTATTA
TCCCAGTGACGCATCTGTGCAGCAGGATTCGGAGCTGCAGGCCTGGACTG
GCGAGATTTTTGCTCAGGCGTTCCTGGGCCGGGAAAGCTCAGGTTTCCCA
AGCCGGCTGTGCACCCCAGGAGAGATGGTGAAGTTCCTCACTGCAATCAT
CTTCAATTGCTCTGCCCAGCACGCTGCTGTCAACAGTGGGCAGCATGACT
TTGGGGCCTGGATGCCCAATGCTCCATCATCCATGAGGCAGCCCCCACCC
CAGACCAAGGGGACCACCACCCTGAAGACTTACCTAGACACCCTCCCTGA
AGTGAACATCAGCTGTAACAACCTCCTCCTCTTCTGGTTGGTTAGCCAAG
AACCCAAGGACCAGAGGCCCCTGGGCACCTACCCAGATGAGCACTTCACA
GAGGAGGCCCCGAGGCGGAGCATCGCCGCCTTCCAGAGCCGCCTGGCCCA
GATCTCAAGGGACATCCAGGAGCGGAACCAGGGTCTGGCACTGCCCTACA
CCTACCTGGACCCTCCCCTCATTGAGAACAGTGTCTCCATCTAACCACCC
CCAAATACCACCCAAGAAGAAAGAAAGGTCCAAGCATGAGGAGGACCAGT
TCCTCAGGTCCTCCAGACCCTTCCATCCTCCCTGTTCTCAGTTCACCTGA
ACCTTCTCTTCTGCACATGGAGACTTTTGCAGCCAAGATGGCTCTGACAT
CATACAAACTGGGCCCTGAGCTGTGAGAGACCAGCACAGCAGCGTCCAGG
TTAAAAGCCGCTGACCAAAGTCCAATGCACAATAGCCCCTCCGAAAGGAA
GGAACCGCTTCACTTCTTGCCCCACTTGGGGCAGCCTCTTGTTCCAGCCT
CTTGGPATGCCCAGCTTGGGTTTCTGAGCTTTTCTCCCTCATCCTCCCCC
ATCCCCKAACTCCTTCTCCTACCATGCCTTTCTACGTTCTCTTTCTTCCA
AGCCTAGAGCCACCAGCCCAGCTTCCTTCTCTGGAAAAGCCTGGAAACTG
GGCACAGAAGGACTGTGTGCCTGGGTCTAACATGTGGTCCCCTTTGTCCC
TAGCACCTTTAAGGGGAGGGGAAGAATTGGAGGGCAGCTTGCCTGGACCC
CTAACGGCTGTTCTCAGGAACAGGTTCCCAGGCCTGGGGTGTTTGTGGAG
RTCTGTCTTTCTCCAAAGWTTTCATCCAACTCCCCTTTCWTCCCMCTCCC
TTTCWTCCCATTTTTTTCTTTCTGTCCTTGAGCCCAGTGAGTTCAATAAA
AACCAAAATATTTGGCTATC

The human 46638 sequence (SEQ ID NO:22), is approximately 3320 nucleotides long. The nucleic acid sequence includes an initiation codon (ATG) and a termination codon (TAA) which are underscored above. The region between and inclusive of the initiation codon and the termination codon is a methionine-initiated coding sequence of about 2136 nucleotides, including the termination codon (nucleotides 459-2594 of SEQ ID NO:22; SEQ ID NO:24). The coding sequence encodes a 711 amino acid protein (SEQ ID NO:23), which is recited as follows:

(SEQ ID NO:23)

MAVYRLCVTTGPYLRAGTLDNISVTLVGTCGESPKQRLDRMGRDFAPGSV
QKYKVRCTAELGELLLLRVHKERYAFFRKDSWYCSRICVTEPDGSVSHFP
CYQWIEGYCTVELRPGTARTICQDSLPLLLDHRTRELRARQECYRWKIYA
PGFPCMVDVNSFQEMESDKKFALTKTTTCVDQGDSSGNRYLPGFPMKDIP
SLMYMEPNVRYSATKTJSLLFNAWASLGMKLRGLLDRKGSWKKLDDMQNI
FWCHKTFTTKYVTEHWCEDHFFGYQYLNGVNPVMLHCISSLPSKLPVTND
MVAPLLGQDTCLQTELERGNIFLADYWILAEAPTHCLNGRQQYVAAPLCL
LWLSPQGALVPLAIQLSQTPGPDSPIFLPTDWEWDWLLAKTWVRNSEFLV
HENNTHFLCTHLLCEAFAMATLRQLPLCHPIYKLLLPHTRYTLQVNTIAR
ATLLNPEGLVDQVTSIGRQGLIYLMSTGLAHFTYTNFCLPDSLRARGVLA
IPNTHYRDDGLKIWAAIESFVSEIVGYYYPSDASVQQDSELQAWTGEIFA
QAFLGRESSGRPSRLCTPGEMVKFLTAIIFNCSAQHAAVNSGQHDFGAWM
PNAPSSMRQPPPQTKGTTTLKTYLDTLPEVNISCNNLLLFWLVSQEPKDQ
RPLGTYPDEHFTEEAPRRSIAAFQSRLAQISRDIQERNQGLALPYTYLDP
PLIENSVSI

### Example 8

### Tissue Distribution of 46638 mRNA by TaqMan Analysis

Endogenous human 46638 gene expression was determined using the Perkin-Elmer/ABI 7700 Sequence Detection System which employs TaqMan technology. Briefly, TaqMan technology relies on standard RT-PCR with the addition of a third gene-specific oligonucleotide (referred to as a probe) which has a fluorescent dye coupled to its 5′ end (typically 6-FAM) and a quenching dye at the 3′ end (typically TAMRA). When the fluorescently tagged oligonucleotide is intact, the fluorescent signal from the 5′ dye is quenched. As PCR proceeds, the 5′ to 3′ nucleolytic activity of Taq polymerase digests the labeled primer, producing a free nucleotide labeled with 6-FAM, which is now detected as a fluorescent signal. The PCR cycle where fluorescence is first released and detected is directly proportional to the starting amount of the gene of interest in the test sample, thus providing a quantitative measure of the initial template concentration. Samples can be internally controlled by the addition of a second set of primers/probe specific for a housekeeping gene such as GAPDH which has been labeled with a different fluorophore on the 5′ end (typically VIC).

To determine the level of 46638 in various human tissues a primer/probe set was designed. Total RNA was prepared from a series of human tissues using an RNeasy kit from Qiagen. First strand cDNA was prepared from 1 μg total RNA using an oligo-dT primer and Superscript II reverse transcriptase (Gibco/BRL). cDNA obtained from approximately 50 ng total RNA was used per TaqMan reaction. Tissues tested include the human tissues and several cell lines shown in the following tables.

Table 3 below depicts the expression of 46638 mRNA in a panel of normal and tumor human tissues, including breast, ovary, lung, and bronchial epithelial cells using TaqMan analysis. The following tissues are shown: normal breast; breast tumors; normal ovary; ovarian tumor; normal lung and lung tumors (PDNSCCL =poorly differentiated non-small cell carcinoma; SCC=small cell carcinoma). Elevated expression of the 46638 mRNA was detected in normal human bronchial epithelial cells (NHBE), with lower expression levels detected in ovary tumor cell lines.

TABLE 3
Expression of 46638 mRNA in normal human
bronchial epithelial cells and ovarian tumors.

| Tissue Type | Relative Expression |
|---|---|
| PIT 400 Breast Normal | 0 |
| PIT 372 Breast Normal | 0 |
| PIT 56 Breast Normal | 0 |
| MDA 106 Breast Tumor | 0 |
| MDA 234 Breast Tumor | 0 |
| NDR 57 Breast Tumor | 0 |
| MDA 304 Breast Tumor | 0 |
| NDR 58 Breast Tumor | 0 |
| NDR 132 Breast Tumor | 0 |
| NDR 07 Breast Tumor | 0 |
| NDR 12 Breast Tumor | 0 |
| PIT 208 Ovary Normal | 0 |
| CHT 620 Ovary Normal | 0 |
| CHT 619 Ovary Normal | 0 |
| CLN 03 Ovary Tumor | 0 |
| CLN 05 Ovary Tumor | 0 |
| CLN 17 Ovary Tumor | 0 |
| CLN 07 Ovary Tumor | 0 |
| CLN 08 Ovary Tumor | 0 |
| MDA 216 Ovary Tumor | 0 |
| MDA 25 Ovary Tumor | 0 |
| MDA 183 Lung Normal | 0 |
| CLN 930 Lung Normal | 0 |
| MDA 185 Lung Normal | 0 |
| CHT 816 Lung Normal | 0 |
| MPI 215 Lung Tumor—SmC | 0 |
| MDA 259 Lung Tumor—PDNSCCL | 0 |
| CHT 832 Lung Tumor—PDNSCCL | 0 |
| MDA 253 Lung Tumor—PDNSCCL | 0 |
| CHT 814 Lung Tumor—SCC | 0 |
| CHT 793 Lung Tumor—ACA | 0 |
| MDA 262 Lung Tumor—SCC | 0 |
| CHT 211 Lung Tumor—AC | 0 |
| NHBE | 0.123 |
| MDA 127 Normal Ovarian Epithelial Cells | 0 |
| MDA 224 Normal Ovarian Epithelial Cells | 0 |
| MDA 124 Ovarian Ascites Tumor | 0.003 |
| MDA 126 Ovarian Ascites Tumor | 0 |
| CLN 012 Ovary Tumor | 0.0023 |

Table 4 below depicts the expression of 46638 mRNA in a second panel of normal and tumor human tissues, also including breast, ovary, lung, and bronchial epithelial cells using TaqMan analysis. Again, elevated expression of the 46638 mRNA was detected in normal human bronchial epithelial cells (NHBE), with lower expression levels detected in some ovary tumor cell lines.

TABLE 4
Expression of 46638 in normal human
bronchial epithelial cells an ovarian tumor.

| Tissue Type | Relative Expression |
|---|---|
| PIT 400 Breast Normal | 0 |
| PIT 372 Breast Normal | 0 |
| MDA 106 Breast Tumor | 0 |
| MDA 234 Breast Tumor | 0 |
| NDR 57 Breast Tumor | 0 |
| MDA 304 Breast Tumor | 0 |
| NDR 58 Breast Tumor | 0 |
| NDR 132 Breast Tumor | 0 |
| NDR 07 Breast Tumor | 0 |
| NDR 12 Breast Tumor | 0 |
| PIT 208 Ovary Normal | 0 |
| CHT 620 Ovary Normal | 0 |
| CHT 619 Ovary Normal | 0 |
| CLN 03 Ovary Tumor | 0 |
| CLN 17 Ovary Tumor | 0 |
| CLN 07 Ovary Tumor | 0 |
| CLN 08 Ovary Tumor | 0 |
| MDA 216 Ovary Tumor | 0 |
| CLN 012 Ovary Tumor | 0 |
| MDA 25 Ovary Tumor | 0 |
| MDA 183 Lung Normal | 0 |
| CLN 930 Lung Normal | 0 |
| MDA 185 Lung Normal | 0 |
| CHT 816 Lung Nonnal | 0 |
| MPI 215 Lung T—SmC | 0 |
| MDA 259 Lung Tumor—PDNSCCL | 0 |
| CHT 832 Lung Tumor—PDNSCCL | 0 |
| MDA 253 Lung Tumor—PDNSCCL | 0 |
| CHT 911 Lung Tumor—SCC | 0 |
| CHT 793 Lung Tumor—ACA (?) | 0 |
| MDA 262 Lung Tumor—SCC | 0 |
| CHT 211 Lung Tumor—AC | 0 |
| NHBE | 2.15 |
| MDA 127 Normal Ovarian Epithelial Cells | 0.01 |
| MDA 224 Normal Ovarian Epithelial Cells | 0.00 |
| MDA 124 Ovarian Ascites | 0.01 |
| MDA 126 Ovarian Ascites | 0.01 |

Table 5 below the expression of 46638 RNA in a panel of normal and malignant human tissues, including normal colon, colon tumors, liver metastatic, normal liver, human microvesicular endothelial cells proliferating (HMVEC-Prol), placenta, and hemangioma. Elevated expression was detected primarily in the normal colon, placenta, and a liver metastatic cell line.

TABLE 5
46638 Expression in normal colon, placenta and metastatic liver cells.

| Tissue Type | Relative Expression |
|---|---|
| CHT 523 Colon Normal | 0 |
| NDR 104 Colon Normal | 0.03 |
| CHT 416 Colon Normal | 0 |
| CHT 452 Colon Normal | 0 |
| NDR 210 Colon Tumor | 0 |
| CHT 398 Colon Tumor | 0 |
| CHT 382 Colon Tumor | 0 |
| CHT 944 Colon Tumor | 0 |
| CHT 528 Colon Tumor | 0 |
| CHT 1365 Colon Tumor | 0 |
| CHT 372 Colon Tumor | 0 |
| CLN 609 Colon Tumor | 0 |
| CHT 01 Liver Metastatic | 0 |
| NDR 100 Liver Metastatic | 0.01 |
| CHT 340 Liver Metastatic | 0 |
| NDR 217 Liver Metastatic | 0 |
| PIT 260 Liver Normal | 0 |
| CHT 320 Liver Normal | 0 |
| C48 HMVEC-Prol | 0 |
| ONC 102 Hemangioma | 0 |
| CHT 50 Placenta | 0.12 |

Table 6 below depicts the expression of 46638 mRNA in a panel of normal and tumor human tissues, using TaqMan analysis. Elevated expression was detected in the following tissues: normal heart, normal brain cortex, normal brain hypothalamus, breast tumor, colon tumor, lung tumor, prostate epithelial cells, and normal skin. Expression of 46638 was highest in brain cortex, brain hypothalamus, and prostate epithelial cells.

TABLE 6
Expression of 46638 in Human Tissues.

| Tissue Type | Relative Expression |
|---|---|
| Aorta/normal | 0 |
| Fetal heart/normal | 0 |
| Heart normal | 0.14567087 |
| Heart/CHF | 0 |
| Vein/Normal | 0 |
| Spinal cord/Normal | 0 |
| Brain cortex/Normal | 8.9431575 |
| Brain hypothalamus/Normal | 1.63669362 |
| Glial cells (Astrocytes) | 0 |
| Brain/Glioblastoma | 0 |
| Breast/Normal | 0 |
| Breast tumor/IDC | 0.22779126 |
| OVARY/Normal | 0 |
| OVARY/Tumor | 0 |
| Pancreas | 0 |
| Prostate/Normal | 0 |
| Prostate/Tumor | 0 |
| Colon/normal | 0 |
| Colon/tumor | 0.08366594 |
| Colon/IBD | 0 |
| Kidney/normal | 0 |
| Liver/normal | 0 |
| Liver fibrosis | 0 |
| Fetal Liver/normal | 0 |
| Lung/normal | 0 |
| Lung/tumor | 0.06772275 |
| Lung/COPD | 0 |
| Spleen/normal | 0 |
| Tonsil/normal | 0 |
| Lymph node/normal | 0 |
| Thymus/normal | 0 |
| Epithelial Cells (prostate) | 10.6721895 |
| Endothelial Cells (aortic) | 0 |
| Skeletal Muscle/Normal | 0 |
| Fibroblasts (Dermal) | 0 |
| Skin/normal | 0.42213732 |
| Adipose/Normal | 0 |
| Osteoblasts (primary) | 0 |
| Osteoblasts (Undiff) | 0 |
| Osteoblasts (Diff) | 0 |
| Osteoclasts | 0 |
| Aortic SMC Early | 0 |
| Aortic SMC Late | 0 |
| shear HUVEC | 0 |
| static HUVEC | 0 |
| osteoclasts undiff | 0 |

### Example 9

### Tissue Distribution of 46638 mRNA by Northern Analysis

Northern blot hybridizations with various RNA samples can be performed under standard conditions and washed under stringent conditions, i.e., 0.2× SSC at 65° C. A DNA probe corresponding to all or a portion of the 46638 cDNA (SEQ ID NO:22) can be used. The DNA was radioactively labeled with ³²P-dCTP using the Prime-It Kit (Stratagene, La Jolla, Calif.) according to the instructions of the supplier. Filters containing mRNA from mouse hematopoietic and endocrine tissues, and cancer cell lines (Clontech, Palo Alto, Calif.) can be probed in ExpressHyb hybridization solution (Clontech) and washed at high stringency according to manufacturer's recommendations.

### Example 10

### Recombinant Expression of 46638 in Bacterial Cells

In this example, 46638 is expressed as a recombinant glutathione-S-transferase (GST) fusion polypeptide in E. coli and the fusion polypeptide is isolated and characterized. Specifically, 46638 is fused to GST and this fusion polypeptide is expressed in E. coli, e.g., strain PEB199. Expression of the GST-46638 fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 11

### Expression of Recombinant 46638 Protein in COS Cells

To express the 46638 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire 46638 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the 46638 DNA sequence is amplified by PCR using two primers. The 5′ primer contains the restriction site of interest followed by approximately twenty nucleotides of the 46638 coding sequence starting from the initiation codon; the 3′ end sequence contains complementary sequences to the other restriction site of interest, a translation stop codon, the HA tag or FLAG tag and the last 20 nucleotides of the 46638 coding sequence. The PCR amplified fragment and the pcDNA/Amp vector are digested with the appropriate restriction enzymes and the vector is dephosphorylated using the CIAP enzyme (New England Biolabs, Beverly, Mass.). Preferably the two restriction sites chosen are different so that the 46638_ene is inserted in the correct orientation. The ligation mixture is transformed into E. coli cells (strains HB 101, DH5 cc, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the 46638-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. (1989) Molecular Cloning: A Laboratory Manual. 2nd, ed., Cold Spring Harbor Laboratory, Cold Spring Harbor Laboratory Press, Cold Spring Harbor, N.Y. The expression of the 46638 polypeptide is detected by radiolabelling (³⁵S-methionine or ³⁵S-cysteine available from NEN, Boston, Mass., can be used) and immunoprecipitation (Harlow, E. and Lane, D. (1988) Antibodies: A Laboratory Manual, Cold Spring Harbor Laboratory Press, Cold Spring Harbor, N.Y.) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the 46638 coding sequence is cloned directly into the polylinker of the pcDNA/Amp vector using the appropriate restriction sites. The resulting plasmid is transfected into COS cells in the manner described above, and the expression of the 46638 polypeptide is detected by radiolabelling and immunoprecipitation using a 46638 specific monoclonal antibody.

### Examples for 50090

### Example 12

### Characterization of Human 50090 cDNA

The human 50090 nucleic acid sequence is recited as follows:

(SEQ ID NO:28)

ACGGACTGGGCCTGGCCTGGGGCGTCCCCGCGAAGCCTGGGCCTGTCAGG
CGGTTCCGTCCGGGTCTCGGCCACCGTCGAGTTCCGTCGAGTTCCGTCCC
GGCCCTGCTCACAGCAGCGCCCTCGGAGCGCCCAGCACCTGCGGCCGGCC
AGGCAGCGCGATCCTGCGGCGTCTGGCCATCCCGAATGCTATGGCCGCCG
TCGCCGTCTTGCGGGCCTTCGGGGCAAGTGGGCCCATGTGTCTCCGGCGC
GGCCCCTGGGCCCAGCTCCCCGCCCGCTTCTGCAGCCGGGACCCGGCCGG
GGCGGGGCGGCGGGAGTCGGAGCCGCGGCCCACCAGCGCGCGGCAGCTGG
ACGGCATAAGGAACATCGTCTTGAGCAATCCCAAGAAGAGGAACACGTTG
TCACTTGCAATGCTGAAATCTCTCCAAAGTGACATTCTTCATGACGCTGA
CAGCAACGATCTGAAAGTCATTATCATCTCGGCTGAGGGGCCTGTGTTTT
CTTCTGGGCATGACTTAAAGGAGCTGACAGAGGAGCAAGGCCGTGATTAC
CATGCCGAAGTATTTCAGACCTGTTCCAAGGTCATGATGCACATCCGGAA
CCACCCCGTCCCCGTCATTGCCATGGTCAATGGCCTGGCCACGGCTGCCG
GCTGTCAACTGGTTGCCAGCTGCAACATTGCCGTGGCGAGCGACAAGTCC
TCTTTTGCCACTCCTGGGGTGAACGTCGGGCTCTTCTGTTCTACCCCTGG
GGTTGCCTTGGCAAGAGCAGTGCCTAGAAAGGTGGCCTTGGAGATGCTCT
TTACTGGTGAGCCCATTTCTGCCCAGGAGGCCCTGCTCCACGGGCTGCTT
AGCAAGGTGGTGCCAGAGGCGGAGCTGCAGGAGGAGACCATGCGGATCGC
TAGGAAGATCGCGTCACTGAGCCGTCCGGTGGTGTCCCTGGGCAAAGCCA
CCTTCTACAAGCAGCTGCCCCAGGACCTGGGGACGGCTTACTACCTCACC
TCCCAGGCCATGGTGGACAACCTGGCCCTGCGGGACGGGCAGGAGGGCAT
CACGGCCTTCCTCCAGAAGAGAAAACCTGTCTGGTCACACGAGCCAGTGT
GAGTGGAGGCAGAGGAGTGAGGCCCACGGGCAGCGCCCAGGAGCCCACCT
TCCCCTCTGGCCCAGCCACCACTGCCTCTCAGCTTCAACAGGTGACAGGC
TGCTTTCGTGACTTGATATTGGTGTCATAGCATTTGGCCTACATTAAAAG
CCACAATTTCATGGGGAAAGGACAAAATGGAGAGTGACTGAGGTGCTGAC
CTCAGTGCAAGGCTGGTGAACCCTGCAGCGGGCCAGCTATGGTGGGAAGC
CTGGCATTTGGGGTGCTCCTTGCAACGTCTTAAGCAAGCGACCCCCCTGA
CATAGCAAAAGGTGGCAACCCATGGAGGCAGAAAGAAGGACGCCAGCCTG
ACCCTTATCTGAAACGTCCTAAGCAGAGTTAATCCTGGCTGCTCAGGAGA
GGCGACACATTTCAAATCTCCACGAGATATTCTCCACACAGAAAATCTTC
TTGATTCTATAGAGACTTAATCATGCCTATGGCTTTGAATAATCTTATGT
GATTTAAATAAATTAAATCTTTATAGAGAAAAAAAAAAA.

The human 50090 sequence (SEQ ID NO:28) is approximately 1639 nucleotides long including untranslated region. The nucleic acid sequence includes an initiation codon (ATG) and a termination codon (TGA), which are underscored above. The region between and inclusive of the initiation codon and the termination codon is a methionine-initiated coding sequence of about 912 nucleotides, including the termination codon (nucleotides indicated as “coding” of SEQ ID NO:28; SEQ ID NO:30). The coding sequence encodes a 303 amino acid protein (SEQ ID NO:29), which is recited as follows:

(SEQ ID NO:29)

MAAVAVLRAFGASGPMCLRRGPWAQLPARFCSRDPAGAGRRESEPRPTSA
RQLDGIRNIVLSNPKKRNTLSLAMLKSLQSDLLHDADSNDLKVIIISAEG
PVFSSGHDLKIELTEEQGRDYHAEVFQTCSKVMMHIRNHPVPVIAMVNGL
ATAAGCQLVASCNIAVASDKSSFATPGVNVGLFCSTPGVALARAVPRKVA
LEMLFTGEPISAQIEALLHGLLSKVVPEAELQEETMRIARKIASLSRPVV
SLGKATFYKQLPQDLGTAYYLTSQAMVDNLALRDGQEGITAFLQKRKIPV
WSHEPV

### Example 13

### Tissue Distribution of 50090 mRNA by Large-Scale Tissue-Specific Library Sequencing and by Northern Blot Hybridization

This Example describes the tissue distribution of 50090 mRNA.

Northern blot hybridizations with various RNA samples can be performed under standard conditions and washed under stringent conditions, i.e., 0.2× SSC at 65° C. A DNA probe corresponding to all or a portion of the 50090 cDNA (SEQ ID NO:28) can be used. The DNA can be radioactively labeled with ³²P-dCTP using the Prime-It Kit (Stratagene, La Jolla, Calif.) according to the instructions of the supplier. Filters containing mRNA from mouse hematopoietic and endocrine tissues, and cancer cell lines (Clontech, Palo Alto, Calif.) can be probed in ExpressHyb hybridization solution (Clontech) and washed at high stringency according to manufacturer's recommendations.

### Example 14

### Recombinant Expression of 50090 in Bacterial Cells

In this example, 50090 is expressed as a recombinant glutathione-S-transferase (GST) fusion polypeptide in E. coli and the fusion polypeptide is isolated and characterized. Specifically, 50090 is fused to GST and this fusion polypeptide is expressed in E. coli, e.g., strain PEB199. Expression of the GST-50090 fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 15

### Expression of Recombinant 50090 Protein in COS Cells

To express the 50090 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire 50090 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the 50090 DNA sequence is amplified by PCR using two primers. The 5′ primer contains the restriction site of interest followed by approximately twenty nucleotides of the 50090 coding sequence starting from the initiation codon; the 3′ end sequence contains complementary sequences to the other restriction site of interest, a translation stop codon, the HA tag or FLAG tag and the last 20 nucleotides of the 50090 coding sequence. The PCR amplified fragment and the pcDNA/Amp vector are digested with the appropriate restriction enzymes and the vector is dephosphorylated using the CIAP enzyme (New England Biolabs, Beverly, Mass.). Preferably the two restriction sites chosen are different so that the 50090 gene is inserted in the correct orientation. The ligation mixture is transformed into E. coli cells (strains HB 101, DH5a, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the 50090-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the 50090 polypeptide is detected by radiolabelling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the 50090 coding sequence is cloned directly into the polylinker of the pcDNA/Amp vector using the appropriate restriction sites. The resulting plasmid is transfected into COS cells in the manner described above, and the expression of the 50090 polypeptide is detected by radiolabelling and immunoprecipitation using a 50090 specific monoclonal antibody.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. An isolated nucleic acid molecule selected from the group consisting of:
a) a nucleic acid comprising the nucleotide sequence of SEQ ID NO: 1, 3, 4, 6, 7, 9, 13, 15, 16, 18, 22, 24, 28, or 30; and
b) a nucleic acid molecule which encodes a polypeptide comprising the amino acid sequence of SEQ ID NO:2, 5, 8, 14, 17, 23, or 29.

2. The nucleic acid molecule of claim 1, further comprising vector nucleic acid sequences.

3. The nucleic acid molecule of claim 1; further comprising nucleic acid sequences encoding a heterologous polypeptide.

4. A host cell which contains the nucleic acid molecule of claim 1.

5. An isolated polypeptide comprising the amino acid sequence of SEQ ID NO:2, 5, 8, 14, 17, 23, or 29.

6. The polypeptide of claim 5 further comprising heterologous amino acid sequences.

7. An antibody or antigen-binding fragment thereof that selectively binds to a polypeptide of claim 5.

8. A method for producing a polypeptide comprising the amino acid sequence of SEQ ID NO:2, 5, 8, 14, 17, 23, or 29, the method comprising culturing the host cell of claim 4 under conditions in which the nucleic acid molecule is expressed.

9. A method for detecting the presence of a polypeptide of claim 5 in a sample, comprising:
a) contacting the sample with a compound which selectively binds to the polypeptide; and
b) determining whether the compound binds to the polypeptide in the sample.

10. The method of claim 9, wherein the compound which binds to the polypeptide is an antibody.

11. A kit comprising a compound which selectively binds to a polypeptide of claim 5 and instructions for use.

12. A method for detecting the presence of a nucleic acid molecule of claim 1 in a sample, comprising the steps of:
a) contacting the sample with a nucleic acid probe or primer which selectively hybridizes to the nucleic acid molecule; and
b) determining whether the nucleic acid probe or primer binds to a nucleic acid molecule in the sample.

13. The method of claim 12, wherein the sample comprises mRNA molecules and is contacted with a nucleic acid probe.

14. A kit comprising a compound which selectively hybridizes to a nucleic acid molecule of claim 1 and instructions for use.

15. A method for identifying a compound which binds to a polypeptide of claim 5 comprising the steps of:
a) contacting a polypeptide, or a cell expressing a polypeptide of claim 5 with a test compound; and
b) determining whether the polypeptide binds to the test compound.

16. A method for modulating the activity of a polypeptide of claim 5, comprising contacting a polypeptide or a cell expressing a polypeptide of claim 5 with a compound which binds to the polypeptide in a sufficient concentration to modulate the activity of the polypeptide.

17. A method of inhibiting aberrant activity of a 33312, 33303, 32579, 21509, 33770, 46638, or 50090-expressing cell, comprising contacting a 33312, 33303, 32579, 21509, 33770, 46638, or 50090-expressing cell with a compound that modulates the activity or expression of a polypeptide of claim 5, in an amount which is effective to reduce or inhibit the aberrant activity of the cell.

18. The method of claim 17, wherein the compound is selected from the group consisting of a peptide, a phosphopeptide, a small organic molecule, and an antibody.

19. A method of treating or preventing a disorder characterized by aberrant activity of a 33312, 33303, 32579, 21509, 33770, 46638, or 50090-expressing cell, in a subject, comprising:
administering to the subject an effective amount of a compound that modulates the activity or expression of a nucleic acid molecule of claim 1, such that the aberrant activity of the 33312, 33303, 32579, 21509, 33770, 46638, or 50090-expressing cell is reduced or inhibited.
